(19) United States
(12) Patent Application Publication
(10) Pub. No.: US 2004/0156854 A1
(43) Pub. Date: Aug. 12, 2004

(54) Methods for the identification, assessment, and treatment of patients with proteasome inhibition therapy

(75) Inventors: George Mulligan, Lexington, MA (US); Barbara M. Bryant, Cambridge, MA (US); Michael P. Morrissey, Brighton, MA (US); Andrew Bolt, Somerville, MA (US); Andrew I. Damokosh, West Hartford, CT (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/728,055

(22) Filed: Dec. 4, 2003

Related U.S. Application Data
(60) Provisional application No. 60/431,514, filed on Dec. 6, 2002.

(51) Int. Cl. C12Q 1/68; G01N 33/574; A61K 39/395

(52) U.S. Cl. 424/155.1; 435/6

(57) ABSTRACT

The present invention is directed to the identification of markers that can be used to determine whether patients with cancer are clinically responsive or non-responsive to a therapeutic regimen prior to treatment. In particular, the present invention is directed to the use of certain combinations of markers, wherein the expression of the markers correlates with responsiveness or non-responsiveness to a therapeutic regimen comprising proteasome inhibition. Thus, by examining the expression levels of individual markers and those comprising a marker set, it is possible to determine whether a therapeutic agent, or combination of agents, will be most likely to reduce the growth rate of tumors in a clinical setting.

### CROSS-REFERENCES TO RELATED APPLICATIONS

This application claims the benefit of U.S. Provisional Application No. 60/431,514, filed Dec. 6, 2002, the contents of which are incorporated herein by this reference.

### BACKGROUND OF THE INVENTION

Proteasome inhibition represents an important recently developed strategy in cancer treatment. The proteasome is a multi-enzyme complex present in all cells which plays a role in degradation of proteins involved in regulation of the cell cycle. For example, King et al., demonstrated that the ubiquitin-proteasome pathway plays an essential role in regulating cell cycle, neoplastic growth and metastasis. A number of key regulatory proteins, including p53, cyclins, and the cyclin-dependent kinases p21 and p27^{KIP1}, are temporally degraded during the cell cycle by the ubiquitin-proteasome pathway. The ordered degradation of these proteins is required for the cell to progress through the cell cycle and to undergo mitosis. See, e.g., Science 274:1652-1659 (1996). Furthermore, the ubiquitin-proteasome pathway is required for transcriptional regulation. Palombella et al., teach that the activation of the transcription factor NF-kB is regulated by proteasome-mediated degradation of the inhibitor protein IkB. See International Patent Application Publication No. WO 95/25533. In turn, NF-kB plays a central role in the regulation of genes involved in the immune and inflammatory responses. For example, Read et al. demonstrated that the ubiquitin-proteasome pathway is required for expression of cell adhesion molecules, such as E-selectin, ICAM-1, and VCAM-1. See Immunity 2:493-506 (1995). Additional findings further support the role for proteasome inhibition in cancer therapy, as Zetter found that cell adhesion molecules are involved in tumor metastasis and angiogenesis in vivo, by directing the adhesion and extravastation of tumor cells to and from the vasculature to distant tissue sites within the body. See, e.g., Seminars in Cancer Biology 4:219-229 (1993). Moreover, Beg and Baltimore, found that NF-kB is an anti-apoptotic factor, and inhibition of NF-kB activation makes cells more sensitive to environmental stress and cytotoxic agents. See Science 274:782 (1996).

Adams et al. have described peptide boronic ester and acid compounds useful as proteasome inhibitors. See, e.g., U.S. Pat. No. 5,780,454 (1998), U.S. Pat. No. 6,066,730 (2000), and U.S. Pat. No. 6,083,903 (2000). They describe the use of the disclosed boronic ester and boronic acid compounds to reduce the rate of muscle protein degradation, to reduce the activity of NF-kB in a cell, to reduce the rate of degradation of p53 protein in a cell, to inhibit cyclin degradation in a cell, to inhibit the growth of a cancer cell, and to inhibit NF-kB dependent cell adhesion. Adams et al. have described one of the compounds, N-pyrazinecarbonyl-L-phenylalanine-L-leucineboronic acid (PS-341, now know as bortezomib) as having demonstrated antitumor activity in human tumor xenograft models. This particular compound has recently received approval for treatment of patients having relapsed refractory multiple myeloma, and is presently undergoing clinical trials in additional indications, including additional hematological cancers as well as solid tumors.

Because the proteasome plays a pervasive role in normal physiology as well as pathology, it is important to optimize (e.g., avoid excessive) proteasome inhibition when using proteasome inhibitors as therapeutic agents. Moreover, one of the continued problems with therapy in cancer patients is individual differences in response to therapies. With the narrow therapeutic index and the toxic potential of many available cancer therapies, this potentially contributes to many patients undergoing unnecessary ineffective and even harmful therapy regimens. If a designed therapy could be optimized to treat individual patients, such situations could be reduced or even eliminated. Accordingly, there is a need to identify particular cancer patients against which proteasome inhibitors are particularly effective, either alone or in combination with other chemotherapies. Also, there is a need to identify particular patients who respond well to treatment with a proteasome inhibitor (responders) versus those patient who do not respond to proteasome treatment (non-responders). It would therefore be beneficial to provide for the diagnosis, staging, prognosis, and monitoring of cancer patients, including, e.g., hematological cancer patients (e.g., multiple myeloma, leukemias, lymphoma, etc) as well as solid tumor cancer patients, who would benefit from proteasome inhibition therapies; or to indicate a predisposition of such patients to such preventative measures. The present invention is directed towards these needs.

### DESCRIPTION OF THE INVENTION

The present invention is directed to the methods of identifying or selecting a cancer patient who is responsive to a therapeutic regimen comprising proteasome inhibition therapy. Additionally provided are methods of identifying a patient who is non-responsive to such a therapeutic regimen. These methods typically include the determining the level of expression of one or more predictive markers in a patient's tumor (e.g., a patient's cancer cells), and identifying whether expression in the sample includes a pattern or profile of expression of a selected predictive marker or marker set which correlates with response or non-response to proteasome inhibition therapy.

Additionally provided methods include therapeutic methods which further include the step of beginning, continuing, or commencing, or stopping, discontinuing or halting a proteasome inhibition therapy accordingly where a patient's predictive marker profile indicates that the patient would respond or not respond to the therapeutic regimen. In another embodiment, methods are provided for analysis of a patient not yet being treated with a proteasome inhibition therapy and identification and prediction that the patient would not be a responder to the therapeutic agent and such patient should not be treated with the proteasome inhibition therapy when the patient's marker profile indicates that the patient is a non-responder. Thus, the provided methods of the invention can eliminate ineffective or inappropriate use of proteasome inhibition therapy regimens.

The present invention is also directed to methods of treating a cancer patient, with a proteasome inhibition regimen, (e.g., a proteasome inhibitor agent, alone, or in combination with an additional agent such as a chemotherapeutic agent) which includes the step of selecting a patient whose predictive marker profile indicates that the patient will respond to the therapeutic agent, and treating the patient with the proteasome inhibition therapy regimen.

The present methods and compositions are designed for use in diagnostics and therapeutics for a patient suffering from cancer. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkins lymphoma). Solid tumors can originate in organs, and include cancers such as lung, breast, prostate, ovary, colon, kidney, and liver.

Therapeutic agents for use in the methods of the invention include a new class of therapeutic agents known as proteosome inhibitors. One example of a proteosome inhibitor that was recently approved for treatment of relapsed refractory multiple myeloma patients and is presently being tested in clinical trials for additional indications is bortezomib. Other examples of proteosome inhibitors are known in the art and are described in further detail herein. Proteasome inhibition therapy regimens can also include additional therapeutic agents such as chemotherapeutic agents. Some examples of traditional chemotherapeutic agents are set forth in Table A. Alternatively or in combination with these chemotherapeutic agents, newer classes of chemotherapeutic agents can also be used in proteasome inhibition therapy.

One embodiment of the invention provides methods for determining a proteasome inhibition-based regimen for treating a tumor in a patient. Such methods comprise measuring the level of expression of at least one predictive marker in the patient's tumor and determining a proteasome inhibition based regimen for treating the tumor based on the expression level of the predictive marker or markers, as relevant. A significant expression level of predictive marker or markers in the patient sample can be an indication that the patient is a responsive patient and would benefit from proteasome inhibition therapy when the predictive marker or marker set provided herein indicate such responsiveness. Additionally, a significant expression level of a predictive marker or markers in a patient can be an indication that the patient is a non-responsive patient and would not benefit from proteasome inhibition therapy when the marker or markers provided herein indicate such non-responsiveness.

The invention further provides methods for determining whether a patient will be responsive to a proteasome inhibition-based regimen for treating a tumor. Such methods comprise measuring the level of expression of at least one predictive marker in the patient's tumor and determining a proteasome inhibition based regimen for treating the tumor based on the expression level of the predictive marker or marker set. A significant expression level of a predictive marker in the patient sample is an indication that the patient is a responsive patient and would benefit from proteasome inhibition therapy. A significant expression level of a predictive marker set in the patient is an indication that the patient is a responsive patient and would benefit from proteasome inhibition therapy when the marker or markers provided herein indicate such responsiveness. Selected predictive markers for use in the methods comprise responsive predictive markers as indicated in Table 1, Table 2, and Table 3.

Still further, the invention further provides methods for determining whether a patient will be non-responsive to a proteasome inhibition-based regimen for treating a tumor. Such methods comprise measuring the level of expression of at least one predictive marker in the patient's tumor and determining a proteasome inhibition based regimen for treating the tumor based on the expression level of the predictive marker or marker set. A significant expression level of a predictive marker in the patient sample is an indication that the patient is a non-responsive patient and would benefit from proteasome inhibition therapy. A significant expression level of a predictive marker set in the patient is an indication that the patient is a non-responsive patient and would not benefit from proteasome inhibition therapy when the selected marker or marker set provided herein indicate such non-responsiveness. Selected predictive markers for use in the methods comprise non-responsive predictive markers as indicated in Table 1 Table 2 and Table 3.

Another embodiment of the invention provides methods for treating a tumor in a patient with proteasome inhibition therapy. Such therapeutic methods comprise measuring the level of expression of at least one predictive marker in a patient's tumor; determining whether a proteasome inhibition based regimen for treating the tumor is appropriate based on the expression level of the predictive marker or markers, and treating a patient with a proteasome inhibition therapy when the patient's expression level indicates a responsive patient. A significant expression level of predictive marker in the patient sample is an indication that the patient is a responsive patient and would benefit from proteasome inhibition therapy when the predictive marker or marker set provided herein indicate the patient is a responsive patient.

In certain aspects, the level of expression of predictive marker in the patient's tumor can be measured by isolating a sample of the tumor and performing analysis on the isolated sample, or a portion thereof. In another aspect, the level of expression of predictive marker in the patient's tumor can be measured using in vivo imaging techniques.

In certain aspects, determining the level of expression comprises detection of mRNA. Such detection can be carried out by any relevant method, including e.g., PCR, northern, nucleotide array detection, in vivo imaging using nucleic acid probes. In other aspects, determining the level of expression of the predictive marker comprises detection of protein. Such detection can be carried out using any relevant method for protein detection, including w.g., ELISA, western blot, immunoassay, protein array detection, in vivo imaging using peptide probes.

Determining the level of expression of a predictive marker can be compared to a predetermined standard control level of expression in order to evaluate if expression of a marker or marker set is significant and make an assessment for determining whether the patient is responsive or non-responsive. Additionally, determining the level of expression of a predictive marker can be compared to an internal control marker level of expression which is measured at the same time as the predictive marker in order to make an assessment for determining whether the patient is responsive or non-responsive. The level of expression may be determined as significantly over-expressed in certain aspects. The level of expression may be under-expressed in other aspects. In still other aspects, the level of expression is determined against a pre-determined standard as determined by the methods provided herein.

Methods of the invention can use at least one of the predictive markers set forth in any one of Table 1, Table 2, Table 3, Table 4, Table 5, Table 6, or Table 7. Additionally, the methods provided can use two, three, four, five, six, or more markers to form a predictive marker set. For example, marker sets selected from the markers in Table 1, Table 2 and/or Table 3 can be generated using the methods provided herein and can comprise between two, and all of the markers set forth in Table 1, Table 2 or Table 3 and each and every combination in between (e.g., four selected markers, 16 selected markers, 74 selected markers, etc.). In one embodiment, the markers comprise those set forth in Table 4, Table 5 or Table 6.

Methods of the invention further provide the ability to construct marker sets from the individual predictive markers set forth in Table 1 Table 2 and Table 3 using the methods described in further detail herein. In a further aspect, more than one marker set can be used in combination for the diagnostic, prognostic and treatment methods provided.

The methods of the invention can be performed such that determination of the level of expression of a predictive marker is measured prior to tumor therapy in order to identify whether the patient will be responsive to a proteasome inhibition therapy.

In addition, the methods of the invention can be performed concurrently with ongoing tumor therapy to determine if the patient is either responding to present proteasome inhibition therapy or will respond to additional therapy comprising proteasome inhibition therapy.

Still further, the methods of the invention can be performed after tumor therapy has been carried out in order to assess whether the patient will be responsive to future course of proteasome inhibition therapy.

Whether the methods are performed during ongoing tumor therapy or after a course of tumor therapy, the tumor therapy can comprise proteasome inhibition therapy or alternative forms of cancer therapy. The methods provided are designed to determine if the patient will benefit from additional or future proteasome inhibition therapy, and can include such proteasome inhibition therapy alone or in combination with additional therapeutic agents.

The invention also relates to various reagents and kits for diagnosing, staging, prognosing, monitoring and treating a cancer patient.

Provided are marker sets and methods for identification of marker sets comprising at least two isolated predictive markers set forth in Table 1, Table 2 and Table 3. The marker sets comprise reagents for detection of the relevant predictive markers set forth in Table 1, Table 2 and Table 3. Such reagents include nucleic acid probes, primers, antibodies, antibody derivatives, antibody fragments, and peptide probes.

Further provided are kits for use in determining a proteasome inhibition based regimen for treating a tumor in a patient. The kits of the invention include reagents for assessing predictive markers (e.g., at least one predictive marker) and predictive marker sets (e.g., at least two, three, four or more markers selected from Table 1, Table 2 and Table 3), as well as instructions for use in accordance with the methods provided herein. In certain aspects, the kits provided contain nucleic acid probes for assessment of predictive markers. In still other aspects, the kits provided contain antibody, antibody derivative antibody fragment, or peptide reagents for assessment of predictive markers.

According to the invention, the markers and marker sets are selected such that the positive predictive value of the methods of the invention is at least about 10%, preferably about 25%, more preferably about 50% and most preferably about 75%, 80%, 85%, or 90% or greater. Also preferred for use in the methods of the invention are markers that are differentially expressed in tumors, as compared to normal cells, by at least one-and-a-half-fold and preferably at least two-fold in at least about 20%, more preferably about 50%, and most preferably about 75% or more of any of the following conditions: partial responders, complete responders, minimal responders, and non-responders to proteasome inhibition therapy.

The present invention further provides previously unknown or unrecognized targets for the development of anti-cancer agents, e.g., chemotherapeutic compounds. The predictive markers and marker sets provided by the present invention also provide new targets either alone or in combination, which can be used for the development of novel therapeutics for cancers. Thus, nucleic acids and proteins represented by each of the markers provided can be used as targets in developing treatments (either single agent or multiple agent) for cancers, including e.g, hematological malignancies or solid tumor malignancies.

Thus, additionally provided are methods for use of the identified predictive markers, as well as the corresponding nucleic acid and polypeptides for screening methods for identification of novel compounds for use as anti-cancer therapeutics. Such newly identified compounds can be useful alone, or in combination with proteasome inhibition therapy as a complementary therapeutic.

The present invention is based, in part, on the identification of individual markers and marker sets that can be used to determine whether a tumor may be effectively treated by treatment with a proteasome inhibition therapy. For example, the compositions and methods provided herein can be used to determine whether a patient will be responsive or non-responsive to a proteasome inhibition therapeutic agent. Based on these identifications, the present invention provides, without limitation: 1) methods and compositions for determining whether a proteasome inhibition therapy will or will not be effective in stopping or slowing tumor growth; 2) methods and compositions for monitoring the effectiveness of a proteasome inhibition therapy (a proteasome inhibitor agent or a combination of agents) used for the treatment of tumors; 3) methods and compositions for identifying combinations of therapeutic agents for use in treating tumors; 4) methods and compositions for identifying specific therapeutic agents and combinations of therapeutic agents that are effective for the treatment of tumors in specific patients; 5) methods and compositions for identifying new targets for therapeutic agents for the treatment of tumors; and 6) methods and compositions for identifying new therapeutic agents for the treatment of tumors.

Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. The content of all GenBank or RefSeq database records cited throughout this application (including the Tables) are also hereby incorporated by reference. In the case of conflict, the present specification, including definitions, will control.

The articles “a” and “an” are used herein to refer to one or to more than one (i.e. to at least one) of the grammatical object of the article. By way of example, “an element” means at least one element and can include more than one element.

A “marker” is a naturally-occurring polymer corresponding to at least one of the nucleic acids or proteins associated with Affymetrix probe set identifiers listed in any one of Table 1, Table 2 or Table 3 For example, markers include, without limitation, sense and anti-sense strands of genomic DNA (i.e. including any introns occurring therein), RNA generated by transcription of genomic DNA (i.e. prior to splicing), RNA generated by splicing of RNA transcribed from genomic DNA, and proteins generated by translation of spliced RNA (i.e. including proteins both before and after cleavage of normally cleaved regions such as transmembrane signal sequences). As used herein, “marker” may also include a cDNA made by reverse transcription of an RNA generated by transcription of genomic DNA (including spliced RNA). “marker set” is a group of markers. Markers of the present invention include the predictive markers identified in Table 1, Table 2, and Table 3.

A “Predictive Marker” or “predictive marker” as used herein, includes a marker which has been identified as having differential expression in tumor cells of a patient and is representative of a characteristic of a patient which is responsive in either a positive or negative manner to treatment with a proteasome inhibitor regimen. For example, a predictive marker includes a marker which is upregulated in a non-responsive patient; alternatively a predictive marker includes a marker which is upregulated in a responsive patient. Similarly, a predictive marker is intended to include those markers which are down-regulated in a non-responsive patient as well as those markers which are down-regulated in a responsive patient. Thus, as used herein, predictive marker is intended to include each and every one of these possibilities, and further can include each one individually as a predictive marker; or alternatively can include one or more, or all of the characteristics collectively when reference is made to “predictive markers” or “predictive marker sets.”

As used herein, a “naturally-occurring” nucleic acid molecule refers to an RNA or DNA molecule having a nucleotide sequence that occurs in nature (e.g. encodes a natural protein).

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

The “normal” level of expression of a marker is the level of expression of the marker in cells in a similar environment or response situation, in a patient not afflicted with cancer. A normal level of expression of a marker may also refer to the level of expression of a “control sample”, (e.g., sample from a healthy subjects not having the marker associated disease). A control sample may be comprised of a control database. Alternatively, a “normal” level of expression of a marker is the level of expression of the marker in non-tumor cells in a similar environment or response situation from the same patient that the tumor is derived from.

“Over-expression” and “under-expression” of a marker refer to expression of the marker of a patient at a greater or lesser level, respectively, than normal level of expression of the marker (e.g. more than one and a half-fold, at least two-fold, at least three-fold, greater or lesser level etc.).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A marker is “fixed” to a substrate if it is covalently or non-covalently associated with the substrate such the substrate can be rinsed with a fluid (e.g. standard saline citrate, pH 7.4) without a substantial fraction of the marker dissociating from the substrate.

As used herein, “significant” expression, or a marker “significantly” expressed is intended to refer to differential expression of a predictive marker which is indicative of responsiveness or non-responsiveness. A marker or marker set in a patient is “significantly” expressed at a higher (or lower) level than the normal level of expression of a marker or marker set if the level of expression of the marker or marker set is greater or less, respectively, than the normal level by an amount greater than the standard error of the assay employed to assess expression. Preferably a significant expression level is at least twice, and more preferably three, four, five or ten times that amount. Alternately, expression of the marker or marker set in the patient can be considered “significantly” higher or lower than the normal level of expression if the level of expression is at least about two, and preferably at least about three, four, or five times, higher or lower, respectively, than the normal level of expression of the marker or marker set. Still further, a “significant” expression level may refer to level which either meets or is above or below a pre-determined score for a predictive marker set as determined by methods provided herein.

A cancer or tumor is treated or diagnosed according to the present methods. “Cancer” or “tumor” is intended to include any neoplastic growth in a patient, including an inititial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkins lymphoma,). Solid tumors can originate in organs, and include cancers such as lung, breast, prostate, ovary, colon, kidney, and liver. As used herein, cancer cells, including tumor cells, refer to cells that divide at an abnormal (increased) rate. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large Bcell lymphoma, malignant lymphoma, plasmocytoma, reticulum cell sarcoma, or Hodgkins disease); and tumors of the nervous system including glioma, meningoma, medulloblastoma, schwannoma or epidymoma.

A cancer is “responsive” to a therapeutic agent if its rate of growth is inhibited as a result of contact with the therapeutic agent, compared to its growth in the absence of contact with the therapeutic agent. Growth of a cancer can be measured in a variety of ways, for instance, the size of a tumor or the expression of tumor markers appropriate for that tumor type may be measured. For example, the response definitions used to identify markers associated with myeloma and its response to proteasome inhibition therapy, the Southwestern Oncology Group (SWOG) criteria as described in Blade et al., Br J Haematol. September, 1998;102(5):1115-23 were used (also see e.g., Table C). The quality of being responsive to a proteasome inhibition therapy is a variable one, with different cancers exhibiting different levels of “responsiveness” to a given therapeutic agent, under different conditions. Still further, measures of responsiveness can be assessed using additional criteria beyond growth size of a tumor, including patient quality of life, degree of metastases, etc. In addition, clinical prognostic markers and variables can be assessed (e.g., M protein in myeloma, PSA levels in prostate cancer) in applicable situations.

A cancer is “non-responsive” to a therapeutic agent if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured. For example, the response definitions used to identify markers associated with non-response of multiple myeloma to therapeutic agents, the Southwestern Oncology Group (SWOG) criteria as described in Blade et. al. were used in the experiments described herein. The quality of being non-responsive to a therapeutic agent is a highly variable one, with different cancers exhibiting different levels of “non-responsiveness” to a given therapeutic agent, under different conditions. Still further, measures of non-responsiveness can be assessed using additional criteria beyond growth size of a tumor, including patient quality of life, degree of metastases, etc. In addition, clinical prognostic markers and variables can be assessed (e.g., M protein in myeloma, PSA levels in prostate cancer) in applicable situations.

“Treatment” shall mean preventing or inhibiting further tumor growth, as well as causing shrinkage of a tumor. Treatment is also intended to include prevention of metastasis of tumor. A tumor is “inhibited” or “treated” if at least one symptom (as determined by responsiveness/non-responsiveness indicators known in the art and described herein) of the cancer or tumor is alleviated, terminated, slowed, minimized, or prevented. Any amelioration of any symptom, physical or otherwise, of a tumor pursuant to treatment using any proteasome inhibitor, is within the scope of the invention.

As used herein, the term “agent” is defined broadly as anything that cancer cells, including tumor cells, may be exposed to in a therapeutic protocol. In the context of the present invention, such agents include, but are not limited to, proteasome inhibition agents, as well as chemotherapeutic agents as described in further detail herein.

“Proteasome inhibitor” shall mean any substance which directly or indirectly inhibits the 20S or 26S proteasome or the activity thereof. Preferably, such inhibition is specific, i.e., the proteasome inhibitor inhibits proteasome activity at a concentration that is lower than the concentration of the inhibitor required to produce another, unrelated biological effect. Preferably, the concentration of the proteasome inhibitor required for proteasome inhibition is at least 2-fold lower, more preferably at least 5-fold lower, even more preferably at least 10-fold lower, and most preferably at least 20-fold lower than the concentration required to produce an unrelated biological effect. Proteasome inhibitors include peptide aldehydes, peptide boronic acids, lactacystin and lactacystin analogues, vinyl sulfones, and alpha.‘.beta.’-epoxyketones. Proteasome inhibitors are described in further detail herein.

A kit is any article of manufacture (e.g. a package or container) comprising at least one reagent, e.g. a probe, for specifically detecting a marker or marker set of the invention. The article of manufacture may be promoted, distributed, or sold as a unit for performing the methods of the present invention. The reagents included in such a kit comprise probes/primers and/or antibodies for use in detecting responsive and non-predictive marker expression. In addition, the kits of the present invention may preferably contain instructions which describe a suitable detection assay. Such kits can be conveniently used, e.g., in clinical settings, to diagnose and evaluate patients exhibiting symptoms of cancer, in particular patients exhibiting the possible presence of an a cancer capable of treatment with proteasome inhibition therapy, including, e.g., hematological cancers e.g., myelomas (e.g., multiple myeloma), lymphomas (e.g., non-hodgkins lymphoma), leukemias, and solid tumors (e.g., lung, breast, ovarian, etc.).

The markers of the present invention, whose expression correlates with the response to an agent, are identified in Table 1, Table 2, Table 3, Table 4, Table 5, Table 6, and Table 7. By examining the expression of one or more of the identified markers or marker sets in a tumor, it is possible to determine which therapeutic agent or combination of agents will be most likely to reduce the growth rate of the cancer cells. By examining the expression of one or more of the identified markers or marker sets in a cancer, it is also possible to determine which therapeutic agent or combination of agents will be the least likely to reduce the growth rate of cancer cells. By examining the expression of one or more of the identified markers or marker sets, it is therefore possible to eliminate ineffective or inappropriate therapeutic agents It is also possible to identify new targets for anti-cancer agents by examining the expression of one or more markers or marker sets. Thus, in one embodiment, the tumor cells used in the methods of the present invention are from a bone marrow sample. Importantly, these determinations can be made on a patient by patient basis or on an agent by agent basis. Thus, one can determine whether or not a particular therapeutic treatment is likely to benefit a particular patient or group/class of patients, or whether a particular treatment should be continued.

Table 1 lists markers identified using statistical analysis applied to genes from 44 myeloma patient samples. The markers in Table 1 are significantly expressed in samples from patients that are either responsive or non-responsive to treatment with the proteasome inhibitor bortezomib. Thus, one would appreciate that the markers identified can function in a predictive model to prospectively identify patients' response to proteasome inhibition therapy, including response to bortezomib or other proteasome inhibition therapies known in the art as well as those described in further detail herein. In particular, the markers in Table 1 are correlated with a positive response to therapy (referred to herein as “non-predictive markers, (NR)”). A patient with a positive response (either complete, partial or minimal; see Table C) to therapy is hereinafter referred to as a “responder”. Additionally, the predictive markers in Table 1 are correlated with a negative or poor response to an agent (referred to herein as “non-predictive markers, (NR)”). A patient with a poor response (called a progressive or refractory disease; see Table C) to treatment is hereinafter referred to as a “non-responder”. A patient with no response to treatment is hereinafter referred to as “stable” (see Table C).

Table 2 lists markers identified using statistical analysis applied using a Cox proportional hazard analysis to determine predictors of time until disease progression (TTP) in patients with relapsed and refractory multiple myeloma. These markers are useful as additional predictive markers which are significantly expressed in patients who are likely to progress in disease at a faster rate, and less likely to be responsive to therapy than other patients. These predictive markers will serve as an additional factor in identification of patients likely to be responsive to proteasome inhibition therapy.

Table 3 lists markers identified using statistical analysis applied to genes from 44 myeloma samples. The predictive markers in Table 2 are significantly expressed in samples from myeloma patients whose disease is refractory to treatment with the proteasome inhibitor bortezomib. These predictive markers will further serve to distinguish refractory patients from those who will be either stable or responsive to treatment.

The invention also relates to various reagents and kits for diagnosing, staging, prognosing, monitoring and treating a cancer patient, (e.g., a patient with a liquid tumor or a solid tumor as described in further detail herein), with proteasome inhibition therapy.

According to the invention, the markers are selected such that the positive predictive value of the methods of the invention is at least about 10%, preferably about 25%, more preferably about 50% and most preferably about 90%. Also preferred for use in the methods of the invention are markers that are differentially expressed, as compared to normal cells, by at least two-fold in at least about 20%, more preferably about 50%, and most preferably about 75% of any of the following conditions: responsive patients (e.g., complete response, partial response, minimal response); and non-responsive patients (e.g., no change, relapse from response).

Identification of Responsive and Non-Predictive Markers

The present invention provides markers that are expressed in a tumor that is responsive to proteasome inhibition therapy and whose expression correlates with responsiveness to that therapeutic agent. The present invention also provides markers that are expressed in a tumor that is non-responsive to proteasome inhibition therapy and whose expression correlates with non-responsiveness to such therapy. Accordingly, one or more of the markers can be used to identify cancers that can be successfully treated by proteasome inhibition therapy. In one embodiment, one or more of the markers of the present invention can be used to identify patients that can be successfully treated using proteasome inhibition therapy. In addition, the markers of the present invention can be used to identify a patient that has become or is at risk of becoming refractory to treatment with proteasome inhibition therapy. The invention also features combinations of markers, referred to herein as “marker sets,” that can predict patients that are likely to respond or not to respond to a proteasome inhibition therapy regimen.

Table 1 identifies markers whose expression correlates with responsiveness to a proteasome inhibitor. It is preferable to determine the expression of at least one, two or more of the identified predictive markers; or three or more of the identified predictive markers comprising a set of the identified predictive markers. Thus, it is preferable to assess the expression of a set or panel of predictive markers, i.e., the expression profile of a predictive marker set.

Determining Responsiveness or Non-Responsiveness to an Agent

The expression level (including protein level) of the identified responsive and non-predictive markers may be used to: 1) determine if a patient can be treated by an agent or combination of agents; 2) determine if a patient is responding to treatment with an agent or combination of agents; 3) select an appropriate agent or combination of agents for treating a patient; 4) monitor the effectiveness of an ongoing treatment; 5) identify new proteasome inhibition therapy treatments (either single agent proteasome inhibitor agents or complementary agents which can be used alternatively or in combination with proteasome inhibition agents); 6) differentiate early versus late recurrence of a cancer; and 7) select an appropriate agent or combination of agents in treating early and late recurrence of a cancer. In particular, the identified responsive and non-predictive markers may be utilized to determine appropriate therapy, to monitor clinical therapy and human trials of a drug being tested for efficacy, and to develop new agents and therapeutic combinations.

In one embodiment of the invention, a cancer may be predisposed to respond to an agent if one or more of the corresponding predictive markers identified in Table 1, Table 2 and Table 3 are significantly expressed. In another embodiment of the invention, the predisposition of a cancer to be responsive to an agent is determined by the methods of the present invention, wherein significant expression of the individual predictive markers of the marker sets identified in Table 4, Table 5, or Table 6 is evaluated. Likewise, the predisposition of a patient to be responsive to an agent is determined by the methods of the present invention, wherein a marker set generated using to the methods described herein wherein the markers comprising the marker set include predictive markers set forth in Table 1, Table 2, and/or Table 3, and the expression of the marker set is evaluated.

In another embodiment of the invention, a cancer may be predisposed to non-responsiveness to an agent if one or more of the corresponding non-predictive markers are significantly expressed. In another embodiment of the invention, a cancer may be predisposed to non-responsiveness to an agent if one or more of the corresponding predictive markers identified in Table 1, Table 2 and Table 3 are significantly expressed. In another embodiment of the invention, the predisposition of a cancer to be non-responsive to an agent is determined by the methods of the present invention, wherein significant expression of the individual predictive markers of the marker sets identified in Table 4, Table 5, or Table 6 is evaluated. Likewise, the predisposition of a patient to be non-responsive to an agent is determined by the methods of the present invention, wherein a marker set is generated using the methods described herein wherein the markers comprising the marker set include predictive markers set forth in Table 1, Table 2, and/or Table 3, and the expression of the marker set is evaluated.

The present invention provides methods for determining whether a proteasome inhibition therapy e.g., a proteasome inhibitor agent, can be used to reduce the growth rate of a tumor comprising the steps of:

(a) evaluating expression of at least one individual predictive marker in a tumor sample; and

(b) identifying that proteasome inhibition therapy is or is not appropriate to reduce the growth rate of the tumor based on the evaluation.

In another embodiment, the invention provides a method for determining whether an proteasome inhibition therapeutic regimen (e.g., a proteasome inhibitor agent (e.g., bortezomib) alone or in combination with another chemotherapeutic agent) can be used to reduce the growth rate of a tumor comprising the steps of:

(a) determining the expression profile of a predictive marker or predictive marker set; and

(b) identifying that a proteasome inhibition therapeutic agent is or is not appropriate to reduce the growth rate of the myeloma cells based on the expression profile.

In one aspect, the predictive marker or markers evaluated are selected from those set forth in Table 1. In yet another aspect the predictive marker or markers evaluated are selected from those set forth in Table 2. In still another aspect the predictive marker or markers evaluated are selected from those set forth in Table 3. Still a further aspect contemplates markers set forth in either Table 1 alone or in combination with markers set for the in Table 2 and/or Table 3, or alternatively, those markers set forth in Table 2 alone or in combination with Table 1 and/or Table 3.

In another embodiment, the invention provides a method for determining whether a proteasome inhibitor therapy can be used to reduce the growth of a tumor, comprising the steps of:

(a) obtaining a sample of tumor cells;

(b) evaluating the expression of one or more individual markers of a marker set, both in tumor cells exposed to the agent and in tumor cells that have not been exposed to the proteasome inhibition therapy; and

(c) identifying that an agent is or is not appropriate to treat the tumor based on the evaluation.

In such methods, a proteasome inhibition therapy regimen is determined appropriate to treat the tumor when the expression profile of the marker set demonstrates increased responsiveness or decreased non-responsiveness according to the expression profile of the predictive markers in the presence of the agent

In a preferred embodiment, the predictive markers are selected from those set forth in Table 1, Table 2 or Table 3.

In another embodiment, the invention provides a method for determining whether treatment with an anti-cancer agent should be continued in an multiple myeloma patient, comprising the steps of:

(a) obtaining two or more samples of tumor cells from a patient at different times during the course of an proteasome inhibition therapy treatment;

(b) evaluating the expression of the individual markers of a marker set, in the two or more samples; and

(c) continuing or discontinuing the treatment based on the evaluation.

In a preferred embodiment, the marker set is selected from those set forth in Table 1 or Table 2 or Table 3. According to the methods, proteasome inhibition therapy would be continued where the expression profile indicates continued responsiveness, or decreased non-responsiveness using the evaluation methods described herein.

In another embodiment, the invention provides a method for determining whether treatment with a proteasome inhibition therapy regimen should be continued in an myeloma patient, comprising the steps of:

(a) obtaining two or more samples of myeloma cells from a patient at different times during the course of anti-cancer agent treatment;

(b) determining the expression profile a predictive marker set, in the two or more samples; and

(c) continuing the treatment when the expression profile of the predictive marker set does not demonstrate decreased responsiveness and/or does not demonstrate increased non-responsive during the course of treatment.

Alternatively, in step (c), the treatment is discontinued when the expression profile of the marker set demonstrates decreased responsiveness and/or increased non-responsiveness during the course of treatment. In a preferred embodiment, the marker set is selected from those set forth in Table 1, Table 2 or Table 3.

The present invention further provides methods for determining whether an agent, e.g., a chemotherapeutic agent, can be used to reduce the growth rate of multiple myeloma comprising the steps of:

(a) obtaining a sample of cancer cells;

In another embodiment, the invention provides a method for determining whether treatment with an anti-cancer agent should be continued in an multiple myeloma patient, comprising the steps of:

obtaining two or more samples of myeloma cells from a patient at different times during the course of anti-cancer agent treatment;

determining the level of expression in the myeloma cells of one or more genes which correspond to markers identified in any of Table 1, Table 2 or Table 3 in the two or more samples; and

continuing the treatment is continued when the expression profile of the predictive markers identified in any one of Table 1, Table 2, and Table 3 is indicative of a responsive patient during the course of treatment.

Alternatively, in step (c), the treatment is discontinued when the expression profile of the predictive markers identified in any one of Table 1, Table 2 and Table 3 is indicative of a non-responsive patient during the course of treatment

In another embodiment, the invention provides a method for determining whether treatment with bortezomib should be continued in an multiple myeloma patient, comprising the steps of:

obtaining two or more samples of myeloma cells from a patient at different times during the course of treatment with bortezomib;

determining the expression profile in the myeloma cells of one or more genes which correspond to markers identified in Table 1 Table 2 or Table 3 in the two or more samples; and

continuing the treatment when the expression profile of the predictive markers identified in Table 1 Table 2 or Table 3 is indicative of a responsive patient. Alternatively, the treatment is discontinued when the expression profile of the predictive markers identified in Table 1 Table 2 and/or Table 3 is indicative of a non-responsive patient during the course of treatment

The markers and marker sets of the present invention are predictive of proteasome inhibition therapy regimens, generally. Proteasome inhibition therapy, generally comprises at least an agent which inhibition proteasome activity in a cell, and can comprise additional therapeutic agents. In one embodiment of the invention, the agent used in methods of the invention is a proteasome inhibitor. In certain aspects, the proteasome inhibitor is bortezomib, or other related proteasome inhibitor agents as described in further detail herein. Still other aspects, the proteasome inhibition therapy comprises a proteasome inhibitor agent in conjunction with a chemotherapeutic agent. Chemotherapeutic agents are known in the art and described in further detail herein.

In another embodiment of the invention, the expression of predictive marker or markers identified in Table 1, Table 2, and Table 3 is detected by measuring mRNA which corresponds to the predictive marker. In yet another embodiment of the invention, the expression of markers which correspond to markers or marker sets identified in Table 1 Table 2 and Table 3, is detected by measuring protein which corresponds to the marker.

In another embodiment, the invention provides a method of treating a patient with cancer by administering to the patient a compound which has been identified as being effective against a cancer by the methods of the invention described herein.

The source of the cancer cells used in the present method will be based on how the method of the present invention is being used. For example, if the method is being used to determine whether a patient's cancer can be treated with an agent, or a combination of agents, then the preferred source of cancer cells will be cancer cells obtained from a tumor from the patient, e.g., a tumor biopsy (including a solid or a liquid tumor), a blood sample. Alternatively, a cancer cell line similar to the type of cancer being treated can be assayed. For example if multiple myeloma is being treated, then a myeloma cell line can be used. If the method is being used to predict or monitor the effectiveness of a therapeutic protocol, then a tissue or blood sample from the patient being treated is the preferred source. If the method is being used to identify new therapeutic agents or combinations, any cancer cells, e.g., cells of a cancer cell line, can be used.

A skilled artisan can readily select and obtain the appropriate cancer cells that are used in the present method. For cancer cell lines, sources such as The National Cancer Institute, for the NCI-60 cells, are preferred. For cancer cells obtained from a patient, standard biopsy methods, such as a needle biopsy, can be employed.

Myeloma samples were used to identify the markers of the present invention. Further, the expression level of markers can be evaluated in other tissue types including disorders of related hematological cell types, including, e.g., Waldenstroms macrogobulinemia, Myelodysplastic syndrome and other hematological cancers including lymphomas, leukemias, as well as tumors of various solid tissues. It will thus be appreciated that cells from other hematologic malignancies including, e.g., B-cell Lymphomas, Non-Hodgkins Lymphoma, Waldenstrom's syndrome, or other leukemias will be useful in the methods of the present invention. Still further, the predictive markers predicting disease aggressiveness as well as responsiveness and non-responsiveness to proteasome inhibition therapeutic agents in solid tumors (e.g., lung, breast, prostate, ovary, colon, kidney, and liver), can also be useful in the methods of the present invention.

In the methods of the present invention, the level of expression of one or more predictive markers selected from the group consisting of the markers identified in Table 1 Table 2 and Table 3, is determined. As used herein, the level or amount of expression refers to the absolute level of expression of an mRNA encoded by the marker or the absolute level of expression of the protein encoded by the marker (i.e., whether or not expression is or is not occurring in the cancer cells).

Generally, it is preferable to determine the expression of two or more of the identified responsive or non-predictive markers, or three or more of the identified responsive or non-predictive markers, or still further a larger a set of the identified responsive and/or non-predictive markers, selected from the predictive markers identified in Table 1, Table 2 and Table 3. For example, Table 4, Table 5 and Table 6 set forth marker sets identified using the methods described herein and can be used in the methods of the present invention. Still further, additional and/or alternative marker sets comprising the predictive markers identified herein can be generated using the methods and predictive markers provided. Thus, it is possible to assess the expression of a panel of responsive and non-predictive markers using the methods and compositions provided herein.

As an alternative to making determinations based on the absolute expression level of selected markers, determinations may be based on normalized expression levels. Expression levels are normalized by correcting the absolute expression level of a responsive or non-predictive marker by comparing its expression to the expression of a control marker that is not a responsive or non-predictive marker, e.g., a housekeeping gene that is constitutively expressed. Suitable markers for normalization include housekeeping genes, such as the actin gene. Constitutively expressed genes are known in the art and can be identified and selected according to the relevant tissue and/or situation of the patient and the analysis methods. Such normalization allows one to compare the expression level in one sample, e.g., a tumor sample, to another sample, e.g., a non-tumor sample, or between samples from different sources.

Further, the expression level can be provided as a relative expression level. To determine a relative expression level of a marker or marker set, the level of expression of the predictive marker or marker set is determined for 10 or more individual samples, preferably 50 or more individual samples in order to establish a baseline, prior to the determination of the expression level for the sample in question. To establish a baseline measurement, mean expression level of each of the predictive markers or marker sets assayed in the larger number of samples is determined and this is used as a baseline expression level for the predictive markers or marker sets in question. The expression level of the marker or marker set determined for the test sample (absolute level of expression) is then divided by the mean expression value obtained for that marker or marker set. This provides a relative expression level and aids in identifying extreme cases of responsive or non-responsive-ness.

Preferably, the samples used will be from similar tumors or from non-cancerous cells of the same tissue origin as the tumor in question. The choice of the cell source is dependent on the use of the relative expression level data. For example, using tumors of similar types for obtaining a mean expression score allows for the identification of extreme cases of responsive or non-responsive-ness. Using expression found in normal tissues as a mean expression score aids in validating whether the responsive/non-predictive marker or marker set assayed is tumor specific (versus normal cells). Such a later use is particularly important in identifying whether a responsive or non-predictive marker or marker set can serve as a target marker or marker set. In addition, as more data is accumulated, the mean expression value can be revised, providing improved relative expression values based on accumulated data.

Still further, as outlined above, there are various methods available to examine the expression of the markers, including gene array/chip technology, RT-PCR, in-situ hybridization, immunohistochemistry, immunoblotting, FISH (flouresence in-situ hybridization), FACS analyses, northern blot, southern blot or cytogenetic analyses. A skilled artisan can select from these or other appropriate and available methods based on the nature of the marker(s), tissue sample and disease in question. Different methods or combinations of methods could be appropriate in different cases or, for instance in different solid or hematological tumor types.

Detection Assays

An exemplary method for detecting the presence or absence of a polypeptide or nucleic acid corresponding to a marker of the invention in a biological sample involves obtaining a biological sample (e.g. a tumor sample) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations. in situ hybridizations, and TaqMan assays (Applied Biosystems) under GLP approved laboratory conditions. In vitro techniques for detection of a polypeptide corresponding to a marker of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay. One example of such an embodiment includes use of an array or chip which contains a predictive marker or marker set anchored for expression analysis of the sample.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl Oct. 10, 1997;699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction and TaqMan analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cancer cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a control gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the markers and marker sets assayed in the larger number of samples is determined and this is used as a baseline expression level for the marker. The expression level of the marker determined for the test sample (absolute level of expression) is then divided by the mean expression value obtained for that marker. This provides a relative expression level.

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether cancer cells express a marker of the present invention.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker of the invention in a biological sample (e.g. an ovary-associated body fluid such as a urine sample). Such kits can be used to determine if a subject is suffering from or is at increased risk of developing cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention; (2) a pair of primers useful for amplifying a nucleic acid molecule corresponding to a marker of the invention; or (3) a marker set comprising oligonucleotides which hybridize to at least two nucleic acid sequences encoding polypeptide predictive markers of the invention. The kit can also comprise, e.g., a buffering agent, a preservative, or a protein stabilizing agent. The kit can further comprise components necessary for detecting the detectable label (e.g., an enzyme or a substrate). For marker sets, the kit can comprise a marker set array or chip for use in detecting the predictive markers. The kit can also contain a control sample or a series of control samples which can be assayed and compared to the test sample. Each component of the kit can be enclosed within an individual container and all of the various containers can be within a single package, along with instructions for interpreting the results of the assays performed using the kit.

Monitoring the Effectiveness of an Anti-Cancer Agent

As discussed above, the identified responsive and non-predictive markers can be used as pharmacodynamic markers to assess whether the tumor has become refractory to an ongoing treatment (e.g., a proteasome inhibition therapy). When the cancer is not responding to a treatment the expression profile of the tumor cells will change: the level or relative expression of one or more of the predictive markers (e.g., those predictive markers identified in Table 1, Table 2, Table 3) such that the expression profile represents a non-responsive patient.

In one such use, the invention provides methods for determining whether a proteasome inhibition treatment should be continued in a cancer patient, comprising the steps of:

determining the expression of at least one predictive marker of a marker set, wherein the markers are selected from those set forth in any of Table 1, Table 2 or Table 3, in a tumor sample of a patient exposed to a proteasome inhibition therapy; and

continuing treatment when the expression profile of the marker or marker set demonstrates responsiveness to the agent being used.

In another such use, the invention provides methods for determining whether a proteasome inhibition therapy should be discontinued in a cancer patient, comprising the steps of:

determining the expression of at least one predictive marker of a marker set, wherein the markers are selected from those set forth in any of Table 1, Table 2 or Table 3 in a tumor sample of a patient expose to a proteasome inhibition therapy; and

discontinuing or altering treatment when the expression profile of the markers identified in any one of Table 1 Table 2 or Table 3 demonstrates non-responsiveness to the agent being used.

As used herein, a patient refers to any subject undergoing proteasome inhibition therapy for cancer treatment. In one embodiment, the subject will be a human patient undergoing proteasome inhibition using a sole proteasome inhibition agent (e.g., bortezomib or other related agent). In another embodiment, the subject is a human patient undergoing proteasome inhibition using a proteasome inhibition agent in conjunction with another agent (e.g., a chemotherapy treatment). This embodiment of the present invention can also include comparing two or more samples obtained from a patient undergoing anti-cancer treatment including proteasome inhibition therapy. In general, it is conceivable to obtain a first sample from the patient prior to beginning therapy and one or more samples during treatment. In such a use, a baseline of expression prior to therapy is determined, then changes in the baseline state of expression is monitored during the course of therapy. Alternatively, two or more successive samples obtained during treatment can be used without the need of a pre-treatment baseline sample. In such a use, the first sample obtained from the subject is used as a baseline for determining whether the expression of a particular marker or marker set is increasing or decreasing.

In general, when monitoring the effectiveness of a therapeutic treatment, two or more samples from a patient are examined. In another aspect, three or more successively obtained samples are used, including at least one pretreatment sample.

Electronic Apparatus Readable Arrays

Electronic apparatus readable arrays comprising at least one predictive marker orof the present invention is also provided. As used herein, “electronic apparatus readable media” refers to any suitable medium for storing, holding or containing data or information that can be read and accessed directly by an electronic apparatus. As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems. As used herein, “recorded” refers to a process for storing or encoding information on the electronic apparatus readable medium. Those skilled in the art can readily adopt any of the presently known methods for recording information on known media to generate manufactures comprising the markers of the present invention.

The array can be used to assay expression of one or more predictive markers or predictive marker sets in the array. In one embodiment, the array can be used to assay predictive marker or marker set expression in a tissue to ascertain tissue specificity of markers in the array. In this manner, up to about 44,000 markers can be simultaneously assayed for expression. This allows a profile to be developed showing a battery of markers specifically expressed in one or more tissues.

The array is also useful for ascertaining differential expression patterns of one or more markers in normal and abnormal (e.g., tumor) cells. This provides a battery of predictive markers that could serve as a tool for ease of identification of responsive and non-responsive patients.

In addition to such qualitative determination, the invention allows the quantitation of marker expression. Thus, predictive markers can be grouped on the basis of marker sets or responsive and non-responsive indications by the level of expression in the sample. This is useful, for example, in ascertaining the responsive or non-responsive indication of the sample by virtue of scoring the expression levels according to the methods provided herein.

In another embodiment, the array can be used to monitor the time course of expression of one or more predictive markers in the array.

The array is also useful for ascertaining the effect of the expression of a marker on the expression of other predictive markers in the same cell or in different cells. This provides, for example, a selection of alternate molecular targets for therapeutic intervention if the proteasome inhibition regimen is non-responsive.

Therapeutic Agents

The markers of the present invention are shown to be predictive of patients who are responsive or non-responsive (sensitive or resistant) to proteasome inhibition therapy. Proteasome inhibition therapy can comprise treatment of a cancer patient with a proteasome inhibitor agent, alone or in combination with additional agents, such as chemotherapeutic agents.

The examples described herein entail use of the proteasome inhibitor N-pyrazinecarbonyl-L-phenylalanine-L-leucineboronic acid, bortezomib ((VELCADE™); formerly known as MLN341 or PS-341). The language “proteasome inhibitor” is intended to include bortezomib, compounds which are structurally similar to bortezomib and/or analogs of bortezomib. The language “proteasome inhibitor” can also include “mimics”. “Mimics” is intended to include compounds which may not be structurally similar to bortezomib but mimic the therapeutic activity of bortezomib or structurally similar compounds in vivo. Proteasome inhibitor compounds of this invention are those compounds which are useful for inhibiting tumor growth, (e.g., multiple myeloma tumor growth, other hematological or solid tumors as described in further detail herein) in patients. Proteasome inhibitor also is intended to include pharmaceutically acceptable salts of the compounds.

Proteasome inhibitors for use in the practice of the invention include additional peptide boronic acids such as those disclosed in Adams et al., U.S. Pat. No. 5,780,454 (1998), U.S. Pat. No. 6,066,730 (2000), U.S. Pat. No. 6,083,903 (2000), U.S. Pat. No. 6,548,668 (2003), and Siman et al. WO 91/13904, each of which is hereby incorporated by reference in its entirety, including all compounds and formulae disclosed therein. Preferably, a boronic acid compound for use in the present invention is selected from the group consisting of: N-(4-morpholine)carbonyl-.beta.-(1-naphthyl)-L-alanine-L-leucine boronic acid; N-(8-quinoline)sulfonyl-.beta.-(1-naphthyl)-L-alanine-L-alanine-L-leucine boronic acid; N-(2-pyrazine)carbonyl-L-phenylalanine-L-leucine boronic acid, and N-(4-morpholine)carbonyl-[O-(2-pyridylmethyl)]-L-tyrosine-L-leucine boronic acid.

Additionally, proteasome inhibitors include peptide aldehyde proteasome inhibitors such as those disclosed in Stein et al. U.S. Pat. No. 5,693,617 (1997), and International patent publications WO 95/24914 published Sep. 21, 1995 and Siman et al. WO 91/13904 published Sep. 19, 1991; Iqbal et al. J. Med. Chem. 38:2276-2277 (1995), as well as Bouget et al. Bioorg Med Chem 17:4881-4889 (2003) each of which is hereby incorporated by reference in its entirety, including all compounds and formulae disclosed therein.

Further, proteasome inhibitors include lactacystin and lactacycstin analogs which have been disclosed in Fentany et al, U.S. Pat. No. 5,756,764 (1998), and U.S. Pat. No. 6,147,223(2000), Schreiber et al U.S. Pat. No. 6,645,999 (2003), and Fenteany et al. Proc. Natl. Acad. Sci. USA (1994) 91:3358, each of which is hereby incorporated by reference in its entirety, including all compounds and formulae disclosed therein.

Additionally, synthetic peptide vinyl sulfone proteasome inhibitors and epoxyketone proteasome inhibitors have been disclosed and are useful in the methods of the invention. See, e.g., Bogyo et al., Proc. Natl. Acad. Sci. 94:6629 (1997); Spaltensteinet al. Tetrahedron Lett. 37:1343 (1996); Meng L, Proc. Natl. Acad Sci 96: 10403 (1999); and Meng L H, Cancer Res 59: 2798 (1999), each of which is hereby incorporated by reference in its entirety.

Still further, natural compounds have been recently shown to have proteasome inhibition activity can be used in the present methods. For example, TMC-95A, a cyclic peptide, or Gliotoxin, both fungal metabolites or polyphenols compounds found in green tea have been identified as proteasome inhibitors. See, e.g., Koguchi Y, Antibiot (Tokyo) 53:105. (2000); Kroll M, Chem Biol 6:689 (1999); and Nam S, J. Biol Chem 276: 13322(2001), each of which is hereby incorporated by reference in its entirety.

Further to the above, the language, proteasome inhibition therapy can also include additional agents in addition to proteasome inhibition agents, including chemotherapeutic agents. A “chemotherapeutic agent” is intended to include chemical reagents which inhibit the growth of proliferating cells or tissues wherein the growth of such cells or tissues is undesirable. Chemotherapeutic agents such as anti-metabolic agents, e.g., Ara AC, 5-FU and methotrexate, antimitotic agents, e.g., taxane, vinblastine and vincristine, alkylating agents, e.g., melphanlan, BCNU and nitrogen mustard, Topoisomerase II inhibitors, e.g., VW-26, topotecan and Bleomycin, strand-breaking agents, e.g., doxorubicin and DHAD, cross-linking agents, e.g., cisplatin and CBDCA, radiation and ultraviolet light. In a preferred embodiment, the agent is a proteasome inhibitor (e.g., bortezomib or other related compounds).are well known in the art (see e.g., Gilman A. G., et al., The Pharmacological Basis of Therapeutics, 8th Ed., Sec 12:1202-1263 (1990)), and are typically used to treat neoplastic diseases. The chemotherapeutic agents generally employed in chemotherapy treatments are listed below in Table A.

TABLE A

| | | NONPROPRIETARY |
|---|---|---|
| | | NAMES |
| CLASS | TYPE OF AGENT | (OTHER NAMES) |
| Alkylating | Nitrogen Mustards | Mechlorethamine (HN₂) |
| | | Cyclophosphamide |
| | | Ifosfamide |
| | | Melphalan (L-sarcolysin) |
| | | Chlorambucil |
| | Ethylenimines | Hexamethylmelamine |
| | And Methylmelamines | Thiotepa |
| | Alkyl Sulfonates | Busulfan |
| Alkylating | Nitrosoureas | Carmustine (BCNU) |
| | | Lomustine (CCNU) |
| | | Semustine (methyl-CCNU) |
| | | Streptozocin (streptozotocin) |
| Alkylating | Triazenes | Decarbazine |
| | | (DTIC; |
| | | dimethyltriazenoimidazolecarboxamide) |
| | Alkylator | cis-diamminedichloroplatinum II (CDDP) |
| Antimetabolites | Folic Acid Analogs | Methotrexate (amethopterin) |
| | Pyrimidine | Fluorouracil (′5-fluorouracil; 5-FU) |
| | Analogs | Floxuridine (fluorode-oxyuridine; FUdR) |
| | | Cytarabine (cytosine arabinoside) |
| | Purine Analogs and | Mercaptopuine (6-mercaptopurine; 6-MP) |
| | Related | Thioguanine (6-thioguanine; TG) |
| | Inhibitors | Pentostatin (2′-deoxycoformycin) |
| Natural | Vinca Alkaloids | Vinblastin (VLB) |
| Products | | Vincristine |
| | Topoisomerase | Etoposide |
| | Inhibitors | Teniposide |
| | | Camptothecin |
| | | Topotecan |
| | | 9-amino-campotothecin CPT-11 |
| | Antibiotics | Dactinomycin (actinomycin D) |
| | | Adriamycin |
| | | Daunorubicin (daunomycin; |
| | | rubindomycin) |
| | | Doxorubicin |
| | | Bleomycin |
| | | Plicamycin (mithramycin) |
| | | Mitomycin (mitomycin C) |
| | | TAXOL |
| | | Taxotere |
| | Enzymes | L-Asparaginase |
| Natural Products | Biological Response | Interfon alfa |
| | Modifiers | Interleukin 2 |
| Miscellaneous | Platinum Coordination | cis-diamminedichloroplatinum II |
| Agents | Complexes | (CDDP) |
| | | Carboplatin |
| | Anthracendione | Mitoxantrone |
| | Substituted Urea | Hydroxyurea |
| | Methyl Hydraxzine | Procarbazine |
| | Derivative | (N-methylhydrazine, (MIH) |
| | Adrenocortical | Mitotane (o,p'-DDD) |
| | Suppressant | Aminoglutethimide |
| Hormones and | Adrenocorticosteroids | Prednisone |
| Antagonists | Progestins | Hydroxyprogesterone caproate |
| | | Medroxyprogesterone acetate |
| | | Megestrol acetate |
| | Estrogens | Diethylstilbestrol |
| | | Ethinyl estradiol |
| | Antiestrogen | Tamoxifen |
| | Androgens | Testosterone propionate |
| | | Fluoxymesterone |
| | Antiandrogen | Flutamide |
| | Gonadotropin-releasing | Leuprolide |
| | Hormone analog | |

The agents tested in the present methods can be a single agent or a combination of agents. For example, the present methods can be used to determine whether a single chemotherapeutic agent, such as methotrexate, can be used to treat a cancer or whether a combination of two or more agents can be used in combination with a proteasome inhibitor. Preferred combinations will include agents that have different mechanisms of action, e.g., the use of an anti-mitotic agent in combination with an alkylating agent and a proteasome inhibitor.

The agents disclosed herein may be administered by any route, including intradermally, subcutaneously, orally, intraarterially or intravenously. Preferably, administration will be by the intravenous route. Preferably parenteral administration may be provided in a bolus or by infusion.

The concentration of a disclosed compound in a pharmaceutically acceptable mixture will vary depending on several factors, including the dosage of the compound to be administered, the pharmacokinetic characteristics of the compound(s) employed, and the route of administration. Effective amounts of agents for treating ischemia or reperfusion injury would broadly range between about 10 μg and about 50 mg per Kg of body weight of a recipient mammal. The agent may be administered in a single dose or in repeat doses. Treatments may be administered daily or more frequently depending upon a number of factors, including the overall health of a patient, and the formulation and route of administration of the selected compound(s).

Isolated Nucleic Acid Molecules, Vectors and Host Cells

One aspect of the invention pertains to isolated nucleic acid molecules that correspond to a predictive marker of the invention, including nucleic acids which encode a polypeptide corresponding to a predictive marker of the invention or a portion of such a polypeptide. Isolated nucleic acids of the invention also include nucleic acid molecules sufficient for use as hybridization probes to identify nucleic acid molecules that correspond to a predictive marker of the invention, including nucleic acids which encode a polypeptide corresponding to a predictive marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule of the present invention, e.g., a nucleic acid encoding a protein corresponding to a marker listed in any one of Table 1, Table 2, and/or Table 3, can be isolated and manipulated (e.g., amplified, cloned, synthesized, etc.) using standard molecular biology techniques and the sequence information in the database records described herein. (e.g., described in Sambrook et al., ed., Molecular Cloning: A Laboratory Manual, 2nd ed., Cold Spring Harbor Laboratory Press, Cold Spring Harbor, N.Y., 1989).

Moreover, a nucleic acid molecule of the invention can comprise only a portion of a nucleic acid sequence, wherein the full length nucleic acid sequence comprises a predictive marker of the invention or which encodes a polypeptide corresponding to a marker of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more predictive markers of the invention. The probe comprises a label group attached thereto, e.g., a radioisotope, a fluorescent compound, an enzyme, or an enzyme co-factor. Such probes can be used as part of a diagnostic test kit for identifying cells or tissues which express the protein, such as by measuring levels of a nucleic acid molecule encoding the protein in a sample of cells from a subject, e.g., detecting mRNA levels or determining whether a gene encoding the protein has been mutated or deleted.

In addition to the nucleotide sequences described in the database records described herein, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the terms “gene” and “recombinant gene” refer to nucleic acid molecules comprising an open reading frame encoding a polypeptide corresponding to a marker of the invention, including, e.g., sequences which differ, due to degeneracy of the genetic code, from the nucleotide sequence of nucleic acids encoding a protein which corresponds to a marker of the invention, and thus encode the same protein.

As used herein, the phrase “allelic variant” refers to a nucleotide sequence which occurs at a given locus or to a polypeptide encoded by the nucleotide sequence. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another aspect, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a marker of the invention, such that the molecular beacon is useful for quantitating the presence of the predictive marker of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

Vectors, preferably expression vectors, containing a nucleic acid encoding a polypeptide corresponding to a predictive marker of the invention can be used for production of nucleic acid and proteins corresponding to predictive markers of the invention; as well as for production of compositions relating to the predictive markers. Useful vectors further comprise promoter and/or regulatory sequences for effective expression of the nucleic acid and/or protein corresponding to the predictive marker of interest. In certain instances, promoters can include constitutive promoter/regulatory sequences, inducible promoter/regulatory sequences, tissue specific promoter/regulatory sequences, or the natural endogenous promoter/regulatory sequences corresponding to the predictive marker of interest, as required. Various expression vectors are well known in the art and can be adapted to suit the particular system for expression. For example, recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

A host cell of the invention, such as a prokaryotic or eukaryotic host cell in culture, can be used to produce a polypeptide corresponding to a marker of the invention. Accordingly, the invention further provides methods for producing a polypeptide corresponding to a marker of the invention using the host cells of the invention. In one embodiment, the method comprises culturing the host cell of invention (into which a recombinant expression vector encoding a polypeptide of the invention has been introduced) in a suitable medium such that the marker is produced. In another embodiment, the method further comprises isolating the marker polypeptide from the medium or the host cell.

Isolated Proteins and Antibodies

One aspect of the invention pertains to isolated proteins which correspond to predictive markers of the invention, and biologically active portions thereof, as well as polypeptide fragments suitable for use as immunogens to raise antibodies directed against a polypeptide corresponding to a predictive marker of the invention. Polypeptides for use in the invention can be isolated, purified, or produced using the gene identification information provided herein in combination with routine molecular biology, protein purification and recombinant DNA techniques well known in the art.

Biologically active portions of a polypeptide corresponding to a marker of the invention include polypeptides comprising amino acid sequences sufficiently identical to or derived from the amino acid sequence of the protein corresponding to the predictive marker, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence listed in the one of the GenBank and NUC database records described herein. Other useful proteins are substantially identical (e.g., at least about 50%, preferably 70%, 80%, 90%, 95%, or 99%) to one of these sequences and retain the functional activity of the protein of the corresponding naturally-occurring protein yet differ in amino acid sequence due to natural allelic variation or mutagenesis.

The determination of percent identity between two sequences can be accomplished using a mathematical algorithm determining the number of identical positions shared between two sequences. Determination can be carried out using any known method in the art for comparison of identity and similarity. Examples of methods used can include for example, a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2. The percent identity between two sequences can be determined using techniques similar to those described above, with or without allowing gaps. In calculating percent identity, only exact matches are counted.

The invention also provides chimeric or fusion proteins corresponding to a marker of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention. Useful fusion proteins can include GST, c-myc, FLAG, HA, and any other well known heterologous tag for use in fusion protein production. Such fusion proteins can facilitate the purification of a recombinant polypeptide of the invention.

In addition, fusion proteins can include a signal sequence from another protein such as gp67, melittin, human placental alkaline phosphatase, and phoA. In yet another aspect, the fusion protein is an immunoglobulin fusion protein in which all or part of a polypeptide corresponding to a predictive marker of the invention is fused to sequences derived from a member of the immunoglobulin protein family. The immunoglobulin fusion proteins of the invention can be used as immunogens to produce antibodies directed against a polypeptide of the invention in a subject, to purify ligands and in screening assays to identify molecules which inhibit the interaction of receptors with ligands.

An isolated polypeptide corresponding to a predictive marker of the invention, or a fragment thereof, can be used as an immunogen to generate antibodies using standard techniques for polyclonal and monoclonal antibody preparation. For example, an immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies.

Polyclonal antibodies can be prepared as described above by immunizing a suitable subject with a polypeptide of the invention as an immunogen. Preferred polyclonal antibody compositions are ones that have been selected for antibodies directed against a predictive marker or markers of the invention. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction.

Alternatively, antibodies specific for a protein or polypeptide of the invention can be selected or (e.g., partially purified) or purified by, e.g., affinity chromatography to obtain substantially purified and purified antibody. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

Additionally, monoclonal antibodies directed to the predictive markers can be prepared for use in the methods of the present invention. Methods for generation of monoclonal antibodies are well known in the art and can be produced using any method. For example, at an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide corresponding to a predictive marker of the invention (e.g., a monoclonal antibody) can be used to detect the predictive marker (e.g., in a cellular sample) in order to evaluate the level and pattern of expression of the predictive marker. The antibodies can also be used diagnostically to monitor protein levels in tissues or body fluids (e.g. in an tumor sample) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified antibodies or fragments thereof, and non-human antibodies or fragments thereof, which antibodies or fragments specifically bind to a polypeptide comprising an amino acid sequence encoded by a predictive marker identified herein. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence which is encoded by a nucleic acid molecule of a predictive marker of the invention. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention.

The invention also provides a kit containing an antibody of the invention conjugated to a detectable substance, and instructions for use. Still another aspect of the invention is a diagnostic composition comprising an antibody of the invention and a pharmaceutically acceptable carrier. In preferred embodiments, the diagnostic composition contains an antibody of the invention, a detectable moiety, and a pharmaceutically acceptable carrier.

Screening Assays

The invention also provides methods (also referred to herein as “screening assays”) for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

Test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a marker or biologically active portion thereof. In another embodiment, the invention provides assays for screening candidate or test compounds which bind to a marker or biologically active portion thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker or a biologically active portion thereof. In all likelihood, the marker can, in vivo, interact with one or more molecules, such as but not limited to, peptides, proteins, hormones, cofactors and nucleic acids. For the purposes of this discussion, such cellular and extracellular molecules are referred to herein as “binding partners” or marker “substrate”. One necessary embodiment of the invention in order to facilitate such screening is the use of the marker to identify its natural in vivo binding partners. Many of the known binding partners or substrates of the identified predictive markers are either known in the art, or can be identified using standard methodologies known in the art (e.g., two hybrid screening, etc.).

In a further embodiment, assays may be devised through the use of the invention for the purpose of identifying compounds which modulate (e.g., affect either positively or negatively) interactions between a marker and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is an predictive marker identified herein, the known binding partner and/or substrate of same, and the test compound. Test compounds can be supplied from any source.

The basic principle of the assay systems used to identify compounds that interfere with the interaction between the marker and its binding partner involves preparing a reaction mixture containing the marker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker and its binding partner. Control reaction mixtures are incubated without the test compound or with a placebo. The formation of any complexes between the marker and its binding partner is then detected. The formation of a complex in the control reaction, but less or no such formation in the reaction mixture containing the test compound, indicates that the compound interferes with the interaction of the marker and its binding partner. Conversely, the formation of more complex in the presence of compound than in the control reaction indicates that the compound may enhance interaction of the marker and its binding partner.

The assay for compounds that interfere with the interaction of the marker with its binding partner may be conducted in a heterogeneous or homogeneous format. Heterogeneous assays involve anchoring either the marker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the markers and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques.

Other techniques for immobilizing proteins on matrices can also be used in the screening assays of the invention. For example, either a marker or a marker binding partner can be immobilized utilizing conjugation of biotin and streptavidin. Biotinylated marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci August, 1993;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker and its binding partner.

Also within the scope of the present invention are methods for direct detection of interactions between the marker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays.

In another embodiment, modulators of marker expression are identified in a method wherein a cell is contacted with a candidate compound and the expression of mRNA or protein, corresponding to a marker in the cell, is determined. The level of expression of mRNA or protein in the presence of the candidate compound is compared to the level of expression of mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein.

Still further, in cell based assays, where a cell expressing a predictive marker of interest is used for screening therapeutic candidate agents, the activity or viability of the cell is monitored to determine the ability of the test compound to alter the activity of the predictive marker or markers. Such assays are carried in tandem with a control assay utilizing similar or identical cell lines which do not express the predictive marker or markers of interest, in order to determine specificity of the action of the test compound.

In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an marker modulating agent, an antisense marker nucleic acid molecule, an marker-specific antibody, or an marker-binding partner) can be used in an animal model to determine the efficacy, toxicity, or side effects of treatment with such an agent. Alternatively, an agent identified as described herein can be used in an animal model to determine the mechanism of action of such an agent.

### SPECIFIC EXAMPLES

Treatment Dosage and Administration

Drug Supply and Storage

Bortezomib for injection (VELCADE™ Millennium Pharmaceuticals, Inc., Cambridge, Mass.), a sterile lyophilized powder for reconstitution, was supplied in vials containing 2.5 mg bortezomib and 25 mg mannitol USP. Each vial was reconstituted with 2.5 mL of normal (0.9%) saline, Sodium Chloride Injection USP, such that the reconstituted solution contained bortezomib at a concentration of 1 mg/mL. The reconstituted solution was clear and colorless with a final pH between 5 and 6. Vials containing lyophilized bortezomib for Injection were stored refrigerated at 2 to 8° C.

TABLE B
Drug Information

| | |
|---|---|
| Chemical Name | N-Pyrazinecarbonyl-L- |
| | phenylalanine-L-leucineboronic acid |
| Research Name | MLN341 or PS-341 |
| Generic Name | bortezomib |
| Proprietary Name | VELCADE ™ |
| CAS Registry Number | 179324-69-7 |
| U.S. Pat. No. | 5,780,454 |
| Classification | Proteasome Inhibitor |
| Molecular Formula | C₁₉H₂₅BN₄O₄ |
| Molecular Weight | 384.25 |
| Structure | Boronic acid derivative |
| | of a leucine phenylalanine dipeptide |

An Open-Label Phase II Study of Bortezomib in Patients with Myeloma Who Have Relapsed Following Front-Line Therapy and are Refractory to their Most Recent Therapy

Pharmacodynamic/Pharmacogenomic/Pharmacokinetic Data Collected

A multicenter, open-label, non-randomized Phase 2 trial was conducted, wherein enrolled were patients with relapsed myeloma that was refractory to therapy. Patients were treated with 1.3 mg of bortezomib per square meter of body surface area, twice weekly for two weeks, followed by one week without treatment, for up to eight cycles (24 weeks).

The following evaluations were conducted to assess the pharmacodynamics and pharmacogenomics of bortezomib.

Proteasome inhibition assay (blood for this ex vivo assay was collected before and one hour after dosing on Day 1 and Day 11 of Cycles 1, 7, and, if applicable, the cycle in which dexamethasone was started and one hour after dosing on Day 11 of Cycle 8). Some patients had an additional sample collected for the proteasome inhibition assay at 24 hours after dosing on Day 1, Cycle 1.

Pharmacogenomic data (blood and bone marrow samples for evaluation of the expression of global mRNA levels; these procedures were conducted only in patients who consented to participate via a separate consent form).

Population pharmacokinetics (blood for determination of population pharmacokinetics was collected from all patients before and one to six hours after study drug administration on Day 1, Cycle 1, and before and one to six hours after study drug administration on Day 11 of Cycles 1, 2, 7, and 8 and, if applicable, the cycle in which dexamethasone was started). Pre-dose blood samples were collected at the same time as those for clinical laboratory evaluations.

Individual pharmacokinetics: blood for determination of plasma bortezomib levels was collected immediately before and at 2, 5, 10, 15, 30, 60, and 120 minutes and 24 hours after bortezomib administration on Day 1, Cycle 1.

Statistical Procedures

Statistical analysis focused on the need to estimate response rates within specified limits of accuracy in order to determine if either of the two dose levels 1.0 or 1.3 mg/m²/dose alone or in combination with dexamethasone are sufficiently efficacious to warrant further clinical study. This study was noncomparative in nature; therefore efficacy comparisons between the two doses of bortezomib were not performed. In addition, this study provided safety data that helped to characterize the potential toxicity of treatment at the two evaluated dose levels for up to eight cycles of therapy.

Summary tabulations were presented that displayed the number of observations, mean, standard deviation, median, minimum, and maximum for continuous variables, and the number and percent per category for categorical data. The categories for summarization were the two assigned dose groups.

A formal statistical analysis plan was developed and finalized prior to database lock. The primary efficacy analyses were performed on the intent-to-treat (ITT) population. The primary efficacy analysis were performed on the rates of responders, where a responder was defined as a CR, PR, or MR using the criteria prospectively established in Table C. Two-sided 90% confidence limits on proportions of responders in each dose group were established, corresponding to a 95% one-sided lower limit.

TABLE C
Disease Response Criteria¹

| Response | Criteria for response |
|---|---|
| Complete response (CR)² | Requires all of the following: |
| | Disappearance of the original monoclonal protein from the blood and |
| | urine on at least two determinations for a minimum of six weeks by |
| | immunofixation studies. |
| | <5% plasma cells in the bone marrow on at least two determinations |
| | for a minimum of six weeks. |
| | No increase in the size or number of lytic bone lesions (development |
| | of a compression fracture does not exclude response). |
| | Disappearance of soft tissue plasmacytomas for at least six weeks. |
| Partial response (PR)³ | PR includes patients in whom some, but not all, criteria for CR are |
| | fulfilled providing the remaining criteria satisfy the requirements for |
| | PR. |
| | Requires all of the following: |
| | ≧50% reduction in the level of serum monoclonal protein for at least |
| | two determinations six weeks apart. |
| | If present, reduction in 24-hour urinary light chain excretion by either |
| | ≧90% or to <200 mg for at least two determinations six weeks apart. |
| | ≧50% reduction in the size of soft tissue plasmacytomas (by clinical |
| | or radiographic examination) for at least six weeks. |
| | No increase in size or number of lytic bone lesions (development of |
| | compression fracture does not exclude response). |
| Minimal response (MR) | MR includes patients in whom some, but not all, criteria for PR are |
| | fulfilled providing the remaining criteria satisfy the requirements for |
| | MR. |
| | Requires all of the following: |
| | ≧25% to ≦49% reduction in the level of serum monoclonal protein |
| | for at least two determinations six weeks apart. |
| | If present, a 50 to 89% reduction in 24-hour light chain excretion, |
| | which still exceeds 200 mg/24 h, for at least two determinations |
| | six weeks apart. |
| | For patients with non-secretory myeloma only, a 25 to 49% reduction |
| | in plasma cells in the bone marrow for a minimum of six weeks. |
| | 25-49% reduction in the size of plasmacytomas (by clinical or |
| | radiographic examination) for at least six weeks. |
| | No increase in size or number of lytic bone lesions (development of |
| | compression fracture does not exclude response). |
| No change (NC) | Not meeting the criteria for MR or PD. |
| Progressive disease (PD) | Requires one or more of the following: |
| (for patients not in CR) | >25% increase in the level of serum monoclonal paraprotein, which |
| | must also be an absolute increase of at least 5 g/L and confirmed on a |
| | repeat investigation. |
| | >25% increase in 24-hour urinary light chain excretion, which must |
| | also be an absolute increase of at least 200 mg/24 h and confirmed on |
| | a repeat investigation. |
| | >25% increase in plasma cells in a bone marrow aspirate or on |
| | trephine biopsy, which must also be an absolute increase of at least |
| | 10%. |
| | Definite increase in the size of existing lytic bone lesions or soft |
| | tissue plasmacytomas. |
| | Development of new bone lesions or soft tissue plasmacytomas (not |
| | including compression fracture). |
| | Development of hypercalcemia (corrected serum calcium |
| | >11.5 mg/dL or 2.8 mmol/L not attributable to any other cause). |
| Relapse from CR | Requires at least one of the following: |
| | Reappearance of serum or urinary paraprotein on immunofixation or |
| | routine electrophoresis confirmed by at least one follow-up and |
| | excluding oligoclonal immune reconstitution. |
| | ≧5% plasma cells in the bone marrow aspirate or biopsy. |
| | Development of new lytic bone lesions or soft tissue plasmacytomas |
| | or definite increase in the size of residual bone lesions (not including |
| | compression fracture). |
| | Development of hypercalcemia (corrected serum calcium |
| | >11.5 mg/dL or 2.8 mmol/L not attributable to any other cause). |

Based on the criteria reported by Kraut et al., J. Clin. Oncol. 16(2): 589-592 (1998) and Blade et al., Br. J. Haematol. 102(5): 1115-1123 (1998). In patients with CR, bone marrow was analyzed using PCR for verification of CR at the molecular level. Patients who met all criteria for PR but who exhibit a ≧75% reduction in the level of serum monoclonal protein for at least two determinations six weeks apart were termed in ‘Remission’ (R).

Quality of Life assessment was analyzed to determine if response to therapy was accompanied by measurable improvement in quality of life. Analysis was performed on summary scores as well as individual items, with specific analytical methods outlined in a formal statistical analysis plan developed prior to database lock.

Pharmacodynamic data (20S proteasome) were descriptively analyzed in order to characterize the degree of proteasome inhibition, and to investigate any correlation between degree of inhibition and therapeutic response and toxicity.

For those patients who participated in the pharmacogenomic portion of the study, correlation between RNA expression levels and response to therapy were evaluated descriptively. In addition, duration of response, time to disease progression, and overall patient survival may be analyzed using RNA expression as a factor.

A total of 202 patients were enrolled in the study. The overall response rate to PS-341 alone was 35% (CR+PR rate of 27%) prior to any patients receiving added dexamethasone for non-optimal response. These patients had all received at least two prior treatment regimens for their disease and their disease had progressed on their most recent therapy. This patient population has a very poor prognosis and no available standard therapy. Kamofsky Performance Status (KPS) was ≦70 in 25% of patients, and Durie-Salmon stage was reported as IIA or IIIB in 79% of patients. Approximately 39% of the patients had β₂ microglobulin ≧4 mg/L at Baseline, with 22% of patients having this indicator of disease severity ≧6 mg/L. The majority of the patients had relapsed after all conventional, high-dose, and novel therapies, with 74% progressing despite prior treatment with thalidomide.

The dose of 1.3 mg/m² twice weekly for two weeks followed by a 10-day rest was well tolerated. Over 80% of the 78 patients completed 2 or more cycles of treatment, 62% completed 4 or more cycles, and 27% completed 8 cycles.

The Independent Review Committee (IRC) evaluation of confirmed response to treatment with bortezomib alone is provided in Table D; further categorization of response for those patients who experienced partial remission is provided in Table E. This independent panel panel of three medical oncologists reviewed all data for 193 evaluable patients in the trial and assigned response using Blade criteria (Table C). The IRC determined that 35% of these 193 patients with relapsed/refractory multiple myeloma had a response to treatment (CR+PR+MR) with bortezomib alone, with 53 (27%) of the 193 patients experiencing a complete or partial remission to therapy and an additional 14 patients with a minimal response. An additional 46 (24%) of patients had evidence for stable disease (NC, no change) in response to bortezomib alone, which reflects an improvement in status for these patients who were progressing at the time of study entry. Based on the IRC assessment, 38 (20%) of the 193 patients had progressive disease and an additional 42 patients (22%) were considered not evaluable for response by the IRC. These data have been published. See Richardson PG, et al., New Eng. J. Med.; 348: 2609-17 (2003).

All pharmacogenomic analyses relied on the Independent Review Committee's judgement of response category.

TABLE D
Summary of IRC Confirmed Response to Treatment
with bortezomib Alone (N = 193)

| Confirmed Response Category | Response to bortezomib^{a} |
|---|---|
| Complete + Partial + Minor Responses | 67 (35%) |
| Complete + Partial Remissions | 53 (27%) |
| Complete + Near Complete Remissions (NCR) | 19 (10%) |
| Complete Remission (CR) | 19 (4%) |
| Partial Remission (PR) | 34 (23%) |
| Minor Response (MR) | 14 (5%) |
| No Change | 46 (27%) |
| Progressive Disease | 38 (20%) |
| Not Evaluable | 42 (22%) |

^{a}Response to treatment while patients were receiving bortezomib alone. (N = 193)

Identification of Responsive and Non-Predictive Markers

44 multiple myeloma patients had high quality gene expression data.

Candidate markers that are correlated with the outcome of multiple myeloma patients to a proteasome inhibition (e.g., bortezomib) therapy were selected by using a combination of marker ranking algorithms. Supervised learning and feature selection algorithms were then used to identify the markers of the present invention.

Data Analysis

A data set, comprised of 44 discovery samples, was classified as responders (N_{R}=17), stable disease (N_{S}=12), or progressive disease (N_{P}=15), based on the assignments of the IRC. For marker identification, the three response classes were further grouped into responders (N_{R}=17) vs non-responders (N_{NR}=27), or refractory/progressive disease (N_{P}=15) vs others (N=29). For each sample, 44,928 gene transcripts (Affymetrix probe sets) were profiled on the two Affymetrix U133 microarrays according to manufacturer's directions. Total RNA was isolated from homogenized tissue by Triazol™ (Life Technologies, Inc.) following the manufacturer's recommendations. RNA was stored at 80° C. in diethyl pyrocarbonate-treated deionized water. Detailed methods for labeling the samples and subsequent hybridization to the arrays are available from Affymetrix (Santa Clara, Calif.). Briefly, 5.0 μg of total RNA was converted to double-stranded cDNA (Superscript; Life Technologies, Inc.) priming the first-strand synthesis with a T7-(dT)24 primer containing a T7 polymerase promoter (Affymetrix Inc.). All of the double-stranded cDNA was subsequently used as a template to generate biotinylated cRNA using the incorporated T7 promoter sequence in an in vitro transcription system (Megascript kit; Ambion and Bio-11-CTP and Bio-16-UTP; Enzo). Control oligonucleotides and spikes were added to 10 μg of cRNA, which was then hybridized to U133 oligonucleotide arrays for 16 h at 45° C. with constant rotation. The arrays were then washed and stained on an Affymetrix fluidics station using the EUKGE-WS1 protocol and scanned on an Affymetrix GeneArray scanner.

Normalization and Logarithmic Transformation.

Expression values for all markers on each microarray were normalized to a trimmed mean of 150. Expression values were determined using MAS5 gene expression analysis data processing software (Affymetrix, Santa Clara, Calif.). These values will be referred to as the “normalized expression” in the remainder of this section. In a further processing step, each normalized expression value was divided by 150, and added to 1. The natural logarithm was taken of the resulting number, and this value will be referred to as the “log expression” in the remainder of this section.

Single Marker Selection.

Single gene transcripts that appear associated with sample classes can be identified using the feature ranking and filtering methodology described below. Single marker identification of Predictive Markers using the methodology described herein are set forth in Table 1 Table 2 and Table 3.

Model Selection.

A set of one or more gene transcripts that together classify samples into sensitive and resistant groups (or responsive and non-responsive), in the context if a particular classifier algorithm, is referred to as a “model.” The gene transcripts are referred to as “features.” Determining which combination of gene transcript(s) best classifies samples into sensitive and resistant groups is referred to as “model selection.” The following section describes the process of how the models of the present invention were identified. Exemplary models are set forth in Table 4, Table 5, and Table 6. The methods provided herein along with the single marker identification or Predictive markers can be used to identify. additional models comprising markers of the invention.

Summary of the Data Provided in the Tables

The following terms are used throughout the Tables:

“No.” or “Number” corresponds to an identification number for the markers.

“Probeset ID” corresponds to the Affymetrix (Santa Clara, Calif.) identifier from the Human Genome U133 set oligonucleotide arrays which were used;

“Sequence Derived from” or “Genbank” or “RefSeq” corresponds to the public database accession information for the markers.

“RefSeq” corresponds to the Reference Sequence Nucleic Accession Number;

“Genbank” corresponds to the GenBank accession number assigned to the particular sequence. All referenced GenBank sequences are expressly incorporated herein by reference;

“Title” corresponds to a common description, where available;

“Gene symbol” corresponds to a symbol the gene is commonly known by;

“Unigene” corresponds to the unique gene identifier;

“Rank ______” corresponds to the process of determining which individual markers may be used in combination to group or classify a sample, for example, as responsive(R) or non-responsive(NR). Rank and the relative scoring method used for various ranking is indicated, as is the lowest rank score identified among all the methods for each of the predictive markers. Four different feature selection methods were utilized for determining the best classifier: (1) Signal-to-Noise Ratio (“SNR”), (2) Class-Based Threshold (“CBT”), (3) Pooled Fold Change (“PFC”), and (4) the Wilcoxon Rank-Sum Test;

Additional titles correspond to scored and parameters used in each of the methods described in the following exemplification, including “Hazard,” “Decision Boundary,” “Weight,” “Vote Weight,” “Vote,” “Confidence,” “Expression,” “Gene Expression,” “Log Gene Expression,” “Normalized Expression,” and “Normalization Factor;”

“Supplemental Annotation” and “Biological Category” correspond to additional characterization and categorization not set forth in the title;

For Table 8, cell lines were designated as Sensitive “S” or Resistant “R;” and “Ratio of Sensitive/Resistant” indicates relative expression of marker indicated.

Feature Ranking and Filtering

The first step in model selection is to filter the 44,928 features down to a smaller number which show a correspondence with the sample classifications. Filtering involves first ranking the features by a scoring method, and then taking only the highest ranking features for further analysis. The filtering algorithms used in the present invention were: (1) Signal-to-Noise Ratio (“SNR”), (2) Class-Based Threshold (“CBT”), (3) Pooled Fold Change (“PFC”), and (4) the Wilcoxon Rank-Sum Test. In preferred embodiments, SNR was used to identify genes showing a small but consistent change in levels, and CBT was used to identify genes that were “off” in one class, but “on” in a fraction of the other class.

SNR is computed from the log expression values as absolute value of the difference in class means divided by the sum of the class standard deviations, and has been used to analyze expression data before; for example, see the definition of P(g,c), a measure of correlation between expression of gene g and class vector c, in Golub et al., “Molecular Classification of Cancer: Class discovery and class prediction by marker expression monitoring,” Science, 286:531-537 (1999), the contents of which are incorporated herein by reference. To use SNR for filtering, the features with the top 100 SNR scores were retained and the remainder discarded from consideration.

CBT is computed from the normalized expression values, and defines one class (“class A”) as the “off” class, and the other class (“class B”) as the “on” class. In the present studies, the “off” class, class A is Responders; and the “on” class, class B, is Non-Responders. The CBT score may be computed in one of two ways: (1) Threshold each class B value to the average class A expression value for that feature. CBT is the difference between the average thresholded class B expression and the average class A expression, divided by the standard deviation of the class A expression:

$$CBT = \frac{\frac{1}{{N}_{B}} [ {\sum }_{i = 1}^{{N}_{B}} max ( {x}_{i} , {μ}_{A} ) ] - {μ}_{A}}{{σ}_{A}}$$

where μ_{A} is the average class A expression value, σ_{A} is the standard deviation of the class A expression values, and xᵢ represent the N_{B} individual class B expression values. (2) CBT is the percentage of class B samples which exceed a fixed multiple of the maximum (or other percentile value) of expression values in class A. In either method, a constant value may be added to the class A threshold value to compensate for noise. In preferred embodiments, method 1 was utilized, and the top 100 features were selected.

The Pooled Fold Change (“PFC”) method is a measure of differential expression between two groups of samples, arbitrarily designated “control” and “tester.” PFC finds genes with higher expression in the tester than in the control samples. The analysis was performed looking at both Responders as “tester” (PFC-R) and Non-Responders as “tester” (PFC-NR). To qualify as having higher expression, tester samples must be above the k^{th} percentile control sample. The fold-change values of tester samples are subjected to a nonlinear transformation that rises to a user-specified asymptote, in order to distinguish moderate levels of fold-change, but not make distinctions between very large fold-changes. The squashed fold-change values of the over-expressed tester samples are averaged to get the POOF score. In particular, PFC for gene g is computed as the average across tester samples of the compressed tester:control ratio R(s,g). For a given tester sample s and gene g, R(s,g)=C(x_{gs}/(k+x_{g}^{Q})), where

C(x) is the compression function C(z)=A(1−e^{-z/A}) for z≧T, and C(z)=0 for z<T, where T is a threshold value no less than 1.0.

A is an upper asymptote on the fold-change value (we used 5),

k is a constant reflecting the additive noise in the data, i.e., the fixed component of the variance in repeated measurements. We derived a value of 30 for this parameter from calibration experiments.

x_{gs} is the expression value of gene g in sample s,

x_{g}^{Q} is the Qth percentile of the control samples' expression value.

Also, a minimum fraction f of the tester samples must have R(s,g) greater than 0; if this does not hold true, then the value of R(s,g) is set to 0.

We used the following parameters in two runs of this algorithm:

| Parameter | Value in run 1 | Value in run 2 |
|---|---|---|
| Q | 1.0 | 0.8 |
| f | 0.2 | 0.4 |
| T | 1.25 | 1.25 |

The Wilcoxon Rank-Sum test is a standard statistical technique. See, for example, Conover, W. J. 1980. Practical Nonparametric Statistics. 2nd ed. New York: John Wiley & Sons, which is incorporated herein by reference. This test is also known as the Mann-Whitney U test. The goal is to test the null hypothesis that the population distributions corresponding to two random samples are identical against the alternative hypothesis that they are different. Only the rank of the samples' expression values is examined, not the values themselves.

Markers using the 44,928 probe sets were analyzed for differential expression across the 44 patient samples using the methods described in the above. In particular, we applied PFC (run 1), PFC (run 2), SNR, the Wilcoxon rank-sum test and the Class-Based Threshold as described above. The first three methods were run in each direction, to look for genes up in responders and then up in non-responders. The Wilcoxon rank-sum test was bidirectional and identified genes up in either responders or non-responders. Thus, there were 7 runs of the methods. In each case, the probe sets were sorted based on their score, and ranked. The top 100 ranked probe sets from each method were selected for Table 1. The last column in the table identifies the minimum rank across the methods.

TABLE 1
PREDICTIVE MARKER IDENTIFICATION

| | | | | | | Rank | | Rank | | Rank | | | |
|---|---|---|---|---|---|---|---|---|---|---|---|---|---|
| | Probeset | Sequence Derived | | Gene | Rank | R | Rank | R | Rank NR | R | Rank Wilcoxon | Rank | |
| No. | ID | From | Title | Symbol | NR PFC-1 | PFC-1 | NR PFC-2 | PFC-1 | SNR | SNR | rank-sum test | CBT | Minimum rank |
| 1 | 204298_{—} | NM_002317.1 | lysyl oxidase | LOX | 44928 | 44928 | 44928 | 44928 | 44855 | 74 | 112 | >100 | 74 |
| | s_at | | | | | | | | | | | | |
| 2 | 205884_{—} | NM_000885.2 | integrin, alpha 4 (antigen | ITGA4 | 44928 | 44928 | 86 | 44928 | 949 | 43980 | 2675 | >100 | 86 |
| | at | | CD49D, alpha 4 subunit | | | | | | | | | | |
| | | | of VLA-4 receptor) | | | | | | | | | | |
| 3 | 228841_{—} | AW299250 | Homo sapiens cDNA | — | 44928 | 44928 | 91 | 44928 | 95 | 44834 | 197 | >100 | 91 |
| | at | | FLJ32429 fis, clone | | | | | | | | | | |
| | | | SKMUS2001014. | | | | | | | | | | |
| 4 | 243366_{—} | AI936034 | integrin, alpha 4 (antigen | ITGA4 | 44928 | 44928 | 98 | 44928 | 1896 | 43033 | 6343 | >100 | 98 |
| | s_at | | CD49D, alpha 4 subunit | | | | | | | | | | |
| | | | of VLA-4 receptor) | | | | | | | | | | |
| 5 | 214265_{—} | AI193623 | integrin, alpha 8 | ITGA8 | 14 | 44928 | 25 | 44928 | 924 | 44005 | 4689 | 16 | 14 |
| | at | | | | | | | | | | | | |
| 6 | 203949_{—} | NM_000250.1 | myeloperoxidase | MPO | 44928 | 2 | 44928 | 25 | 44178 | 751 | 2599 | >100 | 2 |
| | at | | | | | | | | | | | | |
| 7 | 207341_{—} | NM_002777.2 | proteinase 3 (serine | PRTN3 | 44928 | 4 | 44928 | 44928 | 43054 | 1875 | 17751 | >100 | 4 |
| | at | | proteinase, neutrophil, | | | | | | | | | | |
| | | | Wegener granulomatosis | | | | | | | | | | |
| | | | autoantigen) | | | | | | | | | | |
| 8 | 203948_{—} | J02694.1 | myeloperoxidase | MPO | 44928 | 11 | 44928 | 44928 | 42466 | 2463 | 17515 | >100 | 11 |
| | s_at | | | | | | | | | | | | |
| 9 | 224461_{—} | BC006121.1 | apoptosis-inducing | AMID | 59 | 44928 | 44928 | 44928 | 360 | 44569 | 2121 | >100 | 59 |
| | s_at | | factor (AIF)- | | | | | | | | | | |
| | | | homologous | | | | | | | | | | |
| | | | mitochondrion- | | | | | | | | | | |
| | | | associated inducer of | | | | | | | | | | |
| | | | death | | | | | | | | | | |
| 10 | 206056_{—} | X52075 | sialophorin (gpL115, | SPN | 44928 | 44928 | 44928 | 82 | 44735 | 194 | 304 | >100 | 82 |
| | x_at | | leukosialin, CD43) | | | | | | | | | | |
| 11 | 203489_{—} | NM_006427.2 | CD27-binding (Siva) | SIVA | 44928 | 44928 | 44928 | 44928 | 86 | 44843 | 281 | >100 | 86 |
| | at | | protein | | | | | | | | | | |
| 12 | 226507_{—} | AU154408 | p21/Cdc42/Rac1- | PAK1 | 90 | 44928 | 44928 | 44928 | 974 | 43955 | 3521 | >100 | 90 |
| | at | | activated kinase 1 | | | | | | | | | | |
| | | | (STE20 homolog, yeast) | | | | | | | | | | |
| 13 | 216055_{—} | AK022920.1 | platelet-derived growth | PDGFB | 44928 | 44928 | 44928 | 44928 | 44829 | 100 | 224 | >100 | 100 |
| | at | | factor beta polypeptide | | | | | | | | | | |
| | | | (simian sarcoma viral | | | | | | | | | | |
| | | | (v-sis) oncogene | | | | | | | | | | |
| | | | homolog) | | | | | | | | | | |
| 14 | 209942_{—} | BC000340.1 | melanoma antigen, | MAGEA3 | 44928 | 44928 | 2 | 44928 | 217 | 44712 | 602 | >100 | 2 |
| | x_at | | family A, 3 | | | | | | | | | | |
| 15 | 214612_{—} | U10691 | — | — | 44928 | 44928 | 4 | 44928 | 357 | 44572 | 2061 | >100 | 4 |
| | x_at | | | | | | | | | | | | |
| 16 | 217969_{—} | NM_013265.2 | melanoma antigen, | MAGED1 | 8 | 44928 | 55 | 44928 | 197 | 44732 | 2165 | 4 | 4 |
| | at | | family D, 1 | | | | | | | | | | |
| 17 | 215733_{—} | AJ012833.1 | cancer/testis antigen 2 | CTAG2 | 18 | 44928 | 5 | 44928 | 922 | 44007 | 28547 | 36 | 5 |
| | x_at | | | | | | | | | | | | |
| 18 | 210546_{—} | U87459.1 | cancer/testis antigen 1 | CTAG1 | 13 | 44928 | 7 | 44928 | 1278 | 43651 | 12645 | 32 | 7 |
| | x_at | | | | | | | | | | | | |
| 19 | 211674_{—} | AF038567.1 | cancer/testis antigen 1 | CTAG1 | 21 | 44928 | 8 | 44928 | 1185 | 43744 | 27104 | 25 | 8 |
| | x_at | | | | | | | | | | | | |
| 20 | 223313_{—} | BC001207.1 | MAGE-E1 protein | MAGE- | 44928 | 44928 | 44928 | 12 | 42615 | 2314 | 9805 | >100 | 12 |
| | s_at | | | E1 | | | | | | | | | |
| 21 | 210467_{—} | BC003408.1 | melanoma antigen, | MAGEA12 | 44928 | 44928 | 21 | 44928 | 2258 | 42671 | 10757 | >100 | 21 |
| | x_at | | family A, 12 | | | | | | | | | | |
| 22 | 220057_{—} | NM_020411.1 | G antigen, family D, 2 | GAGED2 | 44928 | 44928 | 24 | 44928 | 2785 | 42144 | 10634 | >100 | 24 |
| | at | | | | | | | | | | | | |
| 23 | 236152_{—} | AW135330 | PAGE-5 protein | PAGE-5 | 40 | 44928 | 44928 | 44928 | 908 | 44021 | 8811 | >100 | 40 |
| | at | | | | | | | | | | | | |
| 24 | 233831_{—} | AI246052 | Homo sapiens | — | 44928 | 44928 | 44928 | 44928 | 44874 | 55 | 142 | >100 | 55 |
| | at | | serologically defined | | | | | | | | | | |
| | | | breast cancer antigen | | | | | | | | | | |
| | | | NY-BR-40 mRNA, | | | | | | | | | | |
| | | | partial cds | | | | | | | | | | |
| 25 | 206427_{—} | U06654.1 | melan-A | MLANA | 44928 | 44928 | 44928 | 44928 | 44873 | 56 | 159 | >100 | 56 |
| | s_at | | | | | | | | | | | | |
| 26 | 206218_{—} | NM_002364.1 | melanoma antigen, | MAGEB2 | 63 | 44928 | 44928 | 44928 | 3637 | 41292 | 38186 | >100 | 63 |
| | at | | family B, 2 | | | | | | | | | | |
| 27 | 203386_{—} | AI650848 | TBC1 domain family, | TBC1D4 | 44928 | 44928 | 44928 | 44928 | 44844 | 85 | 439 | >100 | 85 |
| | at | | member 4 | | | | | | | | | | |
| 28 | 201457_{—} | AF081496.1 | BUB3 budding | BUB3 | 44928 | 44928 | 61 | 44928 | 62 | 44867 | 113 | 14 | 14 |
| | x_at | | uninhibited by | | | | | | | | | | |
| | | | benzimidazoles 3 | | | | | | | | | | |
| | | | homolog (yeast) | | | | | | | | | | |
| 29 | 213348_{—} | N33167 | cyclin-dependent kinase | CDKN1C | 44928 | 31 | 44928 | 44928 | 44846 | 83 | 147 | >100 | 31 |
| | at | | inhibitor 1C (p57, Kip2) | | | | | | | | | | |
| 30 | 204170_{—} | NM_001827.1 | CDC28 protein kinase | CKS2 | 44928 | 44928 | 34 | 44928 | 464 | 44465 | 828 | >100 | 34 |
| | s_at | | regulatory subunit 2 | | | | | | | | | | |
| 31 | 206205_{—} | NM_022782.1 | M-phase phosphoprotein 9 | MPHOS | 44928 | 44928 | 44928 | 44928 | 40 | 44889 | 72 | >100 | 40 |
| | at | | | PH9 | | | | | | | | | |
| 32 | 208796_{—} | BC000196.1 | cyclin G1 | CCNG1 | 44928 | 44928 | 68 | 44928 | 250 | 44679 | 517 | >100 | 68 |
| | s_at | | | | | | | | | | | | |
| 33 | 204460_{—} | AF074717.1 | RAD1 homolog(S. | RAD1 | 44928 | 44928 | 44928 | 44928 | 71 | 44858 | 128 | >100 | 71 |
| | s_at | | pombe) | | | | | | | | | | |
| 34 | 224918_{—} | AI220117 | microsomal glutathione | MGST1 | 28 | 44928 | 44928 | 44928 | 10617 | 34312 | 19002 | >100 | 28 |
| | x_at | | S-transferase 1 | | | | | | | | | | |
| 35 | 205998_{—} | NM_017460.2 | cytochrome P450, | CYP3A4 | 44928 | 44928 | 44928 | 44928 | 44852 | 77 | 87 | >100 | 77 |
| | x_at | | subfamily IIIA | | | | | | | | | | |
| | | | (niphedipine oxidase), | | | | | | | | | | |
| | | | polypeptide 4 | | | | | | | | | | |
| 36 | 239476_{—} | AW152166 | Homo sapiens cDNA | — | 44928 | 44928 | 44928 | 44928 | 44925 | 4 | 9 | >100 | 4 |
| | at | | FLJ36491 fis, clone | | | | | | | | | | |
| | | | THYMU2018197. | | | | | | | | | | |
| 37 | 211298_{—} | AF116645.1 | albumin | ALB | 44928 | 44928 | 44928 | 44928 | 44914 | 15 | 95 | >100 | 15 |
| | s_at | | | | | | | | | | | | |
| 38 | 216835_{—} | AF035299.1 | docking protein 1, | DOK1 | 44928 | 44928 | 44928 | 44928 | 44921 | 8 | 42 | >100 | 8 |
| | s_at | | 62 kDa (downstream of | | | | | | | | | | |
| | | | tyrosine kinase 1) | | | | | | | | | | |
| 39 | 213891_{—} | AI927067 | Homo sapiens cDNA | — | 44928 | 44928 | 44928 | 20 | 43578 | 1351 | 1063 | >100 | 20 |
| | s_at | | FLJ11918 fis, clone | | | | | | | | | | |
| | | | HEMBB1000272. | | | | | | | | | | |
| 40 | 212387_{—} | AK021980.1 | Homo sapiens cDNA | — | 44928 | 44928 | 44928 | 31 | 43365 | 1564 | 393 | >100 | 31 |
| | at | | FLJ11918 fis, clone | | | | | | | | | | |
| | | | HEMBB1000272. | | | | | | | | | | |
| 41 | 212382_{—} | AK021980.1 | Homo sapiens cDNA | — | 44928 | 40 | 44928 | 44928 | 37843 | 7086 | 9000 | >100 | 40 |
| | at | | FLJ11918 fis, clone | | | | | | | | | | |
| | | | HEMBB1000272. | | | | | | | | | | |
| 42 | 203753_{—} | NM_003199.1 | transcription factor 4 | TCF4 | 44928 | 44928 | 44928 | 42 | 43376 | 1553 | 1580 | >100 | 42 |
| | at | | | | | | | | | | | | |
| 43 | 212386_{—} | AK021980.1 | Homo sapiens cDNA | — | 44928 | 44928 | 44928 | 64 | 42346 | 2583 | 1261 | >100 | 64 |
| | at | | FLJ11918 fis, clone | | | | | | | | | | |
| | | | HEMBB1000272. | | | | | | | | | | |
| 44 | 211709_{—} | BC005810.1 | stem cell growth factor; | SCGF | 44928 | 44928 | 44928 | 99 | 44282 | 647 | 1192 | >100 | 99 |
| | s_at | | lymphocyte secreted C- | | | | | | | | | | |
| | | | type lectin | | | | | | | | | | |
| 45 | 217020_{—} | X04014 | — | — | 44928 | 44928 | 44928 | 44928 | 44917 | 12 | 71 | >100 | 12 |
| | at | | | | | | | | | | | | |
| 46 | 217786_{—} | NM_006109.1 | SKB1 homolog (S. | SKB1 | 44928 | 44928 | 44928 | 44928 | 34 | 44895 | 17 | >100 | 17 |
| | at | | pombe) | | | | | | | | | | |
| 47 | 206109_{—} | NM_000148.1 | fucosyltransferase 1 | FUT1 | 44928 | 44928 | 44928 | 44928 | 44907 | 22 | 41 | >100 | 22 |
| | at | | (galactoside 2-alpha-L- | | | | | | | | | | |
| | | | fucosyltransferase, | | | | | | | | | | |
| | | | Bombay phenotype | | | | | | | | | | |
| | | | included) | | | | | | | | | | |
| 48 | 227798_{—} | AU146891 | ESTs | — | 44928 | 44928 | 23 | 44928 | 2520 | 42409 | 6771 | >100 | 23 |
| | at | | | | | | | | | | | | |
| 49 | 208743_{—} | BC001359.1 | tyrosine 3- | YWHAB | 44928 | 44928 | 44928 | 44928 | 51 | 44878 | 100 | >100 | 51 |
| | s_at | | monooxygenase/tryptop | | | | | | | | | | |
| | | | han 5-monooxygenase | | | | | | | | | | |
| | | | activation protein, beta | | | | | | | | | | |
| | | | polypeptide | | | | | | | | | | |
| 50 | 225239_{—} | AI355441 | ESTs, Moderately | — | 44928 | 44928 | 44928 | 57 | 44845 | 84 | 226 | >100 | 57 |
| | at | | similar to hypothetical | | | | | | | | | | |
| | | | protein FLJ20958 | | | | | | | | | | |
| | | | [Homo sapiens] | | | | | | | | | | |
| | | | [H. sapiens] | | | | | | | | | | |
| 51 | 215551_{—} | AI073549 | estrogen receptor 1 | ESR1 | 44928 | 44928 | 44928 | 44928 | 44868 | 61 | 109 | >100 | 61 |
| | at | | | | | | | | | | | | |
| 52 | 215067_{—} | AU147942 | Homo sapiens cDNA | — | 44928 | 44928 | 44928 | 72 | 43871 | 1058 | 2063 | >100 | 72 |
| | x_at | | FLJ12333 fis, clone | | | | | | | | | | |
| | | | MAMMA1002198, | | | | | | | | | | |
| | | | highly similar to | | | | | | | | | | |
| | | | THIOREDOXIN | | | | | | | | | | |
| | | | PEROXIDASE 1. | | | | | | | | | | |
| 53 | 210993_{—} | U54826.1 | MAD, mothers against | MADH1 | 44928 | 44928 | 100 | 44928 | 3077 | 41852 | 5470 | >100 | 100 |
| | s_at | | decapentaplegic | | | | | | | | | | |
| | | | homolog 1 (Drosophila) | | | | | | | | | | |
| 54 | 209374_{—} | BC001872.1 | immunoglobulin heavy | IGHM | 2 | 44928 | 44928 | 44928 | 1769 | 43160 | 31220 | 66 | 2 |
| | s_at | | constant mu | | | | | | | | | | |
| 55 | 224342_{—} | L14452.1 | immunoglobulin lambda | IGL@ | 4 | 44928 | 44928 | 44928 | 2837 | 42092 | 28929 | 29 | 4 |
| | x_at | | locus | | | | | | | | | | |
| 56 | 212827_{—} | X17115.1 | immunoglobulin heavy | IGHM | 6 | 44928 | 44928 | 44928 | 3364 | 41565 | 36442 | >100 | 6 |
| | at | | constant mu | | | | | | | | | | |
| 57 | 234366_{—} | AF103591.1 | immunoglobulin lambda | IGL@ | 44928 | 44928 | 44928 | 26 | 30154 | 14775 | 21162 | >100 | 26 |
| | x_at | | locus | | | | | | | | | | |
| 58 | 216986_{—} | D78261.1 | interferon regulatory | IRF4 | 44928 | 44928 | 44928 | 44928 | 43 | 44886 | 129 | >100 | 43 |
| | s_at | | factor 4 | | | | | | | | | | |
| 59 | 205098_{—} | AI421071 | chemokine (C-C motif) | CCR1 | 46 | 44928 | 44928 | 44928 | 2037 | 42892 | 13544 | >100 | 46 |
| | at | | receptor 1 | | | | | | | | | | |
| 60 | 239237_{—} | AI798822 | ESTs | — | 120 | 44928 | 79 | 44928 | 4324 | 40605 | 22488 | >100 | 79 |
| | at | | | | | | | | | | | | |
| 61 | 205099_{—} | NM_001295.1 | chemokine (C-C motif) | CCR1 | 85 | 44928 | 44928 | 44928 | 3294 | 41635 | 13545 | >100 | 85 |
| | s_at | | receptor 1 | | | | | | | | | | |
| 62 | 223472_{—} | AF071594.1 | Wolf-Hirschhorn | WHSC1 | 44928 | 44928 | 44928 | 2 | 43897 | 1032 | 6635 | >100 | 2 |
| | at | | syndrome candidate 1 | | | | | | | | | | |
| 63 | 222778_{—} | AI770166 | Wolf-Hirschhorn | WHSC1 | 44928 | 44928 | 44928 | 3 | 42704 | 2225 | 7936 | >100 | 3 |
| | s_at | | syndrome candidate 1 | | | | | | | | | | |
| 64 | 209054_{—} | AF083389.1 | Wolf-Hirschhorn | WHSC1 | 44928 | 44928 | 44928 | 4 | 44524 | 405 | 444 | >100 | 4 |
| | s_at | | syndrome candidate 1 | | | | | | | | | | |
| 65 | 222777_{—} | AI770166 | Wolf-Hirschhorn | WHSC1 | 44928 | 44928 | 44928 | 5 | 41834 | 3095 | 13244 | >100 | 5 |
| | s_at | | syndrome candidate 1 | | | | | | | | | | |
| 66 | 209053_{—} | AF083389.1 | Wolf-Hirschhorn | WHSC1 | 44928 | 44928 | 44928 | 7 | 42426 | 2503 | 10341 | >100 | 7 |
| | s_at | | syndrome candidate 1 | | | | | | | | | | |
| 67 | 200921_{—} | NM_001731.1 | B-cell translocation gene | BTG1 | 75 | 44928 | 27 | 44928 | 260 | 44669 | 787 | 24 | 24 |
| | s_at | | 1, anti-proliferative | | | | | | | | | | |
| 68 | 209052_{—} | AF083389.1 | Wolf-Hirschhorn | WHSC1 | 44928 | 44928 | 44928 | 24 | 42989 | 1940 | 4673 | >100 | 24 |
| | s_at | | syndrome candidate 1 | | | | | | | | | | |
| 69 | 213940_{—} | AU145053 | formin binding protein 1 | FNBP1 | 44928 | 44928 | 43 | 44928 | 7005 | 37924 | 11991 | >100 | 43 |
| | s_at | | | | | | | | | | | | |
| 70 | 213732_{—} | BE962186 | transcription factor 3 | TCF3 | 44928 | 44928 | 44928 | 44928 | 44876 | 53 | 200 | >100 | 53 |
| | at | | (E2A immunoglobulin | | | | | | | | | | |
| | | | enhancer binding factors | | | | | | | | | | |
| | | | E12/E47) | | | | | | | | | | |
| 71 | 213047_{—} | AI278616 | SET translocation | SET | 44928 | 44928 | 74 | 44928 | 85 | 44844 | 207 | >100 | 74 |
| | x_at | | (myeloid leukemia- | | | | | | | | | | |
| | | | associated) | | | | | | | | | | |
| 72 | 200631_{—} | NM_003011.1 | SET translocation | SET | 130 | 44928 | 44928 | 44928 | 175 | 44754 | 642 | 81 | 81 |
| | s_at | | (myeloid leukemia- | | | | | | | | | | |
| | | | associated) | | | | | | | | | | |
| 73 | 205068_{—} | BE671084 | GTPase regulator | GRAF | 44928 | 44928 | 44928 | 44928 | 44830 | 99 | 190 | >100 | 99 |
| | s_at | | associated with focal | | | | | | | | | | |
| | | | adhesion kinase | | | | | | | | | | |
| | | | pp125(FAK) | | | | | | | | | | |
| 74 | 220146_{—} | NM_016562.1 | toll-like receptor 7 | TLR7 | 10 | 44928 | 44928 | 44928 | 961 | 43968 | 9515 | >100 | 10 |
| | at | | | | | | | | | | | | |
| 75 | 232304_{—} | AK026714.1 | pellino homolog 1 | PELI1 | 44928 | 44928 | 44928 | 13 | 44623 | 306 | 766 | >100 | 13 |
| | at | | (Drosophila) | | | | | | | | | | |
| 76 | 232213_{—} | AU147506 | pellino homolog 1 | PELI1 | 44928 | 44928 | 44928 | 18 | 44653 | 276 | 1025 | >100 | 18 |
| | at | | (Drosophila) | | | | | | | | | | |
| 77 | 218319_{—} | NM_020651.2 | pellino homolog 1 | PELI1 | 44928 | 44928 | 44928 | 38 | 41381 | 3548 | 3985 | >100 | 38 |
| | at | | (Drosophila) | | | | | | | | | | |
| 78 | 215744_{—} | AW514140 | fusion, derived from | FUS | 44928 | 44928 | 44928 | 44928 | 44853 | 76 | 158 | >100 | 76 |
| | at | | t(12;16) malignant | | | | | | | | | | |
| | | | liposarcoma | | | | | | | | | | |
| 79 | 206363_{—} | NM_005360.2 | v-maf | MAF | 44928 | 44928 | 44928 | 8 | 34192 | 10737 | 7331 | >100 | 8 |
| | at | | musculoaponeurotic | | | | | | | | | | |
| | | | fibrosarcoma oncogene | | | | | | | | | | |
| | | | homolog (avian) | | | | | | | | | | |
| 80 | 202768_{—} | NM_006732.1 | FBJ murine | FOSB | 44928 | 44928 | 44928 | 51 | 43123 | 1806 | 2597 | >100 | 51 |
| | at | | osteosarcoma viral | | | | | | | | | | |
| | | | oncogene homolog B | | | | | | | | | | |
| 81 | 202647_{—} | NM_002524.2 | neuroblastoma RAS | NRAS | 78 | 44928 | 52 | 44928 | 169 | 44760 | 691 | >100 | 52 |
| | s_at | | viral (v-ras) oncogene | | | | | | | | | | |
| | | | homolog | | | | | | | | | | |
| 82 | 209640_{—} | M79462. 1 | promyelocytic leukemia | PML | 44928 | 44928 | 44928 | 44928 | 44851 | 78 | 115 | >100 | 78 |
| | at | | | | | | | | | | | | |
| 140 | 232231_{—} | AL353944.1 | Runt domain | RUNX2 | 1 | 44928 | 1 | 44928 | 17 | 44912 | 212 | 1 | 1 |
| | at | | transcription factor 2 | | | | | | | | | | |
| 83 | 201575_{—} | NM_012245.1 | SKI-interacting protein | SNW1 | 44928 | 44928 | 44928 | 44928 | 3 | 44926 | 12 | >100 | 3 |
| | at | | | | | | | | | | | | |
| 84 | 224985_{—} | BE964484 | Homo sapiens, clone | — | 31 | 44928 | 13 | 44928 | 54 | 44875 | 130 | 6 | 6 |
| | at | | IMAGE: 3446533, | | | | | | | | | | |
| | | | mRNA | | | | | | | | | | |
| 85 | 204602_{—} | NM_012242.1 | dickkopf homolog 1 | DKK1 | 44928 | 44928 | 10 | 44928 | 2757 | 42172 | 9868 | >100 | 10 |
| | at | | (Xenopus laevis) | | | | | | | | | | |
| 86 | 201653_{—} | NM_005776.1 | cornichon homolog | CNIH | 44928 | 44928 | 45 | 44928 | 16 | 44913 | 26 | 94 | 16 |
| | at | | (Drosophila) | | | | | | | | | | |
| 87 | 234021_{—} | AK024984.1 | Homo sapiens cDNA: | — | 44928 | 44928 | 44928 | 44928 | 44909 | 20 | 16 | >100 | 16 |
| | at | | FLJ21331 fis, clone | | | | | | | | | | |
| | | | COL02520. | | | | | | | | | | |
| 88 | 212063_{—} | BE903880 | CD44 antigen (homing | CD44 | 44928 | 44928 | 18 | 44928 | 2720 | 42209 | 8726 | 62 | 18 |
| | at | | function and Indian | | | | | | | | | | |
| | | | blood group system) | | | | | | | | | | |
| 89 | 204489_{—} | NM_000610.1 | CD44 antigen (homing | CD44 | 34 | 44928 | 54 | 44928 | 3784 | 41145 | 21033 | >100 | 34 |
| | s_at | | function and Indian | | | | | | | | | | |
| | | | blood group system) | | | | | | | | | | |
| 90 | 227167_{—} | AW511319 | Homo sapiens | — | 44928 | 44928 | 37 | 44928 | 155 | 44774 | 430 | >100 | 37 |
| | s_at | | mesenchymal stem cell | | | | | | | | | | |
| | | | protein DSC96 mRNA, | | | | | | | | | | |
| | | | partial cds | | | | | | | | | | |
| 91 | 202290_{—} | NM_014891.1 | PDGFA associated | PDAP1 | 44928 | 44928 | 44928 | 44928 | 78 | 44851 | 108 | >100 | 78 |
| | at | | protein 1 | | | | | | | | | | |
| 92 | 215499_{—} | AA780381 | mitogen-activated | MAP2K3 | 44928 | 44928 | 44928 | 78 | 44259 | 670 | 1433 | >100 | 78 |
| | at | | protein kinase kinase 3 | | | | | | | | | | |
| 93 | 200047_{—} | NM_003403.2 | YY1 transcription factor | YY1 | 44928 | 44928 | 44928 | 44928 | 135 | 44794 | 193 | 95 | 95 |
| | s_at | | | | | | | | | | | | |
| 94 | 222555_{—} | AI338045 | mitochondrial ribosomal | MRPL44 | 44928 | 44928 | 44928 | 44928 | 4 | 44925 | 11 | >100 | 4 |
| | s_at | | protein L44 | | | | | | | | | | |
| 95 | 212694_{—} | NM_000532.1 | propionyl Coenzyme A | PCCB | 44928 | 44928 | 44928 | 44928 | 7 | 44922 | 19 | >100 | 7 |
| | s_at | | carboxylase, beta | | | | | | | | | | |
| | | | polypeptide | | | | | | | | | | |
| 96 | 222530_{—} | AF275813.1 | McKusick-Kaufman | MKKS | 69 | 44928 | 129 | 44928 | 13 | 44916 | 15 | 42 | 13 |
| | s_at | | syndrome | | | | | | | | | | |
| 97 | 200869_{—} | NM_000980.1 | ribosomal protein L18a | RPL18A | 20 | 44928 | 97 | 44928 | 723 | 44206 | 2697 | 76 | 20 |
| | at | | | | | | | | | | | | |
| 98 | 200023_{—} | NM_003754.1 | eukaryotic translation | EIF3S5 | 29 | 44928 | 65 | 44928 | 178 | 44751 | 992 | 21 | 21 |
| | s_at | | initiation factor 3, | | | | | | | | | | |
| | | | subunit 5 epsilon, 47 kDa | | | | | | | | | | |
| 99 | 200812_{—} | NM_006429.1 | chaperonin containing | CCT7 | 44928 | 44928 | 44928 | 44928 | 22 | 44907 | 25 | >100 | 22 |
| | at | | TCP1, subunit 7 (eta) | | | | | | | | | | |
| 100 | 225190_{—} | AW402660 | ribosomal protein L35a | RPL35A | 27 | 44928 | 44928 | 44928 | 423 | 44506 | 1445 | 27 | 27 |
| | x_at | | | | | | | | | | | | |
| 101 | 200023_{—} | NM_003754.1 | eukaryotic translation | EIF3S5 | 58 | 44928 | 51 | 44928 | 182 | 44747 | 332 | 31 | 31 |
| | s_at | | initiation factor 3, | | | | | | | | | | |
| | | | subunit 5 epsilon, 47 kDa | | | | | | | | | | |
| 102 | 217919_{—} | BE782148 | mitochondrial ribosomal | MRPL42 | 44928 | 44928 | 82 | 44928 | 60 | 44869 | 34 | >100 | 34 |
| | s_at | | protein L42 | | | | | | | | | | |
| 103 | 211972_{—} | AI953822 | ribosomal protein, large, | RPLP0 | 92 | 44928 | 44928 | 44928 | 378 | 44551 | 420 | 38 | 38 |
| | x_at | | P0 | | | | | | | | | | |
| 104 | 200024_{—} | NM_001009.1 | ribosomal protein S5 | RPS5 | 118 | 44928 | 93 | 44928 | 122 | 44807 | 333 | 41 | 41 |
| | at | | | | | | | | | | | | |
| 105 | 200715_{—} | BC000514.1 | ribosomal protein L13a | RPL13A | 47 | 44928 | 114 | 44928 | 2857 | 42072 | 9548 | >100 | 47 |
| | x_at | | | | | | | | | | | | |
| 106 | 201258_{—} | NM_001020.1 | ribosomal protein S16 | RPS16 | 99 | 44928 | 99 | 44928 | 185 | 44744 | 738 | 51 | 51 |
| | at | | | | | | | | | | | | |
| 107 | 200003_{—} | NM_000991.1 | ribosomal protein L28 | RPL28 | 56 | 44928 | 44928 | 44928 | 2488 | 42441 | 9320 | >100 | 56 |
| | s_at | | | | | | | | | | | | |
| 108 | 221726_{—} | BE250348 | ribosomal protein L22 | RPL22 | 44928 | 44928 | 115 | 44928 | 206 | 44723 | 657 | 64 | 64 |
| | at | | | | | | | | | | | | |
| 109 | 200041_{—} | NM_004640.1 | HLA-B associated | BAT1 | 44928 | 44928 | 44928 | 70 | 33237 | 11692 | 18501 | >100 | 70 |
| | s_at | | transcript 1 | | | | | | | | | | |
| 110 | 211937_{—} | NM_001417.1 | eukaryotic translation | EIF4B | 44928 | 44928 | 71 | 44928 | 794 | 44135 | 2480 | >100 | 71 |
| | at | | initiation factor 4B | | | | | | | | | | |
| 111 | 200082_{—} | AI805587 | ribosomal protein S7 | RPS7 | 72 | 44928 | 84 | 44928 | 468 | 44461 | 1272 | 85 | 72 |
| | s_at | | | | | | | | | | | | |
| 112 | 214167_{—} | AA555113 | ribosomal protein, large, | RPLP0 | 44928 | 44928 | 107 | 44928 | 239 | 44690 | 326 | 73 | 73 |
| | s_at | | P0 | | | | | | | | | | |
| 113 | 200024_{—} | NM_001009.1 | ribosomal protein S5 | RPS5 | 152 | 44928 | 44928 | 44928 | 156 | 44773 | 546 | 77 | 77 |
| | at | | | | | | | | | | | | |
| 114 | 217719_{—} | NM_016091.1 | eukaryotic translation | EIF3S6IP | 44928 | 44928 | 44928 | 44928 | 532 | 44397 | 951 | 78 | 78 |
| | at | | initiation factor 3, | | | | | | | | | | |
| | | | subunit 6 interacting | | | | | | | | | | |
| | | | protein | | | | | | | | | | |
| 115 | 225797_{—} | AV707568 | mitochondrial ribosomal | MRPL54 | 166 | 44928 | 138 | 44928 | 108 | 44821 | 312 | 83 | 83 |
| | at | | protein L54 | | | | | | | | | | |
| 116 | 200937_{—} | NM_000969.1 | ribosomal protein L5 | RPL5 | 44928 | 44928 | 89 | 44928 | 1188 | 43741 | 3462 | >100 | 89 |
| | s_at | | | | | | | | | | | | |
| 117 | 208985_{—} | BC002719.1 | eukaryotic translation | EIF3S1 | 105 | 44928 | 44928 | 44928 | 90 | 44839 | 199 | >100 | 90 |
| | s_at | | initiation factor 3, | | | | | | | | | | |
| | | | subunit 1 alpha, 35 kDa | | | | | | | | | | |
| 118 | 200834_{—} | NM_001024.1 | ribosomal protein S21 | RPS21 | 109 | 44928 | 136 | 44928 | 870 | 44059 | 4275 | 98 | 98 |
| | s_at | | | | | | | | | | | | |
| 119 | 216153_{—} | AK022897.1 | reversion-inducing- | RECK | 44928 | 3 | 44928 | 9 | 44724 | 205 | 1125 | >100 | 3 |
| | x_at | | cysteine-rich protein | | | | | | | | | | |
| | | | with kazal motifs | | | | | | | | | | |
| 120 | 217687_{—} | AA224446 | adenylate cyclase 2 | ADCY2 | 44928 | 44928 | 44928 | 44928 | 44923 | 6 | 28 | >100 | 6 |
| | at | | (brain) | | | | | | | | | | |
| 121 | 222632_{—} | AA843132 | leucine zipper | LZTFL1 | 44928 | 44928 | 22 | 44928 | 559 | 44370 | 962 | >100 | 22 |
| | s_at | | transcription factor-like 1 | | | | | | | | | | |
| 122 | 236623_{—} | AI367432 | hypothetical protein | MGC16179 | 44928 | 33 | 44928 | 44928 | 43090 | 1839 | 11437 | >100 | 33 |
| | at | | MGC16179 | | | | | | | | | | |
| 123 | 221899_{—} | AI809961 | hypothetical protein | CG005 | 44928 | 41 | 44928 | 44928 | 40910 | 4019 | 11859 | >100 | 41 |
| | at | | from BCRA2 region | | | | | | | | | | |
| 124 | 221691_{—} | AB042278.1 | nucleophosmin | NPM1 | 43 | 44928 | 44928 | 44928 | 926 | 44003 | 3231 | >100 | 43 |
| | x_at | | (nucleolar | | | | | | | | | | |
| | | | phosphoprotein B23, | | | | | | | | | | |
| | | | numatrin) | | | | | | | | | | |
| 125 | 209030_{—} | NM_014333.1 | immunoglobulin | IGSF4 | 44928 | 44928 | 44 | 44928 | 2842 | 42087 | 9276 | >100 | 44 |
| | s_at | | superfamily, member 4 | | | | | | | | | | |
| 126 | 222762_{—} | AU144259 | LIM domains containing 1 | LIMD1 | 44928 | 44928 | 57 | 44928 | 1570 | 43359 | 4714 | >100 | 57 |
| | x_at | | | | | | | | | | | | |
| 127 | 240983_{—} | AW292273 | cysteinyl-tRNA | CARS | 44928 | 44928 | 80 | 44928 | 1536 | 43393 | 2413 | >100 | 80 |
| | s_at | | synthetase | | | | | | | | | | |
| 128 | 200713_{—} | NM_012325.1 | microtubule-associated | MAPRE1 | 44928 | 44928 | 44928 | 44928 | 96 | 44833 | 300 | >100 | 96 |
| | s_at | | protein, RP/EB family, | | | | | | | | | | |
| | | | member 1 | | | | | | | | | | |
| 129 | 200814_{—} | NM_006263.1 | proteasome (prosome, | PSME1 | 44928 | 44928 | 130 | 44928 | 14 | 44915 | 31 | 44 | 14 |
| | at | | macropain) activator | | | | | | | | | | |
| | | | subunit 1 (PA28 alpha) | | | | | | | | | | |
| 130 | 201532_{—} | NM_002788.1 | proteasome (prosome, | PSMA3 | 76 | 44928 | 30 | 44928 | 19 | 44910 | 22 | 26 | 19 |
| | at | | macropain) subunit, | | | | | | | | | | |
| | | | alpha type, 3 | | | | | | | | | | |
| 131 | 218011_{—} | NM_024292.1 | ubiquitin-like 5 | UBL5 | 44928 | 44928 | 94 | 44928 | 39 | 44890 | 90 | 47 | 39 |
| | at | | | | | | | | | | | | |
| 132 | 224747_{—} | AK000617.1 | hypothetical protein | LOC92912 | 44928 | 44928 | 44928 | 44928 | 391 | 44538 | 706 | 45 | 45 |
| | at | | LOC92912 | | | | | | | | | | |
| 133 | 201758_{—} | NM_006292.1 | tumor susceptibility | TSG101 | 44928 | 44928 | 44928 | 44928 | 65 | 44864 | 171 | >100 | 65 |
| | at | | gene 101 | | | | | | | | | | |
| 134 | 200019_{—} | NM_001997.1 | Finkel-Biskis-Reilly | FAU | 156 | 44928 | 44928 | 44928 | 220 | 44709 | 640 | 68 | 68 |
| | s_at | | murine sarcoma virus | | | | | | | | | | |
| | | | (FBR-MuSV) | | | | | | | | | | |
| | | | ubiquitously expressed | | | | | | | | | | |
| | | | (fox derived); ribosomal | | | | | | | | | | |
| | | | protein S30 | | | | | | | | | | |
| 135 | 202346_{—} | NM_005339.2 | huntingtin interacting | HIP2 | 44928 | 44928 | 44928 | 44928 | 79 | 44850 | 255 | >100 | 79 |
| | at | | protein 2 | | | | | | | | | | |
| 136 | 201177_{—} | NM_005499.1 | SUMO-1 activating | UBA2 | 44928 | 44928 | 143 | 44928 | 81 | 44848 | 170 | 87 | 81 |
| | s_at | | enzyme subunit 2 | | | | | | | | | | |
| 137 | 200043_{—} | NM_004450.1 | enhancer of rudimentary | ERH | 44928 | 44928 | 140 | 44928 | 1 | 44928 | 7 | 22 | 1 |
| | at | | homolog (Drosophila) | | | | | | | | | | |
| 138 | 212109_{—} | AK023154.1 | HN1 like | HN1L | 44928 | 44928 | 44928 | 44928 | 44928 | 1 | 4 | >100 | 1 |
| | at | | | | | | | | | | | | |
| 139 | 212190_{—} | AL541302 | serine (or cysteine) | SERPINE2 | 44928 | 44928 | 44928 | 1 | 44650 | 279 | 325 | >100 | 1 |
| | at | | proteinase inhibitor, | | | | | | | | | | |
| | | | clade E (nexin, | | | | | | | | | | |
| | | | plasminogen activator | | | | | | | | | | |
| | | | inhibitor type 1), | | | | | | | | | | |
| | | | member 2 | | | | | | | | | | |
| 141 | 234428_{—} | AL110127.1 | Homo sapiens mRNA; | — | 44928 | 44928 | 44928 | 44928 | 44927 | 2 | 1 | >100 | 1 |
| | at | | cDNA DKFZp564I1316 | | | | | | | | | | |
| | | | (from clone | | | | | | | | | | |
| | | | DKFZp564I1316) | | | | | | | | | | |
| 142 | 235102_{—} | AI684439 | phenylalanine | PAH | 44928 | 1 | 44928 | 6 | 44469 | 460 | 4356 | >100 | 1 |
| | x_at | | hydroxylase | | | | | | | | | | |
| 143 | 200965_{—} | NM_006720.1 | actin binding LIM | ABLIM1 | 44928 | 44928 | 44928 | 44928 | 44919 | 10 | 2 | >100 | 2 |
| | s_at | | protein 1 | | | | | | | | | | |
| 144 | 222783_{—} | NM_022137.1 | SPARC related modular | SMOC1 | 22 | 44928 | 3 | 44928 | 72 | 44857 | 117 | 2 | 2 |
| | s_at | | calcium binding 1 | | | | | | | | | | |
| 145 | 232075_{—} | BF791874 | recombination protein | REC14 | 5 | 44928 | 31 | 44928 | 2 | 44927 | 8 | 3 | 2 |
| | at | | REC14 | | | | | | | | | | |
| 146 | 220565_{—} | NM_016602.1 | G protein-coupled | GPR2 | 3 | 44928 | 14 | 44928 | 304 | 44625 | 851 | 5 | 3 |
| | at | | receptor 2 | | | | | | | | | | |
| 147 | 220572_{—} | NM_018705.1 | hypothetical protein | DKFZp5 | 44928 | 44928 | 44928 | 44928 | 44926 | 3 | 3 | >100 | 3 |
| | at | | DKFZp547G183 | 47G183 | | | | | | | | | |
| 148 | 208263_{—} | NM_018581.1 | — | — | 44928 | 44928 | 44928 | 44928 | 44903 | 26 | 5 | >100 | 5 |
| | at | | | | | | | | | | | | |
| 149 | 221569_{—} | AL136797.1 | hypothetical protein | FLJ20069 | 44928 | 9 | 44928 | 48 | 44924 | 5 | 13 | >100 | 5 |
| | at | | FLJ20069 | | | | | | | | | | |
| 150 | 222427_{—} | AK021413.1 | leucyl-tRNA synthetase | LARS | 12 | 44928 | 76 | 44928 | 5 | 44924 | 36 | 9 | 5 |
| | s_at | | | | | | | | | | | | |
| 151 | 230941_{—} | AI651340 | Homo sapiens, clone | — | 44928 | 5 | 44928 | 44928 | 44738 | 191 | 96 | >100 | 5 |
| | at | | IMAGE: 5271446, | | | | | | | | | | |
| | | | mRNA | | | | | | | | | | |
| 152 | 201682_{—} | NM_004279.1 | peptidase (mitochondrial | PMPCB | 38 | 44928 | 73 | 44928 | 6 | 44923 | 10 | 20 | 6 |
| | at | | processing) beta | | | | | | | | | | |
| 153 | 210258_{—} | AF030107.1 | regulator of G-protein | RGS13 | 44928 | 44928 | 6 | 44928 | 3847 | 41082 | 26318 | >100 | 6 |
| | at | | signalling 13 | | | | | | | | | | |
| 154 | 218438_{—} | NM_025205.1 | endothelial-derived gene 1 | EG1 | 60 | 44928 | 44928 | 44928 | 10 | 44919 | 6 | >100 | 6 |
| | s_at | | | | | | | | | | | | |
| 155 | 227341_{—} | AW195407 | Homo sapiens mRNA; | — | 44928 | 6 | 44928 | 44928 | 43167 | 1762 | 10075 | >100 | 6 |
| | at | | cDNA DKFZp686C072 | | | | | | | | | | |
| | | | (from clone | | | | | | | | | | |
| | | | DKFZp686C072) | | | | | | | | | | |
| 156 | 202075_{—} | NM_006227.1 | phospholipid transfer | PLTP | 44928 | 7 | 44928 | 44928 | 39569 | 5360 | 20579 | >100 | 7 |
| | s_at | | protein | | | | | | | | | | |
| 157 | 216288_{—} | AU159276 | cysteinyl leukotriene | CYSLTR1 | 44928 | 44928 | 44928 | 44928 | 44922 | 7 | 46 | >100 | 7 |
| | at | | receptor 1 | | | | | | | | | | |
| 158 | 217915_{—} | NM_016304.1 | chromosome 15 open | C15orf15 | 33 | 44928 | 35 | 44928 | 11 | 44918 | 14 | 7 | 7 |
| | s_at | | reading frame 15 | | | | | | | | | | |
| 159 | 222968_{—} | NM_016947.1 | chromosome 6 open | C6orf48 | 7 | 44928 | 11 | 44928 | 107 | 44822 | 481 | 43 | 7 |
| | at | | reading frame 48 | | | | | | | | | | |
| 160 | 202567_{—} | NM_004175.1 | small nuclear | SNRPD3 | 44928 | 44928 | 28 | 44928 | 8 | 44921 | 32 | 28 | 8 |
| | at | | ribonucleoprotein D3 | | | | | | | | | | |
| | | | polypeptide 18 kDa | | | | | | | | | | |
| 161 | 213510_{—} | AW194543 | TL132 protein | LOC220594 | 44928 | 8 | 44928 | 34 | 44098 | 831 | 2375 | >100 | 8 |
| | x_at | | | | | | | | | | | | |
| 162 | 225065_{—} | AI826279 | hypothetical protein | MGC40157 | 41 | 44928 | 33 | 44928 | 68 | 44861 | 92 | 8 | 8 |
| | x_at | | MGC40157 | | | | | | | | | | |
| 163 | 204287_{—} | NM_004711.1 | synaptogyrin 1 | SYNGR1 | 44928 | 44928 | 44928 | 44928 | 44920 | 9 | 24 | >100 | 9 |
| | at | | | | | | | | | | | | |
| 164 | 206762_{—} | NM_002234.1 | potassium voltage-gated | KCNA5 | 9 | 44928 | 44928 | 44928 | 1038 | 43891 | 20489 | >100 | 9 |
| | at | | channel, shaker-related | | | | | | | | | | |
| | | | subfamily, member 5 | | | | | | | | | | |
| 165 | 210250_{—} | AF067854.1 | adenylosuccinate lyase | ADSL | 44928 | 44928 | 44928 | 44928 | 9 | 44920 | 27 | >100 | 9 |
| | x_at | | | | | | | | | | | | |
| 166 | 210497_{—} | BC002818.1 | synovial sarcoma, X | SSX2 | 44928 | 44928 | 9 | 44928 | 651 | 44278 | 3927 | >100 | 9 |
| | x_at | | breakpoint 2 | | | | | | | | | | |
| 167 | 223358_{—} | AW269834 | Homo sapiens cDNA | — | 54 | 44928 | 39 | 44928 | 99 | 44830 | 366 | 10 | 10 |
| | s_at | | FLJ33024 fis, clone | | | | | | | | | | |
| | | | THYMU1000532, | | | | | | | | | | |
| | | | moderately similar to | | | | | | | | | | |
| | | | HIGH-AFFINITY | | | | | | | | | | |
| | | | CAMP-SPECIFIC 3′,5′- | | | | | | | | | | |
| | | | CYCLIC | | | | | | | | | | |
| | | | PHOSPHODIESTERAS | | | | | | | | | | |
| | | | E (EC 3.1.4.17). | | | | | | | | | | |
| 168 | 225767_{—} | AL531684 | ESTs, Weakly similar to | — | 44928 | 10 | 44928 | 44928 | 31271 | 13658 | 34008 | >100 | 10 |
| | at | | T02345 hypothetical | | | | | | | | | | |
| | | | protein KIAA0324- | | | | | | | | | | |
| | | | human (fragment) | | | | | | | | | | |
| | | | [H. sapiens] | | | | | | | | | | |
| 169 | 232169_{—} | AK002110.1 | NADH dehydrogenase | NDUFS8 | 44928 | 44928 | 44928 | 10 | 44849 | 80 | 245 | >100 | 10 |
| | x_at | | (ubiquinone) Fe-S | | | | | | | | | | |
| | | | protein 8, 23 kDa | | | | | | | | | | |
| | | | (NADH-coenzyme Q | | | | | | | | | | |
| | | | reductase) | | | | | | | | | | |
| 170 | 216287_{—} | AK021930.1 | — | — | 44928 | 44928 | 44928 | 44928 | 44918 | 11 | 52 | >100 | 11 |
| | at | | | | | | | | | | | | |
| 171 | 228332_{—} | AA526939 | selenoprotein H | SELH | 55 | 44928 | 149 | 44928 | 38 | 44891 | 67 | 11 | 11 |
| | s_at | | | | | | | | | | | | |
| 172 | 242903_{—} | AI458949 | ESTs | — | 44928 | 44928 | 44928 | 11 | 44599 | 330 | 1363 | >100 | 11 |
| | at | | | | | | | | | | | | |
| 173 | 244114_{—} | AI003508 | ESTs | — | 11 | 44928 | 44928 | 44928 | 3539 | 41390 | 33890 | >100 | 11 |
| | x_at | | | | | | | | | | | | |
| 174 | 223490_{—} | AF281132.1 | exosome component | RRP40 | 44928 | 44928 | 44928 | 44928 | 12 | 44917 | 29 | >100 | 12 |
| | s_at | | Rrp40 | | | | | | | | | | |
| 175 | 224496_{—} | BC006292.1 | hypothetical protein | MGC10744 | 44928 | 12 | 44928 | 44 | 40920 | 4009 | 11871 | >100 | 12 |
| | s_at | | MGC10744 | | | | | | | | | | |
| 176 | 226243_{—} | BF590958 | hypothetical protein | MGC11266 | 44928 | 44928 | 12 | 44928 | 97 | 44832 | 49 | 49 | 12 |
| | at | | MGC11266 | | | | | | | | | | |
| 177 | 231045_{—} | H29876 | selenoprotein H | SELH | 44928 | 44928 | 121 | 44928 | 28 | 44901 | 39 | 12 | 12 |
| | x_at | | | | | | | | | | | | |
| 178 | 206978_{—} | NM_000647.2 | chemokine (C-C motif) | CCR2 | 82 | 44928 | 20 | 44928 | 818 | 44111 | 2153 | 13 | 13 |
| | at | | receptor 2 | | | | | | | | | | |
| 179 | 212062_{—} | AB014511.1 | ATPase, Class II, type | ATP9A | 44928 | 13 | 44928 | 44928 | 44776 | 153 | 45 | >100 | 13 |
| | at | | 9A | | | | | | | | | | |
| 180 | 227692_{—} | AU153866 | guanine nucleotide | GNAI1 | 44928 | 44928 | 44928 | 44928 | 44916 | 13 | 21 | >100 | 13 |
| | at | | binding protein (G | | | | | | | | | | |
| | | | protein), alpha inhibiting | | | | | | | | | | |
| | | | activity polypeptide 1 | | | | | | | | | | |
| 181 | 200710_{—} | NM_000018.1 | acyl-Coenzyme A | ACADVL | 44928 | 14 | 44928 | 69 | 44212 | 717 | 2804 | >100 | 14 |
| | at | | dehydrogenase, very | | | | | | | | | | |
| | | | long chain | | | | | | | | | | |
| 182 | 216529_{—} | AL049244.1 | Homo sapiens mRNA; | — | 44928 | 44928 | 44928 | 44928 | 44915 | 14 | 75 | >100 | 14 |
| | at | | cDNA DKEZp564C163 | | | | | | | | | | |
| | | | (from clone | | | | | | | | | | |
| | | | DKFZp564C163) | | | | | | | | | | |
| 183 | 233437_{—} | AF238869.1 | gamma-aminobutyric | GABRA 4 | 44928 | 36 | 44928 | 14 | 44817 | 112 | 455 | >100 | 14 |
| | at | | acid (GABA) A | | | | | | | | | | |
| | | | receptor, alpha 4 | | | | | | | | | | |
| 184 | 202591_{—} | NM_003143.1 | single-stranded DNA | SSBP1 | 44928 | 44928 | 44928 | 44928 | 15 | 44914 | 69 | 75 | 15 |
| | s_at | | binding protein | | | | | | | | | | |
| 185 | 206632_{—} | NM_004900.1 | apolipoprotein B mRNA | APOBEC3B | 61 | 44928 | 15 | 44928 | 386 | 44543 | 1554 | 65 | 15 |
| | s_at | | editing enzyme, catalytic | | | | | | | | | | |
| | | | polypeptide-like 3B | | | | | | | | | | |
| 186 | 213975_{—} | AV711904 | lysozyme (renal | LYZ | 44928 | 44928 | 44928 | 15 | 39536 | 5393 | 16729 | >100 | 15 |
| | s_at | | amyloidosis) | | | | | | | | | | |
| 187 | 224493_{—} | BC006280.1 | hypothetical protein | MGC11386 | 44928 | 15 | 44928 | 44928 | 44792 | 137 | 450 | >100 | 15 |
| | x_at | | MGC11386 | | | | | | | | | | |
| 188 | 226392_{—} | AI888503 | Homo sapiens cDNA: | — | 112 | 44928 | 69 | 44928 | 80 | 44849 | 94 | 15 | 15 |
| | at | | FLJ21652 fis, clone | | | | | | | | | | |
| | | | COL08582. | | | | | | | | | | |
| 189 | 235666_{—} | AA903473 | ESTs, Weakly similar to | — | 15 | 44928 | 44928 | 44928 | 2414 | 42515 | 6329 | 58 | 15 |
| | at | | hypothetical protein | | | | | | | | | | |
| | | | FLJ20489 [Homo | | | | | | | | | | |
| | | | sapiens] [H. sapiens] | | | | | | | | | | |
| 190 | 205807_{—} | NM_020127.1 | tuftelin 1 | TUFT1 | 44928 | 44928 | 44928 | 44928 | 44913 | 16 | 44 | >100 | 16 |
| | s_at | | | | | | | | | | | | |
| 191 | 206121_{—} | NM_000036.1 | adenosine | AMPD1 | 44928 | 44928 | 16 | 44928 | 236 | 44693 | 516 | 23 | 16 |
| | at | | monophosphate | | | | | | | | | | |
| | | | deaminase 1 (isoform | | | | | | | | | | |
| | | | M) | | | | | | | | | | |
| 192 | 207697_{—} | NM_005874.1 | leukocyte | LILRB2 | 44928 | 16 | 44928 | 44928 | 43348 | 1581 | 11408 | >100 | 16 |
| | x_at | | immunoglobulin-like | | | | | | | | | | |
| | | | receptor, subfamily B | | | | | | | | | | |
| | | | (with TM and ITIM | | | | | | | | | | |
| | | | domains), member 2 | | | | | | | | | | |
| 193 | 207912_{—} | NM_004081.2 | deleted in azoospermia | DAZ | 16 | 44928 | 44928 | 44928 | 1052 | 43877 | 10620 | >100 | 16 |
| | s_at | | | | | | | | | | | | |
| 194 | 222315_{—} | AW972855 | ESTs | — | 44928 | 44928 | 44928 | 16 | 40968 | 3961 | 5887 | >100 | 16 |
| | at | | | | | | | | | | | | |
| 195 | 58367_{—} | AA429615 | hypothetical protein | FLJ23233 | 44928 | 44928 | 44928 | 44928 | 44912 | 17 | 53 | >100 | 17 |
| | s_at | | FLJ23233 | | | | | | | | | | |
| 196 | 214657_{—} | AU134977 | Human clone 137308 | — | 44928 | 17 | 44928 | 21 | 44515 | 414 | 1432 | >100 | 17 |
| | s_at | | mRNA, partial cds. | | | | | | | | | | |
| 197 | 217466_{—} | L48784 | — | — | 44928 | 44928 | 17 | 44928 | 527 | 44402 | 1267 | 18 | 17 |
| | x_at | | | | | | | | | | | | |
| 198 | 220232_{—} | NM_024906.1 | hypothetical protein | FLJ21032 | 44928 | 44928 | 44928 | 17 | 44432 | 497 | 1066 | >100 | 17 |
| | at | | FLJ21032 | | | | | | | | | | |
| 199 | 225698_{—} | BF314746 | TIGA1 | TIGA1 | 53 | 44928 | 46 | 44928 | 342 | 44587 | 1351 | 17 | 17 |
| | at | | | | | | | | | | | | |
| 200 | 232010_{—} | AA129444 | hypothetical protein | DKFZp566D234 | 17 | 44928 | 44928 | 44928 | 614 | 44315 | 6850 | 86 | 17 |
| | at | | DKFZp566D234 | | | | | | | | | | |
| 201 | 219429_{—} | NM_024306.1 | fatty acid hydroxylase | FAAH | 44928 | 44928 | 44928 | 44928 | 44863 | 66 | 18 | >100 | 18 |
| | at | | | | | | | | | | | | |
| 202 | 225981_{—} | AW139549 | chromosome 17 open | C17orf28 | 44928 | 44928 | 44928 | 44928 | 44911 | 18 | 83 | >100 | 18 |
| | at | | reading frame 28 | | | | | | | | | | |
| 203 | 229483_{—} | AA760738 | ESTs | — | 44928 | 18 | 44928 | 44928 | 44712 | 217 | 612 | >100 | 18 |
| | at | | | | | | | | | | | | |
| 204 | 235940_{—} | AW983691 | hypothetical protein | MGC10999 | 71 | 44928 | 66 | 44928 | 18 | 44911 | 40 | 84 | 18 |
| | at | | MGC10999 | | | | | | | | | | |
| 205 | 204836_{—} | NM_000170.1 | glycine dehydrogenase | GLDC | 19 | 44928 | 44928 | 44928 | 2228 | 42701 | 23086 | 99 | 19 |
| | at | | (decarboxylating; | | | | | | | | | | |
| | | | glycine decarboxylase, | | | | | | | | | | |
| | | | glycine cleavage system | | | | | | | | | | |
| | | | protein P) | | | | | | | | | | |
| 206 | 210800_{—} | BC005236.1 | hypothetical protein | MGC12262 | 44928 | 44928 | 44928 | 44928 | 44910 | 19 | 62 | >100 | 19 |
| | at | | MGC12262 | | | | | | | | | | |
| 207 | 222465_{—} | AF165521.1 | chromosome 15 open | C15orf15 | 44928 | 44928 | 83 | 44928 | 46 | 44883 | 82 | 19 | 19 |
| | at | | reading frame 15 | | | | | | | | | | |
| 208 | 222784_{—} | NM_022137.1 | SPARC related modular | SMOC1 | 44928 | 44928 | 19 | 44928 | 1100 | 43829 | 4324 | >100 | 19 |
| | at | | calcium binding 1 | | | | | | | | | | |
| 209 | 225710_{—} | H99792 | Homo sapiens cDNA | — | 44928 | 44928 | 44928 | 19 | 44375 | 554 | 688 | >100 | 19 |
| | at | | FLJ34013 fis, clone | | | | | | | | | | |
| | | | FCBBF2002111. | | | | | | | | | | |
| 210 | 229170_{—} | AW024437 | tetratricopeptide repeat- | LOC118491 | 44928 | 19 | 44928 | 92 | 43950 | 979 | 5702 | >100 | 19 |
| | s_at | | containing protein | | | | | | | | | | |
| 211 | 219373_{—} | NM_018973.1 | dolichyl-phosphate | DPM3 | 44928 | 20 | 44928 | 44928 | 38207 | 6722 | 15777 | >100 | 20 |
| | at | | mannosyltransferase | | | | | | | | | | |
| | | | polypeptide 3 | | | | | | | | | | |
| 212 | 221532_{—} | AF309553.1 | recombination protein | REC14 | 44928 | 44928 | 132 | 44928 | 25 | 44904 | 20 | 88 | 20 |
| | s_at | | REC14 | | | | | | | | | | |
| 213 | 226882_{—} | AI861913 | WD repeat domain 4 | WDR4 | 44928 | 44928 | 26 | 44928 | 20 | 44909 | 38 | >100 | 20 |
| | x_at | | | | | | | | | | | | |
| 214 | 222410_{—} | AF121856.1 | sorting nexin 6 | SNX6 | 173 | 44928 | 50 | 44928 | 21 | 44908 | 35 | 39 | 21 |
| | s_at | | | | | | | | | | | | |
| 215 | 225177_{—} | AA143793 | Rab coupling protein | RCP | 44928 | 21 | 44928 | 44928 | 43188 | 1741 | 4334 | >100 | 21 |
| | at | | | | | | | | | | | | |
| 216 | 243178_{—} | AW969703 | ESTs, Weakly similar to | — | 44928 | 44928 | 44928 | 44928 | 44908 | 21 | 50 | >100 | 21 |
| | at | | hypothetical protein | | | | | | | | | | |
| | | | FLJ20489 [Homo | | | | | | | | | | |
| | | | sapiens] [H. sapiens] | | | | | | | | | | |
| 217 | 205671_{—} | NM_002120.1 | major histocompatibility | HLA- | 44928 | 25 | 44928 | 22 | 44677 | 252 | 596 | >100 | 22 |
| | s_at | | complex, class II, DO | DOB | | | | | | | | | |
| | | | beta | | | | | | | | | | |
| 218 | 232538_{—} | AK027226.1 | Homo sapiens cDNA: | — | 44928 | 22 | 44928 | 29 | 44459 | 470 | 2019 | >100 | 22 |
| | at | | FLJ23573 fis, clone | | | | | | | | | | |
| | | | LNG12520. | | | | | | | | | | |
| 219 | 208151_{—} | NM_030881.1 | DEAD/H (Asp-Glu-Ala- | DDX17 | 44928 | 44928 | 44928 | 23 | 42362 | 2567 | 8455 | >100 | 23 |
| | x_at | | Asp/His) box | | | | | | | | | | |
| | | | polypeptide 17, 72 kDa | | | | | | | | | | |
| 220 | 214246_{—} | AI859060 | misshapen/NIK-related | MINK | 44928 | 23 | 44928 | 93 | 44744 | 185 | 1197 | >100 | 23 |
| | x_at | | kinase | | | | | | | | | | |
| 221 | 223996_{—} | AF151083.1 | mitochondrial ribosomal | MRPL30 | 44928 | 44928 | 44928 | 44928 | 23 | 44906 | 37 | >100 | 23 |
| | s_at | | protein L30 | | | | | | | | | | |
| 222 | 224330_{—} | AB049647.1 | mitochondrial ribosomal | MRPL27 | 44928 | 44928 | 59 | 44928 | 31 | 44898 | 23 | >100 | 23 |
| | s_at | | protein L27 | | | | | | | | | | |
| 223 | 227174_{—} | Z98443 | ESTs | — | 23 | 44928 | 44928 | 44928 | 1433 | 43496 | 8774 | >100 | 23 |
| | at | | | | | | | | | | | | |
| 224 | 235875_{—} | BF510711 | ESTs | — | 44928 | 44928 | 44928 | 44928 | 44906 | 23 | 65 | >100 | 23 |
| | at | | | | | | | | | | | | |
| 225 | 201520_{—} | NM_002092.1 | G-rich RNA sequence | GRSF1 | 44928 | 44928 | 102 | 44928 | 24 | 44905 | 61 | >100 | 24 |
| | s_at | | binding factor 1 | | | | | | | | | | |
| 226 | 211276_{—} | AF063606.1 | my048 protein | my048 | 44928 | 24 | 44928 | 44928 | 44693 | 236 | 186 | >100 | 24 |
| | at | | | | | | | | | | | | |
| 227 | 223395_{—} | AB056106.1 | DKFZP586L2024 | NESHBP | 24 | 44928 | 44928 | 44928 | 4177 | 40752 | 26522 | >100 | 24 |
| | at | | protein | | | | | | | | | | |
| 228 | 237429_{—} | AI677858 | ESTs | — | 44928 | 44928 | 44928 | 44928 | 44905 | 24 | 99 | >100 | 24 |
| | at | | | | | | | | | | | | |
| 229 | 215604_{—} | AK023783.1 | — | — | 44928 | 44928 | 44928 | 44928 | 44904 | 25 | 148 | >100 | 25 |
| | x_at | | | | | | | | | | | | |
| 230 | 239092_{—} | BF939224 | ESTs, Highly similar to | — | 25 | 44928 | 44928 | 44928 | 151 | 44778 | 1162 | >100 | 25 |
| | at | | ITA8_HUMAN Integrin | | | | | | | | | | |
| | | | alpha-8 [H. sapiens] | | | | | | | | | | |
| 231 | 211747_{—} | BC005938.1 | LSM5 homolog, U6 | LSM5 | 122 | 44928 | 44928 | 44928 | 26 | 44903 | 54 | 50 | 26 |
| | s_at | | small nuclear RNA | | | | | | | | | | |
| | | | associated (S. | | | | | | | | | | |
| | | | cerevisiae) | | | | | | | | | | |
| 232 | 216274_{—} | N99438 | signal peptidase | SPC18 | 26 | 44928 | 44928 | 44928 | 102 | 44827 | 359 | 34 | 26 |
| | s_at | | complex (18 kD) | | | | | | | | | | |
| 233 | 236427_{—} | BF830560 | ESTs | — | 44928 | 26 | 44928 | 44928 | 44074 | 855 | 2194 | >100 | 26 |
| | at | | | | | | | | | | | | |
| 234 | 203058_{—} | AW299958 | 3′-phosphoadenosine 5′- | PAPSS2 | 44928 | 27 | 44928 | 44928 | 44761 | 168 | 593 | >100 | 27 |
| | s_at | | phosphosulfate synthase 2 | | | | | | | | | | |
| 235 | 200043_{—} | NM_004450.1 | enhancer of rudimentary | ERH | 44928 | 44928 | 47 | 44928 | 27 | 44902 | 63 | 40 | 27 |
| | at | | homolog (Drosophila) | | | | | | | | | | |
| 236 | 234087_{—} | AK022343.1 | EST, Moderately similar | — | 44928 | 29 | 44928 | 44928 | 44902 | 27 | 79 | >100 | 27 |
| | at | | to hypothetical protein | | | | | | | | | | |
| | | | FLJ20294 [Homo | | | | | | | | | | |
| | | | sapiens] [H. sapiens] | | | | | | | | | | |
| 237 | 242311_{—} | H37943 | ESTs, Weakly similar to | — | 44928 | 44928 | 44928 | 27 | 44590 | 339 | 667 | >100 | 27 |
| | x_at | | hypothetical protein | | | | | | | | | | |
| | | | FLJ20489 [Homo | | | | | | | | | | |
| | | | sapiens] [H. sapiens] | | | | | | | | | | |
| 238 | 213307_{—} | AB028945.1 | SH3 and multiple | SHANK2 | 44928 | 44928 | 44928 | 44928 | 44901 | 28 | 43 | >100 | 28 |
| | at | | ankyrin repeat domains 2 | | | | | | | | | | |
| 239 | 237414_{—} | H70477 | coagulation factor VII | F7 | 44928 | 44928 | 44928 | 28 | 44539 | 390 | 2002 | >100 | 28 |
| | at | | (serum prothrombin | | | | | | | | | | |
| | | | conversion accelerator) | | | | | | | | | | |
| 240 | 239555_{—} | W87626 | ESTs | — | 44928 | 28 | 44928 | 44928 | 40008 | 4921 | 12979 | >100 | 28 |
| | at | | | | | | | | | | | | |
| 241 | 222893_{—} | AI609064 | hypothetical protein | FLJ13150 | 44928 | 44928 | 44928 | 44928 | 29 | 44900 | 47 | >100 | 29 |
| | s_at | | FLJ13150 | | | | | | | | | | |
| 242 | 225647_{—} | AI246687 | cathepsin C | CTSC | 44928 | 44928 | 29 | 44928 | 56 | 44873 | 30 | >100 | 29 |
| | s_at | | | | | | | | | | | | |
| 243 | 233876_{—} | AK000677.1 | Homo sapiens cDNA | — | 44928 | 44928 | 44928 | 44928 | 44900 | 29 | 105 | >100 | 29 |
| | at | | FLJ20670 fis, clone | | | | | | | | | | |
| | | | KAIA4743. | | | | | | | | | | |
| 244 | 201554_{—} | NM_004130.1 | glycogenin | GYG | 128 | 44928 | 40 | 44928 | 67 | 44862 | 387 | 30 | 30 |
| | x_at | | | | | | | | | | | | |
| 245 | 203561_{—} | NM_021642.1 | Fc fragment of IgG, low | FCGR2A | 44928 | 44928 | 44928 | 97 | 44899 | 30 | 74 | >100 | 30 |
| | at | | affinity IIa, receptor for | | | | | | | | | | |
| | | | (CD32) | | | | | | | | | | |
| 246 | 214594_{—} | BG252666 | ATPase, Class I, type | ATP8B1 | 44928 | 44928 | 44928 | 30 | 44816 | 113 | 236 | >100 | 30 |
| | x_at | | 8B, member 1 | | | | | | | | | | |
| 247 | 219030_{—} | NM_016058.1 | CGI-121 protein | CGI-121 | 44928 | 44928 | 44928 | 44928 | 30 | 44899 | 56 | >100 | 30 |
| | at | | | | | | | | | | | | |
| 248 | 219233_{—} | NM_018530.1 | hypothetical protein | PRO2521 | 44928 | 30 | 44928 | 44928 | 44418 | 511 | 1342 | >100 | 30 |
| | s_at | | PRO2521 | | | | | | | | | | |
| 249 | 242135_{—} | AA927533 | Homo sapiens cDNA | — | 30 | 44928 | 44928 | 44928 | 661 | 44268 | 3000 | >100 | 30 |
| | at | | FLJ32537 fis, clone | | | | | | | | | | |
| | | | SMINT2000400, highly | | | | | | | | | | |
| | | | similar to Homo sapiens | | | | | | | | | | |
| | | | FRG1 mRNA. | | | | | | | | | | |
| 250 | 228726_{—} | AW512196 | ESTs, Weakly similar to | — | 44928 | 42 | 44928 | 44928 | 44898 | 31 | 84 | >100 | 31 |
| | at | | hypothetical protein | | | | | | | | | | |
| | | | FLJ20489 [Homo | | | | | | | | | | |
| | | | sapiens] [H. sapiens] | | | | | | | | | | |
| 251 | 208642_{—} | AA205834 | X-ray repair | XRCC5 | 44928 | 44928 | 161 | 44928 | 32 | 44897 | 70 | 74 | 32 |
| | s_at | | complementing | | | | | | | | | | |
| | | | defective repair in | | | | | | | | | | |
| | | | Chinese hamster cells 5 | | | | | | | | | | |
| | | | (double-strand-break | | | | | | | | | | |
| | | | rejoining; Ku | | | | | | | | | | |
| | | | autoantigen, 80 kDa) | | | | | | | | | | |
| 252 | 220725_{—} | NM_025095.1 | hypothetical protein | FLJ23558 | 44928 | 32 | 44928 | 44928 | 44060 | 869 | 2613 | >100 | 32 |
| | x_at | | FLJ23558 | | | | | | | | | | |
| 253 | 220755_{—} | NM_016947.1 | chromosome 6 open | C6orf48 | 32 | 44928 | 64 | 44928 | 431 | 44498 | 1780 | 35 | 32 |
| | s_at | | reading frame 48 | | | | | | | | | | |
| 254 | 229269_{—} | BF976372 | myo-inositol 1- | ISYNA1 | 44928 | 44928 | 32 | 44928 | 809 | 44120 | 3681 | >100 | 32 |
| | x_at | | phosphate synthase A1 | | | | | | | | | | |
| 255 | 232659_{—} | AU146864 | Homo sapiens cDNA | — | 44928 | 44928 | 44928 | 44928 | 44897 | 32 | 178 | >100 | 32 |
| | at | | FLJ12017 fis, clone | | | | | | | | | | |
| | | | HEMBB1001735. | | | | | | | | | | |
| 256 | 244042_{—} | AA883831 | ESTs | — | 44928 | 44928 | 44928 | 32 | 44833 | 96 | 120 | >100 | 32 |
| | x_at | | | | | | | | | | | | |
| 257 | 204518_{—} | NM_000943.1 | peptidylprolyl isomerase | PPIC | 44928 | 44928 | 44928 | 33 | 44763 | 166 | 841 | >100 | 33 |
| | s_at | | C (cyclophilin C) | | | | | | | | | | |
| 258 | 205500_{—} | NM_001735.1 | complement component 5 | C5 | 44928 | 44928 | 44928 | 44928 | 44896 | 33 | 86 | >100 | 33 |
| | at | | | | | | | | | | | | |
| 259 | 209345_{—} | AL561930 | phosphatidylinositol 4- | PI4KII | 44928 | 44928 | 44928 | 44928 | 44890 | 39 | 33 | >100 | 33 |
| | s_at | | kinase type II | | | | | | | | | | |
| 260 | 222531_{—} | AW137526 | chromosome 14 open | C14orf108 | 44928 | 44928 | 41 | 44928 | 33 | 44896 | 111 | 54 | 33 |
| | s_at | | reading frame 108 | | | | | | | | | | |
| 261 | 224709_{—} | AF131831.1 | non-kinase Cdc42 | SPEC2 | 143 | 44928 | 62 | 44928 | 280 | 44649 | 857 | 33 | 33 |
| | s_at | | effector protein SPEC2 | | | | | | | | | | |
| 262 | 209427_{—} | AF064238.3 | smoothelin | SMTN | 44928 | 44928 | 44928 | 44928 | 44895 | 34 | 59 | >100 | 34 |
| | at | | | | | | | | | | | | |
| 263 | 236254_{—} | BE048857 | hypothetical protein | MGC45726 | 44928 | 34 | 44928 | 44928 | 44254 | 675 | 2739 | >100 | 34 |
| | at | | MGC45726 | | | | | | | | | | |
| 264 | 201056_{—} | N53479 | Homo sapiens cDNA | — | 44928 | 44928 | 44928 | 44928 | 44894 | 35 | 66 | >100 | 35 |
| | at | | FLJ37232 fis, clone | | | | | | | | | | |
| | | | BRAMY2001114. | | | | | | | | | | |
| 265 | 205644_{—} | NM_003096.1 | small nuclear | SNRPG | 155 | 44928 | 44928 | 44928 | 35 | 44894 | 77 | 37 | 35 |
| | s_at | | ribonucleoprotein | | | | | | | | | | |
| | | | polypeptide G | | | | | | | | | | |
| 266 | 228919_{—} | AA601031 | ESTs, Highly similar to | — | 44928 | 44928 | 44928 | 35 | 41176 | 3753 | 12711 | >100 | 35 |
| | at | | cell division cycle 2-like | | | | | | | | | | |
| | | | 1, isoform 1; Cell | | | | | | | | | | |
| | | | division cycle 2-like 1; | | | | | | | | | | |
| | | | PITSLRE protein kinase | | | | | | | | | | |
| | | | alpha; p58/GTA protein | | | | | | | | | | |
| | | | kinase; | | | | | | | | | | |
| | | | galactosyltransferase | | | | | | | | | | |
| | | | associated protein | | | | | | | | | | |
| | | | kinase; CDC-related | | | | | | | | | | |
| | | | protein kinase p58; | | | | | | | | | | |
| | | | PITSLRE B [Homo | | | | | | | | | | |
| | | | sapiens] [H. sapiens] | | | | | | | | | | |
| 267 | 231131_{—} | AA909330 | hypothetical protein | FLJ37659 | 35 | 44928 | 44928 | 44928 | 1469 | 43460 | 6555 | 71 | 35 |
| | at | | FLJ37659 | | | | | | | | | | |
| 268 | 240587_{—} | AI478814 | ESTs | — | 44928 | 35 | 44928 | 44928 | 36474 | 8455 | 27078 | >100 | 35 |
| | x_at | | | | | | | | | | | | |
| 269 | AFFX- | M10098 | — | — | 44928 | 44928 | 44928 | 36 | 25931 | 18998 | 37580 | >100 | 36 |
| | HUMR | | | | | | | | | | | | |
| | GE/M10098_{—} | | | | | | | | | | | | |
| | M_at | | | | | | | | | | | | |
| 270 | 212238_{—} | AL117518.1 | additional sex combs | ASXL1 | 44928 | 44928 | 44928 | 44928 | 44893 | 36 | 80 | >100 | 36 |
| | at | | like 1 (Drosophila) | | | | | | | | | | |
| 271 | 221434_{—} | NM_031210.1 | hypothetical protein | DC50 | 44928 | 44928 | 44928 | 44928 | 36 | 44893 | 103 | >100 | 36 |
| | s_at | | DC50 | | | | | | | | | | |
| 272 | 223029_{—} | AL136921.1 | ring finger and WD | RFWD1 | 39 | 44928 | 36 | 44928 | 104 | 44825 | 1374 | >100 | 36 |
| | s_at | | repeat domain 1 | | | | | | | | | | |
| 273 | 227641_{—} | AI613010 | hypothetical protein | MGC33974 | 36 | 44928 | 105 | 44928 | 124 | 44805 | 313 | >100 | 36 |
| | at | | MGC33974 | | | | | | | | | | |
| 274 | 206323_{—} | NM_002547.1 | oligophrenin 1 | OPHN1 | 44928 | 44928 | 44928 | 37 | 44545 | 384 | 324 | >100 | 37 |
| | x_at | | | | | | | | | | | | |
| 275 | 211424_{—} | AF113007.1 | DKFZP586A0522 | DKFZP586A0522 | 44928 | 37 | 44928 | 77 | 44775 | 154 | 575 | >100 | 37 |
| | x_at | | protein | | | | | | | | | | |
| 276 | 215322_{—} | AL080190.1 | Homo sapiens mRNA; | — | 44928 | 44928 | 44928 | 44928 | 44892 | 37 | 116 | >100 | 37 |
| | at | | cDNA DKFZp434A202 | | | | | | | | | | |
| | | | (from clone | | | | | | | | | | |
| | | | DKFZp434A202) | | | | | | | | | | |
| 277 | 222713_{—} | AF181995.1 | Fanconi anemia, | FANCF | 160 | 44928 | 154 | 44928 | 37 | 44892 | 151 | >100 | 37 |
| | s_at | | complementation groupF | | | | | | | | | | |
| 278 | 228496_{—} | AW243081 | cysteine-rich motor | CRIM1 | 37 | 44928 | 44928 | 44928 | 5459 | 39470 | 29457 | >100 | 37 |
| | s_at | | neuron 1 | | | | | | | | | | |
| 279 | 221223_{—} | NM_013324.2 | cytokine inducible SH2- | CISH | 44928 | 44928 | 44928 | 44928 | 44891 | 38 | 57 | >100 | 38 |
| | x_at | | containing protein | | | | | | | | | | |
| 280 | 224673_{—} | AI613244 | — | — | 44928 | 38 | 44928 | 67 | 44728 | 201 | 561 | >100 | 38 |
| | at | | | | | | | | | | | | |
| 281 | 224841_{—} | BF316352 | Homo sapiens mRNA; | — | 104 | 44928 | 38 | 44928 | 1040 | 43889 | 3386 | 46 | 38 |
| | x_at | | cDNA | | | | | | | | | | |
| | | | DKFZp564D0164 (from | | | | | | | | | | |
| | | | clone | | | | | | | | | | |
| | | | DKFZp564D0164) | | | | | | | | | | |
| 282 | 237266_{—} | BE552347 | Kv channel interacting | KCNIP2 | 44928 | 39 | 44928 | 44928 | 43140 | 1789 | 11320 | >100 | 39 |
| | at | | protein 2 | | | | | | | | | | |
| 283 | 244357_{—} | T90760 | ESTs | — | 44928 | 44928 | 44928 | 39 | 43992 | 937 | 3272 | >100 | 39 |
| | at | | | | | | | | | | | | |
| 284 | 228434_{—} | AA806965 | Homo sapiens, Similar | — | 44928 | 44928 | 44928 | 40 | 44467 | 462 | 1357 | >100 | 40 |
| | at | | to hypothetical protein | | | | | | | | | | |
| | | | B430208I01, clone | | | | | | | | | | |
| | | | IMAGE:5181522, | | | | | | | | | | |
| | | | mRNA, partial cds | | | | | | | | | | |
| 285 | 232746_{—} | BE552368 | Homo sapiens cDNA | — | 44928 | 44928 | 44928 | 44928 | 44889 | 40 | 64 | >100 | 40 |
| | at | | FLJ13445 fis, clone | | | | | | | | | | |
| | | | PLACE1002962. | | | | | | | | | | |
| 286 | 37793_{—} | AF034956 | RAD51-like 3 (S. | RAD51L3 | 44928 | 44928 | 44928 | 44928 | 44888 | 41 | 126 | >100 | 41 |
| | r_at | | cerevisiae) | | | | | | | | | | |
| 287 | 203408_{—} | NM_002971.1 | special AT-rich | SATB1 | 44928 | 44928 | 44928 | 41 | 43257 | 1672 | 1941 | >100 | 41 |
| | s_at | | sequence binding protein | | | | | | | | | | |
| | | | 1 (binds to nuclear | | | | | | | | | | |
| | | | matrix/scaffold- | | | | | | | | | | |
| | | | associating DNA's) | | | | | | | | | | |
| 288 | 207124_{—} | NM_006578.1 | guanine nucleotide | GNB5 | 44928 | 44928 | 44928 | 44928 | 41 | 44888 | 184 | >100 | 41 |
| | s_at | | binding protein (G | | | | | | | | | | |
| | | | protein), beta 5 | | | | | | | | | | |
| 289 | 208844_{—} | BC002456.1 | — | — | 44928 | 44928 | 44928 | 44928 | 44887 | 42 | 137 | >100 | 42 |
| | at | | | | | | | | | | | | |
| 290 | 218139_{—} | NM_018229.1 | chromosome 14 open | C14orf108 | 44928 | 44928 | 44928 | 44928 | 42 | 44887 | 55 | >100 | 42 |
| | s_at | | reading frame 108 | | | | | | | | | | |
| 291 | 224579_{—} | AK024263.1 | Homo sapiens cDNA | — | 44928 | 44928 | 42 | 44928 | 400 | 44529 | 757 | 52 | 42 |
| | at | | FLJ14201 fis, clone | | | | | | | | | | |
| | | | NT2RP3002955. | | | | | | | | | | |
| 292 | 244359_{—} | H28915 | ESTs | — | 42 | 44928 | 44928 | 44928 | 3802 | 41127 | 28000 | >100 | 42 |
| | s_at | | | | | | | | | | | | |
| 293 | 53987_{—} | AL041852 | KIAA1464 protein | KIAA1464 | 44928 | 44928 | 44928 | 44928 | 44886 | 43 | 127 | >100 | 43 |
| | at | | | | | | | | | | | | |
| 294 | 212307_{—} | BF001665 | O-linked N- | OGT | 44928 | 43 | 44928 | 44928 | 33355 | 11574 | 18158 | >100 | 43 |
| | s_at | | acetylglucosamine | | | | | | | | | | |
| | | | (GlcNAc) transferase | | | | | | | | | | |
| | | | (UDP-N- | | | | | | | | | | |
| | | | acetylglucosamine: polyp | | | | | | | | | | |
| | | | eptide-N- | | | | | | | | | | |
| | | | acetylglucosaminyl | | | | | | | | | | |
| | | | transferase) | | | | | | | | | | |
| 295 | 232098_{—} | AK025142.1 | ESTs | — | 44928 | 44928 | 44928 | 43 | 42790 | 2139 | 2890 | >100 | 43 |
| | at | | | | | | | | | | | | |
| 296 | 215908_{—} | AF009267.1 | Homo sapiens full | — | 44928 | 44 | 44928 | 44928 | 44462 | 467 | 1470 | >100 | 44 |
| | at | | length insert cDNA | | | | | | | | | | |
| | | | YU79F10 | | | | | | | | | | |
| 297 | 217294_{—} | U88968.1 | enolase 1, (alpha) | ENO1 | 44 | 44928 | 44928 | 44928 | 47 | 44882 | 135 | >100 | 44 |
| | s_at | | | | | | | | | | | | |
| 298 | 220852_{—} | NM_014099.1 | PRO1768 protein | PRO1768 | 44928 | 44928 | 44928 | 44928 | 44885 | 44 | 102 | >100 | 44 |
| | at | | | | | | | | | | | | |
| 299 | 225402_{—} | BG339450 | chromosome 20 open | C20orf64 | 44928 | 44928 | 44928 | 44928 | 44 | 44885 | 78 | >100 | 44 |
| | at | | reading frame 64 | | | | | | | | | | |
| 300 | 212923_{—} | AK024828.1 | hypothetical protein | LOC221749 | 44928 | 44928 | 44928 | 44928 | 44884 | 45 | 123 | >100 | 45 |
| | s_at | | LOC221749 | | | | | | | | | | |
| 301 | 222714_{—} | BC000878.1 | CGI-83 protein | CGI-83 | 44928 | 44928 | 44928 | 44928 | 45 | 44884 | 104 | >100 | 45 |
| | s_at | | | | | | | | | | | | |
| 302 | 229050_{—} | AL533103 | Homo sapiens cDNA | — | 45 | 44928 | 44928 | 44928 | 2495 | 42434 | 6112 | >100 | 45 |
| | s_at | | FLJ30346 fis, clone | | | | | | | | | | |
| | | | BRACE2007527. | | | | | | | | | | |
| 303 | 240593_{—} | R98767 | ESTs, Weakly similar to | — | 44928 | 45 | 44928 | 44928 | 39771 | 5158 | 14507 | >100 | 45 |
| | x_at | | hypothetical protein | | | | | | | | | | |
| | | | FLJ20378 [Homo | | | | | | | | | | |
| | | | sapiens] [H. sapiens] | | | | | | | | | | |
| 304 | 241722_{—} | BF724558 | ESTs, Moderately | — | 44928 | 44928 | 44928 | 45 | 43069 | 1860 | 3871 | >100 | 45 |
| | x_at | | similar to T02670 | | | | | | | | | | |
| | | | probable thromboxane | | | | | | | | | | |
| | | | A2 receptor isoform beta — | | | | | | | | | | |
| | | | human [H. sapiens] | | | | | | | | | | |
| 305 | 212110_{—} | D31887.1 | KIAA0062 protein | KIAA0062 | 44928 | 46 | 44928 | 44928 | 27676 | 17253 | 28338 | >100 | 46 |
| | at | | | | | | | | | | | | |
| 306 | 215628_{—} | AL049285.1 | Homo sapiens mRNA; | — | 44928 | 44928 | 44928 | 46 | 44499 | 430 | 654 | >100 | 46 |
| | x_at | | cDNA DKFZp564M193 | | | | | | | | | | |
| | | | (from clone | | | | | | | | | | |
| | | | DKFZp564M193) | | | | | | | | | | |
| 307 | 236946_{—} | AI220134 | ESTs | — | 44928 | 44928 | 44928 | 44928 | 44883 | 46 | 204 | >100 | 46 |
| | at | | | | | | | | | | | | |
| 308 | 210992_{—} | U90939.1 | Fc fragment of IgG, low | FCGR2A | 44928 | 44928 | 44928 | 47 | 43239 | 1690 | 3640 | >100 | 47 |
| | x_at | | affinity IIa, receptor for | | | | | | | | | | |
| | | | (CD32) | | | | | | | | | | |
| 309 | 217527_{—} | AI478300 | Homo sapiens, clone | — | 44928 | 47 | 44928 | 44928 | 40926 | 4003 | 14691 | >100 | 47 |
| | s_at | | IMAGE: 3659798, | | | | | | | | | | |
| | | | mRNA | | | | | | | | | | |
| 310 | 219183_{—} | NM_013385.2 | pleckstrin homology, | PSCD4 | 44928 | 44928 | 44928 | 44928 | 44882 | 47 | 101 | >100 | 47 |
| | s_at | | Sec7 and coiled/coil | | | | | | | | | | |
| | | | domains 4 | | | | | | | | | | |
| 311 | 200826_{—} | NM_004597.3 | small nuclear | SNRPD2 | 165 | 44928 | 44928 | 44928 | 48 | 44881 | 221 | 89 | 48 |
| | at | | ribonucleoprotein D2 | | | | | | | | | | |
| | | | polypeptide 16.5 kDa | | | | | | | | | | |
| 312 | 203663_{—} | NM_004255.1 | cytochrome c oxidase | COX5A | 44928 | 44928 | 110 | 44928 | 52 | 44877 | 48 | >100 | 48 |
| | s_at | | subunit Va | | | | | | | | | | |
| 313 | 209049_{—} | BC001004.1 | protein kinase C binding | PRKCBP1 | 44928 | 48 | 44928 | 44928 | 39921 | 5008 | 15023 | >100 | 48 |
| | s_at | | protein 1 | | | | | | | | | | |
| 314 | 209486_{—} | BC004546.1 | disrupter of silencing 10 | SAS10 | 79 | 44928 | 48 | 44928 | 144 | 44785 | 600 | 57 | 48 |
| | at | | | | | | | | | | | | |
| 315 | 213345_{—} | AI624015 | nuclear factor of | NFATC4 | 44928 | 44928 | 44928 | 44928 | 44881 | 48 | 51 | >100 | 48 |
| | at | | activated T-cells, | | | | | | | | | | |
| | | | cytoplasmic, | | | | | | | | | | |
| | | | calcineurin-dependent 4 | | | | | | | | | | |
| 316 | 223076_{—} | BC001041.1 | hypothetical protein | FLJ20303 | 48 | 44928 | 44928 | 44928 | 566 | 44363 | 2838 | 69 | 48 |
| | s_at | | FLJ20303 | | | | | | | | | | |
| 317 | 224364_{—} | AF251049.1 | peptidylprolyl isomerase | PPIL3 | 139 | 44928 | 44928 | 44928 | 121 | 44808 | 368 | 48 | 48 |
| | s_at | | (cyclophilin)-like 3 | | | | | | | | | | |
| 318 | 212750_{—} | AB020630.1 | protein phosphatase 1, | PPP1R16B | 44928 | 44928 | 49 | 44928 | 953 | 43976 | 2373 | >100 | 49 |
| | at | | regulatory (inhibitor) | | | | | | | | | | |
| | | | subunit 16B | | | | | | | | | | |
| 319 | 219203_{—} | NM_016049.1 | CGI-112 protein | CGI-112 | 44928 | 44928 | 44928 | 44928 | 49 | 44880 | 271 | >100 | 49 |
| | at | | | | | | | | | | | | |
| 320 | 224741_{—} | BG329175 | Homo sapiens mRNA; | — | 49 | 44928 | 70 | 44928 | 1470 | 43459 | 5688 | 53 | 49 |
| | x_at | | cDNA | | | | | | | | | | |
| | | | DKFZp564D0164 (from | | | | | | | | | | |
| | | | clone | | | | | | | | | | |
| | | | DKFZp564D0164) | | | | | | | | | | |
| 321 | 227062_{—} | AU155361 | plectin 1, intermediate | PLEC1 | 44928 | 44928 | 44928 | 49 | 44613 | 316 | 708 | >100 | 49 |
| | at | | filament binding protein | | | | | | | | | | |
| | | | 500 kDa | | | | | | | | | | |
| 322 | 232516_{—} | AU150385 | YY1 associated protein | YAP | 44928 | 44928 | 44928 | 101 | 44880 | 49 | 153 | >100 | 49 |
| | x_at | | | | | | | | | | | | |
| 323 | 207573_{—} | NM_006476.1 | ATP synthase, H+ | ATP5L | 50 | 44928 | 44928 | 44928 | 168 | 44761 | 305 | 56 | 50 |
| | x_at | | transporting | | | | | | | | | | |
| | | | mitochondrial F0 | | | | | | | | | | |
| | | | Complex, subunit g | | | | | | | | | | |
| 324 | 212644_{—} | AI671747 | chromosome 14 open | C14orf32 | 44928 | 44928 | 44928 | 44928 | 50 | 44879 | 89 | >100 | 50 |
| | s_at | | reading frame 32 | | | | | | | | | | |
| 325 | 231825_{—} | AK025060.1 | activating transcription | ATF7IP | 44928 | 44928 | 44928 | 44928 | 44879 | 50 | 152 | >100 | 50 |
| | x_at | | factor 7 interacting | | | | | | | | | | |
| | | | protein | | | | | | | | | | |
| 326 | 239331_{—} | AW954199 | ESTs | — | 44928 | 44928 | 44928 | 50 | 42943 | 1986 | 4181 | >100 | 50 |
| | at | | | | | | | | | | | | |
| 327 | 209733_{—} | AL034399 | hypothetical protein | LOC286440 | 44928 | 44928 | 44928 | 44928 | 44878 | 51 | 283 | >100 | 51 |
| | at | | LOC286440 | | | | | | | | | | |
| 328 | 230876_{—} | AI827906 | hypothetical protein | LOC169834 | 51 | 44928 | 44928 | 44928 | 658 | 44271 | 3954 | >100 | 51 |
| | at | | LOC169834 | | | | | | | | | | |
| 329 | 216750_{—} | AK024871.1 | amyloid beta (A4) | APBB2 | 44928 | 44928 | 44928 | 44928 | 44877 | 52 | 277 | >100 | 52 |
| | at | | precursor protein- | | | | | | | | | | |
| | | | binding, family B, | | | | | | | | | | |
| | | | member 2 (Fe65-like) | | | | | | | | | | |
| 330 | 228728_{—} | BF724137 | hypothetical protein | FLJ21986 | 52 | 44928 | 85 | 44928 | 215 | 44714 | 1139 | >100 | 52 |
| | at | | FLJ21986 | | | | | | | | | | |
| 331 | 230014_{—} | BF515592 | ESTs | — | 44928 | 44928 | 44928 | 52 | 41139 | 3790 | 8523 | >100 | 52 |
| | at | | | | | | | | | | | | |
| 332 | 210715_{—} | AF027205.1 | serine protease inhibitor, | SPINT2 | 44928 | 44928 | 44928 | 53 | 40070 | 4859 | 8720 | >100 | 53 |
| | s_at | | Kunitz type, 2 | | | | | | | | | | |
| 333 | 218467_{—} | NM_020232.1 | hepatocellular | HCCA3 | 44928 | 44928 | 44928 | 44928 | 53 | 44876 | 149 | 100 | 53 |
| | at | | carcinoma susceptibility | | | | | | | | | | |
| | | | protein | | | | | | | | | | |
| 334 | AFFX- | M97935 | — | — | 44928 | 44928 | 53 | 44928 | 708 | 44221 | 1068 | >100 | 53 |
| | HUMI | | | | | | | | | | | | |
| | SGF3A/ | | | | | | | | | | | | |
| | M97935_{—} | | | | | | | | | | | | |
| | MA_{—} | | | | | | | | | | | | |
| | at | | | | | | | | | | | | |
| 335 | 204227_{—} | NM_004614.1 | thymidine kinase 2, | TK2 | 44928 | 44928 | 44928 | 44928 | 44875 | 54 | 114 | >100 | 54 |
| | s_at | | mitochondrial | | | | | | | | | | |
| 336 | 232138_{—} | AW276914 | Homo sapiens clone | — | 44928 | 44928 | 44928 | 54 | 44534 | 395 | 1280 | >100 | 54 |
| | at | | IMAGE: 713177, mRNA | | | | | | | | | | |
| | | | sequence | | | | | | | | | | |
| 337 | 204517_{—} | BE962749 | peptidylprolyl isomerase | PPIC | 44928 | 44928 | 44928 | 55 | 44402 | 527 | 978 | >100 | 55 |
| | at | | C (cyclophilin C) | | | | | | | | | | |
| 338 | 211275_{—} | AF087942.1 | glycogenin | GYG | 131 | 44928 | 44928 | 44928 | 369 | 44560 | 1427 | 55 | 55 |
| | s_at | | | | | | | | | | | | |
| 339 | 226888_{—} | BG104860 | casein kinase 1, gamma 1 | CSNK1G1 | 44928 | 44928 | 44928 | 44928 | 55 | 44874 | 58 | >100 | 55 |
| | at | | | | | | | | | | | | |
| 340 | AFFX- | M97935 | — | — | 44928 | 44928 | 56 | 44928 | 454 | 44475 | 523 | >100 | 56 |
| | HUMI | | | | | | | | | | | | |
| | SGF3A/ | | | | | | | | | | | | |
| | M97935_{—} | | | | | | | | | | | | |
| | MB_{—} | | | | | | | | | | | | |
| | at | | | | | | | | | | | | |
| 341 | 225373_{—} | BE271644 | PP2135 protein | PP2135 | 44928 | 44928 | 44928 | 56 | 44814 | 115 | 372 | >100 | 56 |
| | at | | | | | | | | | | | | |
| 342 | 205618_{—} | NM_000950.1 | proline-rich Gla (G- | PRRG1 | 44928 | 44928 | 44928 | 44928 | 44872 | 57 | 81 | >100 | 57 |
| | at | | carboxyglutamic acid) | | | | | | | | | | |
| | | | polypeptide 1 | | | | | | | | | | |
| 343 | 200030_{—} | NM_002635.1 | solute carrier family 25 | SLC25A3 | 44928 | 44928 | 44928 | 44928 | 57 | 44872 | 91 | 67 | 57 |
| | s_at | | (mitochondrial carrier; | | | | | | | | | | |
| | | | phosphate carrier), | | | | | | | | | | |
| | | | member 3 | | | | | | | | | | |
| 344 | 228400_{—} | AW025141 | ESTs | — | 57 | 44928 | 44928 | 44928 | 223 | 44706 | 1047 | >100 | 57 |
| | at | | | | | | | | | | | | |
| 345 | 201491_{—} | NM_012111.1 | chromosome 14 open | C14orf3 | 44928 | 44928 | 44928 | 44928 | 58 | 44871 | 107 | >100 | 58 |
| | at | | reading frame 3 | | | | | | | | | | |
| 346 | 209031_{—} | NM_014333.1 | immunoglobulin | IGSF4 | 44928 | 44928 | 58 | 44928 | 2854 | 42075 | 8458 | >100 | 58 |
| | at | | superfamily, member 4 | | | | | | | | | | |
| 347 | 222529_{—} | BG251467 | mitochondrial solute | MSCP | 44928 | 44928 | 44928 | 58 | 27388 | 17541 | 33137 | >100 | 58 |
| | at | | carrier protein | | | | | | | | | | |
| 348 | 244142_{—} | D60329 | ESTs | — | 44928 | 44928 | 44928 | 44928 | 44871 | 58 | 125 | >100 | 58 |
| | at | | | | | | | | | | | | |
| 349 | 226227_{—} | BF185165 | Homo sapiens, clone | — | 73 | 44928 | 44928 | 44928 | 675 | 44254 | 1792 | 59 | 59 |
| | x_at | | IMAGE: 5285034, | | | | | | | | | | |
| | | | mRNA | | | | | | | | | | |
| 350 | 226830_{—} | BG339245 | Homo sapiens cDNA | — | 44928 | 44928 | 44928 | 44928 | 59 | 44870 | 166 | >100 | 59 |
| | x_at | | FLJ14030 fis, clone | | | | | | | | | | |
| | | | HEMBA1004086. | | | | | | | | | | |
| 351 | 233234_{—} | AB037738.1 | KIAA1317 protein | KIAA1317 | 44928 | 44928 | 44928 | 59 | 44197 | 732 | 15108 | >100 | 59 |
| | at | | | | | | | | | | | | |
| 352 | 243147_{—} | AW118707 | ESTs, Weakly similar to | — | 44928 | 44928 | 44928 | 44928 | 44870 | 59 | 68 | >100 | 59 |
| | x_at | | YYY1_HUMAN Very | | | | | | | | | | |
| | | | very hypothetical protein | | | | | | | | | | |
| | | | RMSA-1 [H. sapiens] | | | | | | | | | | |
| 353 | 221458_{—} | NM_000866.1 | 5-hydroxytryptamine | HTR1F | 44928 | 44928 | 44928 | 44928 | 44869 | 60 | 106 | >100 | 60 |
| | at | | (serotonin) receptor 1F | | | | | | | | | | |
| 354 | 225084_{—} | BG170743 | SEC10-like 1 (S. | SEC10L1 | 44928 | 44928 | 122 | 44928 | 69 | 44860 | 141 | 60 | 60 |
| | at | | cerevisiae) | | | | | | | | | | |
| 355 | 227598_{—} | AI762857 | hypothetical protein. | LOC113763 | 44928 | 44928 | 44928 | 44928 | 76 | 44853 | 60 | >100 | 60 |
| | at | | BC011406 | | | | | | | | | | |
| 356 | 235113_{—} | AA742244 | peptidylprolyl isomerase | PPIL5 | 44928 | 44928 | 60 | 44928 | 200 | 44729 | 456 | >100 | 60 |
| | at | | (cyclophilin) like 5 | | | | | | | | | | |
| 357 | 242749_{—} | AI022173 | ESTs | — | 44928 | 44928 | 44928 | 60 | 43605 | 1324 | 4746 | >100 | 60 |
| | at | | | | | | | | | | | | |
| 358 | AFFX- | M10098 | — | — | 44928 | 44928 | 44928 | 61 | 24464 | 20465 | 33430 | >100 | 61 |
| | HUMR | | | | | | | | | | | | |
| | GE/M1 | | | | | | | | | | | | |
| | 0098_{—} | | | | | | | | | | | | |
| | M_at | | | | | | | | | | | | |
| 359 | 225281_{—} | AL117573.1 | DKFZP434F2021 | DKFZP434F2021 | 44928 | 44928 | 44928 | 44928 | 132 | 44797 | 194 | 61 | 61 |
| | at | | protein | 34F2021 | | | | | | | | | |
| 360 | 234942_{—} | AK025220.1 | — | — | 44928 | 44928 | 44928 | 44928 | 61 | 44868 | 248 | >100 | 61 |
| | s_at | | | | | | | | | | | | |
| 361 | 213873_{—} | D29810.1 | endothelial and smooth | ESDN | 44928 | 44928 | 44928 | 44928 | 44867 | 62 | 73 | >100 | 62 |
| | at | | muscle cell-derived | | | | | | | | | | |
| | | | neuropilin-like protein | | | | | | | | | | |
| 362 | 216524_{—} | AL049260.1 | Homo sapiens mRNA; | — | 44928 | 44928 | 44928 | 62 | 44161 | 768 | 1958 | >100 | 62 |
| | x_at | | cDNA DKFZp564E233 | | | | | | | | | | |
| | | | (from clone | | | | | | | | | | |
| | | | DKFZp564E233) | | | | | | | | | | |
| 363 | 231265_{—} | AI126453 | cytochrome c oxidase | COX7B2 | 62 | 44928 | 44928 | 44928 | 2009 | 42920 | 21140 | >100 | 62 |
| | at | | subunit VIIb2 | | | | | | | | | | |
| 364 | 201264_{—} | NM_007263.1 | coatomer protein | COPE | 80 | 44928 | 96 | 44928 | 176 | 44753 | 739 | 63 | 63 |
| | at | | complex, subunit epsilon | | | | | | | | | | |
| 365 | 222510_{—} | AI809203 | makorin, ring finger | MKRN2 | 44928 | 44928 | 44928 | 44928 | 63 | 44866 | 110 | >100 | 63 |
| | s_at | | protein, 2 | | | | | | | | | | |
| 366 | 226179_{—} | N63920 | Homo sapiens, clone | — | 44928 | 44928 | 44928 | 63 | 27539 | 17390 | 31921 | >100 | 63 |
| | at | | IMAGE: 5294823, | | | | | | | | | | |
| | | | mRNA | | | | | | | | | | |
| 367 | 226835_{—} | BG330520 | Homo sapiens, clone | — | 44928 | 44928 | 63 | 44928 | 1324 | 43605 | 4164 | >100 | 63 |
| | s_at | | IMAGE: 5285034, | | | | | | | | | | |
| | | | mRNA | | | | | | | | | | |
| 368 | 228159_{—} | N45312 | Homo sapiens cDNA | — | 44928 | 44928 | 44928 | 44928 | 44866 | 63 | 290 | >100 | 63 |
| | at | | FLJ38039 fis, clone | | | | | | | | | | |
| | | | CTONG2013934. | | | | | | | | | | |
| 369 | 202026_{—} | NM_003002.1 | succinate dehydrogenase | SDHD | 44928 | 44928 | 44928 | 44928 | 64 | 44865 | 189 | >100 | 64 |
| | at | | complex, subunit D, | | | | | | | | | | |
| | | | integral membrane | | | | | | | | | | |
| | | | protein | | | | | | | | | | |
| 370 | 220534_{—} | NM_024114.1 | tripartite motif- | TRIM48 | 44928 | 44928 | 44928 | 44928 | 44865 | 64 | 124 | >100 | 64 |
| | at | | containing 48 | | | | | | | | | | |
| 371 | 239294_{—} | AA810265 | ESTs | — | 64 | 44928 | 44928 | 44928 | 867 | 44062 | 3303 | 82 | 64 |
| | at | | | | | | | | | | | | |
| 372 | 224298_{—} | BC004528 | phosphoglycerate | PHGDH | 65 | 44928 | 44928 | 44928 | 1198 | 43731 | 15433 | >100 | 65 |
| | s_at | | dehydrogenase like 1 | L1 | | | | | | | | | |
| 373 | 224558_{—} | BG483939 | PRO1073 protein | PRO1073 | 44928 | 44928 | 44928 | 65 | 40007 | 4922 | 10881 | >100 | 65 |
| | s_at | | | | | | | | | | | | |
| 374 | 244172_{—} | AA931562 | ESTs, Weakly similar to | — | 44928 | 44928 | 44928 | 85 | 44864 | 65 | 143 | >100 | 65 |
| | at | | hypothetical protein | | | | | | | | | | |
| | | | FLJ20489 [Homo | | | | | | | | | | |
| | | | sapiens] [H. sapiens] | | | | | | | | | | |
| 375 | 205370_{—} | NM_001918.1 | dihydrolipoamide | DBT | 44928 | 44928 | 44928 | 66 | 44434 | 495 | 1851 | >100 | 66 |
| | x_at | | branched chain | | | | | | | | | | |
| | | | transacylase (E2 | | | | | | | | | | |
| | | | component of branched | | | | | | | | | | |
| | | | chain keto acid | | | | | | | | | | |
| | | | dehydrogenase complex; | | | | | | | | | | |
| | | | maple syrup urine | | | | | | | | | | |
| | | | disease) | | | | | | | | | | |
| 376 | 222789_{—} | BE888593 | hypothetical protein | FLJ11220 | 44928 | 44928 | 44928 | 44928 | 66 | 44863 | 76 | >100 | 66 |
| | at | | FLJ11220 | | | | | | | | | | |
| 377 | 226558_{—} | BE856637 | ESTs | — | 66 | 44928 | 44928 | 44928 | 751 | 44178 | 2501 | >100 | 66 |
| | at | | | | | | | | | | | | |
| 378 | 215109_{—} | R02172 | ESTs, Moderately | — | 44928 | 44928 | 44928 | 44928 | 44862 | 67 | 203 | >100 | 67 |
| | at | | similar to hypothetical | | | | | | | | | | |
| | | | protein FLJ20234 | | | | | | | | | | |
| | | | [Homo sapiens] | | | | | | | | | | |
| | | | [H. sapiens] | | | | | | | | | | |
| 379 | 224740_{—} | BE613001 | Homo sapiens, clone | — | 44928 | 44928 | 67 | 44928 | 426 | 44503 | 263 | 70 | 67 |
| | at | | IMAGE: 4620009, | | | | | | | | | | |
| | | | mRNA | | | | | | | | | | |
| 380 | 226265_{—} | AW294894 | hypothetical protein | FLJ21924 | 67 | 44928 | 44928 | 44928 | 145 | 44784 | 397 | >100 | 67 |
| | at | | FLJ21924 | | | | | | | | | | |
| 381 | 217188_{—} | AC007182 | chromosome 14 open | C14orf1 | 68 | 44928 | 44928 | 44928 | 245 | 44684 | 508 | >100 | 68 |
| | s_at | | reading frame 1 | | | | | | | | | | |
| 382 | 229466_{—} | AU144187 | hypothetical protein | LOC256273 | 44928 | 44928 | 44928 | 44928 | 44861 | 68 | 139 | >100 | 68 |
| | at | | LOC256273 | | | | | | | | | | |
| 383 | 242619_{—} | H82831 | ESTs | — | 44928 | 44928 | 44928 | 68 | 44810 | 119 | 408 | >100 | 68 |
| | x_at | | | | | | | | | | | | |
| 384 | 220073_{—} | NM_018173.1 | hypothetical protein | FLJ10665 | 44928 | 44928 | 44928 | 44928 | 44860 | 69 | 361 | >100 | 69 |
| | s_at | | FLJ10665 | | | | | | | | | | |
| 385 | 210092_{—} | AF067173.1 | mago-nashi homolog, | MAGOH | 44928 | 44928 | 44928 | 44928 | 70 | 44859 | 157 | >100 | 70 |
| | at | | proliferation-associated | | | | | | | | | | |
| | | | (Drosophila) | | | | | | | | | | |
| 386 | 213371_{—} | AI803302 | LIM domain binding 3 | LDB3 | 44928 | 44928 | 44928 | 44928 | 44859 | 70 | 132 | >100 | 70 |
| | at | | | | | | | | | | | | |
| 387 | 229655_{—} | N66656 | hypothetical protein | CLONE25003 | 70 | 44928 | 44928 | 44928 | 4007 | 40922 | 24679 | >100 | 70 |
| | at | | CLONE25003 | | | | | | | | | | |
| 388 | 228866_{—} | BF514864 | Homo sapiens cDNA | — | 44928 | 44928 | 44928 | 71 | 43995 | 934 | 494 | >100 | 71 |
| | at | | FLJ13825 fis, clone | | | | | | | | | | |
| | | | THYRO1000558. | | | | | | | | | | |
| 389 | 244795_{—} | AV693986 | ESTs | — | 44928 | 44928 | 44928 | 44928 | 44858 | 71 | 273 | >100 | 71 |
| | at | | | | | | | | | | | | |
| 390 | 204610_{—} | NM_006848.1 | hepatitis delta antigen- | DIPA | 44928 | 44928 | 72 | 44928 | 1914 | 43015 | 8164 | >100 | 72 |
| | s_at | | interacting protein A | | | | | | | | | | |
| 391 | 225218_{—} | AA205754 | hypothetical protein | FLJ32919 | 44928 | 44928 | 44928 | 44928 | 44857 | 72 | 169 | >100 | 72 |
| | at | | FLJ32919 | | | | | | | | | | |
| 392 | 225904_{—} | N64686 | Homo sapiens cDNA | — | 87 | 44928 | 78 | 44928 | 1309 | 43620 | 4215 | 72 | 72 |
| | at | | FLJ25935 fis, clone | | | | | | | | | | |
| | | | JTH06710. | | | | | | | | | | |
| 393 | 206992_{—} | NM_015684.1 | ATP synthase, H+ | ATP5S | 44928 | 44928 | 44928 | 44928 | 73 | 44856 | 145 | >100 | 73 |
| | s_at | | transporting, | | | | | | | | | | |
| | | | mitochondrial F0 | | | | | | | | | | |
| | | | complex, subunit s | | | | | | | | | | |
| | | | (factor B) | | | | | | | | | | |
| 394 | 226944_{—} | AW518728 | serine protease HTRA3 | HTRA3 | 44928 | 44928 | 44928 | 44928 | 44856 | 73 | 196 | >100 | 73 |
| | at | | | | | | | | | | | | |
| 395 | 227084_{—} | AW339310 | dystrobrevin, alpha | DTNA | 44928 | 44928 | 44928 | 73 | 44615 | 314 | 833 | >100 | 73 |
| | at | | | | | | | | | | | | |
| 396 | 209703_{—} | BC004492.1 | DKFZP586A0522 | DKFZP586A0522 | 44928 | 44928 | 44928 | 74 | 42035 | 2894 | 1118 | >100 | 74 |
| | x_at | | protein | | | | | | | | | | |
| 397 | 210154_{—} | M55905.1 | malic enzyme 2, | ME2 | 44928 | 44928 | 44928 | 44928 | 74 | 44855 | 98 | >100 | 74 |
| | at | | NAD(+)-dependent, | | | | | | | | | | |
| | | | mitochondrial | | | | | | | | | | |
| 398 | 226050_{—} | AL576117 | chromosome 13 open | C13orf11 | 74 | 44928 | 44928 | 44928 | 1168 | 43761 | 5900 | >100 | 74 |
| | at | | reading frame 11 | | | | | | | | | | |
| 399 | 209340_{—} | S73498.1 | UDP-N- | UAP1 | 124 | 44928 | 75 | 44928 | 2926 | 42003 | 12143 | 79 | 75 |
| | at | | acteylglucosamine | | | | | | | | | | |
| | | | pyrophosphorylase 1 | | | | | | | | | | |
| 400 | 215504_{—} | AF131777.1 | Homo sapiens clone | — | 44928 | 44928 | 44928 | 75 | 44199 | 730 | 1434 | >100 | 75 |
| | x_at | | 25061 mRNA sequence | | | | | | | | | | |
| 401 | 219878_{—} | NM_015995.1 | Kruppel-like factor 13 | KLF13 | 44928 | 44928 | 44928 | 44928 | 75 | 44854 | 175 | >100 | 75 |
| | s_at | | | | | | | | | | | | |
| 402 | 221978_{—} | BE138825 | major histocompatibility | HLA-F | 44928 | 44928 | 44928 | 44928 | 44854 | 75 | 176 | >100 | 75 |
| | at | | complex, class I, F | | | | | | | | | | |
| 403 | 226051_{—} | BF973568 | selenoprotein SelM | SELM | 44928 | 44928 | 44928 | 76 | 43355 | 1574 | 2394 | >100 | 76 |
| | at | | | | | | | | | | | | |
| 404 | 208690_{—} | BC000915.1 | PDZ and LIM domain 1 | PDLIM1 | 77 | 44928 | 124 | 44928 | 1120 | 43809 | 3441 | >100 | 77 |
| | s_at | | (elfin) | | | | | | | | | | |
| 405 | 213738_{—} | AI587323 | ATP synthase, H+ transporting, | ATP5A1 | 44928 | 44928 | 44928 | 44928 | 77 | 44852 | 191 | >100 | 77 |
| | s_at | | mitochondrial F1 | | | | | | | | | | |
| | | | complex, alpha subunit, | | | | | | | | | | |
| | | | isoform 1, cardiac | | | | | | | | | | |
| | | | muscle | | | | | | | | | | |
| 406 | 226276_{—} | BF439522 | hypothetical protein | LOC153339 | 44928 | 44928 | 77 | 44928 | 781 | 44148 | 909 | >100 | 77 |
| | at | | LOC153339 | | | | | | | | | | |
| 407 | 39313_{—} | AB002342 | protein kinase, lysine | PRKWNK1 | 44928 | 44928 | 44928 | 44928 | 44850 | 79 | 343 | >100 | 79 |
| | at | | deficient 1 | | | | | | | | | | |
| 408 | 222109_{—} | AA558583 | hypothetical protein | FLJ10613 | 44928 | 44928 | 44928 | 79 | 44834 | 95 | 310 | >100 | 79 |
| | at | | FLJ10613 | | | | | | | | | | |
| 409 | 211474_{—} | BC004948.1 | serine (or cysteine) | SERPINB6 | 44928 | 44928 | 44928 | 80 | 44692 | 237 | 648 | >100 | 80 |
| | s_at | | proteinase inhibitor, | | | | | | | | | | |
| | | | clade B (ovalbumin), | | | | | | | | | | |
| | | | member 6 | | | | | | | | | | |
| 410 | 224915_{—} | AV756131 | Homo sapiens, clone | — | 89 | 44928 | 44928 | 44928 | 726 | 44203 | 1875 | 80 | 80 |
| | x_at | | IMAGE: 5285034, | | | | | | | | | | |
| | | | mRNA | | | | | | | | | | |
| 411 | 215528_{—} | AL049390.1 | Homo sapiens mRNA; | — | 44928 | 44928 | 44928 | 44928 | 44848 | 81 | 223 | >100 | 81 |
| | at | | cDNA | | | | | | | | | | |
| | | | DKFZp586O1318 (from | | | | | | | | | | |
| | | | clone | | | | | | | | | | |
| | | | DKFZp586O1318) | | | | | | | | | | |
| 412 | 222428_{—} | D84223.1 | leucyl-tRNA synthetase | LARS | 44928 | 44928 | 81 | 44928 | 598 | 44331 | 1689 | >100 | 81 |
| | s_at | | | | | | | | | | | | |
| 413 | 232369_{—} | AF339768.1 | Homo sapiens clone | — | 44928 | 44928 | 44928 | 81 | 44430 | 499 | 864 | >100 | 81 |
| | at | | IMAGE: 119716, mRNA | | | | | | | | | | |
| | | | sequence | | | | | | | | | | |
| 414 | 233849_{—} | AK023014.1 | Rho GTPase activating | ARHGAP5 | 81 | 44928 | 44928 | 44928 | 577 | 44352 | 1929 | >100 | 81 |
| | s_at | | protein 5 | | | | | | | | | | |
| 415 | 204173_{—} | NM_002475.1 | myosin light chain 1 | MLC1SA | 44928 | 44928 | 44928 | 44928 | 82 | 44847 | 146 | >100 | 82 |
| | at | | slow a | | | | | | | | | | |
| 416 | 213632_{—} | M94065.1 | dihydroorotate | DHODH | 44928 | 44928 | 44928 | 44928 | 44847 | 82 | 155 | >100 | 82 |
| | at | | dehydrogenase | | | | | | | | | | |
| 417 | 225086_{—} | BF679966 | hypothetical protein | FLJ38426 | 83 | 44928 | 123 | 44928 | 408 | 44521 | 610 | >100 | 83 |
| | at | | FLJ38426 | | | | | | | | | | |
| 418 | 225468_{—} | AI761804 | tripartite motif- | TRIM14 | 44928 | 44928 | 44928 | 44928 | 83 | 44846 | 136 | >100 | 83 |
| | at | | containing 14 | | | | | | | | | | |
| 419 | 236617_{—} | AW663083 | Homo sapiens, clone | — | 44928 | 44928 | 44928 | 83 | 44770 | 159 | 217 | >100 | 83 |
| | at | | IMAGE: 5285945, | | | | | | | | | | |
| | | | mRNA | | | | | | | | | | |
| 420 | 210453_{—} | AL050277.1 | ATP synthase, H+ | ATP5L | 84 | 44928 | 44928 | 44928 | 531 | 44398 | 1585 | >100 | 84 |
| | x_at | | transporting, | | | | | | | | | | |
| | | | mitochondrial F0 | | | | | | | | | | |
| | | | complex, subunit g | | | | | | | | | | |
| 421 | 216977_{—} | AJ130972.1 | small nuclear | SNRPA1 | 44928 | 44928 | 44928 | 44928 | 84 | 44845 | 187 | >100 | 84 |
| | x_at | | ribonucleoprotein | | | | | | | | | | |
| | | | polypeptide A′ | | | | | | | | | | |
| 422 | 237475_{—} | AI151104 | selenoprotein P, plasma, 1 | SEPP1 | 44928 | 44928 | 44928 | 84 | 43126 | 1803 | 2926 | >100 | 84 |
| | x_at | | | | | | | | | | | | |
| 423 | 211794_{—} | AF198052.1 | FYN binding protein | FYB | 44928 | 44928 | 44928 | 44928 | 44160 | 769 | 85 | >100 | 85 |
| | at | | (FYB-120/130) | | | | | | | | | | |
| 424 | 201892_{—} | NM_000884.1 | IMP (inosine | IMPDH2 | 86 | 44928 | 44928 | 44928 | 3337 | 41592 | 14262 | >100 | 86 |
| | s_at | | monophosphate) | | | | | | | | | | |
| | | | dehydrogenase 2 | | | | | | | | | | |
| 425 | 218901_{—} | NM_020353.1 | phospholipid scramblase 4 | PLSCR4 | 44928 | 44928 | 44928 | 44928 | 44843 | 86 | 121 | >100 | 86 |
| | at | | | | | | | | | | | | |
| 426 | 241997_{—} | AA700817 | ESTs, Weakly similar to | — | 44928 | 44928 | 44928 | 86 | 42689 | 2240 | 6135 | >100 | 86 |
| | at | | hypothetical protein | | | | | | | | | | |
| | | | FLJ20234 [Homo | | | | | | | | | | |
| | | | sapiens] [H. sapiens] | | | | | | | | | | |
| 427 | 208463_{—} | NM_000809.1 | gamma-aminobutyric | GABRA4 | 44928 | 44928 | 44928 | 87 | 44731 | 198 | 377 | >100 | 87 |
| | at | | acid (GABA) A | | | | | | | | | | |
| | | | receptor, alpha 4 | | | | | | | | | | |
| 428 | 220071_{—} | NM_018097.1 | hypothetical protein | FLJ10460 | 44928 | 44928 | 44928 | 91 | 44842 | 87 | 322 | >100 | 87 |
| | x_at | | FLJ10460 | | | | | | | | | | |
| 429 | 222646_{—} | AW268365 | ERO1-like (S. | ERO1L | 44928 | 44928 | 44928 | 44928 | 87 | 44842 | 150 | >100 | 87 |
| | s_at | | cerevisiae) | | | | | | | | | | |
| 430 | 234875_{—} | AJ224082 | — | — | 44928 | 44928 | 87 | 44928 | 845 | 44084 | 2407 | >100 | 87 |
| | at | | | | | | | | | | | | |
| 431 | 207300_{—} | NM_000131.2 | coagulation factor VII | F7 | 44928 | 44928 | 44928 | 44928 | 44782 | 147 | 88 | >100 | 88 |
| | s_at | | (serum prothrombin | | | | | | | | | | |
| | | | conversion accelerator) | | | | | | | | | | |
| 432 | 209083_{—} | U34690.1 | coronin, actin binding | CORO1A | 88 | 44928 | 44928 | 44928 | 7864 | 37065 | 30105 | >100 | 88 |
| | at | | protein, 1A | | | | | | | | | | |
| 433 | 216644_{—} | AK000185.1 | Homo sapiens cDNA | — | 44928 | 44928 | 44928 | 44928 | 44841 | 88 | 270 | >100 | 88 |
| | at | | FLJ20178 fis, clone | | | | | | | | | | |
| | | | COL09990. | | | | | | | | | | |
| 434 | 218920_{—} | NM_019057.1 | hypothetical protein | FLJ10404 | 44928 | 44928 | 44928 | 88 | 44757 | 172 | 446 | >100 | 88 |
| | at | | FLJ10404 | | | | | | | | | | |
| 435 | 224518_{—} | BC006436.1 | hypothetical protein | MGC13105 | 44928 | 44928 | 88 | 44928 | 450 | 44479 | 1018 | >100 | 88 |
| | s_at | | MGC13105 | | | | | | | | | | |
| 436 | 227916_{—} | AA747303 | exosome component | RRP40 | 44928 | 44928 | 44928 | 44928 | 88 | 44841 | 227 | >100 | 88 |
| | x_at | | Rrp40 | | | | | | | | | | |
| 437 | 202232_{—} | NM_006360.1 | dendritic cell protein | GA17 | 44928 | 44928 | 44928 | 44928 | 89 | 44840 | 254 | >100 | 89 |
| | s_at | | | | | | | | | | | | |
| 438 | 215916_{—} | AL157418.1 | misshapen/NIK-related | MINK | 44928 | 44928 | 44928 | 44928 | 44840 | 89 | 402 | >100 | 89 |
| | at | | kinase | | | | | | | | | | |
| 439 | 228818_{—} | BF110792 | Homo sapiens cDNA | — | 44928 | 44928 | 44928 | 89 | 43849 | 1080 | 3023 | >100 | 89 |
| | at | | FLJ12727 fis, clone | | | | | | | | | | |
| | | | NT2RP2000027. | | | | | | | | | | |
| 440 | 200903_{—} | NM_000687.1 | S-adenosylhomocysteine | AHCY | 44928 | 44928 | 90 | 44928 | 142 | 44787 | 237 | 97 | 90 |
| | s_at | | hydrolase | | | | | | | | | | |
| 441 | 206790_{—} | NM_004545.1 | NADH dehydrogenase | NDUFB1 | 126 | 44928 | 92 | 44928 | 352 | 44577 | 1766 | 90 | 90 |
| | s_at | | (ubiquinone) 1 beta | | | | | | | | | | |
| | | | subcomplex, 1, 7 kDa | | | | | | | | | | |
| 442 | 208013_{—} | NM_020115.1 | acrosomal vesicle | ACRV1 | 44928 | 44928 | 44928 | 44928 | 44839 | 90 | 162 | >100 | 90 |
| | s_at | | protein 1 | | | | | | | | | | |
| 443 | 224254_{—} | AF116695.1 | — | — | 44928 | 44928 | 44928 | 90 | 42695 | 2234 | 2842 | >100 | 90 |
| | x_at | | | | | | | | | | | | |
| 444 | 201825_{—} | AL572542 | CGI-49 protein | CGI-49 | 91 | 44928 | 44928 | 44928 | 921 | 44008 | 4114 | >100 | 91 |
| | s_at | | | | | | | | | | | | |
| 445 | 204795_{—} | NM_025263.1 | CAT56 protein | CAT56 | 44928 | 44928 | 44928 | 44928 | 91 | 44838 | 256 | >100 | 91 |
| | at | | | | | | | | | | | | |
| 446 | 218332_{—} | NM_018476.1 | brain expressed, X- | BEX1 | 44928 | 44928 | 44928 | 44928 | 44838 | 91 | 201 | >100 | 91 |
| | at | | linked 1 | | | | | | | | | | |
| 447 | 222975_{—} | AB020692.1 | NRAS-related gene | D1S155E | 44928 | 44928 | 113 | 44928 | 119 | 44810 | 177 | 91 | 91 |
| | s_at | | | | | | | | | | | | |
| 448 | 215806_{—} | M13231.1 | T cell receptor gamma | TRGC2 | 44928 | 44928 | 44928 | 44928 | 44837 | 92 | 321 | >100 | 92 |
| | x_at | | constant 2 | | | | | | | | | | |
| 449 | 200037_{—} | NM_016587.1 | chromobox homolog 3 | CBX3 | 44928 | 44928 | 135 | 44928 | 233 | 44696 | 448 | 92 | 92 |
| | s_at | | (HP1 gamma homolog, | | | | | | | | | | |
| | | | Drosophila) | | | | | | | | | | |
| 450 | 225892_{—} | BF438417 | Homo sapiens mRNA; | — | 44928 | 44928 | 108 | 44928 | 92 | 44837 | 164 | >100 | 92 |
| | at | | cDNA | | | | | | | | | | |
| | | | DKFZp564D1164 (from | | | | | | | | | | |
| | | | clone | | | | | | | | | | |
| | | | DKFZp564D1164) | | | | | | | | | | |
| 451 | 209786_{—} | BC001282.1 | high mobility group | HMGN4 | 44928 | 44928 | 44928 | 44928 | 267 | 44662 | 484 | 93 | 93 |
| | at | | nucleosomal binding | | | | | | | | | | |
| | | | domain 4 | | | | | | | | | | |
| 452 | 215056_{—} | AI267546 | ESTs | — | 44928 | 44928 | 44928 | 44928 | 44836 | 93 | 160 | >100 | 93 |
| | at | | | | | | | | | | | | |
| 453 | 223433_{—} | AF226046.1 | GK003 protein | GK003 | 44928 | 44928 | 44928 | 44928 | 93 | 44836 | 122 | >100 | 93 |
| | at | | | | | | | | | | | | |
| 454 | 225304_{—} | BE741920 | NADH-ubiquinone | NDUFA11 | 44928 | 44928 | 152 | 44928 | 146 | 44783 | 93 | >100 | 93 |
| | s_at | | oxidoreductase subunit | | | | | | | | | | |
| | | | B14.7 | | | | | | | | | | |
| 455 | 234462_{—} | S51397 | — | — | 93 | 44928 | 44928 | 44928 | 4340 | 40589 | 28484 | >100 | 93 |
| | at | | | | | | | | | | | | |
| 456 | 205119_{—} | NM_002029.1 | formyl peptide receptor 1 | FPR1 | 44928 | 44928 | 44928 | 44928 | 44835 | 94 | 257 | >100 | 94 |
| | s_at | | | | | | | | | | | | |
| 457 | 224872_{—} | AB040896.1 | KIAA1463 protein | KIAA1463 | 44928 | 44928 | 44928 | 44928 | 94 | 44835 | 451 | >100 | 94 |
| | at | | | | | | | | | | | | |
| 458 | 224952_{—} | BF115054 | putative ankyrin-repeat | DKFZP564D166 | 44928 | 44928 | 44928 | 94 | 43286 | 1643 | 7694 | >100 | 94 |
| | at | | containing protein | | | | | | | | | | |
| 459 | 226756_{—} | AA191741 | Homo sapiens cDNA | — | 94 | 44928 | 44928 | 44928 | 776 | 44153 | 2397 | >100 | 94 |
| | at | | FLJ11436 fis, clone | | | | | | | | | | |
| | | | HEMBA 1001213. | | | | | | | | | | |
| 460 | 202250_{—} | NM_015726.1 | H326 | H326 | 44928 | 44928 | 44928 | 95 | 42923 | 2006 | 6207 | >100 | 95 |
| | s_at | | | | | | | | | | | | |
| 461 | 223334_{—} | AL136941.1 | hypothetical protein | DKFZp586C1924 | 44928 | 44928 | 95 | 44928 | 240 | 44689 | 704 | >100 | 95 |
| | at | | DKFZp586C1924 | | | | | | | | | | |
| 462 | 226789_{—} | W84421 | Human S6 H-8 mRNA | — | 95 | 44928 | 44928 | 44928 | 2994 | 41935 | 15082 | >100 | 95 |
| | at | | expressed in | | | | | | | | | | |
| | | | chromosome 6- | | | | | | | | | | |
| | | | suppressed melanoma | | | | | | | | | | |
| | | | cells. | | | | | | | | | | |
| 463 | 208742_{—} | U78303.1 | sin3-associated | SAP18 | 44928 | 44928 | 44928 | 44928 | 242 | 44687 | 599 | 96 | 96 |
| | s_at | | polypeptide, 18 kDa | | | | | | | | | | |
| 464 | 231810_{—} | BG106919 | BRI3 binding protein | BRI3BP | 96 | 44928 | 44928 | 44928 | 929 | 44000 | 3396 | >100 | 96 |
| | at | | | | | | | | | | | | |
| 465 | 244495_{—} | AL521157 | hypothetical protein | MGC11386 | 44928 | 44928 | 44928 | 96 | 41892 | 3037 | 4559 | >100 | 96 |
| | x_at | | MGC11386 | | | | | | | | | | |
| 466 | 205260_{—} | NM_001107.1 | acylphosphatase 1, | ACYP1 | 44928 | 44928 | 44928 | 44928 | 136 | 44793 | 97 | >100 | 97 |
| | s_at | | erythrocyte (common) | | | | | | | | | | |
| | | | type | | | | | | | | | | |
| 467 | 213746_{—} | AW051856 | filamin A, alpha (actin | FLNA | 97 | 44928 | 44928 | 44928 | 4383 | 40546 | 25901 | >100 | 97 |
| | s_at | | binding protein 280) | | | | | | | | | | |
| 468 | 215601_{—} | AK023895.1 | — | — | 44928 | 44928 | 44928 | 44928 | 44832 | 97 | 932 | >100 | 97 |
| | at | | | | | | | | | | | | |
| 469 | 202565_{—} | NM_003174.2 | supervillin | SVIL | 98 | 44928 | 44928 | 44928 | 8543 | 36386 | 44011 | >100 | 98 |
| | s_at | | | | | | | | | | | | |
| 470 | 209596_{—} | AF245505.1 | adlican | DKFZp564I1922 | 44928 | 44928 | 44928 | 44928 | 44831 | 98 | 239 | >100 | 98 |
| | at | | | | | | | | | | | | |
| 471 | 225470_{—} | AL529634 | mitotic phosphoprotein44 | LOC129401 | 44928 | 44928 | 44928 | 44928 | 98 | 44831 | 265 | >100 | 98 |
| | at | | | | | | | | | | | | |
| 472 | 243450_{—} | T40707 | ESTs | — | 44928 | 44928 | 44928 | 98 | 36175 | 8754 | 15508 | >100 | 98 |
| | at | | | | | | | | | | | | |
| 473 | 209036_{—} | BC001917.1 | malate dehydrogenase 2, | MDH2 | 44928 | 44928 | 44928 | 44928 | 100 | 44829 | 258 | >100 | 100 |
| | s_at | | NAD (mitochondrial) | | | | | | | | | | |
| 474 | 216380_{—} | AC005011 | — | — | 100 | 44928 | 131 | 44928 | 1371 | 43558 | 4699 | >100 | 100 |
| | x_at | | | | | | | | | | | | |
| 475 | 236646_{—} | BE301029 | hypothetical protein | FLJ31166 | 44928 | 44928 | 44928 | 100 | 40827 | 4102 | 1539 | >100 | 100 |
| | at | | FLJ31166 | | | | | | | | | | |

A Cox proportional hazard analysis was performed to determine predictors of time until disease progression (TTP) in patients with relapsed and refractory multiple myeloma after treatment with bortezomib. This methodology is designed to analyze time to event data where some of the data may be censored (see E. T. Lee, Statistical Methods for Survival Data Analysis, 2^{nd} ed. 1992, John Wiley & Sons, Inc.). The statistical package SAS was used to perform the analysis. We first examined clinical and prognostic factors to identify which combination of factors showed the greatest association with TTP. This was accomplished by use of the score method for best subset selection. This method provides score chi-squared statistics for all possible model sizes ranging from one predictor to the total number of explanatory variables under consideration. Thus, the method first provides the best single predictor models in order of the highest chi-squared statistics. If there are significant single predictor models (p<0.05), the procedure goes on to the next step of estimating all two predictor models and ranking them by the highest chi-squared statistic.

To assess if a 2 predictor model is a better fit than a single predictor model, the difference in the chi-squared statistics is calculated. This is a one degree of freedom chi-square test and can be assessed for statistical significance. If the difference proves to be significant at p<0.05, we conclude the two predictor model is a better fit, the second variable is significantly associated with TTP after taking into account the first variable, and the process continues by estimating all three predictor models. The three predictor model is compared to the two predictor model in the same way as the two predictor model was assessed against the single predictor model. This process is continued until the difference chi-square test fails, that is p>0.05 for adding in an additional variable to the model. By using this process, we found that the best model contained 3 significant prognostic or clinical factors, abnormal cytogentics, β2-microglobulin, and c-reactive protein. We defined this as our best prognostic variable model.

The next step was to determine if there were any genomic markers that were significantly associated with TTP after accounting for the prognostic factors. We first filtered the genomic data set, made up of some 44,000 transcripts from the Affymetrics U133A and U133B human array chips, to those genes which had at least one present call using the Affymetrix detection system for determining if a transcript is reliably detected or not. This left 13,529 transcripts for analysis. We then estimated Cox proportional hazard models for each of the 13,529 transcripts where each model also contained the 3 prognostic factors discussed above. That is, 13,529 models were estimated where each model contained 1 transcript and the three prognostic factors. From each model, we obtained estimates of relative risk, 95% confidence intervals and p values for the association of each transcript to TTP. From the 13,529 models, we found 834 transcripts which had p values of less than 0.05. That is, we found 834 transcripts that were significantly and independently, from the prognostic factors, associated with TTP. These are listed in Table 2

TABLE 2
Predictive markers Associated with Time to Disease Progression (TTP)

| | | Seq. Derived | | | |
|---|---|---|---|---|---|
| | | From | | | |
| | | (RefSeq/ | | | |
| | | Genbank | | Gene | Haz- |
| No. | Probe set ID | Accession) | Title | Symbol | ard |
| 83 | 201575_at | NM_012245.1 | SKI-interacting protein | SNW1 | >1 |
| 81 | 202647_s_at | NM_002524.2 | neuroblastoma RAS viral (v-ras) oncogene homolog | NRAS | >1 |
| 234 | 203058_s_at | AW299958 | 3′-phosphoadenosine 5′-phosphosulfate synthase 2 | PAPSS2 | <1 |
| 42 | 203753_at | NM_003199.1 | transcription factor 4 | TCF4 | <1 |
| 415 | 204173_at | NM_002475.1 | myosin light chain 1 slow a | MLC1SA | >1 |
| 191 | 206121_at | NM_000036.1 | adenosine monophosphate deaminase 1 (isoform M) | AMPD1 | >1 |
| 404 | 208690_s_at | BC000915.1 | PDZ and LIM domain 1 (elfin) | PDLIM1 | >1 |
| 53 | 210993_s_at | U54826.1 | MAD, mothers against decapentaplegic homolog 1 (Drosophila) | MADH1 | >1 |
| 305 | 212110_at | D31887.1 | KIAA0062 protein | KIAA0062 | <1 |
| 41 | 212382_at | AK021980.1 | Homo sapiens cDNA FLJ11918 fis, clone HEMBB1000272. | — | <1 |
| 43 | 212386_at | AK021980.1 | Homo sapiens cDNA FLJ11918 fis, clone HEMBB1000272. | — | <1 |
| 40 | 212387_at | AK021980.1 | Homo sapiens cDNA FLJ11918 fis, clone HEMBB1000272. | — | <1 |
| 467 | 213746_s_at | AW051856 | filamin A, alpha (actin binding protein 280) | FLNA | >1 |
| 39 | 213891_s_at | AI927067 | Homo sapiens cDNA FLJ11918 fis, clone HEMBB1000272. | — | <1 |
| 78 | 215744_at | AW514140 | fusion, derived from t(12; 16) malignant liposarcoma | FUS | <1 |
| 77 | 218319_at | NM_020651.2 | pellino homolog 1 (Drosophila) | PELI1 | <1 |
| 201 | 219429_at | NM_024306.1 | fatty acid hydroxylase | FAAH | <1 |
| 126 | 222762_x_at | AU144259 | LIM domains containing 1 | LIMD1 | >1 |
| 376 | 222789_at | BE888593 | hypothetical protein FLJ11220 | FLJ11220 | >1 |
| 341 | 225373_at | BE271644 | PP2135 protein | PP2135 | <1 |
| 209 | 225710_at | H99792 | Homo sapiens cDNA FLJ34013 fis, clone FCBBF2002111. | — | <1 |
| 48 | 227798_at | AU146891 | EST | — | >1 |
| 464 | 231810_at | BG106919 | BRI3 binding protein | BRI3BP | >1 |
| 76 | 232213_at | AU147506 | pellino homolog 1 (Drosophila) | PELI1 | <1 |
| 75 | 232304_at | AK026714.1 | pellino homolog 1 (Drosophila) | PELI1 | <1 |
| 224 | 235875_at | BF510711 | EST | — | <1 |
| 172 | 242903_at | AI458949 | EST | — | <1 |
| 476 | 222788_s_at | BE888593 | hypothetical protein FLJ11220 | FLJ11220 | >1 |
| 477 | 213305_s_at | L42375.1 | protein phosphatase 2, regulatory subunit B (B56), gamma isoform | PPP2R5C | >1 |
| 478 | 204774_at | NM_014210.1 | ecotropic viral integration site 2A | EVI2A | <1 |
| 479 | 200984_s_at | NM_000611.1 | CD59 antigen p18-20 (antigen identified by monoclonal antibodies 16.3A5, | CD59 | <1 |
| | | | EJ16, EJ30, EL32 and G344) | | |
| 480 | 208956_x_at | U62891.1 | dUTP pyrophosphatase | DUT | >1 |
| 481 | 216326_s_at | AF059650 | histone deacetylase 3 | HDAC3 | <1 |
| 482 | 203845_at | AV727449 | p300/CBP-associated factor | PCAF | <1 |
| 483 | 214349_at | AV764378 | Homo sapiens cDNA: FLJ23438 fis, clone HRC13275. | — | >1 |
| 484 | 202332_at | NM_001894.1 | casein kinase 1, epsilon | CSNK1E | >1 |
| 485 | 201020_at | NM_003405.1 | tyrosine 3-monooxygenase tryptophan 5-monooxygenase activation protein, | YWHAH | <1 |
| | | | eta polypeptide | | |
| 486 | 200612_s_at | NM_001282.1 | adaptor-related protein complex 2, beta 1 subunit | AP2B1 | <1 |
| 487 | 212612_at | D31888.1 | REST corepressor | RCOR | >1 |
| 488 | 202963_at | AW027312 | regulatory factor X, 5 (influences HLA class II expression) | RFX5 | <1 |
| 489 | 212463_at | BE379006 | Homo sapiens mRNA; cDNA DKFZp564J0323 (from clone | — | <1 |
| | | | DKFZp564J0323) | | |
| 490 | 202453_s_at | NM_005316.1 | general transcription factor IIH, polypeptide 1, 62 kDa | GTF2H1 | <1 |
| 491 | 209239_at | M55643.1 | nuclear factor of kappa light polypeptide gene enhancer in B-cells 1 (p105) | NFKB1 | <1 |
| 492 | 213405_at | N95443 | Homo sapiens, clone IMAGE: 4831050, mRNA | — | <1 |
| 493 | 200679_x_at | BE311760 | high-mobility group box 1 | HMGB1 | >1 |
| 494 | 205981_s_at | NM_001564.1 | inhibitor of growth family, member 1-like | ING1L | >1 |
| 495 | 211783_s_at | BC006177.1 | metastasis associated 1 | MTA1 | >1 |
| 496 | 227482_at | AI097656 | hypothetical protein LOC57143 | LOC57143 | >1 |
| 497 | 214943_s_at | D38491.1 | KIAA0117 protein | KIAA0117 | >1 |
| 498 | 205504_at | NM_000061.1 | Bruton agammaglobulinemia tyrosine kinase | BTK | <1 |
| 499 | 218216_x_at | NM_016638.1 | ADP-ribosylation-like factor 6 interacting protein 4 | ARL6IP4 | >1 |
| 500 | 221014_s_at | NM_031296.1 | RAB33B, member RAS oncogene family | RAB33B | <1 |
| 501 | 202408_s_at | NM_015629.1 | PRP31 pre-mRNA processing factor 31 homolog (yeast) | PRPF31 | >1 |
| 502 | 217996_at | AA576961 | pleckstrin homology-like domain, family A, member 1 | PHLDA1 | >1 |
| 503 | 229723_at | BF591040 | T-cell activation GTPase activating protein | TAGAP | <1 |
| 504 | 227112_at | AW270037 | KIAA0779 protein | KIAA0779 | <1 |
| 505 | 218224_at | NM_006029.2 | paraneoplastic antigen MA1 | PNMA1 | >1 |
| 506 | 213415_at | AI768628 | chloride intracellular channel 2 | CLIC2 | <1 |
| 507 | 225251_at | AK021761.1 | Homo sapiens cDNA FLJ11699 fis, clone HEMBA1005047, highly similar to | RAB24 | <1 |
| | | | RAS-RELATED PROTEIN RAB-24. | | |
| 508 | 219228_at | NM_018555.2 | zinc finger protein 463 | ZNF463 | <1 |
| 509 | 226979_at | AI125541 | mitogen-activated protein kinase kinase kinase 2 | MAP3K2 | <1 |
| 510 | 227179_at | AK002152.1 | staufen, RNA binding protein, homolog 2 (Drosophila) | STAU2 | >1 |
| 511 | 205621_at | NM_006020.1 | alkB, alkylation repair homolog (E. coli) | ALKBH | >1 |
| 512 | 226421_at | AA707320 | hypothetical protein LOC286505 | LOC286505 | <1 |
| 513 | 219709_x_at | NM_023933.1 | hypothetical protein MGC2494 | MGC2494 | >1 |
| 514 | 217803_at | NM_022130.1 | golgi phosphoprotein 3 (coat-protein) | GOLPH3 | <1 |
| 515 | 228980_at | AI760772 | fring | LOC117584 | <1 |
| 516 | 243020_at | R06738 | EST | — | >1 |
| 517 | 211289_x_at | AF067524.1 | cell division cycle 2-like 2 | CDC2L2 | >1 |
| 518 | 213137_s_at | AI828880 | protein tyrosine phosphatase, non-receptor type 2 | PTPN2 | >1 |
| 519 | 204407_at | AF080255.1 | transcription termination factor, RNA polymerase II | TTF2 | >1 |
| 520 | 224938_at | AU144387 | EST | — | <1 |
| 521 | 225466_at | AI761804 | tripartite motif-containing 14 | TRIM14 | <1 |
| 522 | 208908_s_at | AF327443.1 | calpastatin | CAST | <1 |
| 523 | 222343_at | AA629050 | Homo sapiens full length insert cDNA clone ZA94C02 | — | >1 |
| 524 | 224566_at | AK027191.1 | Homo sapiens cDNA: FLJ23538 fis, clone LNG08010, highly similar to | — | <1 |
| | | | BETA2 Human MEN1 region clone epsilon/beta mRNA. | | |
| 525 | 208297_s_at | NM_005665.1 | — | — | >1 |
| 526 | 213923_at | AW005535 | RAP2B, member of RAS oncogene family | RAP2B | <1 |
| 527 | 228680_at | AW340096 | EST, Moderately similar to hypothetical protein FLJ20489 [Homo sapiens] | — | <1 |
| | | | [H. sapiens] | | |
| 528 | 209204_at | AI824831 | LIM domain only 4 | LMO4 | >1 |
| 529 | 208093_s_at | NM_030808.1 | LIS1-interacting protein NUDEL; endooligopeptidase A | NUDEL | <1 |
| 530 | 200982_s_at | NM_001155.2 | annexin A6 | ANXA6 | <1 |
| 531 | 218249_at | NM_022494.1 | zinc finger, DHHC domain containing 6 | ZDHHC6 | <1 |
| 532 | 203345_s_at | AI566096 | likely ortholog of mouse metal response element binding transcription factor 2 | M96 | >1 |
| 533 | 223141_at | AK022317.1 | uridine-cytidine kinase 1 | UCK1 | >1 |
| 534 | 222444_at | AL121883 | ALEX3 protein | ALEX3 | <1 |
| 535 | 217853_at | NM_022748.1 | tumor endothelial marker 6 | TEM6 | <1 |
| 536 | 220244_at | NM_013343.1 | NAG-7 protein | NAG-7 | <1 |
| 537 | 213995_at | AW195882 | ATP synthase, H+ transporting, mitochondrial F0 complex, subunits (factor | ATP5S | >1 |
| | | | B) | | |
| 538 | 214072_x_at | AA679297 | secreted protein of unknown function | SPUF | >1 |
| 539 | 200950_at | NM_006409.1 | actin related protein 2/3 complex, subunit 1A, 41 kDa | ARPC1A | <1 |
| 540 | 224878_at | N63936 | similar to ubiquitin binding protein | UBPH | >1 |
| 541 | 227294_at | AI474448 | hypothetical protein BC014000 | LOC115509 | >1 |
| 542 | 214334_x_at | N34846 | DAZ associated protein 2 | DAZAP2 | >1 |
| 543 | 214659_x_at | AC007956 | ZAP3 protein | ZAP3 | >1 |
| 544 | 36499_at | D87469 | cadherin, EGF LAG seven-pass G-type receptor 2 (flamingo homolog, | CELSR2 | >1 |
| | | | Drosophila) | | |
| 545 | 229512_at | BE464337 | EST | — | >1 |
| 546 | 206662_at | NM_002064.1 | glutaredoxin (thioltransferase) | GLRX | <1 |
| 547 | 200914_x_at | BF589024 | kinectin 1 (kinesin receptor) | KTN1 | >1 |
| 548 | 214938_x_at | AF283771.2 | high-mobility group box 1 | HMGB1 | >1 |
| 549 | 203243_s_at | NM_006457.1 | LIM protein (similar to rat protein kinase C-binding enigma) | LIM | <1 |
| 550 | 214395_x_at | AI335509 | eukaryotic translation elongation factor 1 delta (guanine nucleotide exchange | EEF1D | >1 |
| | | | protein) | | |
| 551 | 217208_s_at | AL121981 | discs, large (Drosophila) homolog 1 | DLG1 | >1 |
| 552 | 224180_x_at | AF131737.1 | hypothetical protein LOC51057 | LOC51057 | >1 |
| 553 | 218724_s_at | NM_021809.1 | TGFB-induced factor 2 (TALE family homeobox) | TGIF2 | <1 |
| 554 | 210387_at | BC001131.1 | histone 1, H2bg | HIST1H2BG | >1 |
| 555 | 208898_at | AF077614.1 | ATPase, H+ transporting, lysosomal 34 kDa, V1 subunit D | ATP6V1D | >1 |
| 556 | 200645_at | NM_007278.1 | GABA(A) receptor-associated protein | GABARAP | <1 |
| 557 | 200985_s_at | NM_000611.1 | CD59 antigen p18-20 (antigen identified by monoclonal antibodies 16.3A5, | CD59 | <1 |
| | | | EJ16, EJ30, EL32 and G344) | | |
| 558 | 220595_at | NM_013377.1 | hypothetical protein DKFZp434B0417 | DKFZp434B0417 | >1 |
| 559 | 236550_s_at | BF508689 | Homo sapiens mRNA; cDNA DKFZp686I2118 (from clone | ZNF311 | >1 |
| | | | DKFZp686I2118) | | |
| 560 | 202279_at | NM_004894.1 | chromosome 14 open reading frame 2 | C14orf2 | >1 |
| 561 | 234312_s_at | AK000162.1 | acetyl-Coenzyme A synthetase 2 (ADP forming) | ACAS2 | >1 |
| 562 | 213945_s_at | AI867102 | nucleoporin 210 | NUP210 | >1 |
| 563 | 228380_at | BE551193 | EST, Weakly similar to hypothetical protein FLJ20378 [Homo sapiens] | — | <1 |
| | | | [H. sapiens] | | |
| 564 | 203574_at | NM_005384.1 | nuclear factor, interleukin 3 regulated | NFIL3 | >1 |
| 565 | 222146_s_at | AK026674.1 | transcription factor 4 | TCF4 | <1 |
| 566 | 227665_at | BE968576 | Homo sapiens, clone IMAGE: 4152387, mRNA | — | <1 |
| 567 | 207995_s_at | NM_014257.1 | CD209 antigen-like | CD209L | <1 |
| 568 | 201097_s_at | NM_001660.2 | ADP-ribosylation factor 4 | ARF4 | <1 |
| 569 | 203975_s_at | BF000239 | chromatin assembly factor 1, subunit A (p150) | CHAF1A | >1 |
| 570 | 209136_s_at | BG390445 | ubiquitin specific protease 10 | USP10 | >1 |
| 571 | 238086_at | AI288372 | EST | — | >1 |
| 572 | 242388_x_at | AW576600 | EST | — | <1 |
| 573 | 241876_at | AW663060 | EST | — | <1 |
| 574 | 228195_at | BE645119 | EST | — | <1 |
| 575 | 202334_s_at | AA877765 | ubiquitin-conjugating enzyme E2B (RAD6 homolog) | UBE2B | <1 |
| 576 | 201472_at | NM_003372.2 | von Hippel-Lindau binding protein 1 | VBP1 | <1 |
| 577 | 217092_x_at | AL031589 | — | — | >1 |
| 578 | 208744_x_at | BG403660 | heat shock 105 kDa/110 kDa protein 1 | HSPH1 | >1 |
| 579 | 212412_at | AV715767 | Homo sapiens mRNA; cDNA DKFZp564A072 (from clone | — | <1 |
| | | | DKFZp564A072) | | |
| 580 | 217995_at | NM_021199.1 | sulfide quinone reductase-like (yeast) | SQRDL | <1 |
| 581 | 203275_at | NM_002199.2 | interferon regulatory factor 2 | IRF2 | <1 |
| 582 | 207335_x_at | NM_007100.1 | ATP synthase, H+ transporting, mitochondrial F0 complex, subunit e | ATP5I | >1 |
| 583 | 218130_at | NM_024510.1 | hypothetical protein MGC4368 | MGC4368 | >1 |
| 584 | 208914_at | NM_015044.1 | golgi associated, gamma adaptin ear containing, ARF binding protein 2 | GGA2 | <1 |
| 585 | 202985_s_at | NM_004873.1 | BCL2-associated athanogene 5 | BAG5 | >1 |
| 586 | 206587_at | NM_006584.1 | chaperonin containing TCP1, subunit 6B (zeta 2) | CCT6B | <1 |
| 587 | 223419_at | BC004290.1 | hypothetical protein MGC10870 | MGC10870 | >1 |
| 588 | 213102_at | Z78330 | ARP3 actin-related protein 3 homolog (yeast) | ACTR3 | <1 |
| 589 | 226520_at | AI831506 | EST | — | <1 |
| 590 | 201366_at | NM_004034.1 | annexin A7 | ANXA7 | <1 |
| 591 | 213021_at | AI741876 | Homo sapiens mRNA; cDNA DKFZp566B213 (from clone DKFZp566B213) | — | <1 |
| 592 | 201172_x_at | NM_003945.1 | ATPase, H+ transporting, lysosomal 9 kDa, V0 subunit e | ATP6V0E | <1 |
| 593 | 213295_at | AA555096 | Homo sapiens mRNA; cDNA DKFZp586D1122 (from clone | — | <1 |
| | | | DKFZp586D1122) | | |
| 594 | 226406_at | AI823360 | hypothetical protein MGC12909 | MGC12909 | <1 |
| 595 | 210564_x_at | AF009619.1 | CASP8 and FADD-like apoptosis regulator | CFLAR | <1 |
| 596 | 242606_at | AL043482 | EST | — | <1 |
| 597 | 203292_s_at | NM_021729.2 | vacuolar protein sorting 11 (yeast) | VPS11 | >1 |
| 598 | 202579_x_at | NM_006353.1 | high mobility group nucleosomal binding domain 4 | HMGN4 | <1 |
| 599 | 229113_s_at | W16779 | protein kinase C, zeta | PRKCZ | >1 |
| 600 | 244743_x_at | AA114243 | zinc finger protein 138 (clone pHZ-32) | ZNF138 | <1 |
| 601 | 222622_at | BG284709 | hypothetical protein LOC283871 | LOC283871 | >1 |
| 602 | 210312_s_at | BC002640.1 | hypothetical protein LOC90410 | LOC90410 | <1 |
| 603 | 221530_s_at | AB044088.1 | basic helix-loop-helix domain containing, class B, 3 | BHLHB3 | <1 |
| 604 | 201994_at | NM_012286.1 | mortality factor 4 like 2 | MORF4L2 | <1 |
| 605 | 227262_at | BE348293 | Homo sapiens proteoglycan link protein mRNA, complete cds. | — | >1 |
| 606 | 203693_s_at | NM_001949.2 | E2F transcription factor 3 | E2F3 | <1 |
| 607 | 221750_at | BG035985 | 3-hydroxy-3-methylglutaryl-Coenzyme A synthase 1 (soluble) | HMGCS1 | <1 |
| 608 | 214789_x_at | AA524274 | Splicing factor, arginine/serine-rich, 46 kD | SRP46 | <1 |
| 609 | 200761_s_at | NM_006407.2 | vitamin A responsive; cytoskeleton related | JWA | <1 |
| 610 | 212233_at | AL523076 | Homo sapiens cDNA FLJ30550 fis, clone BRAWH2001502. | — | <1 |
| 611 | 209300_s_at | BC002888.1 | DKFZP566B183 protein | DKFZP566B183 | <1 |
| 612 | 213708_s_at | N40555 | transcription factor-like 4 | TCFL4 | <1 |
| 613 | 207467_x_at | NM_001750.2 | calpastatin | CAST | <1 |
| 614 | 225414_at | AL558987 | hypothetical protein LOC284996 | LOC284996 | <1 |
| 615 | 235104_at | BG292389 | EST | — | <1 |
| 616 | 214003_x_at | BF184532 | ribosomal protein S20 | RPS20 | >1 |
| 617 | 201542_at | AY008268.1 | SAR1 protein | SAR1 | <1 |
| 618 | 211316_x_at | AF009616.1 | CASP8 and FADD-like apoptosis regulator | CFLAR | <1 |
| 619 | 221522_at | AL136784.1 | hypothetical protein DKFZp434L0718 | DKFZP434L0718 | <1 |
| 620 | 210844_x_at | D14705.1 | catenin (cadherin-associated protein), alpha 1, 102 kDa | CTNNA1 | <1 |
| 621 | 210448_s_at | U49396.1 | purinergic receptor P2X, ligand-gated ion channel, 5 | P2RX5 | <1 |
| 622 | 212843_at | AA126505 | neural cell adhesion molecule 1 | NCAM1 | <1 |
| 623 | 224284_x_at | AF338193.1 | — | — | >1 |
| 624 | 222650_s_at | BE898559 | SLC2A4 regulator | SLC2A4RG | >1 |
| 625 | 212719_at | AB011178.1 | pleckstrin homology domain containing, family E (with leucine rich repeats) | PLEKHE1 | >1 |
| | | | member 1 | | |
| 626 | 38069_at | Z67743 | chloride channel 7 | CLCN7 | >1 |
| 627 | 233625_x_at | AK021939.1 | hypothetical protein FLJ20542 | FLJ20542 | >1 |
| 628 | 205053_at | NM_000946.1 | primase, polypeptide 1, 49 kDa | PRIM1 | >1 |
| 629 | 239749_at | AW205090 | EST | — | >1 |
| 630 | 34764_at | D21851 | leucyl-tRNA synthetase, mitochondrial | LARS2 | >1 |
| 631 | 205659_at | NM_014707.1 | histone deacetylase 9 | HDAC9 | <1 |
| 632 | 242092_at | AA019300 | EST, Moderately similar to hypothetical protein FLJ20097 [Homo sapiens] | — | >1 |
| | | | [H. sapiens] | | |
| 633 | 203575_at | NM_001896.1 | casein kinase 2, alpha prime polypeptide | CSNK2A2 | >1 |
| 634 | 221297_at | NM_018654.1 | G protein-coupled receptor, family C, group 5, member D | GPRC5D | <1 |
| 635 | 212900_at | BE645231 | SEC24 related gene family, member A (S. cerevisiae) | SEC24A | <1 |
| 636 | 230036_at | BE669858 | hypothetical protein FLJ39885 | FLJ39885 | <1 |
| 637 | 213101_s_at | Z78330 | ARP3 actin-related protein 3 homolog (yeast) | ACTR3 | <1 |
| 638 | 222846_at | AB038995.1 | RAB-8b protein | LOC51762 | <1 |
| 639 | 213455_at | W87466 | pleckstrin homology domain containing, family B (evectins) member 2 | PLEKHB2 | <1 |
| 640 | 242613_at | AI809536 | EST | — | >1 |
| 641 | 218206_x_at | NM_016558.1 | SCAN domain containing 1 | SCAND1 | >1 |
| 642 | 222014_x_at | AI249752 | MTO1 protein | MTO1 | <1 |
| 643 | 212219_at | D38521.1 | proteasome activator 200 kDa | PA200 | <1 |
| 644 | 219806_s_at | NM_020179.1 | FN5 protein | FN5 | <1 |
| 645 | 218875_s_at | NM_012177.1 | F-box only protein 5 | FBXO5 | >1 |
| 646 | 208485_x_at | NM_003879.1 | CASP8 and FADD-like apoptosis regulator | CFLAR | <1 |
| 647 | 218233_s_at | NM_017601.1 | chromosome 6 open reading frame 49 | C6orf49 | >1 |
| 648 | 214130_s_at | AI821791 | phosphodiesterase 4D interacting protein (myomegalin) | PDE4DIP | <1 |
| 649 | 208723_at | BC000350.1 | ubiquitin specific protease 11 | USP11 | >1 |
| 650 | 217814_at | NM_020198.1 | GK001 protein | GK001 | <1 |
| 651 | 208809_s_at | AL136632.1 | hypothetical protein FLJ12619 | FLJ12619 | >1 |
| 652 | 201199_s_at | NM_002807.1 | proteasome (prosome, macropain) 26S subunit, non-ATPase, 1 | PSMD1 | <1 |
| 653 | 242937_at | AV763408 | EST, Moderately similar to ILF1_HUMAN Interleukin enhancer-binding | — | >1 |
| | | | factor 1 (Cellular transcription factor ILF-1) [H. sapiens] | | |
| 654 | 212333_at | AL049943.1 | DKFZP564F0522 protein | DKFZP564F0522 | <1 |
| 655 | 210817_s_at | BC004130.1 | nuclear domain 10 protein | NDP52 | <1 |
| 656 | 212508_at | AK024029.1 | modulator of apoptosis 1 | MOAP1 | >1 |
| 657 | 213603_s_at | BE138888 | ras-related C3 botulinum toxin substrate 2 (rho family, small GTP binding | RAC2 | <1 |
| | | | protein Rac2) | | |
| 658 | 233274_at | AU145144 | — | — | >1 |
| 659 | 218557_at | NM_020202.1 | Nit protein 2 | NIT2 | <1 |
| 660 | 231428_at | BE502947 | EST | — | <1 |
| 661 | 201810_s_at | AL562152 | SH3-domain binding protein 5 (BTK-associated) | SH3BP5 | <1 |
| 662 | 209970_x_at | M87507.1 | caspase 1, apoptosis-related cysteine protease (interleukin 1, beta, convertase) | CASP1 | <1 |
| 663 | 208965_s_at | BG256677 | interferon, gamma-inducible protein 16 | IFI16 | >1 |
| 664 | 203038_at | NM_002844.1 | protein tyrosine phosphatase, receptor type, K | PTPRK | <1 |
| 665 | 202442_at | NM_001284.1 | adaptor-related protein complex 3, sigma 1 subunit | AP3S1 | <1 |
| 666 | 209515_s_at | U38654.3 | RAB27A, member RAS oncogene family | RAB27A | <1 |
| 667 | 201865_x_at | AI432196 | nuclear receptor subfamily 3, group C, member 1 (glucocorticoid receptor) | NR3C1 | <1 |
| 668 | 204786_s_at | L41944.1 | interferon (alpha, beta and omega) receptor 2 | IFNAR2 | >1 |
| 669 | 209508_x_at | AF005774.1 | CASP8 and FADD-like apoptosis regulator | CFLAR | <1 |
| 670 | 200822_x_at | NM_000365.1 | triosephosphate isomerase 1 | TPI1 | >1 |
| 671 | 217322_x_at | AL024509 | — | — | >1 |
| 672 | 203505_at | AF285167.1 | ATP-binding cassette, sub-family A (ABC1), member 1 | ABCA1 | >1 |
| 673 | 223347_at | AL360266.1 | hypothetical protein FLJ22283 | FLJ22283 | >1 |
| 674 | 209765_at | Y13786.2 | a disintegrin and metalloproteinase domain 19 (meltrin beta) | ADAM19 | <1 |
| 675 | 202972_s_at | AW450403 | family with sequence similarity 13, member A1 | FAM13A1 | >1 |
| 676 | 203380_x_at | NM_006925.1 | splicing factor, arginine/serine-rich 5 | SFRS5 | >1 |
| 677 | 212211_at | AI986295 | gene trap ankyrin repeat | GTAR | <1 |
| 678 | 218326_s_at | NM_018490.1 | G protein-coupled receptor 48 | GPR48 | >1 |
| 679 | 217994_x_at | NM_017871.1 | hypothetical protein FLJ20542 | FLJ20542 | >1 |
| 680 | 239835_at | AA669114 | T-cell activation kelch repeat protein | TA-KRP | <1 |
| 681 | 213353_at | BF693921 | ATP-binding cassette, sub-family A (ABC1), member 5 | ABCA5 | <1 |
| 682 | 208710_s_at | AI424923 | adaptor-related protein complex 3, delta 1 subunit | AP3D1 | >1 |
| 683 | 205011_at | NM_014622.1 | loss of heterozygosity, 11, chromosomal region 2, gene A | LOH11CR2A | <1 |
| 684 | 202027_at | NM_012264.1 | chromosome 22 open reading frame 5 | C22orf5 | >1 |
| 685 | 203642_s_at | NM_014900.1 | KIAA0977 protein | KIAA0977 | <1 |
| 686 | 212266_s_at | AW084582 | splicing factor, arginine/serine-rich 5 | SFRS5 | >1 |
| 687 | 238693_at | AA165136 | EST | — | <1 |
| 688 | 219342_at | NM_022900.1 | O-acetyltransferase | CAS1 | <1 |
| 689 | 201769_at | NM_014666.1 | enthoprotin | ENTH | <1 |
| 690 | 243982_at | AA455180 | EST, Weakly similar to KHLX_HUMAN Kelch-like protein X [H. sapiens] | — | >1 |
| 691 | 230490_x_at | AI866717 | hypothetical protein FLJ31034 | FLJ31034 | <1 |
| 692 | 227073_at | N50665 | Homo sapiens cDNA FLJ36574 fis, clone TRACH2012376. | — | <1 |
| 693 | 226858_at | T51255 | chromosome 1 open reading frame 28 | C1orf28 | >1 |
| 694 | 219759_at | NM_022350.1 | aminopeptidase | LOC64167 | <1 |
| 695 | 208325_s_at | NM_006738.1 | A kinase (PRKA) anchor protein 13 | AKAP13 | >1 |
| 696 | 212053_at | AK025504.1 | KIAA0251 protein | KIAA0251 | <1 |
| 697 | 222715_s_at | BE856321 | AP1 gamma subunit binding protein 1 | AP1GBP1 | <1 |
| 698 | 235456_at | AI810266 | Homo sapiens, clone IMAGE: 4819084, mRNA | — | >1 |
| 699 | 235424_at | N66727 | EST | — | <1 |
| 700 | 212407_at | AL049669.1 | CGI-01 protein | CGI-01 | <1 |
| 701 | 227565_at | BE501881 | EST | — | <1 |
| 702 | 228091_at | AI800609 | EST, Weakly similar to D29149 proline-rich protein —mouse (fragment) | — | >1 |
| | | | [M. musculus] | | |
| 703 | 209258_s_at | NM_005445.1 | chondroitin sulfate proteoglycan 6 (bamacan) | CSPG6 | >1 |
| 704 | 222590_s_at | AF180819.1 | nemo-like kinase | NLK | <1 |
| 705 | 212528_at | AL023553 | Homo sapiens, clone IMAGE: 3605655, mRNA | — | <1 |
| 706 | 203981_s_at | AL574660 | ATP-binding cassette, sub-family D (ALD), member 4 | ABCD4 | >1 |
| 707 | 201011_at | NM_002950.1 | ribophorin I | RPN1 | <1 |
| 708 | 244268_x_at | BF435769 | EST, Weakly similar to hypothetical protein FLJ20378 [Homo sapiens] | — | <1 |
| | | | [H. sapiens] | | |
| 709 | 202315_s_at | NM_004327.2 | breakpoint cluster region | BCR | <1 |
| 710 | 227698_s_at | AW007215 | RAB40C, member RAS oncogene family | RAB40C | >1 |
| 711 | 218311_at | NM_003618.1 | mitogen-activated protein kinase kinase kinase kinase 3 | MAP4K3 | <1 |
| 712 | 213931_at | AI819238 | inhibitor of DNA binding 2, dominant negative helix-loop-helix protein | ID2 | >1 |
| 713 | 217997_at | AA576961 | pleckstrin homology-like domain, family A, member 1 | PHLDA1 | >1 |
| 714 | 208951_at | BC002515.1 | aldehyde dehydrogenase 7 family, member A1 | ALDH7A1 | >1 |
| 715 | 225847_at | AB037784.1 | KIAA1363 protein | KIAA1363 | <1 |
| 716 | 202846_s_at | NM_002642.1 | phosphatidylinositol glycan, class C | PIGC | <1 |
| 717 | 200681_at | NM_006708.1 | glyoxalase I | GLO1 | <1 |
| 718 | 202727_s_at | NM_000416.1 | interferon gamma receptor 1 | IFNGR1 | <1 |
| 719 | 222231_s_at | AK025328.1 | hypothetical protein PRO1855 | PRO1855 | <1 |
| 720 | 228482_at | AV702789 | hypothetical protein FLJ36674 | FLJ36674 | >1 |
| 721 | 235056_at | AV722693 | EST | — | <1 |
| 722 | 202010_s_at | NM_021188.1 | likely ortholog of mouse another partner for ARF 1 | APA1 | >1 |
| 723 | 226556_at | BF431260 | Homo sapiens, clone IMAGE: 4815204, mRNA | — | <1 |
| 724 | 215088_s_at | BG110532 | EST, Highly similar to succinate dehydrogenase complex, subunit C | — | >1 |
| | | | precursor; Succinate dehydrogenase complex, subunit C, integral membrane | | |
| | | | protein,; succinate-ubiquinone oxidoreducatase cytochrome B large subunit | | |
| | | | [Homo sapiens] [H. sapiens] | | |
| 725 | 209492_x_at | BC003679.1 | ATP synthase, H+ transporting, mitochondrial F0 complex, subunit e | ATP5I | >1 |
| 726 | 211075_s_at | Z25521.1 | CD47 antigen (Rh-related antigen, integrin-associated signal transducer) | CD47 | <1 |
| 727 | 204552_at | AA355179 | Homo sapiens cDNA FLJ34214 fis, clone FCBBF3021807. | — | <1 |
| 728 | 211862_x_at | AF015451.1 | CASP8 and FADD-like apoptosis regulator | CFLAR | <1 |
| 729 | 201403_s_at | NM_004528.1 | microsomal glutathione S-transferase 3 | MGST3 | <1 |
| 730 | 209899_s_at | AF217197.1 | fuse-binding protein-interacting repressor | SIAHBP1 | >1 |
| 731 | 219023_at | NM_018569.1 | hypothetical protein PRO0971 | PRO0971 | >1 |
| 732 | 236506_at | BF507371 | EST | — | >1 |
| 733 | 205191_at | NM_006915.1 | retinitis pigmentosa 2 (X-linked recessive) | RP2 | <1 |
| 734 | 202146_at | AA747426 | interferon-related developmental regulator 1 | IFRD1 | <1 |
| 735 | 243304_at | AI733824 | hypothetical protein LOC286109 | LOC286109 | >1 |
| 736 | 223658_at | AF134149.1 | potassium channel, subfamily K, member 6 | KCNK6 | <1 |
| 737 | 202074_s_at | NM_021980.1 | optineurin | OPTN | <1 |
| 738 | 203162_s_at | NM_005886.1 | katanin p80 (WD40-containing) subunit B 1 | KATNB1 | >1 |
| 739 | 208841_s_at | AB014560.1 | Ras-GTPase activating protein SH3 domain-binding protein 2 | G3BP2 | <1 |
| 740 | 230128_at | AK025231.1 | Homo sapiens cDNA: FLJ21578 fis, clone COL06726. | — | <1 |
| 741 | 214394_x_at | AI613383 | eukaryotic translation elongation factor 1 delta (guanine nucleotide exchange | EEF1D | >1 |
| | | | protein) | | |
| 742 | 242969_at | AI288679 | EST | — | <1 |
| 743 | 210251_s_at | AF112221.1 | rap2 interacting protein x | RIPX | >1 |
| 744 | 209894_at | U50748.1 | leptin receptor | LEPR | <1 |
| 745 | 204190_at | NM_005800.1 | highly charged protein | D13S106E | >1 |
| 746 | 202438_x_at | BF346014 | Homo sapiens, clone IMAGE: 5278680, mRNA | — | <1 |
| 747 | 211968_s_at | NM_005348.1 | heat shock 90 kDa protein 1, alpha | HSPCA | >1 |
| 748 | 222424_s_at | BC000805.1 | similar to rat nuclear ubiquitous casein kinase 2 | NUCKS | >1 |
| 749 | 226445_s_at | AI743109 | tripartite motif-containing 41 | TRIM41 | >1 |
| 750 | 235061_at | AV706522 | hypothetical protein DKFZp761G058 | DKFZp761G058 | <1 |
| 751 | 34031_i_at | U90268 | cerebral cavernous malformations 1 | CCM1 | <1 |
| 752 | 213160_at | D86964.1 | dedicator of cyto-kinesis 2 | DOCK2 | <1 |
| 753 | 209194_at | BC005334.1 | centrin, EF-hand protein, 2 | CETN2 | <1 |
| 754 | 209240_at | AF070560.1 | O-linked N-acetylglucosamine (GlcNAc) transferase (UDP-N- | OGT | <1 |
| | | | acetylglucosamine: polypeptide-N-acetylglucosaminyl transferase) | | |
| 755 | 218962_s_at | NM_022484.1 | hypothetical protein FLJ13576 | FLJ13576 | <1 |
| 756 | 203525_s_at | AI375486 | adenomatosis polyposis coli | APC | <1 |
| 757 | 219904_at | NM_024303.1 | hypothetical protein MGC4161 | MGC4161 | >1 |
| 758 | 205550_s_at | NM_004899.1 | brain and reproductive organ-expressed (TNFRSF1A modulator) | BRE | <1 |
| 759 | 209932_s_at | U90223.1 | dUTP pyrophosphatase | DUT | >1 |
| 760 | AFFX- | M27830 | — | — | >1 |
| | M27830_M_{—} | | | | |
| | at | | | | |
| 761 | 205297_s_at | NM_000626.1 | CD79B antigen (immunoglobulin-associated beta) | CD79B | <1 |
| 762 | 232297_at | AL049385.1 | Homo sapiens mRNA; cDNA DKFZp586M1418 (from clone | — | <1 |
| | | | DKFZp586M1418) | | |
| 763 | 204019_s_at | NM_015677.1 | likely ortholog of mouse Sh3 domain YSC-like 1 | SH3YL1 | <1 |
| 764 | 230769_at | AI916261 | EST, Weakly similar to PRP1_HUMAN Salivary proline-rich protein | — | >1 |
| | | | precursor (Clones CP3, CP4 and CP5) [Contains: Basic peptide IB-6; Peptide | | |
| | | | P-H] [H. sapiens] | | |
| 765 | 217501_at | AI339732 | Homo sapiens, clone IMAGE: 5268928, mRNA | — | <1 |
| 766 | 205105_at | NM_002372.1 | mannosidase, alpha, class 2A, member 1 | MAN2A1 | <1 |
| 767 | 209514_s_at | BE502030 | RAB27A, member RAS oncogene family | RAB27A | <1 |
| 768 | 203217_s_at | NM_003896.1 | sialyltransferase 9 (CMP-NeuAc: lactosylceramide alpha-2,3-sialyltransferase; | SIAT9 | <1 |
| | | | GM3 synthase) | | |
| 769 | 203176_s_at | BE552470 | transcription factor A, mitochondrial | TFAM | >1 |
| 770 | 208988_at | AK024505.1 | F-box and leucine-rich repeat protein 11 | FBXL11 | <1 |
| 771 | 221500_s_at | AF008936.1 | aminopeptidase-like 1 | NPEPL1 | >1 |
| 772 | 229236_s_at | AI346445 | eukaryotic translation initiation factor 3, subunit 10 theta, 150/170 kDa | EIF3S10 | <1 |
| 773 | 218267_at | NM_016550.1 | cyclin-dependent kinase 2-interacting protein | CINP | >1 |
| 774 | 208129_x_at | NM_001754.1 | runt-related transcription factor 1 (acute myeloid leukemia 1; aml1 oncogene) | RUNX1 | >1 |
| 775 | 208764_s_at | D13119.1 | ATP synthase, H+ transporting, mitochondrial F0 complex, subunit c (subunit | ATP5G2 | >1 |
| | | | 9), isoform 2 | | |
| 776 | 225498_at | AV713673 | chromosome 20 open reading frame 178 | C20orf178 | <1 |
| 777 | 211317_s_at | AF041461.1 | CASP8 and FADD-like apoptosis regulator | CFLAR | <1 |
| 778 | 200760_s_at | N92494 | vitamin A responsive; cytoskeleton related | JWA | <1 |
| 779 | 215483_at | AK000270.1 | A kinase (PRKA) anchor protein (yotiao) 9 | AKAP9 | <1 |
| 780 | 218194_at | NM_015523.1 | small fragment nuclease | DKFZP566E144 | <1 |
| 781 | 201388_at | NM_002809.1 | proteasome (prosome, macropain) 26S subunit, non-ATPase, 3 | PSMD3 | <1 |
| 782 | 34406_at | AB011174 | KIAA0602 protein | KIAA0602 | >1 |
| 783 | 208386_x_at | NM_007068.1 | DMC1 dosage suppressor of mck1 homolog, meiosis-specific homologous | DMC1 | >1 |
| | | | recombination (yeast) | | |
| 784 | 244481_at | BF196523 | EST | — | >1 |
| 785 | 239673_at | AW080999 | EST | — | <1 |
| 786 | 208773_s_at | AL136943.1 | FLJ20288 protein | FLJ20288 | <1 |
| 787 | 222206_s_at | AA781143 | hypothetical protein from EUROIMAGE 2021883 | LOC56926 | >1 |
| 788 | 228658_at | R54042 | Homo sapiens cDNA FLJ25887 fis, clone CBR02996. | — | <1 |
| 789 | 212586_at | BG111635 | type 1 tumor necrosis factor receptor shedding aminopeptidase regulator | ARTS-1 | <1 |
| 790 | 238011_at | BF668314 | Homo sapiens cDNA FLJ37032 fis, clone BRACE2011265. | — | >1 |
| 791 | 204659_s_at | AF124604.1 | growth factor, augmenter of liver regeneration (ERV1 homolog, S. cerevisiae) | GFER | >1 |
| 792 | 200096_s_at | AI862255 | ATPase, H+ transporting, lysosomal 9 kDa, V0 subunit e | ATP6V0E | <1 |
| 793 | 227293_at | AI264003 | Homo sapiens cDNA FLJ34052 fis, clone FCBBF3000175. | — | <1 |
| 794 | 228454_at | AW663968 | KIAA1795 protein | MLR2 | <1 |
| 795 | 209576_at | AL049933.1 | guanine nucleotide binding protein (G protein), alpha inhibiting activity | GNAI1 | <1 |
| | | | polypeptide 1 | | |
| 796 | 201684_s_at | BE783632 | chromosome 14 open reading frame 92 | C14orf92 | >1 |
| 797 | 233068_at | AK023264.1 | EST, Weakly similar to POL2_MOUSE Retrovirus-related POL polyprotein | — | <1 |
| | | | [Contains: Reverse transcriptase; Endonuclease] [M. musculus] | | |
| 798 | 210532_s_at | AF116639.1 | chromosome 14 open reading frame 2 | C14orf2 | >1 |
| 799 | 211911_x_at | L07950.1 | major histocompatibility complex, class I, B | HLA-B | <1 |
| 800 | 208991_at | AA634272 | Homo sapiens cDNA FLJ35646 fis, clone SPLEN2012743. | — | <1 |
| 801 | 226612_at | AW572911 | Homo sapiens cDNA FLJ25076 fis, clone CBL06117. | — | <1 |
| 802 | 223068_at | AV707345 | echinoderm microtubule associated protein like 4 | EML4 | <1 |
| 803 | 227462_at | BE889628 | EST | — | <1 |
| 804 | 224680_at | AL539253 | Homo sapiens, clone IMAGE: 3866125, mRNA | — | <1 |
| 805 | 244075_at | BF224218 | EST | — | >1 |
| 806 | 228220_at | AI627666 | hypothetical protein BC014311 | LOC115548 | <1 |
| 807 | 225729_at | AI870857 | Homo sapiens cDNA: FLJ21560 fis, clone COL06410. | — | <1 |
| 808 | 222771_s_at | NM_016132.1 | myelin gene expression factor 2 | MEF-2 | <1 |
| 809 | 209944_at | BC000330.1 | likely ortholog of mouse another partner for ARF 1 | APA1 | >1 |
| 810 | 224565_at | AK027191.1 | Homo sapiens cDNA: FLJ23538 fis, clone LNG08010, highly similar to | — | <1 |
| | | | BETA2 Human MEN1 region clone epsilon/beta mRNA. | | |
| 811 | 202439_s_at | NM_000202.2 | iduronate 2-sulfatase (Hunter syndrome) | IDS | <1 |
| 812 | 212051_at | AK026913.1 | Homo sapiens cDNA FLJ30463 fis, clone BRACE2009517. | — | <1 |
| 813 | 211969_at | NM_005348.1 | heat shock 90 kDa protein 1, alpha | HSPCA | >1 |
| 814 | 218209_s_at | NM_018170.1 | hypothetical protein FLJ10656 | P15RS | <1 |
| 815 | 208877_at | AF092132.1 | Homo sapiens, clone IMAGE: 6058556, mRNA | — | <1 |
| 816 | 202043_s_at | NM_004595.1 | spermine synthase | SMS | <1 |
| 817 | 209092_s_at | AF061730.1 | CGI-150 protein | CGI-150 | <1 |
| 818 | 225412_at | AA761169 | hypothetical protein FLJ14681 | FLJ14681 | <1 |
| 819 | 201173_x_at | NM_006600.1 | nuclear distribution gene C homolog (A. nidulans) | NUDC | >1 |
| 820 | 201409_s_at | NM_002709.1 | protein phosphatase 1, catalytic subunit, beta isoform | PPP1CB | <1 |
| 821 | 235594_at | AL542578 | EST, Weakly similar to cytokine receptor-like factor 2; cytokine receptor | — | >1 |
| | | | CRL2 precusor [Homo sapiens] [H. sapiens] | | |
| 822 | 218269_at | NM_013235.1 | putative ribonuclease III | RNASE3L | >1 |
| 823 | 213892_s_at | AA927724 | adenine phosphoribosyltransferase | APRT | >1 |
| 824 | 209715_at | L07515.1 | chromobox homolog 5 (HP1 alpha homolog, Drosophila) | CBX5 | >1 |
| 825 | 215001_s_at | AL161952.1 | glutamate-ammonia ligase (glutamine synthase) | GLUL | <1 |
| 826 | 230011_at | AW195720 | hypothetical protein MGC40042 | MGC40042 | <1 |
| 827 | 202623_at | NM_018453.1 | chromosome 14 open reading frame 11 | C14orf11 | >1 |
| 828 | 226749_at | AL582429 | Homo sapiens, clone IMAGE: 4791565, mRNA | — | <1 |
| 829 | 209337_at | AF063020.1 | PC4 and SFRS1 interacting protein 2 | PSIP2 | <1 |
| 830 | 216526_x_at | AK024836.1 | major histocompatibility complex, class I, C | HLA-C | <1 |
| 831 | 212428_at | AB002366.1 | KIAA0368 protein | KIAA0368 | <1 |
| 832 | 222035_s_at | AI984479 | poly(A) polymerase alpha | PAPOLA | >1 |
| 833 | 223277_at | BC000623.1 | hypothetical protein FLJ20211 | FLJ20211 | >1 |
| 834 | 212807_s_at | BE742268 | sortilin 1 | SORT1 | >1 |
| 835 | 212193_s_at | BE881529 | likely ortholog of mouse la related protein | LARP | <1 |
| 836 | 238642_at | AW367571 | Homo sapiens full length insert cDNA clone YB31A06 | — | >1 |
| 837 | 216607_s_at | U40053 | — | — | <1 |
| 838 | 224851_at | AW274756 | Homo sapiens cDNA FLJ31360 fis, clone MESAN2000572. | — | <1 |
| 839 | 53202_at | AA402435 | hypothetical protein MGC2821 | MGC2821 | <1 |
| 840 | 224435_at | BC005871.1 | hypothetical protein MGC4248 | MGC4248 | <1 |
| 841 | 200953_s_at | NM_001759.1 | cyclin D2 | CCND2 | <1 |
| 842 | 240237_at | H23230 | EST, Moderately similar to hypothetical protein FLJ20489 [Homo sapiens] | — | <1 |
| | | | [H. sapiens] | | |
| 843 | 227801_at | N90779 | EST, Weakly similar to hypothetical protein FLJ20378 [Homo sapiens] | — | <1 |
| | | | [H. sapiens] | | |
| 844 | 243217_at | AI681312 | EST | — | <1 |
| 845 | 217742_s_at | NM_016628.1 | WW domain-containing adapter with a coiled-coil region | WAC | <1 |
| 846 | 206472_s_at | NM_005078.1 | transducin-like enhancer of split 3 (E(sp1) homolog, Drosophila) | TLE3 | <1 |
| 847 | 219100_at | NM_024928.1 | hypothetical protein FLJ22559 | FLJ22559 | <1 |
| 848 | 41856_at | AL049370 | Homo sapiens mRNA; cDNA DKFZp586D0918 (from clone | — | >1 |
| | | | DKFZp586D0918) | | |
| 849 | 211921_x_at | AF348514.1 | prothymosin, alpha (gene sequence 28) | PTMA | >1 |
| 850 | 220597_s_at | NM_018694.1 | ADP-ribosylation-like factor 6 interacting protein 4 | ARL6IP4 | >1 |
| 851 | 202461_at | NM_014239.1 | eukaryotic translation initiation factor 2B, subunit 2 beta, 39 kDa | EIF2B2 | >1 |
| 852 | 201734_at | NM_001829.1 | Homo sapiens mRNA; cDNA DKFZp564I0463 (from clone | — | <1 |
| | | | DKFZp564I0463) | | |
| 853 | 200644_at | NM_023009.1 | MARCKS-like protein | MLP | >1 |
| 854 | 223459_s_at | BE222214 | hypothetical protein FLJ20519 | FLJ20519 | >1 |
| 855 | 219215_s_at | NM_017767.1 | solute carrier family 39 (zinc transporter), member 4 | SLC39A4 | >1 |
| 856 | 201811_x_at | NM_004844.1 | SH3-domain binding protein 5 (BTK-associated) | SH3BP5 | <1 |
| 857 | 212264_s_at | D87450.1 | friend of EBNA2 | FOE | <1 |
| 858 | 218668_s_at | NM_021183.1 | hypothetical protein similar to small G proteins, especially RAP-2A | LOC57826 | <1 |
| 859 | 209418_s_at | BC003615.1 | chromosome 22 open reading frame 19 | C22orf19 | >1 |
| 860 | 203028_s_at | NM_000101.1 | cytochrome b-245, alpha polypeptide | CYBA | >1 |
| 861 | 219410_at | NM_018004.1 | hypothetical protein FLJ10134 | FLJ10134 | <1 |
| 862 | 218220_at | NM_021640.1 | chromosome 12 open reading frame 10 | C12orf10 | >1 |
| 863 | 213154_s_at | AB014599.1 | coiled-coil protein BICD2 | BICD2 | >1 |
| 864 | 200920_s_at | AL535380 | B-cell translocation gene 1, anti-proliferative | BTG1 | >1 |
| 865 | 214459_x_at | M12679.1 | Cw1 antigen | HUMMHCW1A | <1 |
| 866 | 205955_at | NM_018336.1 | hypothetical protein FLJ11136 | FLJ11136 | >1 |
| 867 | 218482_at | NM_020189.1 | DC6 protein | DC6 | >1 |
| 868 | 203159_at | NM_014905.1 | glutaminase | GLS | <1 |
| 869 | 217823_s_at | NM_016021.1 | ubiquitin-conjugating enzyme E2, J1 (UBC6 homolog, yeast) | UBE2J1 | <1 |
| 870 | 225445_at | AI332346 | EST | — | <1 |
| 871 | 211368_s_at | U13700.1 | caspase 1, apoptosis-related cysteine protease (interleukin 1, beta, convertase) | CASP1 | <1 |
| 872 | 227811_at | AK000004.1 | FGD1 family, member 3 | FGD3 | >1 |
| 873 | 204116_at | NM_000206.1 | interleukin 2 receptor, gamma (severe combined immunodeficiency) | IL2RG | <1 |
| 874 | 212120_at | BF348067 | ras-like protein TC10 | TC10 | <1 |
| 875 | 37986_at | M60459 | erythropoietin receptor | EPOR | <1 |
| 876 | 242692_at | AI798758 | EST | — | >1 |
| 877 | 209644_x_at | U38945.1 | cyclin-dependent kinase inhibitor 2A (melanoma, p16, inhibits CDK4) | CDKN2A | >1 |
| 878 | 228545_at | AI016784 | EST | — | <1 |
| 879 | 201858_s_at | J03223.1 | proteoglycan 1, secretory granule | PRG1 | <1 |
| 880 | 215823_x_at | U64661 | EST, Highly similar to PAB1_HUMAN Polyadenylate-binding protein 1 | — | >1 |
| | | | (Poly(A)-binding protein 1) (PABP 1) (PABP1) [H. sapiens] | | |
| 881 | 201972_at | AF113129.1 | ATPase, H+ transporting, lysosomal 70 kDa, V1 subunit A, isoform 1 | ATP6V1A1 | <1 |
| 882 | 201951_at | NM_001627.1 | activated leukocyte cell adhesion molecule | ALCAM | <1 |
| 883 | 201986_at | NM_005121.1 | thyroid hormone receptor-associated protein, 240 kDa subunit | TRAP240 | <1 |
| 884 | 202393_s_at | NM_005655.1 | TGFB inducible early growth response | TIEG | >1 |
| 885 | 212118_at | NM_006510.1 | ret finger protein | RFP | <1 |
| 886 | 225910_at | BF514723 | hypothetical protein LOC284019 | LOC284019 | <1 |
| 887 | 218795_at | NM_016361.1 | lysophosphatidic acid phosphatase | ACP6 | >1 |
| 888 | 204985_s_at | NM_024108.1 | hypothetical protein MGC2650 | MGC2650 | >1 |
| 889 | 217436_x_at | M80469 | — | — | <1 |
| 890 | 215690_x_at | AL157437.1 | GPAA1P anchor attachment protein 1 homolog (yeast) | GPAA1 | >1 |
| 891 | 208683_at | M23254.1 | calpain 2, (m/II) large subunit | CAPN2 | <1 |
| 892 | 223638_at | AL136890.1 | hypothetical protein DKFZp434D177 | DKFZp434D177 | <1 |
| 893 | 218079_s_at | NM_024835.1 | C3HC4-type zinc finger protein | LZK1 | <1 |
| 894 | 209250_at | BC000961.2 | degenerative spermatocyte homolog, lipid desaturase (Drosophila) | DEGS | <1 |
| 895 | 238724_at | R63824 | EST | — | >1 |
| 896 | 212809_at | AA152202 | hypothetical protein FLJ14639 | FLJ14639 | >1 |
| 897 | 222391_at | AL080250 | hypothetical protein FLJ10856 | FLJ10856 | <1 |
| 898 | 209533_s_at | AF145020.1 | phospholipase A2-activating protein | PLAA | <1 |
| 899 | 218205_s_at | NM_017572.1 | MAP kinase-interacting serine/threonine kinase 2 | MKNK2 | >1 |
| 900 | 232174_at | AA480392 | Homo sapiens clone 24838 mRNA sequence | — | >1 |
| 901 | 201068_s_at | NM_002803.1 | proteasome (prosome, macropain) 26S subunit, ATPase, 2 | PSMC2 | <1 |
| 902 | 218573_at | NM_014061.1 | APR-1 protein | MAGEH1 | <1 |
| 903 | 216272_x_at | AF209931.1 | hypothetical protein FLJ13511 | 7h3 | >1 |
| 904 | 222309_at | AW972292 | EST | — | >1 |
| 905 | 226461_at | AA204719 | homeo box B9 | HOXB9 | >1 |
| 906 | 214449_s_at | NM_012249.1 | ras-like protein TC10 | TC10 | <1 |
| 907 | 217880_at | AI203880 | cell division cycle 27 | CDC27 | <1 |
| 908 | 213238_at | AI478147 | ATPase, Class V, type 10D | ATP10D | <1 |
| 909 | 228464_at | AI651510 | EST, Weakly similar to T12486 hypothetical protein DKFZp566H033.1 — | — | <1 |
| | | | human [H. sapiens] | | |
| 910 | 203157_s_at | AB020645.1 | glutaminase | GLS | <1 |
| 911 | 204547_at | NM_006822.1 | RAB40B, member RAS oncogene family | RAB40B | >1 |
| 912 | 203067_at | NM_003477.1 | E3-binding protein | PDX1 | <1 |
| 913 | 228289_at | AI131537 | adenylate cyclase 7 | ADCY7 | <1 |
| 914 | 217955_at | NM_015367.1 | BCL2-like 13 (apoptosis facilitator) | BCL2L13 | <1 |
| 915 | 201768_s_at | BC004467.1 | enthoprotin | ENTH | <1 |
| 916 | 217832_at | NM_006372.1 | NS1-associated protein 1 | NSAP1 | <1 |
| 917 | 226923_at | AW205790 | hypothetical protein FLJ39514 | FLJ39514 | <1 |
| 918 | 217939_s_at | NM_017657.1 | hypothetical protein FLJ20080 | FLJ20080 | <1 |
| 919 | 244732_at | R06827 | Homo sapiens, clone IMAGE: 5276307, mRNA | — | >1 |
| 920 | 221718_s_at | M90360.1 | A kinase (PRKA) anchor protein 13 | AKAP13 | >1 |
| 921 | 218970_s_at | NM_015960.1 | CGI-32 protein | CGI-32 | <1 |
| 922 | 214259_s_at | AW074911 | aldo-keto reductase family 7, member A2 (aflatoxin aldehyde reductase) | AKR7A2 | >1 |
| 923 | 204020_at | BF739943 | purine-rich element binding protein A | PURA | <1 |
| 924 | 205565_s_at | NM_000144.1 | Friedreich ataxia | FRDA | <1 |
| 925 | 218768_at | NM_020401.1 | nuclear pore complex protein | NUP107 | >1 |
| 926 | 202011_at | NM_003257.1 | tight junction protein 1 (zona occludens 1) | TJP1 | <1 |
| 927 | 211423_s_at | D85181.1 | sterol-C5-desaturase (ERG3 delta-5-desaturase homolog, fungal)-like | SC5DL | <1 |
| 928 | 202738_s_at | BG149218 | phosphorylase kinase, beta | PHKB | <1 |
| 929 | 228697_at | AW731710 | histidine triad nucleotide binding protein 3 | HINT3 | <1 |
| 930 | 225317_at | AL574669 | hypothetical protein MGC2404 | MGC2404 | >1 |
| 931 | 217368_at | X69909 | — | — | >1 |
| 932 | 201393_s_at | NM_000876.1 | insulin-like growth factor 2 receptor | IGF2R | <1 |
| 933 | 205158_at | NM_002937.1 | ribonuclease, RNase A family, 4 | RNASE4 | <1 |
| 934 | 200734_s_at | BG341906 | ADP-ribosylation factor 3 | ARF3 | >1 |
| 935 | 239586_at | AA085776 | hypothetical protein MGC14128 | MGC14128 | >1 |
| 936 | 225216_at | AI590719 | Homo sapiens cDNA: FLJ21191 fis, clone COL00104. | — | <1 |
| 937 | 203373_at | NM_003877.1 | suppressor of cytokine signaling 2 | SOCS2 | >1 |
| 938 | 218003_s_at | NM_002013.1 | FK506 binding protein 3, 25 kDa | FKBP3 | >1 |
| 939 | 208296_x_at | NM_014350.1 | TNF-induced protein | GG2-1 | <1 |
| 940 | 217716_s_at | NM_013336.1 | protein transport protein SEC61 alpha subunit isoform 1 | SEC61A1 | <1 |
| 941 | 202028_s_at | BC000603.1 | ribosomal protein L38 | RPL38 | >1 |
| 942 | 218231_at | NM_017567.1 | N-acetylglucosamine kinase | NAGK | <1 |
| 943 | 211528_x_at | M90685.1 | HLA-G histocompatibility antigen, class I, G | HLA-G | <1 |
| 944 | 203142_s_at | NM_003664.1 | adaptor-related protein complex 3, beta 1 subunit | AP3B1 | <1 |
| 945 | 230597_at | AI963203 | solute carrier family 7 (cationic amino acid transporter, y+ system), member 3 | SLC7A3 | >1 |
| 946 | 200864_s_at | NM_004663.1 | RAB11A, member RAS oncogene family | RAB11A | <1 |
| 947 | 205541_s_at | NM_018094.1 | G1 to S phase transition 2 | GSPT2 | <1 |
| 948 | 209267_s_at | AB040120.1 | BCG-induced gene in monocytes, clone 103 | BIGM103 | <1 |
| 949 | 207428_x_at | NM_001787.1 | cell division cycle 2-like 1 (PITSLRE proteins) | CDC2L1 | >1 |
| 950 | 205801_s_at | NM_015376.1 | guanine nucleotide exchange factor for Rap1 | GRP3 | <1 |
| 951 | 228614_at | AW182614 | hypothetical protein LOC205251 | LOC205251 | <1 |
| 952 | 230261_at | AA552969 | Homo sapiens, clone IMAGE: 4816784, mRNA | — | <1 |
| 953 | 229194_at | AL045882 | Homo sapiens, clone IMAGE: 5273745, mRNA | — | <1 |
| 954 | 224951_at | BE348305 | hypothetical protein MGC45411 | LOC91012 | >1 |
| 955 | 230026_at | N74662 | mitochondrial ribosomal protein L43 | MRPL43 | >1 |
| 956 | 217975_at | NM_016303.1 | pp21 homolog | LOC51186 | <1 |
| 957 | 212714_at | AL050205.1 | c-Mpl binding protein | LOC113251 | <1 |
| 958 | 212990_at | AB020717.1 | synaptojanin 1 | SYNJ1 | <1 |
| 959 | 211356_x_at | U66495.1 | leptin receptor | LEPR | <1 |
| 960 | 241342_at | BG288115 | hypothetical protein BC017881 | LOC157378 | >1 |
| 961 | 239891_x_at | AA001052 | EST, Weakly similar to RB10_HUMAN Ras-related protein Rab-10 | — | <1 |
| | | | [H. sapiens] | | |
| 962 | 214672_at | AB023215.1 | KIAA0998 protein | KIAA0998 | >1 |
| 963 | 201628_s_at | NM_006570.1 | Ras-related GTP-binding protein | RAGA | <1 |
| 964 | 232761_at | AL117381 | cytochrome c oxidase subunit IV isoform 2 | COX4I2 | >1 |
| 965 | 233164_x_at | AK026955.1 | hypothetical protein DKFZp547E052 | DKFZp547E052 | <1 |
| 966 | 200077_s_at | D87914.1 | ornithine decarboxylase antizyme 1 | OAZ1 | >1 |
| 967 | 219549_s_at | NM_006054.1 | reticulon 3 | RTN3 | <1 |
| 968 | 203560_at | NM_003878.1 | gamma-glutamyl hydrolase (conjugase, folylpolygammaglutamyl hydrolase) | GGH | >1 |
| 969 | 217923_at | NM_012392.1 | PEF protein with a long N-terminal hydrophobic domain (peflin) | PEF | <1 |
| 970 | 201862_s_at | NM_004735.1 | leucine rich repeat (in FLII) interacting protein 1 | LRRFIP1 | <1 |
| 971 | 223400_s_at | AF197569.1 | polybromo 1 | PB1 | <1 |
| 972 | AFFX- | M27830 | — | — | >1 |
| | M27830_M_{—} | | | | |
| | at | | | | |
| 973 | 41220_at | AB023208 | MLL septin-like fusion | MSF | >1 |
| 974 | 209276_s_at | AF162769.1 | glutaredoxin (thioltransferase) | GLRX | <1 |
| 975 | 207627_s_at | NM_005653.1 | transcription factor CP2 | TFCP2 | <1 |
| 976 | 204785_x_at | NM_000874.1 | interferon (alpha, beta and omega) receptor 2 | IFNAR2 | >1 |
| 977 | 222615_s_at | AW206812 | hypothetical protein FLJ13902 | FLJ13902 | >1 |
| 978 | 200949_x_at | NM_001023.1 | ribosomal protein S20 | RPS20 | >1 |
| 979 | 217192_s_at | AL022067 | PR domain containing 1, with ZNF domain | PRDM1 | >1 |
| 980 | 235792_x_at | AU154663 | Homo sapiens mRNA; cDNA DKFZp564L222 (from clone DKFZp564L222) | — | <1 |
| 981 | 213857_s_at | BG230614 | Homo sapiens, clone IMAGE: 4822825, mRNA | — | <1 |
| 982 | 235507_at | AA461195 | similar to hypothetical protein FLJ10883 | LOC115294 | >1 |
| 983 | 218191_s_at | NM_018368.1 | hypothetical protein FLJ11240 | FLJ11240 | <1 |
| 984 | 200649_at | BC002356.1 | nucleobindin 1 | NUCB1 | <1 |
| 985 | 210260_s_at | BC005352.1 | TNF-induced protein | GG2-1 | <1 |
| 986 | 209513_s_at | BC004331.1 | hypothetical protein MGC10940 | MGC10940 | <1 |
| 987 | 211801_x_at | AF329637.1 | mitofusin 1 | MFN1 | <1 |
| 988 | 206875_s_at | NM_014720.1 | Ste20-related serine/threonine kinase | SLK | <1 |
| 989 | 39705_at | AB014600 | SIN3 homolog B, transcriptional regulator (yeast) | SIN3B | <1 |
| 990 | 203658_at | BC001689.1 | solute carrier family 25 (carnitine/acylcarnitine translocase), member 20 | SLC25A20 | <1 |
| 991 | 235566_at | AW591660 | Homo sapiens cDNA FLJ39046 fis, clone NT2RP7010612. | — | <1 |
| 992 | 205089_at | NM_003416.1 | zinc finger protein 7 (KOX 4, clone HF.16) | ZNF7 | >1 |
| 993 | 212040_at | AK025557.1 | Homo sapiens, clone IMAGE: 6057297, mRNA | — | <1 |
| 994 | 210962_s_at | AB019691.1 | A kinase (PRKA) anchor protein (yotiao) 9 | AKAP9 | <1 |
| 995 | 203053_at | NM_005872.1 | breast carcinoma amplified sequence 2 | BCAS2 | >1 |
| 996 | 233867_at | AK000119.1 | EST, Moderately similar to KIAA0737 gene product [Homo sapiens] | — | >1 |
| | | | [H. sapiens] | | |
| 997 | 200993_at | AL137335.1 | EST | — | <1 |
| 998 | 204328_at | NM_007267.2 | epidermodysplasia verruciformis 1 | EVER1 | >1 |
| 999 | 212926_at | AB011166.1 | SMC5 structural maintenance of chromosomes 5-like 1 (yeast) | SMC5L1 | >1 |
| 1000 | 229353_s_at | AW515443 | similar to rat nuclear ubiquitous casein kinase 2 | NUCKS | >1 |
| 1001 | 212455_at | N36997 | KIAA1966 protein | KIAA1966 | <1 |
| 1002 | 202025_x_at | NM_001607.2 | acetyl-Coenzyme A acyltransferase 1 (peroxisomal 3-oxoacyl-Coenzyme A | ACAA1 | >1 |
| | | | thiolase) | | |
| 1003 | 235009_at | AI049791 | hypothetical protein FLJ33215 | FLJ33215 | >1 |
| 1004 | 218306_s_at | NM_003922.1 | hect (homologous to the E6-AP (UBE3A) carboxyl terminus) domain and | HERC1 | <1 |
| | | | RCC1 (CHC1)-like domain (RLD) 1 | | |
| 1005 | 225592_at | D81048 | nurim (nuclear envelope membrane protein) | NRM | >1 |
| 1006 | 238604_at | AA768884 | Homo sapiens cDNA FLJ25559 fis, clone JTH02834. | — | <1 |
| 1007 | 202264_s_at | NM_006114.1 | translocase of outer mitochondrial membrane 40 homolog (yeast) | TOMM40 | >1 |
| 1008 | 239258_at | BE551407 | EST, Moderately similar to hypothetical protein FLJ20234 [Homo sapiens] | — | <1 |
| | | | [H. sapiens] | | |
| 1009 | 210538_s_at | U37546.1 | baculoviral IAP repeat-containing 3 | BIRC3 | <1 |
| 1010 | 202545_at | NM_006254.1 | protein kinase C, delta | PRKCD | <1 |
| 1011 | 212622_at | D26067.1 | KIAA0033 protein | KIAA0033 | <1 |
| 1012 | 207431_s_at | NM_003676.1 | degenerative spermatocyte homolog, lipid desaturase (Drosophila) | DEGS | <1 |
| 1013 | 218549_s_at | NM_016033.1 | CGI-90 protein | CGI-90 | >1 |
| 1014 | 225058_at | AL365404.1 | G protein-coupled receptor 108 | GPR108 | <1 |
| 1015 | 224847_at | AW274756 | Homo sapiens cDNA FLJ20653 fis, clone KAT01739. | — | <1 |
| 1016 | 222024_s_at | AK022014.1 | A kinase (PRKA) anchor protein 13 | AKAP13 | >1 |
| 1017 | 208882_s_at | U69567 | progestin induced protein | DD5 | >1 |
| 1018 | 208937_s_at | D13889.1 | inhibitor of DNA binding 1, dominant negative helix-loop-helix protein | ID1 | >1 |
| 1019 | 200857_s_at | NM_006311.1 | nuclear receptor co-repressor 1 | NCOR1 | <1 |
| 1020 | 219972_s_at | NM_022495.1 | chromosome 14 open reading frame 135 | C14orf135 | >1 |
| 1021 | 226191_at | AW139538 | EST, Highly similar to SMD1_HUMAN Small nuclear ribonucleoprotein Sm | — | <1 |
| | | | D1 (snRNP core protein D1) (Sm-D1) (Sm-D autoantigen) [H. sapiens] | | |
| 1022 | 222129_at | AK026155.1 | hypothetical protein MGC3035 | MGC3035 | <1 |
| 1023 | 201668_x_at | AW163148 | myristoylated alanine-rich protein kinase C substrate | MARCKS | >1 |
| 1024 | 208549_x_at | NM_016171.1 | prothymosin a14 | LOC51685 | >1 |
| 1025 | 242241_x_at | R66713 | EST | — | >1 |
| 1026 | 211671_s_at | U01351.1 | nuclear receptor subfamily 3, group C, member 1 (glucocorticoid receptor) | NR3C1 | <1 |
| 1027 | 221787_at | AF055030.1 | PHD zinc finger protein XAP135 | XAP135 | <1 |
| 1028 | 228600_x_at | BE220330 | Homo sapiens mRNA; cDNA DKFZp686F0810 (from clone | — | <1 |
| | | | DKFZp686F0810) | | |
| 1029 | 213620_s_at | AA126728 | intercellular adhesion molecule 2 | ICAM2 | <1 |
| 1030 | 204267_x_at | NM_004203.1 | membrane-associated tyrosine- and threonine-specific cdc2-inhibitory kinase | PKMYT1 | >1 |
| 1031 | 205443_at | NM_003082.1 | small nuclear RNA activating complex, polypeptide 1, 43 kDa | SNAPC1 | >1 |
| 1032 | 218408_at | NM_012456.1 | translocase of inner mitochondrial membrane 10 homolog (yeast) | TIMM10 | >1 |
| 1033 | 221897_at | AA205660 | tripartite motif-containing 52 | TRIM52 | <1 |
| 1034 | 201970_s_at | NM_002482.1 | nuclear autoantigenic sperm protein (histone-binding) | NASP | >1 |
| 1035 | 227701_at | AK024739.1 | CTCL tumor antigen L14-2 | FLJ10188 | <1 |
| 1036 | 228549_at | AI491983 | EST, Moderately similar to hypothetical protein FLJ20378 [Homo sapiens] | — | <1 |
| | | | [H. sapiens] | | |
| 1037 | 211404_s_at | BC004371.1 | amyloid beta (A4) precursor-like protein 2 | APLP2 | >1 |
| 1038 | 218905_at | NM_017864.1 | hypothetical protein FLJ20530 | FLJ20530 | >1 |
| 1039 | 203774_at | NM_000254.1 | 5-methyltetrahydrofolate-homocysteine methyltransferase | MTR | <1 |
| 1040 | 200759_x_at | NM_003204.1 | nuclear factor (erythroid-derived 2)-like 1 | NFE2L1 | <1 |
| 1041 | 242674_at | T82467 | Homo sapiens cDNA FLJ41014 fis, clone UTERU2018674. | — | >1 |
| 1042 | AFFX- | X00351 | actin, beta | ACTB | <1 |
| | HSAC07/X00351_{—} | | | | |
| | M_at | | | | |
| 1043 | 201025_at | NM_015904.1 | translation initiation factor IF2 | IF2 | <1 |
| 1044 | 226344_at | AI741051 | KIAA1789 protein | KIAA1789 | <1 |
| 1045 | 227854_at | BE620258 | hypothetical protein FLJ10335 | FLJ10335 | <1 |
| 1046 | 220202_s_at | NM_018835.1 | membrane-associated nucleic acid binding protein | MNAB | <1 |
| 1047 | 203158_s_at | AF097493.1 | glutaminase | GLS | <1 |
| 1048 | 233186_s_at | AK001039.1 | BTG3 associated nuclear protein | BANP | >1 |
| 1049 | 205569_at | NM_014398.1 | lysosomal-associated membrane protein 3 | LAMP3 | <1 |
| 1050 | 222680_s_at | AK001261.1 | RA-regulated nuclear matrix-associated protein | RAMP | >1 |
| 1051 | 208523_x_at | NM_003525.1 | histone 1, H2bi | HIST1H2BI | >1 |
| 1052 | 207761_s_at | NM_014033.1 | DKFZP586A0522 protein | DKFZP586A0522 | <1 |
| 1053 | 220547_s_at | NM_019054.1 | hypothetical protein MGC5560 | MGC5560 | <1 |
| 1054 | 224912_at | BE205790 | tetratricopeptide repeat domain 7 | TTC7 | <1 |
| 1055 | 211367_s_at | U13699.1 | caspase 1, apoptosis-related cysteine protease (interleukin 1, beta, convertase) | CASP1 | <1 |
| 1056 | 209376_x_at | AW084759 | splicing factor, arginine/serine-rich 2, interacting protein | SFRS2IP | >1 |
| 1057 | 213932_x_at | AI923492 | major histocompatibility complex, class I, A | HLA-A | <1 |
| 1058 | 202261_at | NM_005997.1 | transcription factor-like 1 | TCFL1 | >1 |
| 1059 | 213811_x_at | BG393795 | transcription factor 3 (E2A immunoglobulin enhancer binding factors | TCF3 | >1 |
| | | | E12/E47) | | |
| 1060 | 212833_at | M74089.1 | hypothetical protein BC017169 | LOC91137 | <1 |
| 1061 | 216540_at | X61072.1 | T cell receptor alpha locus | TRA@ | >1 |
| 1062 | 215284_at | AF070575.1 | Homo sapiens clone 24407 mRNA sequence | — | <1 |
| 1063 | 239395_at | AA835887 | Homo sapiens, clone IMAGE: 5286379, mRNA | — | >1 |
| 1064 | 209388_at | BC000927.1 | poly(A) polymerase alpha | PAPOLA | >1 |
| 1065 | 235038_at | BF665176 | HIV-1 rev binding protein 2 | HRB2 | >1 |
| 1066 | 235745_at | AV704183 | hypothetical protein FLJ30999 | FLJ30999 | <1 |
| 1067 | 242048_at | BE905316 | EST | — | >1 |
| 1068 | 239250_at | BE966038 | hypothetical protein LOC147947 | LOC147947 | >1 |
| 1069 | 213828_x_at | AA477655 | H3 histone, family 3A | H3F3A | >1 |
| 1070 | 222593_s_at | AA584308 | hypothetical protein FLJ13117 | FLJ13117 | >1 |
| 1071 | 229075_at | AI754871 | EST | — | <1 |
| 1072 | 219978_s_at | NM_018454.1 | nucleolar protein ANKT | ANKT | >1 |
| 1073 | 211676_s_at | AF056979.1 | interferon gamma receptor 1 | IFNGR1 | <1 |
| 1074 | 234347_s_at | AF038554.1 | density-regulated protein | DENR | >1 |
| 1075 | 209066_x_at | M26700.1 | ubiquinol-cytochrome c reductase binding protein | UQCRB | >1 |
| 1076 | 241435_at | AA702930 | EST | — | >1 |
| 1077 | 219507_at | NM_016625.1 | hypothetical protein LOC51319 | LOC51319 | >1 |
| 1078 | 202284_s_at | NM_000389.1 | cyclin-dependent kinase inhibitor 1A (p21, Cip1) | CDKN1A | <1 |
| 1079 | 218732_at | NM_016077.1 | CGI-147 protein | CGI-147 | <1 |
| 1080 | 207654_x_at | NM_001938.1 | down-regulator of transcription 1, TBP-binding (negative cofactor 2) | DR1 | >1 |
| 1081 | 226671_at | AI150000 | Homo sapiens, clone IMAGE: 4797120, mRNA | — | <1 |
| 1082 | 227637_at | AV712694 | transcription factor CP2 | TFCP2 | >1 |
| 1083 | 201580_s_at | AL544094 | hypothetical protein DJ971N18.2 | DJ971N18.2 | <1 |
| 1084 | 226580_at | AA779684 | breast cancer metastasis-suppressor 1 | BRMS1 | >1 |
| 1085 | 224312_x_at | BC000675.1 | hypothetical protein FLJ20542 | FLJ20542 | >1 |
| 1086 | 227425_at | AI984607 | Homo sapiens cDNA FLJ40165 fis, clone TESTI2015962. | — | <1 |
| 1087 | 202643_s_at | AI738896 | tumor necrosis factor, alpha-induced protein 3 | TNFAIP3 | <1 |
| 1088 | 227080_at | AW003092 | Homo sapiens cDNA: FLJ23366 fis, clone HEP15665. | — | >1 |
| 1089 | 235353_at | AI887866 | KIAA0746 protein | KIAA0746 | >1 |
| 1090 | 209534_x_at | BF222823 | A kinase (PRKA) anchor protein 13 | AKAP13 | >1 |
| 1091 | 235103_at | AA029155 | Homo sapiens mRNA; cDNA DKFZp686H1529 (from clone | — | <1 |
| | | | DKFZp686H1529) | | |
| 1092 | 235474_at | AI241810 | EST, Weakly similar to T31613 hypothetical protein Y50E8A.i — | — | <1 |
| | | | Caenorhabditis elegans [C. elegans] | | |
| 1093 | 218662_s_at | NM_022346.1 | chromosome condensation protein G | HCAP-G | >1 |
| 1094 | 208668_x_at | BC003689.1 | high-mobility group nucleosomal binding domain 2 | HMGN2 | >1 |
| 1095 | 214919_s_at | R39094 | Homo sapiens, clone IMAGE: 3866125, mRNA | — | <1 |
| 1096 | 218976_at | NM_021800.1 | J domain containing protein 1 | JDP1 | <1 |
| 1097 | 241955_at | BE243270 | EST, Weakly similar to C34D4.14.p [Caenorhabditis elegans] [C. elegans] | — | >1 |
| 1098 | 201138_s_at | BG532929 | Sjogren syndrome antigen B (autoantigen La) | SSB | >1 |
| 1099 | 209056_s_at | AW268817 | CDC5 cell division cycle 5-like (S. pombe) | CDC5L | >1 |
| 1100 | 219384_s_at | NM_012091.2 | adenosine deaminase, tRNA-specific 1 | ADAT1 | <1 |
| 1101 | 212886_at | AL080169.1 | DKFZP434C171 protein | DKFZP434C171 | <1 |
| 1102 | 226773_at | AW290940 | Homo sapiens cDNA FLJ35131 fis, clone PLACE6008824. | — | <1 |
| 1103 | 215756_at | AU153979 | Homo sapiens cDNA FLJ14231 fis, clone NT2RP3004470. | — | >1 |
| 1104 | 227994_x_at | AA548838 | chromosome 20 open reading frame 149 | C20orf149 | >1 |
| 1105 | 218120_s_at | D21243.1 | heme oxygenase(decycling)2 | HMOX2 | <1 |
| 1106 | 225092_at | AL550977 | rabaptin-5 | RAB5EP | <1 |
| 1107 | 220696_at | NM_014129.1 | PR00478 protein | PR00478 | >1 |
| 1108 | 210170_at | BC001017.1 | alpha-actinin-2-associated LIM protein | ALP | >1 |
| 1109 | 224648_at | AI860946 | vasculin | DKFZp761C169 | <1 |
| 1110 | 212830_at | BF110421 | EGF-like-domain, multiple 5 | EGFL5 | <1 |
| 1111 | 213410_at | AL050102.1 | DKFZp586F1019 protein | DKFZp586F1019 | >1 |
| 1112 | 212718_at | BG110231 | poly(A) polymerase alpha | PAPOLA | >1 |
| 1113 | 203173_s_at | AW080196 | esophageal cancer associated protein | MGC16824 | >1 |
| 1114 | 229520_s_at | BF060678 | chromosome 14 open reading frame 118 | C14orf118 | >1 |
| 1115 | 203974_at | NM_012080.1 | family with sequence similarity 16, member A, X-linked | FAM16AX | <1 |
| 1116 | 230075_at | AV724323 | RAB39B, member RAS oncogene family | RAB39B | <1 |
| 1117 | 225880_at | BF676081 | Homo sapiens cDNA FLJ11174 fis, clone PLACE1007367. | — | <1 |
| 1118 | 222891_s_at | AI912275 | B-cell CLL/lymphoma 11A (zinc finger protein) | BCL11A | <1 |
| 1119 | 213494_s_at | AA748649 | YY1 transcription factor | YY1 | >1 |
| 1120 | 211366_x_at | U13698.1 | caspase 1, apoptosis-related cysteine protease (interleukin 1, beta, convertase) | CASP1 | <1 |
| 1121 | 221995_s_at | BF195165 | mitochondrial ribosomal protein 63 | MRP63 | >1 |
| 1122 | 203322_at | NM_014913.1 | KIAA0863 protein | KIAA0863 | <1 |
| 1123 | 243051_at | AW135412 | EST | — | >1 |
| 1124 | 207245_at | NM_001077.1 | UDP glycosyltransferase 2 family, polypeptide B17 | UGT2B17 | <1 |
| 1125 | 225651_at | BF431962 | hypothetical protein FLJ25157 | FLJ25157 | <1 |
| 1126 | 232288_at | AK026209.1 | Homo sapiens cDNA: FLJ22556 fis, clone HSI01326. | — | <1 |
| 1127 | 218701_at | NM_016027.1 | CGI-83 protein | CGI-83 | >1 |
| 1128 | 201102_s_at | NM_002626.1 | phosphofructokinase, liver | PFKL | >1 |
| 1129 | 210458_s_at | BC003388.1 | TRAF family member-associated NFKB activator | TANK | <1 |
| 1130 | 226787_at | BF966015 | zinc finger protein 18 (KOX 11) | ZNF18 | <1 |
| 1131 | 218679_s_at | NM_016208.1 | vacuolar protein sorting 28 (yeast) | VPS28 | >1 |
| 1132 | 212232_at | AB023231.1 | formin binding protein 4 | FNBP4 | <1 |
| 1133 | 212221_x_at | AL117536.1 | Homo sapiens, clone IMAGE: 5278680, mRNA | — | <1 |
| 1134 | 200995_at | AL137335.1 | importin 7 | IPO7 | <1 |
| 1135 | 229549_at | AA868461 | calumenin | CALU | <1 |
| 1136 | 227239_at | AV734839 | down-regulated by Ctnnb1, a | DRCTNNB1A | <1 |
| 1137 | 210716_s_at | M97501.1 | restin (Reed-Steinberg cell-expressed intermediate filament-associated | RSN | <1 |
| | | | protein) | | |
| 1138 | 235170_at | T52999 | hypothetical protein FLJ34299 | FLJ34299 | >1 |
| 1139 | 216841_s_at | X15132.1 | superoxide dismutase 2, mitochondrial | SOD2 | >1 |
| 1140 | 204683_at | NM_000873.2 | intercellular adhesion molecule 2 | ICAM2 | <1 |
| 1141 | 228829_at | AI279868 | activating transcription factor 7 | ATF7 | >1 |
| 1142 | 212902_at | BE645231 | SEC24 related gene family, member A (S. cerevisiae) | SEC24A | <1 |
| 1143 | 212542_s_at | BF224151 | pleckstrin homology domain interacting protein | PHIP | >1 |
| 1144 | 201971_s_at | NM_001690.1 | ATPase, H+ transporting, lysosomal 70 kDa, V1 subunit A, isoform 1 | ATP6V1A1 | <1 |
| 1145 | 210266_s_at | AF220137.1 | tripartite motif-containing 33 | TRIM33 | >1 |
| 1146 | 222426_at | BG499947 | mitogen-activated protein kinase associated protein 1 | MAPKAP1 | >1 |
| 1147 | 201840_at | NM_006156.1 | neural precursor cell expressed, developmentally down-regulated 8 | NEDD8 | >1 |
| 1148 | 225282_at | AL137764.1 | hypothetical protein AL133206 | LOC64744 | <1 |
| 1149 | 231931_at | AL355710.1 | Homo sapiens EST from clone 112590, full insert | — | >1 |
| 1150 | 202271_at | AB007952.1 | KIAA0483 protein | KIAA0483 | <1 |
| 1151 | 204215_at | NM_024315.1 | hypothetical protein MGC4175 | MGC4175 | <1 |
| 1152 | 213127_s_at | BG230758 | mediator of RNA polymerase II transcription, subunit 8 homolog (yeast) | MED8 | <1 |
| 1153 | 217826_s_at | NM_016021.1 | ubiquitin-conjugating enzyme E2, J1 (UBC6 homolog, yeast) | UBE2J1 | <1 |
| 1154 | 203943_at | NM_004798.1 | kinesin family member 3B | KIF3B | <1 |
| 1155 | 209384_at | AA176833 | proline synthetase co-transcribed homolog (bacterial) | PROSC | <1 |
| 1156 | 228469_at | BF431902 | peptidylprolyl isomerase D (cyclophilin D) | PPID | <1 |
| 1157 | 209093_s_at | K02920.1 | glucosidase, beta; acid (includes glucosylceramidase) | GBA | >1 |
| 1158 | 239714_at | AA780063 | EST | — | >1 |
| 1159 | 239487_at | AI743261 | EST | — | <1 |
| 1160 | 204565_at | NM_018473.1 | uncharacterized hypothalamus protein HT012 | HT012 | <1 |
| 1161 | 201311_s_at | AL515318 | SH3 domain binding glutamic acid-rich protein like | SH3BGRL | <1 |
| 1162 | 235606_at | AA417117 | Homo sapiens cDNA FLJ31372 fis, clone NB9N42000281. | — | <1 |
| 1163 | 201952_at | NM_001627.1 | activated leukocyte cell adhesion molecule | ALCAM | <1 |
| 1164 | 212223_at | AL117536.1 | Homo sapiens, clone IMAGE: 5278680, mRNA | — | <1 |
| 1165 | 218084_x_at | NM_014164.2 | FXYD domain containing ion transport regulator 5 | FXYD5 | <1 |
| 1166 | 223559_s_at | AF161411.2 | HSPC043 protein | HSPC043 | <1 |
| 1167 | 208445_s_at | NM_023005.1 | bromodomain adjacent to zinc finger domain, 1B | BAZ1B | <1 |
| 1168 | 218423_x_at | NM_016516.1 | tumor antigen SLP-8p | HCC8 | <1 |
| 1169 | 203320_at | NM_005475.1 | lymphocyte adaptor protein | LNK | <1 |
| 1170 | 201618_x_at | NM_003801.2 | GPAA1P anchor attachment protein 1 homolog (yeast) | GPAA1 | >1 |
| 1171 | 229861_at | N66669 | general transcription factor IIH, polypeptide 3, 34 kDa | GTF2H3 | <1 |
| 1172 | 203420_at | NM_016255.1 | family with sequence similarity 8, member A1 | FAM8A1 | <1 |
| 1173 | 239209_at | AA826931 | regenerating islet-derived 1 alpha (pancreatic stone protein, pancreatic thread | REG1A | >1 |
| | | | protein) | | |
| 1174 | 206874_s_at | AL138761 | Ste20-related serine/threonine kinase | SLK | <1 |
| 1175 | 227988_s_at | AW629014 | chorea acanthocytosis | CHAC | <1 |
| 1176 | 238346_s_at | AW973003 | nuclear receptor coactivator 6 interacting protein | NCOA6IP | >1 |
| 1177 | 203707_at | NM_005741.1 | zinc finger protein 263 | ZNF263 | >1 |
| 1178 | 222790_s_at | BE888593 | hypothetical protein FLJ11220 | FLJ11220 | >1 |
| 1179 | 207734_at | NM_017773.1 | hypothetical protein FLJ20340 | LAX | <1 |
| 1180 | 201859_at | NM_002727.1 | proteoglycan 1, secretory granule | PRG1 | <1 |
| 1181 | 216250_s_at | X77598.1 | leupaxin | LPXN | <1 |
| 1182 | 217846_at | NM_005051.1 | glutaminyl-tRNA synthetase | QARS | >1 |
| 1183 | 202862_at | NM_000137.1 | fumarylacetoacetate hydrolase (fumarylacetoacetase) | FAH | <1 |
| 1184 | 209061_at | AF012108.1 | similar to glucosamine-6-sulfatases | SULF2 | <1 |
| 1185 | 203970_s_at | NM_003630.1 | peroxisomal biogenesis factor 3 | PEX3 | <1 |
| 1186 | 235067_at | D81987 | Homo sapiens, clone MGC: 27281 IMAGE: 4656464, mRNA, complete cds | — | <1 |
| 1187 | 228528_at | AI927692 | EST | — | <1 |
| 1188 | 218577_at | NM_017768.1 | hypothetical protein FLJ20331 | FLJ20331 | <1 |
| 1189 | 211089_s_at | Z25434.1 | NIMA (never in mitosis gene a)-related kinase 3 | NEK3 | <1 |
| 1190 | 221778_at | BE217882 | KIAA1718 protein | KIAA1718 | <1 |
| 1191 | 207981_s_at | NM_001438.1 | estrogen-related receptor gamma | ESRRG | <1 |
| 1192 | 219939_s_at | NM_007158.1 | NRAS-related gene | D1S155E | >1 |
| 1193 | 201084_s_at | NM_014739.1 | Bcl-2-associated transcription factor | BTF | <1 |
| 1194 | 209452_s_at | AF035824.1 | vesicle transport through interaction with t-SNAREs homolog 1B (yeast) | VTI1B | >1 |
| 1195 | 214527_s_at | AB041836.1 | polyglutamine binding protein 1 | PQBP1 | <1 |
| 1196 | 222243_s_at | AB051450.1 | transducer of ERBB2, 2 | TOB2 | >1 |
| 1197 | 204192_at | NM_001774.1 | CD37 antigen | CD37 | <1 |
| 1198 | 217775_s_at | NM_016026.1 | retinol dehydrogenase 11 (all-trans and 9-cis) | RDH11 | >1 |
| 1199 | 227685_at | AI767750 | Homo sapiens cDNA FLJ39046 fis, clone NT2RP7010612. | — | <1 |
| 1200 | 225731_at | AB033049.1 | KIAA1223 protein | KIAA1223 | <1 |
| 1201 | 209475_at | AF106069.1 | ubiquitin specific protease 15 | USP15 | <1 |
| 1202 | 213024_at | BF593908 | TATA element modulatory factor 1 | TMF1 | <1 |
| 1203 | 221508_at | AF181985.1 | STE20-like kinase | JIK | <1 |
| 1204 | 212242_at | AL565074 | tubulin, alpha 1 (testis specific) | TUBA1 | <1 |
| 1205 | 200607_s_at | BG289967 | RAD21 homolog (S. pombe) | RAD21 | >1 |
| 1206 | 213671_s_at | AA621558 | methionine-tRNA synthetase | MARS | >1 |
| 1207 | 201697_s_at | NM_001379.1 | DNA (cytosine-5-)-methyltransferase 1 | DNMT1 | >1 |
| 1208 | 202105_at | NM_001551.1 | immunoglobulin (CD79A) binding protein 1 | IGBP1 | >1 |
| 1209 | 241370_at | AA278233 | Homo sapiens cDNA FLJ37785 fis, clone BRHIP2028330. | — | >1 |
| 1210 | 220368_s_at | NM_017936.1 | hypothetical protein FLJ20707 | FLJ20707 | >1 |
| 1211 | 226710_at | AI199072 | ribosomal protein S3A | RPS3A | >1 |
| 1212 | 214317_x_at | BE348997 | ribosomal protein S9 | RPS9 | >1 |
| 1213 | 228341_at | AI809108 | Homo sapiens cDNA FLJ36248 fis, clone THYMU2001989. | — | <1 |
| 1214 | 204523_at | NM_003440.1 | zinc finger protein 140 (clone pHZ-39) | ZNF140 | <1 |
| 1215 | 212465_at | AA524500 | hypothetical protein FLJ23027 | FLJ23027 | >1 |
| 1216 | 203606_at | NM_004553.1 | NADH dehydrogenase (ubiquinone) Fe-S protein 6, 13 kDa (NADH- | NDUFS6 | >1 |
| | | | coenzyme Q reductase) | | |
| 1217 | 211529_x_at | M90684.1 | HLA-G histocompatibility antigen, class I, G | HLA-G | <1 |
| 1218 | 211517_s_at | M96651.1 | interleukin 5 receptor, alpha | IL5RA | <1 |
| 1219 | 220946_s_at | NM_014159.1 | huntingtin interacting protein B | HYPB | >1 |
| 1220 | 204350_s_at | NM_004270.1 | cofactor required for Sp1 transcriptional activation, subunit 9, 33 kDa | CRSP9 | <1 |
| 1221 | 39582_at | AL050166 | Homo sapiens mRNA; cDNA DKFZp586D1122 (from clone | — | <1 |
| | | | DKFZp586D1122) | | |
| 1222 | 204645_at | NM_001241.1 | cyclin T2 | CCNT2 | <1 |
| 1223 | 211136_s_at | BC004865.1 | cleft lip and palate associated transmembrane protein 1 | CLPTM1 | <1 |
| 1224 | 229312_s_at | BF434321 | protein kinase anchoring protein GKAP42 | GKAP42 | >1 |
| 1225 | 226504_at | AA522720 | Homo sapiens, similar to CG12393 gene product, clone IMAGE: 5188623, | — | >1 |
| | | | mRNA, partial cds | | |
| 1226 | 221547_at | BC000794.1 | PRP18 pre-mRNA processing factor 18 homolog (yeast) | PRPF18 | <1 |
| 1227 | 238035_at | N66313 | EST | — | <1 |
| 1228 | 213011_s_at | BF116254 | triosephosphate isomerase 1 | TPI1 | >1 |
| 1229 | 208718_at | Z97056 | Homo sapiens, clone IMAGE: 5264473, mRNA | — | <1 |
| 1230 | 204686_at | NM_005544.1 | insulin receptor substrate 1 | IRS1 | >1 |
| 1231 | 225763_at | AI659418 | hypothetical protein MGC21854 | MGC21854 | <1 |
| 1232 | 212643_at | AI671747 | chromosome 14 open reading frame 32 | C14orf32 | >1 |
| 1233 | 203060_s_at | AF074331.1 | 3′-phosphoadenosine 5′-phosphosulfate synthase 2 | PAPSS2 | <1 |
| 1234 | 206900_x_at | NM_021047.1 | zinc finger protein 253 | ZNF253 | <1 |
| 1235 | 225798_at | AI990891 | hypothetical protein DKFZp761K2222 | DKFZp761K2222 | <1 |
| 1236 | 209619_at | K01144.1 | CD74 antigen (invariant polypeptide of major histocompatibility complex, | CD74 | <1 |
| | | | class II antigen-associated) | | |
| 1237 | 200996_at | NM_005721.2 | ARP3 actin-related protein 3 homolog (yeast) | ACTR3 | <1 |
| 1238 | 228150_at | AI807478 | regucalcin gene promotor region related protein | RGPR | <1 |
| 1239 | 218152_at | NM_018200.1 | high-mobility group 20A | HMG20A | >1 |
| 1240 | 202546_at | NM_003761.1 | vesicle-associated membrane protein 8 (endobrevin) | VAMP8 | <1 |
| 1241 | 218603_at | NM_016217.1 | hHDC for homolog of Drosophila headcase | HDCL | <1 |
| 1242 | 213793_s_at | BE550452 | homer homolog 1 (Drosophila) | HOMER1 | >1 |
| 1243 | 205917_at | NM_003417.1 | — | — | <1 |
| 1244 | 218669_at | NM_021183.1 | hypothetical protein similar to small G proteins, especially RAP-2A | LOC57826 | <1 |
| 1245 | 226381_at | AW450329 | hypothetical protein FLJ20366 | FLJ20366 | <1 |
| 1246 | 211065_x_at | BC006422.1 | phosphofructokinase, liver | PFKL | >1 |
| 1247 | 224848_at | AW274756 | Homo sapiens cDNA FLJ20653 fis, clone KAT01739. | — | <1 |
| 1248 | 212616_at | AB002306.1 | hypothetical protein MGC17528 | MGC17528 | <1 |
| 1249 | 232171_x_at | AK001742.1 | hypothetical protein DKFZp434G0522 | DKFZp434G0522 | >1 |
| 1250 | 237181_at | AI478850 | EST | — | >1 |
| 1251 | 204171_at | NM_003161.1 | ribosomal protein S6 kinase, 70 kDa, polypeptide 1 | RPS6KB1 | <1 |
| 1252 | 201780_s_at | NM_007282.1 | ring finger protein 13 | RNF13 | <1 |
| 1253 | 215148_s_at | AI141541 | amyloid beta (A4) precursor protein-binding, family A, member 3 (X11-like | APBA3 | <1 |
| | | | 2) | | |
| 1254 | 203359_s_at | AL525412 | c-myc binding protein | MYCBP | <1 |
| 1255 | 201788_at | NM_007372.1 | RNA helicase-related protein | RNAHP | <1 |
| 1256 | 235661_at | T99553 | EST | — | <1 |
| 1257 | 202375_at | NM_014822.1 | SEC24 related gene family, member D (S. cerevisiae) | SEC24D | <1 |
| 1258 | 203491_s_at | AI123527 | KIAA0092 gene product | KIAA0092 | >1 |
| 1259 | 221989_at | AW057781 | ribosomal protein L10 | RPL10 | <1 |
| 1260 | 65630_at | AI742455 | SIPL protein | SIPL | <1 |
| 1261 | 214030_at | BE501352 | hypothetical protein DKFZp667G2110 | DKFZp667G2110 | <1 |
| 1262 | 243552_at | AW008914 | EST | — | >1 |
| 1263 | 214615_at | NM_014499.1 | purinergic receptor P2Y, G-protein coupled, 10 | P2RY10 | <1 |
| 1264 | 203404_at | NM_014782.1 | armadillo repeat protein ALEX2 | ALEX2 | <1 |
| 1265 | 212877_at | AA284075 | kinesin 2 60/70 kDa | KNS2 | >1 |
| 1266 | 231059_x_at | AI744643 | SCAN domain containing 1 | SCAND1 | >1 |
| 1267 | 225681_at | AA584310 | collagen triple helix repeat containing 1 | CTHRC1 | >1 |
| 1268 | 227946_at | AI955239 | oxysterol binding protein-like 7 | OSBPL7 | >1 |
| 1269 | 221323_at | NM_025218.1 | UL16 binding protein 1 | ULBP1 | >1 |
| 1270 | 232431_at | AI934556 | Human glucocorticoid receptor alpha mRNA, variant 3′UTR | — | <1 |
| 1271 | 32209_at | AF052151 | Mouse Mammary Turmor Virus Receptor homolog 1 | MTVR1 | <1 |
| 1272 | 201980_s_at | NM_012425.2 | Ras suppressor protein 1 | RSU1 | <1 |
| 1273 | 201558_at | NM_003610.1 | RAE1 RNA export 1 homolog (S. pombe) | RAE1 | >1 |
| 1274 | 221613_s_at | AL136598.1 | protein associated with PRK1 | AWP1 | <1 |
| 1275 | 243570_at | AA921960 | EST, Moderately similar to T12486 hypothetical protein DKFZp566H033.1 — | — | <1 |
| | | | human [H. sapiens] | | |
| 1276 | 214179_s_at | H93013 | nuclear factor (erythroid-derived 2)-like 1 | NFE2L1 | <1 |
| 1277 | 224768_at | AW451291 | hypothetical protein FLJ10006 | FLJ10006 | <1 |
| 1278 | 227518_at | AW051365 | EST, Moderately similar to hypothetical protein FLJ20378 [Homo sapiens] | — | <1 |
| | | | [H. sapiens] | | |
| 1279 | 218850_s_at | NM_014240.1 | LIM domains containing 1 | LIMD1 | >1 |
| 1280 | 201408_at | AI186712 | protein phosphatase 1, catalytic subunit, beta isoform | PPP1CB | <1 |
| 1281 | 214097_at | AW024383 | ribosomal protein S21 | RPS21 | >1 |
| 1282 | 242208_at | AI634543 | EST, Weakly similar to hypothetical protein FLJ20489 [Homo sapiens] | — | <1 |
| | | | [H. sapiens] | | |

Still further, Table 3 sets forth markers which are significantly expressed in myeloma samples from non-responder patients whose disease is refractory (i.e. progressive disease) to treatment with bortezomib. The markers identified in Table 3 were identified similar to the methods described above for Table 1. These markers will serve to distinguish refractory patients from those who will be either stable or responsive to treatment.

TABLE 3
Predictive Markers in Progressive Disease

| | | RefSeq/ | | | |
|---|---|---|---|---|---|
| | Probeset_{—} | Genbank | | Gene | |
| No. | ID | Accession | Title | Symbol | Unigene |
| 1283 | 205124_{—} | NM_005919.1 | MADS box transcription enhancer | MEF2B | Hs.78881 |
| | at | | factor 2, polypeptide B (myocyte | | |
| | | | enhancer factor 2B) | | |
| 1284 | 206626_{—} | BC001003.2 | synovial sarcoma, X breakpoint 1 | SSX1 | Hs.194759 |
| | x_at | | | | |
| 34 | 224918_{—} | AI220117 | microsomal glutathione S- | MGST1 | Hs.355733 |
| | x_at | | transferase 1 | | |
| 1285 | 206640_{—} | NM_001477.1 | G antigen 7B | GAGE7B | Hs.251677 |
| | x_at | | | | |
| 223 | 227174_{—} | Z98443 | | | Hs.86366 |
| | at | | | | |
| 1286 | 227617_{—} | BF315093 | Weakly similar to MUC2_HUMAN Mucin 2 | | Hs.22293 |
| | at | | precursor | | |
| 1287 | 207086_{—} | NM_001474.1 | G antigen 4 | GAGE4 | Hs.183199 |
| | x_at | | | | |
| 1288 | 209732_{—} | BC005254.1 | Similar to C-type (calcium | CLECS | Hs.85201 |
| | at | | dependent, carbohydrate- | F2 | |
| | | | recognition domain) lectin, | | |
| | | | superfamily member 2 (activation- | | |
| | | | induced) | | |
| 1289 | 214596_{—} | T15991 | cholinergic receptor, muscarinic 3 | CHRM3 | Hs.7138 |
| | at | | | | |
| 1290 | 202779_s_{—} | NM_014501.1 | ubiquitin carrier protein (E2-EPF) | E2-EPF | Hs.174070 |
| | at | | | | |
| 1291 | 231568_{—} | AI200804 | similar to Proliferation-associated protein 2G4 | | Hs.98612 |
| | at | | (Cell cycle protein p38-2G4 homolog) | | |
| 1292 | 207480_s_{—} | NM_020149.1 | TALE homeobox protein Meis2e | MEIS2 | Hs.283312 |
| | at | | | | |
| 1293 | 230352_{—} | AI392908 | phosphoribosyl pyrophosphate | PRPS2 | Hs.2910 |
| | at | | synthetase 2 | | |
| 1294 | 202411_{—} | NM_005532.1 | interferon, alpha-inducible protein | IFI27 | Hs.278613 |
| | at | | 27 | | |
| 17 | 215733_{—} | AJ012833.1 | CTL-recognized antigen on | CTAG2 | Hs.87225 |
| | x_at | | melanoma (CAMEL) | | |
| 1295 | 243030_{—} | AA211369 | | | Hs.269493 |
| | at | | | | |
| 18 | 210546_{—} | U87459.1 | autoimmunogenic cancertestis | CTAG1 | Hs.167379 |
| | x_at | | antigen NY-ESO-1 | | |
| 1296 | 202044_{—} | AU159484 | glucocorticoid receptor DNA | GRLF1 | Hs.102548 |
| | at | | binding factor 1 | | |
| 1297 | 217977_{—} | NM_016332.1 | selenoprotein X, 1 | SEPX1 | Hs.279623 |
| | at | | | | |
| 1298 | 231000_{—} | BE350315 | receptor tyrosine kinase-like | ROR2 | Hs.155585 |
| | at | | orphan receptor 2 | | |
| 1299 | 238587_{—} | AI927919 | Nm23-phosphorylated unknown | | Hs.187625 |
| | at | | substrate | | |
| 1300 | 239119_{—} | AW014374 | | | Hs.144849 |
| | at | | | | |
| 1301 | 236741_{—} | AW299463 | | | Hs.208067 |
| | at | | | | |
| 223 | 227174_{—} | Z98443 | | | Hs.86366 |
| | at | | | | |
| 1302 | 206897_{—} | NM_003785.2 | G antigen, family B, 1 (prostate | GAGEB1 | Hs.128231 |
| | at | | associated) | | |
| 205 | 204836_{—} | NM_000170.1 | glycine dehydrogenase | GLDC | Hs.27 |
| | at | | (decarboxylating; glycine | | |
| | | | decarboxylase, glycine cleavage | | |
| | | | system protein P) | | |
| 1303 | 208282_{—} | NM_020363.1 | deleted in azoospermia 2 | DAZ2 | Hs.283813 |
| | x_at | | | | |
| 1304 | 216922_{—} | AF271088.1 | deleted in azoospermia | DAZ | Hs.70936 |
| | x_at | | | | |
| 1305 | 231771_{—} | AI694073 | gap junction protein, beta 6 | GJB6 | Hs.48956 |
| | at | | (connexin 30) | | |
| 267 | 231131_{—} | AA909330 | weakly similar to GAR2 PROTEIN | | Hs.112765 |
| | at | | | | |
| 1306 | 217007_s_{—} | AK000667.1 | a disintegrin and metalloproteinase domain 15 | | Hs.92208 |
| | at | | (metargidin) | | |
| 1307 | 220445_s_{—} | NM_004909.1 | taxol resistance associated gene 3 | TRAG3 | Hs.251377 |
| | at | | | | |
| 1308 | 233216_{—} | AV741116 | | | Hs.283933 |
| | at | | | | |
| 1309 | 211323_s_{—} | L38019.1 | inositol 1,4,5-trisphosphate | ITPR1 | Hs.198443 |
| | at | | receptor type 1 | | |
| 1310 | 224188_s_{—} | BC001208.1 | Similar to hypothetical protein | | Hs.182061 |
| | at | | LOC63929 | | |
| 1311 | 213222_{—} | KIAA0581 | 1-phosphatidylinositol-4,5- | PLCB1 | Hs.41143 |
| | at | | bisphosphate phosphodiesterase | | |
| | | | beta 1 | | |
| 1312 | 201897_s_{—} | AF274941.1 | CDC28 protein kinase 1 | CKS1 | Hs.77550 |
| | at | | | | |
| 1313 | 206012_{—} | NM_003240.1 | endometrial bleeding associated | LEFTB | Hs.25195 |
| | at | | factor (left-right determination, | | |
| | | | factor A; transforming growth | | |
| | | | factor beta superfamily) | | |

Classifiers

Various algorithms are currently available that can be used to classify patient samples into prior defined groups using a given set of features. Therefore, the combination of markers selected through the feature selection process may be used in one of the following classifying algorithms in order to derive a prediction equation as to whether the patient sample is sensitive or resistant. The classifiers used in the present invention were: 1) Weighted Voting (“WV”); and 2) Combination of Thresholded Features (“CTF”).

The Weighted Voting classifier was implemented as described by Golub et al., “Molecular Classification of Cancer: Class discovery and class prediction by marker expression monitoring.” Science, 286:531-537 (1999), the contents of which are incorporated herein by reference. For weighted voting, the classification criterion for each feature used the following formula for the weighted vote of feature j:

$${V}_{j} = \frac{( {\overline{x}}_{R} - {\overline{x}}_{S} )}{{S}_{S} + {S}_{R}} [ {z}_{j} - {( \frac{{\overline{x}}_{R} + {\overline{x}}_{S}}{2} )}_{j} ]$$

where zⱼ represents the log expression value for the j^{th} feature in the set. For the class indicated by the subscript, \overline{x}represents the mean log expression value of the jth feature, and S represents the standard deviation. The first term on the right hand side of the equation is signal-to-noise ratio (the weight given to this feature in the weighted voting), while the subtracted term is called the decision boundary. To determine the class prediction, the weighted votes for all the features in the set are summed. If the result is greater than 0, then the prediction is class R; otherwise, the prediction is class S. For each prediction, a confidence is also computed. To compute the confidence, each feature in the set is labeled as being in agreement or disagreement with the class prediction. Let Vₐ be the sum of the absolute values of the votes of the features in agreement with the class prediction, and let V_{d} be the sum of absolute values of the votes in disagreement with the class prediction. Then the prediction confidence is defined as:

$$C = \frac{{v}_{a}}{{v}_{a} + {v}_{d}}$$

The CTF classifier first chooses a threshold on the normalized expression value for each feature. The CTF threshold is the CBT threshold divided by the CBT feature filtering score, each of which are described above. Expression values are then divided by this threshold, resulting in a “threshold-normalized expression value.” The threshold-normalized expression values of the features in the marker set or model are then combined into a “combined value” using one of these methods: (1) average, (2) maximum. In preferred embodiments, the first approach, average, is used. Finally, a threshold on the combined value is determined as the average value of the combined values in class A, plus some number of standard deviations of the combined values in class A. In preferred embodiments, the number of standard deviations is 2. Using the terminology introduced in the description of the CBT feature filtering method, samples with a combined value below this threshold are classified into class A, and samples with a combined value above this threshold are classified into class B.

Feature Selection

Feature selection is the process of determining the best subset of the 44,928 available features in the dataset, resulting in a combination of features, that form a marker set or model, to classify patients into sensitive and resistant groups. The first step is filtering to the top 100 markers, as described above. Next, for building Weighted Voting (WV) marker sets, a standard feature selection method, sequential forward feature selection, is used (Dash and Liu, “Feature Selection for Classification,” Intelligent Data Analysis 1:131-156, 1997). For building CTF marker sets, two methods were utilized: selection of the top N CBT scored markers (N<=100), and exhaustive search of all one- and two-feature models. We now describe how each of these is applied to our dataset to select features.

For the WV models, the top 100 SNR markers were determined. Sequential forward selection starts with no markers in the set.

At each iteration, a new feature set is formed by adding a feature selected by an evaluation function. Iteration terminates when no feature can be added that improves the evaluation function. The evaluation function has two parts. The first part is the number of samples correctly predicted either (1) by the model built on all of the samples, or (2) in leave-one-out cross-validation (Dash and Liu, 1997). Ties in the first part of the evaluation function are broken by a value equal to the sum of the confidences of the correct predictions less the sum of the confidences of the incorrect predictions. This second part of the evaluation function favors sets that have higher confidence and more correct predictions.

Each probe set was used as a single-marker model to predict bortezomib response. Multiple marker sets were generated by repeated rounds of feature selection, each time removing the features already selected. The score of each model was determined. The probe set comprising the highest-scoring model was selected.

The remaining probe sets were each used one at a time in a model along with the already-selected probe set(s). Each of these models was given a score. If the score of the new model was no higher than the score of the already-selected markers, then marker selection stopped, and the algorithm goes on to final selection by setting aside and continuing with selection of additional set(s) (see below). Otherwise, the probe set that was added to the already-selected markers to obtain the model with the highest score was added to the list of selected markers, and the algorithm returns to selection of additional markers to improve the score.

Upon final selection where no additional marker improves the score, the selected markers are set aside. Marker selection is then initiated as described above. This process is repeated until there are 5 sets of selected markers. These are combined into one complete predictive marker set.

For building CTF marker sets, the top 100 CBT features are considered for use in sets, and all one- and two-feature sets are evaluated exhaustively. The score for a given set is the number of class B samples which are above the CTF threshold (described above) for that set. Ties between CTF marker sets are broken by the best CBT score (described above) of any of the constituent markers in a set.

An example of a weighted voting predictive marker set identified using the WV and SNR scored markers is set forth in Table 4. This procedure is one of many described herein as well as others known in the art, which can be used to identify and select markers for sets predicting proteasome inhibition response in cancer patients. This procedure is the same as the procedure used in cross-validation to determine the predictive accuracy of the method (see Classification Accuracy below:

TABLE 4
Weighted Voting Predictive Marker Set

| | Decision | | | | Gene |
|---|---|---|---|---|---|
| No. | boundary | Weight | Probe set ID | Title | Symbol |
| 143 | 0.5177 | 0.8165 | 200965_s_at | actin binding LIM protein 1 | ABLIM1 |
| 141 | 0.3222 | 0.9174 | 234428_at | Homo sapiens mRNA; cDNA | — |
| | | | | DKFZp564I1316 (from clone | |
| | | | | DKFZp564I1316) | |
| 221 | 1.1666 | −0.8281 | 223996_s_at | mitochondrial ribosomal protein | MRPL30 |
| | | | | L30 | |
| 94 | 0.9622 | −0.8998 | 222555_s_at | mitochondrial ribosomal protein | MRPL44 |
| | | | | L44 | |
| 147 | 0.29 | 0.9019 | 220572_at | hypothetical protein | DKFZp547 |
| | | | | DKFZp547G183 | G183 |
| 242 | 0.8798 | −0.739 | 225647_s_at | cathepsin C | CTSC |
| 180 | 0.3451 | 0.8046 | 227692_at | guanine nucleotide binding protein | GNAI1 |
| | | | | (G protein), alpha inhibiting activity | |
| | | | | polypeptide 1 | |
| 279 | 0.8811 | 0.7428 | 221223_x_at | cytokine inducible SH2-containing | CISH |
| | | | | protein | |
| 163 | 0.4398 | 0.8189 | 204287_at | synaptogyrin 1 | SYNGR1 |
| 38 | 0.4805 | 0.8322 | 216835_s_at | docking protein 1, 62 kDa | DOK1 |
| | | | | (downstream of tyrosine kinase 1) | |
| 277 | 1.0222 | −0.7718 | 222713_s_at | Fanconi anemia, complementation | FANCF |
| | | | | group F | |
| 138 | 0.3196 | 0.9477 | 212109_at | HN1 like | HN1L |
| 36 | 0.4335 | 0.897 | 239476_at | Homo sapiens cDNA FLJ36491 fis, | — |
| | | | | clone THYMU2018197. | |
| 154 | 0.5779 | −0.8579 | 218438_s_at | endothelial-derived gene 1 | EG1 |
| 83 | 0.9308 | −0.9007 | 201575_at | SKI-interacting protein | SNW1 |
| 137 | 2.121 | −0.9414 | 200043_at | enhancer of rudimentary homolog | ERH |
| | | | | (Drosophila) | |
| 165 | 0.8934 | −0.8614 | 210250_x_at | adenylosuccinate lyase | ADSL |
| 251 | 1.5602 | −0.7928 | 208642_s_at | X-ray repair complementing | XRCC5 |
| | | | | defective repair in Chinese hamster | |
| | | | | cells 5 (double-strand-break | |
| | | | | rejoining; Ku autoantigen, 80 kDa) | |
| 120 | 0.3485 | 0.8612 | 217687_at | adenylate cyclase 2 (brain) | ADCY2 |
| 152 | 1.3737 | −0.8783 | 201682_at | peptidase (mitochondrial | PMPCB |
| | | | | processing) beta | |
| 96 | 1.2482 | −0.8447 | 222530_s_at | McKusick-Kaufman syndrome | MKKS |
| 245 | 0.3578 | 0.7543 | 203561_at | Fc fragment of IgG, low affinity IIa, | FCGR2A |
| | | | | receptor for (CD32) | |
| 241 | 0.9737 | −0.8018 | 222893_s_at | hypothetical protein FLJ13150 | FLJ13150 |
| 260 | 1.5048 | −0.792 | 222531_s_at | chromosome 14 open reading frame | C14orf108 |
| | | | | 108 | |
| 311 | 2.3688 | −0.7505 | 200826_at | small nuclear ribonucleoprotein D2 | SNRPD2 |
| | | | | polypeptide 16.5 kDa | |
| 213 | 0.3054 | −0.834 | 226882_x_at | WD repeat domain 4 | WDR4 |
| 224 | 1.2833 | 0.7725 | 235875_at | ESTs | — |
| 290 | 0.8235 | −0.7645 | 218139_s_at | chromosome 14 open reading frame | C14orf108 |
| | | | | 108 | |
| 145 | 1.6774 | −0.9194 | 232075_at | recombination protein REC14 | REC14 |
| 312 | 2.2771 | −0.7446 | 203663_s_at | cytochrome c oxidase subunit Va | COX5A |
| 49 | 1.0533 | −0.7456 | 208743_s_at | tyrosine 3- | YWHAB |
| | | | | monooxygenase/tryptophan 5- | |
| | | | | monooxygenase activation protein, | |
| | | | | beta polypeptide | |
| 160 | 1.1116 | −0.8655 | 202567_at | small nuclear ribonucleoprotein D3 | SNRPD3 |
| | | | | polypeptide 18 kDa | |
| 289 | 0.577 | 0.7398 | 208844_at | — | — |
| 87 | 0.7265 | 0.7845 | 234021_at | Homo sapiens cDNA: FLJ21331 | — |
| | | | | fis, clone COL02520. | |
| 170 | 0.4024 | 0.8105 | 216287_at | — | — |
| 129 | 2.216 | −0.8395 | 200814_at | proteasome (prosome, macropain) | PSMLE1 |
| | | | | activator subunit 1 (PA28 alpha) | |
| 149 | 0.7958 | 0.8846 | 221569_at | hypothetical protein FLJ20069 | FLJ20069 |
| 243 | 0.7858 | 0.7564 | 233876_at | Homo sapiens cDNA FLJ20670 fis, | — |
| | | | | clone KAIA4743. | |
| 195 | 1.1291 | 0.7902 | 58367_s_at | hypothetical protein FLJ23233 | FLJ23233 |
| 190 | 0.7554 | 0.7919 | 205807_s_at | tuftelin 1 | TUFT1 |

Classification Accuracy

To determine the ability of the selected model to predict sensitivity or resistance in an independent group of tumors, five-fold cross-validation was applied. For more information on cross-validation, see for example Kohavi and John, “Wrappers for Feature Subset Selection,” Artificial Intelligence 97 (1-2) (1997) pp. 273-324. Cross-validation provides for repeated division of the data set into training and test sets, building the model each time using only the training set, then evaluating its accuracy on the withheld test set. Five-fold cross-validation means that the training set contains 80% and the test set 20% of the original data set. The filtering, feature selection and model building operations are performed only on the training set, and the resulting models are then applied to the test set. Classification accuracy is measured only on the test sets, across multiple runs of cross-validation.

To determine if the most highly predictive models could be obtained by chance alone, a permutation test was performed. The labels were permuted on the 44 discovery samples 10 times; the entire marker selection procedure was repeated. Using Weighted Voting on the responders vs others comparison, for example, the overall error rate for the permuted models was 50%, compared to 29% for the observed labels. These results suggest that it is unlikely that those models could be identified by chance alone. In the refractory vs others comparisons, we did not see clear improvement of prediction accuracy when compared to permuted sample labels. However, we report here individual markers that have relatively high single-marker SNR or CBT scores.

It will be appreciated that additional marker sets may thus be obtained by employing the methods described herein for identifying models. There are many highly correlated features that could be substituted for each other in the models; these are not all listed.

Specific Application of Class Prediction

Weighted Voting (WV)

Here we illustrate how to apply a Weighted Voting model to obtain a prediction of Response or Non-response for a given patient, using the algorithm described herein. Using the 44 patients classified into Responsive or Nonresponsive groups, the table below shows the SNR scores and decision boundaries for each of the markers in a Weighted Voting predictive set built from the data set. Also indicated is whether the marker is more highly expressed in Responsive (R) or in Non-responsive (NR) patients. For one illustrative Non-responsive patient in the data set, the votes contributed by each marker are shown in Table 5. The sum of the vote weights is less than 0, indicating a prediction of Non-responsive. The confidence in the predicted class (Non-responsive) is 0.8431.

TABLE 5
Weighted Voting Predictive Marker Set

| | | | | | Ex. | | | |
|---|---|---|---|---|---|---|---|---|
| | | Gene | SNR | Decision | patient log | Vote | | |
| No. | Probe Set ID | Symbol | scores | boundary | expression | weight | Vote | Confidence |
| 143 | 200965_s_at | ABLIM1 | 0.8165 | 0.5177 | 0.3085 | −0.1708 | NR | |
| 141 | 234428_at | — | 0.9174 | 0.3222 | 0.201 | −0.1112 | NR | |
| 221 | 223996_s_at | MRPL30 | −0.8281 | 1.1666 | 1.0436 | 0.1019 | R | |
| 94 | 222555_s_at | MRPL44 | −0.8998 | 0.9622 | 1.2401 | −0.2501 | NR | |
| 147 | 220572_at | DKFZp54 | 0.9019 | 0.29 | 0.2731 | −0.0153 | NR | |
| | | 7G183 | | | | | | |
| | Total | | | | | −0.4454 | NR | 0.8431 |

It will be appreciated that similar methods may be employed utilizing the marker sets of the present invention.

Combination of Threshold Features (CTF)

Using the 44 patients classified into Responsive or Nonresponsive groups, the normalization threshold for each of the up-in-Nonpredictive markers in a CTF predictive set was built from our data set. Each marker value for a patient expression is scaled by dividing by a factor which is the mean of the Responsive class divided by the CBT score for that marker. Normalized expression values are summed to determine the combined predictive value for that patient. The threshold above which patients are predicted to be Nonresponsive was determined to be 59.15, by the CTF method described above. Because the average scaled expression value for this patient is 46.81, which is less than 59.15, the patient is predicted to be responsive. See Table 6.

It will be appreciated that similar methods may be employed utilizing one or more markers from the identified marker sets of the present invention in order to generate similar Predictive Marker Sets.

TABLE 6
CTF Predictive Marker Set

| | | RefSeq/ | | | | | Normalized |
|---|---|---|---|---|---|---|---|
| | | Genbank | | Gene | Normalization | gene | gene |
| No. | Probeset ID | Accession | Title | Symbol | factor | expr. | expression |
| 28 | 201457_x_at | AF081496.1 | BUB3 budding uninhibited by benzimidazoles 3 | BUB3 | 250.785036 | 549.1 | 2.18952458 |
| | | | homolog (yeast) | | | | |
| 152 | 201682_at | NM_004279.1 | peptidase (mitochondrial processing) beta | PMPCB | 181.94166 | 373 | 2.05010771 |
| 178 | 206978_at | NM_000647.2 | chemokine (C-C motif) receptor 2 | CCR2 | 248.903364 | 263 | 1.05663498 |
| 5 | 214265_at | AI193623 | integrin, alpha 8 | ITGA8 | 141.445138 | 176.5 | 1.24783363 |
| 197 | 217466_x_at | L48784 | — | — | 197.537832 | 833.4 | 4.21893868 |
| 158 | 217915_s_at | NM_016304.1 | chromosome 15 open reading frame 15 | C15orf15 | 218.690016 | 629.7 | 2.87941814 |
| 16 | 217969_at | NM_013265.2 | melanoma antigen, family D, 1 | MAGED1 | 206.919392 | 426.4 | 2.06070584 |
| 146 | 220565_at | NM_016602.1 | G protein-coupled receptor 2 | GPR2 | 70.449873 | 53.1 | 0.75372741 |
| 150 | 222427_s_at | AK021413.1 | leucyl-tRNA synthetase | LARS | 247.606604 | 721.1 | 2.91228097 |
| 207 | 222465_at | AF165521.1 | chromosome 15 open reading frame 15 | C15orf15 | 404.384832 | 1167.7 | 2.88759594 |
| 144 | 222783_s_at | NM_022137.1 | SPARC related modular calcium binding 1 | SMOC1 | 103.896695 | 119.9 | 1.15403093 |
| 167 | 223358_s_at | AW269834 | Homo sapiens cDNA FLJ33024 fis, clone | — | 131.346515 | 296.2 | 2.25510361 |
| | | | THYMU1000532. | | | | |
| 84 | 224985_at | BE964484 | Homo sapiens, clone IMAGE: 3446533, mRNA | — | 304.941586 | 860.4 | 2.82152399 |
| 162 | 225065_x_at | AI826279 | hypothetical protein MGC40157 | MGC40157 | 386.788155 | 943.5 | 2.43931979 |
| 199 | 225698_at | BF314746 | TIGA1 | TIGA1 | 285.001406 | 1317.3 | 4.62208246 |
| 188 | 226392_at | AI888503 | Homo sapiens cDNA: FLJ21652 fis, clone COL08582. | — | 249.877029 | 421.8 | 1.68803032 |
| 171 | 228332_s_at | AA526939 | selenoprotein H | SELH | 869.698724 | 1647.4 | 1.89421918 |
| 177 | 231045_x_at | H29876 | selenoprotein H | SELH | 620.98954 | 1078.1 | 1.7361001 |
| 145 | 232075_at | BF791874 | recombination protein REC14 | REC14 | 179.443992 | 540.9 | 3.01431101 |
| 140 | 232231_at | AL353944.1 | Runt domain transcription factor 2 | RUNX2 | 32.563013 | 95.4 | 2.92970432 |

| | | |
|---|---|---|
| | sum of normalized expression values | 46.8111936 |
| | threshold of control values | 59.15 |
| (>threshold = nonresponder; <threshold = responder) | Responder or nonresponder? | Responder |

Biological Annotation of Predictive Markers

Among the response genes identified in Table 1 and Table 2, are a subset of genes whose putative biological function or functions are particularly interesting, including function(s) particularly relevant to the use of proteasome inhibitors for the treatment of cancers, including myeloma. Some of the genes are known to be involved in the initiation or progression of myeloma, the growth, survival or signaling of lymphoid cells, the regulation of drug metabolism or apoptotic pathways or encode components of the ubiquitin/proteasome pathway that is directly targeted by proteasome inhibitors. For example, this analysis identified genes in Table 1 that are associated with cellular adhesion (No. 1 to 5), apoptotic signalling (6 to 13), cancer antigen (14 to 27), cell cycle (28 to 33), drug metabolism (34 to 35), drug resistance (36 to 37), growth control, hematopoesis (38 to 44), mitogenic signaling (45-53), myeloma signaling (53 to 61), myeloma translocation (62-73), NFkB pathway (74-77), oncogenes (78 to 82), oncogenic signaling (83 to 93), protein homeostasis (94 to 118), tumor suppressor pathway (119 to 128), and the ubiquitin/proteasome pathway (129 to 136). Additionally, the genes identified in this exercise also correspond to genes also correspond to the predictive markers associated with progressive disease in Table 2. See Table 7.

The identification of such genes strengthens the hypothesis that the genes identified with these methodologies are indeed related to cancer biology and the potential sensitivity of a hematological tumor to the anti-cancer actions of a proteasome inhibitor (e.g., bortezomib). Further, the description of such functional molecules as markers of response could facilitate selection of the most appropriate markers for inclusion in a diagnostic tool. In cases where 2 distinct probesets provide equal predictive information, the inclusion of these or other markers known to be biologically relevant could facilitate uptake and implementation of the diagnostic method. Finally, characterization of these functional molecules and pathways may enable the identification of new and possibly improved markers that act in the same or similar biological pathways.

Further, this analysis indicates additional genomic markers of response may be found in these biological pathways. For example, the “oncogenic signaling” category contains several components of the Wnt signaling pathway. Thus, other genes or proteins that function in the Wnt pathway that may also be employed as response markers. Additional markers in these identified pathways may also function alone or in conjunction with markers shown in Table 1 and Table 2 to effectively predict response to treatment with bortezomib.

TABLE 7
Biological Annotation

| | Probeset | | Gene | R/ | | Biological |
|---|---|---|---|---|---|---|
| No. | ID | Title | Symbol | NR | supplemental annotation | Category |
| 1 | 204298_{—} | lysyl oxidase | LOX | R | lysyl oxidase may play an important role in metastasis of colon, | Adhesion |
| | s_at | | | | espohageal, cardiac, and gastric carcinomas | |
| 2 | 205884_{—} | integrin, alpha 4 (antigen | ITGA4 | NR | Alpha 4 combines with beta 1 (ITGB1) on T-cells to form the | Adhesion |
| | at | CD49D, alpha 4 subunit of | | | integrin very late (activation) antigen 4 (‘VLA-4’) that can bind to | |
| | | VLA-4 receptor) | | | the extracellular matrix molecules fibronectin or thrombospondin, | |
| | | | | | and is also a ligand for the cell surface molecule vascular cell | |
| | | | | | adhesion molecule 1 (‘VCAM-1’). In addition, alpha 4 combines | |
| | | | | | with beta 7 to form the lymphocyte homing receptor known as | |
| | | | | | ‘LPAM-1’ (lymphocyte Peyer Patch adhesion molecule 1). Integrins | |
| | | | | | are also known to participate in cell-surface mediated signalling. | |
| 3 | 228841_{—} | Homo sapiens cDNA | — | NR | An inhibitor of matrix metalloproteinases. Prohibit the degradation | Adhesion |
| | at | fis, FLJ32429 clone | | | of the extracellualr matrix which is often a key step in the | |
| | | SKMUS2001014. | | | metastasis of tumor cells | |
| 4 | 243366_{—} | integrin, alpha 4 (antigen | ITGA4 | NR | Alpha 4 combines with beta 1 (ITGB1) on T-cells to form the | Adhesion |
| | s_at | CD49D, alpha 4 subunit of | | | integrin very late (activation) antigen 4 (‘VLA-4’) that can bind to | |
| | | VLA-4 receptor) | | | the extracellular matrix molecules fibronectin or thrombospondin, | |
| | | | | | and is also a ligand for the cell surface molecule vascular cell | |
| | | | | | adhesion molecule 1 (‘VCAM-1’). In addition, alpha 4 combines | |
| | | | | | with beta 7 to form the lymphocyte homing receptor known as | |
| | | | | | ‘LPAM-1’ (lymphocyte Peyer Patch adhesion molecule 1). Integrins | |
| | | | | | are also known to participate in cell-surface mediated signalling. | |
| 5 | 214265_{—} | integrin, alpha 8 | ITGA8 | NR | | Adhesion |
| | at | | | | | |
| 6 | 203949_{—} | myeloperoxidase | MPO | R | MPO derived oxidants are involved in caspase-3 activation and | Apoptotic |
| | at | | | | apoptosis, also translocations invoving this gene are often found in | signalling |
| | | | | | leukemia | |
| 7 | 207341_{—} | proteinase 3 (serine | PRTN3 | R | Cleavage of p21 waf1 by proteinase-3, a myeloid-specific serine | Apoptotic |
| | at | proteinase, neutrophil, | | | protease, potentiates cell proliferation. Also proteinase-3 mediates | signalling |
| | | Wegener granulomatosis | | | doxorubicin-induced apoptosis in the HL-60 leukemia cell line, and | |
| | | autoantigen) | | | is downregulated in its doxorubicin-resistant variant | |
| 8 | 203948_{—} | myeloperoxidase | MPO | R | MPO derived oxidants are involved in caspase-3 activation and | Apoptotic |
| | s_at | | | | apoptosis, also translocations invoving this gene are often found in | signalling |
| | | | | | leukemia | |
| 9 | 224461_{—} | apoptosis-inducing factor | AMID | NR | Overexpression of this gene has been shown to induce apoptosis. | Apoptotic |
| | s_at | (AIF)-homologous | | | The expression of this gene is found to be induced by tumor | signalling |
| | | mitochondrion-associated | | | suppressor protein p53 in colon caner cells. | |
| | | inducer of death | | | | |
| 10 | 206056_{—} | sialophorin (gpL115, | SPN | R | engagement of CD43 may, presumably through the repressing | Apoptotic |
| | x_at | leukosialin, CD43) | | | transcription, initiate a Bad-dependent apoptotic pathway. | signalling |
| 11 | 203489_{—} | CD27-binding (Siva) protein | SIVA | NR | This protein seems to have an important role in the apoptotic | Apoptotic |
| | at | | | | (programmed cell death) pathway induced by the CD27 antigen, a | signalling |
| | | | | | member of the tumor necrosis factor receptor (TFNR) superfamily, | |
| | | | | | and it also binds to the CD27 antigen cytoplasmic tail. | |
| 12 | 226507_{—} | p21/Cdc42/Rac1-activated | PAK1 | NR | (Pak1, Pak2, Pak3) have been studied in greater detail and shown to | Apoptotic |
| | at | kinase 1 (STE20 homolog, | | | be involved in the regulation of cellular processes such as gene | signalling |
| | | yeast) | | | transcription, cell morphology, motility, and apoptosis. | |
| 13 | 216055_{—} | platelet-derived growth factor | PDGFB | R | Most proliferating cells are programmed to undergo apoptosis | Apoptotic |
| | at | beta polypeptide (simian | | | unless specific survival signals are provided. Platelet-derived | signalling |
| | | sarcoma viral (v-sis) oncogene | | | growth factor promotes cellular proliferation and inhibits apoptosis. | |
| | | homolog) | | | Romashkova and Makarov (1999) showed that PDGF activates the | |
| | | | | | RAS/PIK3/AKT1/IKK/NFKB1 pathway. In this pathway, NFKB1 | |
| | | | | | (164011) does not induce c-myc and apoptosis, but instead induces | |
| | | | | | putative antiapoptotic genes. In response to PDGF, AKT1 (164730) | |
| | | | | | transiently associates with IKK (see 600664) and induces IKK | |
| | | | | | activation. The authors suggested that under certain conditions | |
| | | | | | PIK3 (see 171834) may activate NFKB1 without the involvement | |
| | | | | | of NFKBIA (164008) or NFKBIB (604495) degradation. | |
| 14 | 209942_{—} | melanoma antigen, | MAGEA3 | NR | A cancer antigen that binds to pro-caspase 12 and prevents its | Cancer |
| | x_at | family A, 3 | | | cleavage, therby preventing apoptosis reulting from ER stress, | Antigen |
| | | | | | including the unfolded protein response | |
| 15 | 214612_{—} | Human MAGE-6 antigen | — | NR | A cancer/testis antigen | Cancer |
| | x_at | (MAGE6) gene | | | | Antigen |
| 16 | 217969_{—} | melanoma antigen, | MAGED1 | NR | A cancer/testis antigen | Cancer |
| | at | family D, 1 | | | | Antigen |
| 17 | 215733_{—} | cancer/testis antigen 2 | CTAG2 | NR | A cancer/testis antigen | Cancer |
| | x_at | | | | | Antigen |
| 18 | 210546_{—} | cancer/testis antigen 1 | CTAG1 | NR | A cancer/testis antigen | Cancer |
| | x_at | | | | | Antigen |
| 19 | 211674_{—} | cancer/testis antigen 1 | CTAG1 | NR | A cancer/testis antigen | Cancer |
| | x_at | | | | | Antigen |
| 20 | 223313_{—} | MAGE-E1 protein | MAGE- | R | A cancer/testis antigen | Cancer |
| | s_at | | E1 | | | Antigen |
| 21 | 210467_{—} | melanoma antigen, family A, | MAGEA | NR | A cancer/testis antigen | Cancer |
| | x_at | 12 | 12 | | | Antigen |
| 22 | 220057_{—} | GACED2: G antigen, | GAGED2 | NR | A cancer/testis antigen | Cancer |
| | at | family D, 2 | | | | Antigen |
| 23 | 236152_{—} | PAGE-5 protein | PAGE-5 | NR | A cancer/testis antigen | Cancer |
| | at | | | | | Antigen |
| 24 | 233831_{—} | Homo sapiens serologically | — | R | A breast cancer antigen | Cancer |
| | at | defined breast cancer antigen | | | | Antigen |
| | | NY-BR-40 mRNA, partial cds | | | | |
| 25 | 206427_{—} | melan-A | MLANA | R | A cancer/testis antigen recognized by cytotoxic T-lympohocytes | Cancer |
| | s_at | | | | | Antigen |
| 26 | 206218_{—} | melanoma antigen, | MAGEB2 | NR | A cancer/testis antigen | Cancer |
| | at | family B, 2 | | | | Antigen |
| 27 | 203386_{—} | TBC1 domain family, | TBC1D4 | R | cancer antigen detected first in human sarcoma | Cancer |
| | at | member 4 | | | | Antigen |
| 28 | 201457_{—} | BUB3 budding uninhibited by | BUB3 | NR | mitotic spindle checkpoint component | Cell cycle |
| | x_at | benzimidazoles 3 homolog | | | | |
| | | (yeast) | | | | |
| 29 | 213348_{—} | cyclin-dependent kinase | CDKN1C | R | Cyclin-dependent kinase inhibitor 1C is a tight-binding inhibitor of | Cell cycle |
| | at | inhibitor 1C (p57, Kip2) | | | several G1 cyclin/Cdk complexes and a negative regulator of cell | |
| | | | | | proliferation. Mutations of CDKN1C are implicated in sporadic | |
| | | | | | cancers and Beckwith-Wiedemann syndorome suggesting that it is a | |
| | | | | | tumor suppressor candidate. | |
| 30 | 204170_{—} | CDC28 protein kinase | CKS2 | NR | CKS2 protein binds to the catalytic subunit of the cyclin dependent | Cell cycle |
| | s_at | regulatory subunit 2 | | | kinases and is essential for their biological function. The CKS2 | |
| | | | | | mRNA is found to be expressed in different patterns through the | |
| | | | | | cell cycle in HeLa cells, which reflects specialized role for the | |
| | | | | | encoded protein. | |
| 31 | 206205_{—} | M-phase phosphoprotein 9 | MPHOS | NR | May be involveded in the progression from G2 to M phase in the | Cell cycle |
| | at | | PH9 | | cell cycle | |
| 32 | 208796_{—} | cyclin G1 | CCNG1 | NR | The cyclin G1 gene has been identified as a target for | Cell cycle |
| | s_at | | | | transcriptional activation by the p53 tumor suppressor protein. | |
| 33 | 204460_{—} | RAD1 homolong (S. pombe) | RAD1 | NR | Has strong sequence homology to cell cycle checkpoint gene | Cell cycle |
| | s_at | | | | required for cell cycle arrest and DNA damage repair in response to | |
| | | | | | DNA damage | |
| 34 | 224918_{—} | microsomal glutathione S- | MGST1 | NR | MGST1 is a drug metabolizing enzyme involved in cellular defense | Drug |
| | x_at | transferase 1 | | | against toxic electrophilic compounds. Localized to the | metabolism |
| | | | | | endoplasmic reticulum and outer mitochondrial membrane where it | |
| | | | | | is thought to protect these membranes from oxidative stress. | |
| 35 | 205998_{—} | cytochrome P450, subfamily | CYP3A4 | R | Expression is induced by glucocorticoids and some | Drug |
| | x_at | IIIA (niphedipine oxidase), | | | pharmacological agents. This enzyme is involved in the metabolism | metabolism |
| | | polypeptide 4 | | | of approximately half the drugs which are are used today, including | |
| | | | | | acetaminophen, codeine, cyclosporin A, diazepam and | |
| | | | | | erythromycin. | |
| 36 | 239476_{—} | phosphoinositide-3-kinase, | PIK3R1 | R | PIK3R1: phosphoinositide-3-kinase, regulatory subunit, | Drug |
| | at | regulatory subunit, | | | polypeptide 1 (p85 alpha); pro-apoptotic activity via suppression of | Resistance |
| | | polypeptide 1 (p85 alpha) | | | the AKT survival pathway that is frequently activated in myeloma | |
| 37 | 211298_{—} | albumin | ALB | R | Albumin has been shown to acitivate the AKT signalling pathway | Drug |
| | s_at | | | | and protect B-chronic lymphocytic leukemia patients from | Resistance |
| | | | | | chlorambucil- and radiation-induced apoptosis | |
| 38 | 216835_{—} | docking protein 1, 62 kDa | DOK1 | R | Docking protein 1 is constitutively tyrosine phosphorylated in | Hema- |
| | s_at | (downstream of tyrosine | | | hematopoietic progenitors isolated from chronic myelogenous | topoiesis |
| | | kinase 1) | | | leukemia (CML) patients in the chronic phase. It may be a critical | |
| | | | | | substrate for p210(bcr/abl), a chimeric protein whose presence is | |
| | | | | | associated with CML. | |
| 39 | 213891_{—} | TCF4 | — | R | TCF4 is expressed predominantly in pre-B-cells, it is activated upon | Hema- |
| | s_at | | | | Wnt signalling | topoiesis |
| 40 | 212387_{—} | TCF4 | — | R | TCF4 is expressed predominantly in pre-B-cells, it is activated upon | Hema- |
| | at | | | | Wnt signalling | topoiesis |
| 41 | 212382_{—} | TCF4: Transcription factor 4 | — | R | TCF4 is expressed predominantly in pre-B-cells, it is activated upon | Hema- |
| | at | | | | Wnt signalling | topoiesis |
| 42 | 203753_{—} | transcription factor 4 | TCF4 | R | TCF4 is expressed predominantly in pre-B-cells, it is activated upon | Hema- |
| | at | | | | Wnt signalling | topoiesis |
| 43 | 212386_{—} | transcription factor 4 | TCF4 | R | TCF4 is expressed predominantly in pre-B-cells, it is activated upon | Hema- |
| | at | | | | Wnt signalling | topoiesis |
| 44 | 211709_{—} | stem cell growth factor; | SCGF | R | SCGF is selectively produced by osseous and hematopoietic | Hema- |
| | s_at | lymphocyte secreted C-type | | | stromal cells, and can mediate their proliferative activity on | topoiesis |
| | | lectin | | | primitive hematopoietic progenitor cells. | |
| 45 | 217020_{—} | — | — | R | Binds retinoic acid, the biologically active form of vitamin A which | Mitogenic |
| | at | | | | mediates cellular signalling in embryonic morphogenesis, cell | Signalling |
| | | | | | growth and differentiation. | |
| 46 | 217786_{—} | SKB1 homolog (S. pombe) | SKB1 | NR | may regulate mitosis through binding SHK1 | Mitogenic |
| | at | | | | | Signalling |
| 47 | 206109_{—} | fucosyltransferase 1 | FUT1 | R | an essential component of Notch signalling pathway that regulate | Mitogenic |
| | at | (galactoside 2-alpha-L- | | | cell growth and differentiation | Signalling |
| | | fucosyltransferase, Bombay | | | | |
| | | phenotype included) | | | | |
| 48 | 227798_{—} | MADH1 MAD, mothers | — | NR | Involved in the TGF-beta signalling pathway, an important pathway | Mitogenic |
| | at | against decapentaplegic | | | that regulates cell growth, differentiation and apoptosis and is often | Signalling |
| | | homolog 1 (Drosophila) | | | disrupted in cancer. | |
| 49 | 208743_{—} | tyrosine 3- | YWHAB | NR | This gene encodes a protein belonging to the 14-3-3 family of | Mitogenic |
| | s_at | monooxygenase/tryptophan 5- | | | proteins. It has been shown to interact with RAF1 and CDC25 | Signalling |
| | | monooxygenase activation | | | phosphatases, suggesting that it may play a role in linking | |
| | | protein, beta polypeptide | | | mitogenic signaling and the cell cycle machinery. | |
| 50 | 225239_{—} | ESTs, Moderately similar to | — | R | SPRY4 is an inhibitor of the receptor-transduced mitogen-activated | Mitogenic |
| | at | hypothetical protein FLJ20958 | | | protein kinase (MAPK) signaling pathway, an important growth | Signalling |
| | | [Homo sapiens] [H. sapiens] | | | signalling pathway in cancer. | |
| 51 | 215551_{—} | estrogen receptor 1 | ESR1 | R | Estrogen receptor 1 alpha overexpression is implicated in breast and | Mitogenic |
| | at | | | | ovarian cancers, and activates the cyclin D1 pathway | Signalling |
| 52 | 215067_{—} | PRDX2: peroxiredoxin 2 | — | R | PRDX2 may have a proliferative effect and play a role in cancer | Mitogenic |
| | x_at | | | | development or progression. | Signalling |
| 53 | 210993_{—} | MAD, mothers against | MADH1 | NR | TGFB1 is the prototype of a large family of cytokines that also | Mitogenic |
| | s_at | decapentaplegic homolog 1 | | | includes the activins (e.g., 147290), inhibins (e.g., 147380), bone | Signalling |
| | | (Drosophila) | | | morphogenetic proteins, and Mullerian-inhibiting substance | |
| | | | | | (600957). Members of the TGF-beta family exert a wide range of | |
| | | | | | biologic effects on a large variety of cell types; for example, they | |
| | | | | | regulate cell growth, differentiation, matrix production, and | |
| | | | | | apoptosis. | |
| 54 | 209374_{—} | immunoglobulin heavy | IGHM | NR | A surrogate marker of some types of multiple myeloma | Myeloma |
| | s_at | constant mu | | | | signalling |
| 55 | 224342_{—} | immunoglobulin lambda locus | IGL@ | NR | A surrogate marker of some types of multiple myeloma | Myeloma |
| | x_at | | | | | signalling |
| 56 | 212827_{—} | immunoglobulin heavy | IGHM | NR | A surrogate marker of some types of multiple myeloma | Myeloma |
| | at | constant mu | | | | signalling |
| 57 | 234366_{—} | immunoglobulin lambda locus | IGL@ | R | A surrogate marker of some types of multiple myeloma | Myeloma |
| | x_at | | | | | Signalling |
| 58 | 216986_{—} | interferon regulatory factor 4 | IRF4 | NR | A mutliple myeloma oncogene, has been shown to regualte | Myeloma |
| | s_at | | | | lymphocyte apoptosis by modulating the efficiency of the Fas signal | signalling |
| 59 | 205098_{—} | chemokine (C-C motif) | CCR1 | NR | studies suggest that chemokine receptor expression and the | Myeloma |
| | at | receptor 1 | | | migratory capacity of MM cells to their ligands are relevant for the | signalling |
| | | | | | compartmentalization of MM cells in the bone marrow | |
| 60 | 239237_{—} | ESTs | — | NR | Strong sequence similarity to Ig heavy chain, a surrogate marker for | Myeloma |
| | at | | | | some types of multiple myeloma | signalling |
| 61 | 205099_{—} | chemokine (C-C motif) | CCR1 | NR | studies suggest that chemokine receptor expression and the | Myeloma |
| | s_at | receptor 1 | | | migratory capacity of multiple myeloma cells to their ligands are | signalling |
| | | | | | relevant for the compartmentalization of multiple myeloma cells in | |
| | | | | | the bone marrow | |
| 62 | 223472_{—} | Wolf-Hirschhorn syndrome | WHSC1 | R | WHSC1 is involved in a chromosomal translocation | Myeloma |
| | at | candidate 1 | | | t(4;14)(p16.3;q32.3) in multiple myelomas. | translocation |
| 63 | 222778_{—} | Wolf-Hirschhorn syndrome | WHSC1 | R | WHSC1 is involved in a chromosomal translocation | Myeloma |
| | s_at | candidate 1 | | | t(4;14)(p16.3;q32.3) in multiple myelomas. Also, vv | translocation |
| 64 | 209054_{—} | Wolf-Hirschhorn syndrome | WHSC1 | R | WHSC1 is involved in a chromosomal translocation | Myeloma |
| | s_at | candidate 1 | | | t(4;14)(p16.3;q32.3) in multiple myelomas. | translocation |
| 65 | 222777_{—} | Wolf-Hirschhorn syndrome | WHSC1 | R | WHSC1 is involved in a chromosomal translocation | Myeloma |
| | s_at | candidate 1 | | | t(4;14)(p16.3;q32.3) in multiple myelomas. Also, vv | translocation |
| 66 | 209053_{—} | Wolf-Hirschhorn syndrome | WHSC1 | R | WHSC1 is involved in a chromosomal translocation | Myeloma |
| | s_at | candidate 1 | | | t(4;14)(p16.3;q32.3) in multiple myelomas. Also, vv | translocation |
| 67 | 200921_{—} | B-cell translocation gene 1, | BTG1 | NR | The BTG1 gene locus has been shown to be involved in a | Myeloma |
| | s_at | anti-proliferative | | | t(8;12)(q24;q22) chromosomal translocation in a case of B-cell | translocation |
| | | | | | chronic lymphocytic leukemia. It is a member of a family of | |
| | | | | | antiproliferative genes. BTG1 expression is maximal in the G0/G1 | |
| | | | | | phases of the cell cycle and downregulated when cells progressed | |
| | | | | | through G1. It negatively regulates cell proliferation. | |
| 68 | 209052_{—} | Wolf-Hirschhorn syndrome | WHSC1 | R | WHSC1 is involved in a chromosomal translocation | Myeloma |
| | s_at | candidate 1 | | | t(4;14)(p16.3;q32.3) in multiple myelomas. | translocation |
| 69 | 213940_{—} | formin binding protein | FNBP1 | NR | The human formin-binding protein 17 (FBP17) interacts with | Myeloma |
| | s_at | 1(FBP17) | | | sorting nexin, SNX2, and is an MLL-fusion partner in acute | translocation |
| | | | | | myelogeneous leukemia | |
| 70 | 213732_{—} | transcription factor 3 (E2A | TCF3 | R | The E2A gene maps to 19p13.3-p13.2, a site associated with | Myeloma |
| | at | immunoglobulin enhancer | | | nonrandom translocations in acute lymphoblastic leukemias. | translocation |
| | | binding factors E12/E47) | | | | |
| 71 | 213047_{—} | SET translocation (myeloid | SET | NR | The SET translocation (6;9)(p23q34) is the hallmark of a specific | Myeloma |
| | x_at | leukemia-associated) | | | subtype of acute myeloid leukemia (AML) characterized by a poor | translocation |
| | | | | | prognosis and a young age of onset. SET protein regulates G(2)/M | |
| | | | | | transition by modulating cyclin B-CDK1 activity. | |
| 72 | 200631_{—} | SET translocation (myeloid | SET | NR | The SET translocation (6;9)(p23q34) is the hallmark of a specific | Myeloma |
| | s_at | leukemia-associated) | | | subtype of acute myeloid leukemia (AML) characterized by a poor | translocation |
| | | | | | prognosis and a young age of onset. SET protein regulates G(2)/M | |
| | | | | | transition by modulating cyclin B-CDK1 activity. | |
| 73 | 205068_{—} | GTPase regulator associated | GRAF | R | GTPase regulator associated with the focal adhesion kinase | Myeloma |
| | s_at | with focal adhesion kinase | | | pp125(FAK) is often involved in a translocations with the MLL | translocation |
| | | pp125(FAK) | | | gene in hematologic malignancies | |
| 74 | 220146_{—} | toll-like receptor 7 | TLR7 | NR | Expression of TLR7 may activate NF-kB, an important mediator of | NFkB |
| | at | | | | cell survival, and possible downstream target of proteasome | pathway |
| | | | | | inhibition | |
| 75 | 232304_{—} | pellino homolog 1 | PELI1 | R | Pellino 1 is required for NF kappa B activation and IL-8 gene | NFkB |
| | at | (Drosophila) | | | expression in response to IL-1 | pathway |
| 76 | 232213_{—} | pellino homolog 1 | PELI1 | R | Pellino 1 is required for NF kappa B activation and IL-8 gene | NFkB |
| | at | (Drosophila) | | | expression in response to IL-1 | pathway |
| 77 | 218319_{—} | pellino homolog 1 | PELI1 | R | Pellino 1 is required for NF kappa B activation and IL-8 gene | NFkB |
| | at | (Drosophila) | | | expression in response to IL-1 | pathway |
| 78 | 215744_{—} | fusion, derived from t(12;16) | FUS | R | Proto-oncoprotein resulting from fusion gene in myxoid | Oncogene |
| | at | malignant liposarcoma | | | liposarcoma; derived from t(12;16) malignant liposarcoma. | |
| 79 | 206363_{—} | v-maf musculoaponeurotic | MAF | R | MAF is a protooncogene | Oncogene |
| | at | fibrosarcoma oncogene | | | | |
| | | homolog (avian) | | | | |
| 80 | 202768_{—} | FBJ murine osteosarcoma | FOSB | R | The fos genes encode leucine zipper proteins that can dimerize with | Oncogene |
| | at | viral oncogene homolog B | | | proteins of the JUN family, thereby forming the transcription factor | |
| | | | | | complex AP-1. Thus, the FOS proteins have been implicated as | |
| | | | | | regulators of cell proliferation, differentiation, and oncogenic | |
| | | | | | transformation. | |
| 81 | 202647_{—} | neuroblastoma RAS viral (v- | NRAS | NR | The N-ras oncogene is a member of the RAS gene family. It is | Oncogene |
| | s_at | ras) oncogene homolog | | | mapped on chromosome 1, and it is activated in HL60, a | |
| | | | | | promyelocytic leukemia line. | |
| 82 | 209640_{—} | promyelocytic leukemia | PML | R | The expression of PML is cell-cycle related and it regulates the p53 | Oncogene |
| | at | | | | response to oncogenic signals. The gene is often involved in the | |
| | | | | | translocation with the retinoic acid receptor alpha gene associated | |
| | | | | | with acute promyelocytic leukemia (APL). | |
| 140 | 232231_{—} | Runt domain transcription | RUNX2 | NR | Runt domain transcription factor AML3/RUNX2 is essential for the | Oncogene |
| | at | factor | | | generation and differentiation of osteoblasts, and has been | |
| | | | | | associated with the survival of several types of metastases in bone. | |
| 83 | 201575_{—} | SKI-interacting protein | SNW1 | NR | may be involved in oncogenesis since it interacts with a region of | Oncogenic |
| | at | | | | SKI oncoproteins that is required for transforming | signalling |
| | | | | | activity; overcomes the growth-suppressive activities of pRb | |
| 84 | 224985_{—} | Homo sapiens, clone | — | NR | An oncogene involved in numerous cancers. A member of the RAS | Oncogenic |
| | at | IMAGE: 3446533, mRNA | | | gene family. | signalling |
| 85 | 204602_{—} | dickkopf homolog 1 (Xenopus | DKK1 | NR | A secreted inhibitor of WNT signalling, a pathway known to be | Oncogenic |
| | at | laevis) | | | important to oncogenesis | signalling |
| 86 | 201653_{—} | cornichon homolog | CNIH | NR | may regulate EGF signalling, a pathway known to be involved in | Oncogenic |
| | at | (Drosophila) | | | oncogenesis | signalling |
| 87 | 234021_{—} | Homo sapiens cDNA: | — | R | highly similar to plakophilin 2 which associates with beta-catenin | Oncogenic |
| | at | FLJ21331 fis, clone | | | and up-regulates the oncogenic beta-catenin/T cell factor-signaling | signalling |
| | | COL02520. | | | activity | |
| 88 | 212063_{—} | CD44 antigen (homing | CD44 | NR | The wide prevalence of CD44 cleavage suggests that it plays an | Oncogenic |
| | at | function and Indian blood | | | important role in the pathogenesis of human tumors. | signalling |
| | | group system) | | | | |
| 89 | 204489_{—} | CD44 antigen (homing | CD44 | NR | The wide prevalence of CD44 cleavage suggests that it plays an | Oncogenic |
| | s_at | function and Indian blood | | | important role in the pathogenesis of human tumors. | signalling |
| | | group system) | | | | |
| 90 | 227167_{—} | Homo sapiens mesenchymal | — | NR | The RAS oncogene (MIM 190020) is mutated in nearly one-third | Oncogenic |
| | s_at | stem cell protein DSC96 | | | of all human cancers. Members of the RAS superfamily are plasma | signalling |
| | | mRNA, partial cds | | | membrane GTP-binding proteins that modulate intracellular signal | |
| | | | | | transduction pathways. A subfamily of RAS effectors, including | |
| | | | | | RASSF3, share a RAS association (RA) domain | |
| 91 | 202290_{—} | PDGFA associated protein 1 | PDAP1 | NR | stimulates the inherent ATPase activity of Hsp90, a molecular | Oncogenic |
| | at | | | | chaperone that plays a key role in the conformational maturation of | signalling |
| | | | | | oncogenic signaling proteins | |
| 92 | 215499_{—} | mitogen-activated protein | MAP2K3 | R | Expression of RAS oncogene is found to result in the accumulation | Oncogenic |
| | at | kinase kinase 3 (MAP2K3) | | | of the active form of MAP2K3, which thus leads to the constitutive | signalling |
| | | | | | activation of MAPK14, and confers oncogenic transformation of | |
| | | | | | primary cells. | |
| 93 | 200047_{—} | YY1 transcription factor | YY1 | NR | Some AML patients showed significantly elevated YY1 transcript | Oncogenic |
| | s_at | | | | levels in bone marrow cells. Taken together with mouse data, this | signalling |
| | | | | | suggests involvement in the pathogenesis of AML. | |
| 94 | 222555_{—} | mitochondrial ribosomal | MRPL44 | NR | involved in mitochondrial protein synthesis | Protein |
| | s_at | protein L44 | | | | homeostasis |
| 95 | 212694_{—} | propionyl Coenzyme A | PCCB | NR | may function in protein homeostasis via degradation of brached | Protein |
| | s_at | carboxylase, beta polypeptide | | | chain amino acids | homeostasis |
| 96 | 222530_{—} | McKusick-Kaufman | MKKS | NR | similarity to the chaperonin family of proteins, suggesting a role for | Protein |
| | s_at | syndrome | | | protein processing | homeostasis |
| 97 | 200869_{—} | ribosomal protein L18a | RPL18A | NR | Ribosomes are involved in protein synthesis and thus contribute to | Protein |
| | at | | | | protein homeostasis | homeostasis |
| 98 | 200023_{—} | eukaryotic translation | EIF3S5 | NR | Regulates initiation of protein translation and thus is involved in | Protein |
| | s_at | initiation factor 3, | | | protein homeostasis | homeostasis |
| | | subunit 5 epsilon, 47 kDa | | | | |
| 99 | 200812_{—} | chaperonin containing TCP1, | CCT7 | NR | CCT regulates protein homeostasis via the folding of newly | Protein |
| | at | subunit 7 (eta) | | | translated polypeptide substrates, including cyclin E | homeostasis |
| 100 | 225190_{—} | ribosomal protein L35a | RPL35A | NR | Ribosomes are involved in protein synthesis and thus contribute to | Protein |
| | x_at | | | | protein homeostasis | homeostasis |
| 101 | 200023_{—} | eukaryotic translation | EIF3S5 | NR | Regulates initiation of protein translation and thus is involved in | Protein |
| | s_at | initiation factor 3, | | | protein homeostasis | homeostasis |
| | | subunit 5 epsilon, 47 kDa | | | | |
| 102 | 217919_{—} | mitochondrial ribosomal | MRPL42 | NR | involved in mitochondrial protein synthesis | Protein |
| | s_at | protein L42 | | | | homeostasis |
| 103 | 211972_{—} | ribosomal protein, large, P0 | RPLP0 | NR | Ribosomes are involved in protein synthesis and thus contribute to | Protein |
| | x_at | | | | protein homeostasis | homeostasis |
| 104 | 200024_{—} | ribosomal protein S5 | RPS5 | NR | Ribosomes are involved in protein synthesis and thus contribute to | Protein |
| | at | | | | protein homeostasis | homeostasis |
| 105 | 200715_{—} | ribosomal protein L13a | RPL13A | NR | Ribosomes are involved in protein synthesis and thus contribute to | Protein |
| | x_at | | | | protein homeostasis | homeostasis |
| 106 | 201258_{—} | ribosomal protein S16 | RPS16 | NR | Ribosomes are involved in protein synthesis and thus contribute to | Protein |
| | at | | | | protein homeostasis | homeostasis |
| 107 | 200003_{—} | ribosomal protein L28 | RPL28 | NR | Ribosomes are involved in protein synthesis and thus contribute to | Protein |
| | s_at | | | | protein homeostasis | homeostasis |
| 108 | 221726_{—} | ribosomal protein L22 | RPL22 | NR | Ribosomes are involved in protein synthesis and thus contribute to | Protein |
| | at | | | | protein homeostasis | homeostasis |
| 109 | 200041_{—} | HLA-B associated transcript 1 | BAT1 | R | Members of this family are involved in a number of cellular | Protein |
| | s_at | | | | functions including initiation of translation, RNA splicing, and | homeostasis |
| | | | | | ribosome assembly and thus could have a role in protein | |
| | | | | | homeostasis. | |
| 110 | 211937_{—} | eukaryotic translation | EIF4B | NR | Regulates initiation of protein translation and thus is involved in | Protein |
| | at | factor 4B | initiation | | protein homeostasis | homeostasis |
| 111 | 200082_{—} | ribosomal protein S7 | RPS7 | NR | Ribosomes are involved in protein synthesis and thus contribute to | Protein |
| | s_at | | | | protein homeostasis | homeostasis |
| 112 | 214167_{—} | ribosomal protein, large, P0 | RPLP0 | NR | Ribosomes are involved in protein synthesis and thus contribute to | Protein |
| | s_at | | | | protein homeostasis | homeostasis |
| 113 | 200024_{—} | ribosomal protein S5 | RPS5 | NR | Ribosomes are involved in protein synthesis and thus contribute to | Protein |
| | at | | | | protein homeostasis | homeostasis |
| 114 | 217719_{—} | eukaryotic translation | EIF3S6IP | NR | Regulates initiation of protein translation and thus is involved in | Protein |
| | at | initiation factor 3, subunit 6 | | | protein homeostasis | homeostasis |
| | | interacting protein | | | | |
| 115 | 225797_{—} | mitochondrial ribosomal | MRPL54 | NR | involved in mitochondrial protein synthesis | Protein |
| | at | protein L54 | | | | homeostasis |
| 116 | 200937_{—} | ribosomal protein L5 | RPL5 | NR | Ribosomes are involved in protein synthesis and thus contribute to | Protein |
| | s_at | | | | protein homeostasis | homeostasis |
| 117 | 208985_{—} | eukaryotic translation | EIF3S1 | NR | Regulates initiation of protein translation and thus is involved in | Protein |
| | s_at | initiation factor 3, | | | protein homeostasis | homeostasis |
| | | subunit 1 alpha, | | | | |
| 118 | 200834_{—} | 35 kDa ribosomal protein S21 | RPS21 | NR | Ribosomes are involved in protein synthesis and thus contribute to | Protein |
| | s_at | | | | protein homeostasis | homeostasis |
| 119 | 216153_{—} | reversion-inducing-cysteine- | RECK | R | The protein encoded by this gene is a cysteine-rich, extracellular | Tumor |
| | x_at | rich protein with kazal motifs | | | protein with protease inhibitor-like domains whose expression is | Supressor |
| | | | | | suppressed strongly in many tumors and cells transformed by | Pathway |
| | | | | | various kinds of oncogenes. In normal cells, this membrane- | |
| | | | | | anchored glycoprotein may serve as a negative regulator for matrix | |
| | | | | | metalloproteinase-9, a key enzyme involved in tumor invasion and | |
| | | | | | metastasis. | |
| 120 | 217687_{—} | adenylate cyclase 2 (brain) | ADCY2 | R | Adenylate cyclase signalling regulates cell growth and | Tumor |
| | at | | | | differentiation; it is frequently defective in human tumors. | Supressor |
| | | | | | Activation of human Adenylyl Cyclase protein(s) and inhibition of | Pathway |
| | | | | | human Pde4 protein protein(s) increase apoptosis of acute | |
| | | | | | lymphoblastic leukemia cells | |
| 121 | 222632_{—} | leucine zipper transcription | LZTFL1 | NR | The LZTFL1 gene has been mapped to a putative tumor suppressor | Tumor |
| | s_at | factor-like 1 | | | region (C3CER1) on chromosome 3p21.3 | Supressor |
| | | | | | | Pathway |
| 122 | 236623_{—} | ATPase, Na+/K+ | ATP1A1 | R | Expression regulated by p53, a tumor supressor gene | Tumor |
| | at | transporting, alpha 1 | | | | Supressor |
| | | polypeptide | | | | Pathway |
| 123 | 221899_{—} | hypothetical protein from | CG005 | R | Located in the region of BRCA2, a breast cancer susceptibility gene | Tumor |
| | at | BCRA2 region | | | | Supressor |
| | | | | | | Pathway |
| 124 | 221691_{—} | nucleophosmin (nucleolar | NPM1 | NR | Nucleophosmin regulates the stability and transcriptional activity of | Tumor |
| | x_at | phosphoprotein B23, | | | p53 | Supressor |
| | | numatrin) | | | | Pathway |
| 125 | 209030_{—} | immunoglobulin superfamily, | IGSF4 | NR | TSCL1 has been identified as a potential tumor supressor gene in | Tumor |
| | s_at | member 4 (TSLC1) | | | lung cancer | Supressor |
| | | | | | | Pathway |
| 126 | 222762_{—} | LIM domains containing 1 | LIMD1 | NR | Interstitial deletions of the short arm of chromosome 3 containing | Tumor |
| | x_at | (LIMD1) | | | LILMD1 are found in a large number of tumors. IT may have a role | Supressor |
| | | | | | as a tumor supressor. | Pathway |
| 127 | 240983_{—} | cysteinyl-tRNA synthetase | CARS | NR | This gene is one of several located near the imprinted gene domain | Tumor |
| | s_at | | | | of 11p15.5, an important tumor-suppressor gene region. Alterations | Supressor |
| | | | | | in this region have been associated with the Beckwith-Wiedemann | Pathway |
| | | | | | syndrome, Wilms tumor, rhabdomyosarcoma, adrenocortical | |
| | | | | | carcinoma, and lung, ovarian, and breast cancer. | |
| 128 | 200713_{—} | microtubule-associated | MAPRE1 | NR | MAPRE1 binds to the APC protein which is often mutated in | Tumor |
| | s_at | protein, RP/EB family, | | | familial and sporadic forms of colorectal cancer. This protein | Supressor |
| | | member 1 | | | localizes to microtubules, especially the growing ends, in interphase | Pathway |
| | | | | | cells. During mitosis, the protein is associated with the centrosomes | |
| | | | | | and spindle microtubules. | |
| 129 | 200814_{—} | proteasome (prosome, | PSME1 | NR | subunit of the 11S regulator of the 20S proteasome | Ubiquitin/ |
| | at | macropain) activator subunit 1 | | | | proteasome |
| | | (PA28 alpha) | | | | pathway |
| 130 | 201532_{—} | proteasome (prosome, | PSMA3 | NR | core subunit of the proteasome | Ubiquitin/ |
| | at | macropain) subunit, | | | | proteasome |
| | | alpha type, 3 | | | | pathway |
| 131 | 218011_{—} | ubiquitin-like 5 | UBL5 | NR | Ubiquitin-like proteins (UBLs) are thought to be reversible | Ubiquitin/ |
| | at | | | | modulators of protein function rather than protein degraders like | proteasome |
| | | | | | ubiquitin | pathway |
| 132 | 224747_{—} | hypothetical protein | L0C92912 | NR | Contains a ubiquitin conjugating enzyme domain | Ubiquitin/ |
| | at | LOC92912 | | | | proteasome |
| | | | | | | pathway |
| 133 | 201758_{—} | tumor susceptibility gene 101 | TSG101 | NR | The protein encoded by this gene belongs to a group of apparently | Ubiquitin/ |
| | at | | | | inactive homologs of ubiquitin-conjugating enzymes. The gene | proteasome |
| | | | | | product contains a coiled-coil domain that interacts with stathmin, a | pathway |
| | | | | | cytosolic phosphoprotein implicated in tumorigenesis. The protein | |
| | | | | | may play a role in cell growth and differentiation and act as a | |
| | | | | | negative growth regulator. | |
| 134 | 200019_{—} | Finkel-Biskis-Reilly murine | FAU | NR | A fusion protein consisting of the ubiquitin-like protein fubi at the | Ubiquitin/ |
| | s_at | sarcoma virus (FBR-MuSV) | | | N terminus and ribosomal protein S30 at the C terminus. It has been | proteasome |
| | | ubiquitously expressed (fox | | | proposed that the fusion protein is post-translationally processed to | pathway |
| | | derived); ribosomal protein | | | generate free fubi and free ribosomal protein S30. Fubi is a member | |
| | | S30 | | | of the ubiquitin family, and ribosomal protein S30 belongs to the | |
| | | | | | S30E family of ribosomal proteins. | |
| 135 | 202346_{—} | huntingtin interacting | HIP2 | NR | UBIQUITIN-CONJUGATING ENZYME E2-25 K has been | Ubiquitin/ |
| | at | protein 2 | | | implicated in the degradation of huntingtin and suppression of | proteasome |
| | | | | | apoptosis. | pathway |
| 136 | 201177 | SUMO-1 activating enzyme | UBA2 | NR | ubiquitin-like activating enzyme involved in protein homeostasis | Ubiquitin/ |
| | s_at | subunit 2 | | | | proteasome |
| | | | | | | pathway |
| 154 | 218438_{—} | endothelial-derived gene 1 | EG1 | NR | expressed in tumor-stimulated endothelial cells; may have role in | |
| | s_at | | | | tumor angiogenesis | |
| 157 | 216288_{—} | cysteinyl leukotriene | CYSLTR1 | R | upregulated in colon cancer; affecting survival | |
| | at | receptor 1 | | | | |
| 166 | 210497_{—} | synovial sarcoma, | SSX2 | NR | A cancer antigen involved in a translocation in synovial sarcoma. | |
| | x_at | X breakpoint2 | | | May be involved in transcriptional repression. | |
| 167 | 223358_{—} | phosphodiesterase 7A | PDE7A | NR | Increased PDE7 in T cells correlated with decreased cAMP, | |
| | s_at | | | | increased interleukin-2 expression, and increased proliferation. | |
| 213 | 226882_{—} | WD repeat domain 4 | WDR4 | NR | Members of this family are involved in a variety of cellular | |
| | x_at | | | | processes, including cell cycle progression, signal transduction, | |
| | | | | | apoptosis, and gene regulation. | |
| 242 | 225647_{—} | cathepsin C | CTSC | NR | a lysosomal cysteine proteinase that appears to be a central | |
| | s_at | | | | coordinator for activation of many serine proteinases in | |
| | | | | | immune/inflammatory cells | |
| 251 | 208642_{—} | X-ray repair complementing | XRCC5 | NR | Invoved in DNA repair, a pathway important to cancer. Defects in | |
| | s_at | defective repair in Chinese | | | this pathway can lead to cancer and overactivity of this pathway can | |
| | | hamster cells 5 (double- | | | lead to chemotherapeutic resistance in cancer cells | |
| | | strand-break rejoining; Ku | | | | |
| | | autoantigen, 80 kDa) | | | | |
| 286 | 37793_{—} | RAD51-like 3 (S. cerevisiae) | RAD51L3 | R | Possibly invoved in DNA damage repair based on sequence | |
| | r_at | | | | homology | |
| 333 | 218467_{—} | hepatocellular carcinoma | HCCA3 | NR | A novel full-length cDNA was cloned and differentiated, which was | |
| | at | susceptibility protein | | | highly expressed in liver cancer tissues. | |
| 346 | 209031_{—} | immunoglobulin superfamily, | IGSF4 | NR | | |
| | at | member 4 | | | | |
| 442 | 208013_{—} | acrosomal vesicle protein 1 | ACRV1 | R | a testis differentiation antigen | |
| | s_at | | | | | |

Proteasome Inhibitor Resistant Cell Lines

In order to better understand the specific mechanism(s) by which proteasome inhibitors exert their apoptotic effects, as well as to elucidate mechanisms by which those effects may be subverted, bortezomib resistant tumor cell lines were generated. Tumor cell lines were treated with a very low dose of bortezomib (approximately 1/20 the LD50—a dose that would kill 50% of the cells) for 24 hours. The drug was then removed and surviving cells were allowed to recover for 24 to 72 hours. This process was then repeated for multiple rounds with the bortezomib dose doubled each time. After cells had been dosed with 3-5 times the LD50, several individual cell lines were sub-cloned from single cell colonies. Subsequent analyses demonstrated that these lines exhibit 5-10 fold resistance to bortezomib and that this characteristic is stable over months in culture and unaffected by inhibitors of multi-drug resistance pumps. This strategy was applied to both ovarian tumor cell lines (OVCAR-3) and myeloma tumor cell lines (RPMI8226) and multiple sub-clones were characterized. The resistant cell lines were then subject to gene expression profiling using the Affymetrix U133 microarray. A comparison of genes differentially expressed in sensitive parental (S) versus resistant sub-clones (R) highlighted several genes that were also identified in analysis of sensitive and resistant myeloma biopsies. See table 8. The number identified in Table 8 corresponds to the marker number identification in Table 1. Such results not only highlight a potential relationship between expression of these genes and bortezomib sensitivity, but also support the validity of methods used to define response genes in clinical samples.

TABLE 8
Gene Identification in Proteasome Inhibition
Sensitive/Resistant Cell Lines

| | | | | Ratio |
|---|---|---|---|---|
| | Probeset | | | Resistant/ |
| No. | ID | Title | R/S | Parental |
| 156 | 202075_s_{—} | gb:NM_006227.1/DEF = Homo sapiens phospholipid | S | 0.36 |
| | at | transfer protein (PLTP), mRNA./FEA = mRNA/ | | |
| | | GEN = PLTP/PROD = phospholipid transfer protein/ | | |
| | | DB_XREF = gi:5453913/UG = Hs.283007 phospholipid | | |
| | | transfer protein/FL = gb:L26232.1 gb:NM_006227.1 | | |
| 166 | 210497_{—} | gb:BC002818.1/DEF = Homo sapiens, Similar to | R | 2.82 |
| | x_at | synovial sarcoma, X breakpoint 2, clone MGC:3884, | | |
| | | mRNA, complete cds./FEA = mRNA/PROD = Similar to | | |
| | | synovial sarcoma, X breakpoint 2/ | | |
| | | DB_XREF = gi:12803942/UG = Hs.289105 synovial | | |
| | | sarcoma, X breakpoint 2/FL = gb:BC002818.1 | | |
| 332 | 210715_s_{—} | gb:AF027205.1/DEF = Homo sapiens Kunitz-type | S | 0.37 |
| | at | protease inhibitor (kop) mRNA, complete cds./ | | |
| | | FEA = mRNA/GEN = kop/PROD = | | |
| | | Kunitz-type protease | | |
| | | inhibitor/DB_XREF = | | |
| | | gi:2598967/UG = Hs.31439 serine | | |
| | | protease inhibitor, Kunitz type, 2/FL = gb:AF027205.1 | | |
| 211 | 219373_{—} | gb:NM_018973.1/DEF = Homo sapiens dolichyl- | S | 0.37 |
| | at | phosphate mannosyltransferase polypeptide 3 (DPM3), | | |
| | | mRNA./FEA = mRNA/GEN = DPM3/PROD = dolichyl- | | |
| | | phosphate mannosyltransferasepolypeptide 3/ | | |
| | | DB_XREF = gi:9506552/UG = Hs.110477 dolichyl- | | |
| | | phosphate mannosyltransferase polypeptide 3/ | | |
| | | FL = gb:AF312923.1 gb:AF312922.1 gb:AB028128.1 | | |
| | | gb:NM_018973.1 | | |
| 343 | 200030_s_{—} | gb:NM_002635.1/DEF = Homo sapiens solute carrier | R | 2 |
| | at | family 25 (mitochondrial carrier; phosphate carrier), | | |
| | | member 3 (SLC25A3), nuclear gene encoding | | |
| | | mitochondrial protein, transcript variant 1b, mRNA./ | | |
| | | FEA = mRNA/GEN = SLC25A3/PROD = phosphate | | |
| | | carrier precursor isoform 1b/DB_XREF = gi:4505774/ | | |
| | | UG = Hs.78713 solute carrier family 25 (mitochondrial | | |
| | | carrier; phosphate carrier), member 3/ | | |
| | | FL = gb:BC000998.1 gb:BC001328.1 gb:BC003504.1 | | |
| | | gb:BC004345.1 gb:NM_002635.1 | | |
| 447 | 222975_s_{—} | Consensus includes gb:AI423180/FEA = EST/ | R | 1.16 |
| | at | DB_XREF = gi:4269111/DB_XREF = est:tf32e08.x1/ | | |
| | | CLONE = IMAGE:2097926/UG = Hs.69855 NRAS- | | |
| | | related gene/FL = gb:AB020692.1 | | |
| 280 | 224673_{—} | Consensus includes gb:AI613244/FEA = EST/ | S | 0.44 |
| | at | DB_XREF = gi:4622411/DB_XREF = est:ty35a06.x1/ | | |
| | | CLONE = IMAGE:2281042/UG = Hs.306121 leukocyte | | |
| | | receptor cluster (LRC) encoded novel gene 8 | | |
| 129 | 200814_{—} | gb:NM_006263.1/DEF = Homo sapiens proteasome | R | 2.11 |
| | at | (prosome, macropain) activator subunit 1 (PA28 alpha) | | |
| | | (PSME1), mRNA./FEA = mRNA/GEN = PSME1/ | | |
| | | PROD = proteasome (prosome, macropain) | | |
| | | activatorsubunit 1 (PA28 alpha)/ | | |
| | | DB_XREF = gi:5453989/ | | |
| | | UG = Hs.75348 proteasome (prosome, macropain) | | |
| | | activator subunit 1 (PA28 alpha)/FL = gb:BC000352.1 | | |
| | | gb:L07633.1 gb:NM_006263.1 | | |
| 390 | 204610_s_{—} | gb:NM_006848.1/DEF = Homo sapiens hepatitis delta | R | 2.09 |
| | at | antigen-interacting protein A (DIPA), mRNA./ | | |
| | | FEA = mRNA/GEN = DIPA/PROD = hepatitis delta | | |
| | | antigen-interacting protein A/DB_XREF = gi:5803004/ | | |
| | | UG = Hs.66713 hepatitis delta antigen-interacting protein | | |
| | | A/FL = gb:U63825.1 gb:NM_006848.1 | | |
| 429 | 222646_s_{—} | Consensus includes gb:AW268365/FEA = EST/ | R | 2.74 |
| | at | DB_XREF = gi:6655395/DB_XREF = est:xv50d03.x1/ | | |
| | | CLONE = IMAGE:2816549/UG = Hs.25740 ERO1 (S. | | |
| | | cerevisiae)-like/FL = gb:AF081886.1 gb:NM_014584.1 | | |

Sensitivity Assays

A sample of cancerous cells is obtained from a patient. An expression level is measured in the sample for a marker corresponding to at least one of the predictive markers set forth in Table 1, Table 2 and/or Table 3. Preferably a marker set is utilized comprising markers idenitifed in Table 1, Table 2 and/or Table 3 and put together in a marker set using the methods described herein. For example, marker sets can comprise the marker sets identified in Table 4, Table 5 and/or Table 6 or any marker set prepared by similar methods. Such analysis is used to obtain an expression profile of the tumor in the patient. Evaluation of the expression profile is then used to determine whether the patient is a responsive patient and would benefit from proteasome inhibition therapy (e.g., treatment with a proteasome inhibitor (e.g., bortezomib) alone, or in combination with additional agents). Evaluation can include use of one marker set prepared using any of the methods provided or other similar scoring methods known in the art (e.g., weighted voting, CTF). Still further, evaluation can comprise use of more than one prepared marker set. A proteasome inhibition therapy will be identified as appropriate to treat the cancer when the outcome of the evaluation demonstrates decreased non-responsiveness or increased responsiveness in the presence of the agent.

Examining the expression of one or more of the identified markers or marker sets in a tumor sample taken from a patient during the course of proteasome inhibition treatment, it is also possible to determine whether the therapeutic agent is continuing to work or whether the cancer has become non-responsive (refractory) to the treatment protocol. For example, a patient receiving a treatment of bortezomib would have tumor cells removed and monitored for the expression of the a marker or marker set. If the expression profile of one or more marker sets identified in Table 1, Table 2 and/or Table 3 demonstrates increased responsiveness in the presence of the agent, the treatment with proteasome inhibitor would continue. However, if the expression profile of one or more marker sets identified in Table 1, Table 2 or Table 3 demonstrates increased non-responsiveness in the presence of the agent, then the cancer may have become resistant to proteasome inhibition therapy and another treatment protocol should be initiated to treat the patient.

Importantly, these determinations can be made on a patient by patient basis or on an agent by agent (or combinations of agents). Thus, one can determine whether or not a particular proteasome inhibition therapy is likely to benefit a particular patient or group/class of patients, or whether a particular treatment should be continued.

### Other Embodiments

The present invention is not to be limited in scope by the specific embodiments described that are intended as single illustrations of aspects of the invention. Functionally equivalent methods and components are within the scope of the invention, in addition to those shown and described herein and will become apparent to those skilled in the art from the foregoing description, using no more than routine experimentation. Such equivalents are intended to be encompassed by the following claims.

All references cited herein, including journal articles, patents, and databases are expressly incorporated by reference.

What is claimed is:

1. A method for determining a proteasome inhibition therapy regimen for treating a tumor in a patient comprising:
a) determining the level of expression of at least one Predictive marker; and
b) determining a proteasome inhibition-based regimen for treating the tumor based on the expression of the predictive marker, wherein a significant expression level is indicative that the patient is either a responsive patient or a non-responsive patient.

2. The method of claim 1 wherein the level of expression of the predictive marker is determined by detection of mRNA.

3. The method of claim 1 wherein the level of expression of the predictive marker is determined by detection of protein.

4. The method of claim 1 wherein the predictive marker is selected from at least one of the markers identified in any of Table 1, Table 2, Table 3, Table 4 Table 5, Table 6, or Table 7.

5. The method of claim 1 wherein determining the significant level of expression is determined by comparison with a control marker or by comparison to a predetermined standard.

6. The method of claim 1, wherein the tumor is selected from liquid or solid tumors.

7. The method of claim 1 wherein the liquid tumor is selected from the group consisting of myelomas, multiple myeloma, Non-Hodgkins Lymphoma, B-cell lymphomas, Waldenstrom's syndrome, chronic lymphocytic leukemia, and other leukemias.

8. The method of claim 1 wherein the significant level expression is determined by a predictive marker set comprising two or more predictive markers.

9. The method of claim 1, wherein the proteasome inhibition-based regimen for treating the tumor comprises treatment with bortezomib.

10. The method of claim 1, wherein the patient sample comprising tumor cells is obtained from the subject any time selected from prior to tumor therapy, concurrently with tumor therapy or after tumor therapy.

11. A method for treating a tumor in a patient with a a proteasome inhibition therapy comprising:
a) determining the level of expression of at least one Predictive marker in a patient's tumor; and
b) treating the patient with proteasome inhibition therapy comprising a proteasome inhibitor agent based on the expression of the predictive marker, wherein a significant expression level is indicative that the patient is a responsive patient.

12. The method of claim 11 wherein the level of expression of the predictive marker is determined by detection of mRNA.

13. The method of claim 11 wherein the level of expression of the predictive marker is determined by detection of protein.

14. The method of claim 11 wherein the predictive marker is selected from at least one of the markers identified in any of Table 1, Table 2, Table 3, Table 4 Table 5, Table 6 or Table 7.

15. The method of claim 11 wherein determining the significant level of expression is determined by comparison with a control marker or by comparison to a predetermined standard.

16. The method of claim 11, wherein the tumor is selected from liquid or solid tumors.

17. The method of claim 11 wherein the liquid tumor is selected from the group consisting of multiple myeloma, Non-Hodgkins Lymphoma, B-cell lymphomas, mantle cell lymphoma, Waldenstrom's syndrome, chronic lymphocytic leukemia, and other leukemias.

18. The method of claim 11 wherein the significant level expression is determined by a predictive marker set comprising two or more predictive markers.

19. The method of claim 11, wherein the proteasome inhibition-based regimen for treating the tumor comprises treatment with a proteasome inhibitor is selected from the group consisting of a peptidyl aldehyde, a peptidyl boronic acid, a peptidyl boronic ester, a vinyl sulfone, an epoxyketone, and a lactacystin analog.

20. The method of claim 11, wherein the patient sample comprising tumor cells is obtained from the subject any time selected from prior to tumor therapy, concurrently with tumor therapy or after tumor therapy.

21. A marker set for use in the method of claim 1 comprising at least two isolated nucleic acid molecules selected from Table 1 Table 2 or Table 3.

22. A marker set for use in the method of claim 11 comprising at least two isolated nucleic acid molecules selected from Table 1 Table 2 or Table 3.

23. The marker set of claim 21 comprising a marker set constructed using the weighted voting method.

24. The marker set of claim 22 comprising a marker set constructed using the combination of threshold features model.

23. A kit for determining a proteasome inhibition therapy for treating a tumor in a patient comprising reagents for assessing the expression of at least one predictive marker, and instructions for use.

24. The kit of claim 23 wherein the reagents comprise one or a plurality of nucleic acid probes, wherein the probe specifically binds at least one predictive marker.

25. The kit of claim 23 wherein the reagents comprise at least one detecting reagent selected from the group consisting of an antibody, an antibody derivative, an antibody fragment, and peptide probe, wherein the antibody, antibody derivative, antibody fragment or peptide probe specifically binds to a protein corresponding to at least one predictive marker.

26. A method for identifying a candidate compound for treatment of cancer comprising
a) combining a composition comprising a polypeptide of one a predictive marker with a test compound;
b) determining whether the test compound binds the predictive marker polyptptide; and
c) identifying a compound which binds the predictive marker polypeptide as a candidate compound for treatment of cancer.
